(19) United States
(12) Patent Application Publication
(10) Pub. No.: US 2025/0188481 A1
(43) Pub. Date: Jun. 12, 2025

(54) METHOD OF MODULATING MRNA TRANSLATION

(71) Applicant: Yale University, New Haven, CT (US)

(72) Inventors: Wendy Gilbert, New Haven, CT (US); Carson Thoreen, New Haven, CT (US); Cole Lewis, New Haven, CT (US)

(21) Appl. No.: 18/841,044

(22) PCT Filed: Mar. 7, 2023
(86) PCT No.: PCT/US2023/063837

§ 371 Date: Aug. 23, 2024

Related U.S. Application Data
(60) Provisional application No. 63/317,363, filed on Mar. 7, 2022.

(51) Int. Cl. C12N 15/67; A61K 39/00; C12N 15/81

(52) U.S. Cl. CPC C12N 15/67; C12N 15/81; A61K 39/00

(57) ABSTRACT

Described herein are methods of modulating mRNA translational efficiencies with translational enhancers or silencers that affects ribosomal retention. Also described herein are mRNA molecules including translational enhancers or silencers, as well as modified nucleobases. Also described herein are methods of constructing an mRNA molecule for producing a therapeutic peptide or a therapeutic protein with the identified enhancers.

### CROSS-REFERENCE TO RELATED APPLICATIONS

The present application claims priority under 35 U.S.C. § 119(e) to U.S. Provisional Patent Application No. 63/317,363, filed Mar. 7, 2022, which is incorporated herein by reference in its entirety.

### STATEMENT REGARDING FEDERALLY SPONSORED RESEARCH

This invention was made with government support under CA246118, ES031525, GM101316, GM125955, GM132358 and NS118616 awarded by National Institutes of Health. The government has certain rights in the invention.

### SEQUENCE LISTING

The ASCII text file named “047162-7374WO1(01839)_Seq Listing.xml” created on Mar. 1, 2023, comprising 1,207,787 bytes, is hereby incorporated by reference in its entirety.

### BACKGROUND

The sequences and structures of 5′ untranslated regions (5′ UTRs) of mRNA molecules are known to affect translational efficiencies to a large extent. Specific sequence motifs in the 5′ UTR that increases and decreases translational efficiencies, called translational enhancers and translational silencers, however; remain largely unknown, and high-throughput methods that are able to identify translational enhancers and translational silencers are lacking.

Therefore, there is a need to develop high-throughput screening methods to identify translational enhancers and translational silencers, as well as a need to modulate mRNA translation with the identified enhancers/silencers and to construct mRNA molecules having desirable translational efficiencies. The present invention addresses these needs.

### SUMMARY

In some aspects, the present invention is directed to the following non-limiting embodiments: Method of modulating a translational efficiency of mRNA

In some aspects, the present invention is directed to a method of modulating a translational efficiency of a messenger RNA (mRNA) molecule.

In some embodiments the method includes: modifying a 5′-UTR of the mRNA molecule to include a translational enhancer or a translational silencer.

In some embodiments, modifying the 5′-UTR of the mRNA molecule includes modifying a sequence of a DNA molecule encoding the mRNA molecule.

In some embodiments, the translational enhancer or the translational silence is a translational enhancer or a translational silencer for the translational machinery of a yeast species, optionally a yeast species selected from the group consisting of Saccharomyces cerevisiae, Pichia pastoris, Hansenula polymorpha, Yarrowia lipolytica, Arxula adeninivorans, Kluyveromyces lactis, and Schizosaccharomyces pombe.

In some embodiments, the 5′-UTR of the mRNA molecule is modified to include the translational enhancer, and the translational enhancer includes at least one sequence selected from the group consisting of SEQ ID NOs: 1-3.

In some embodiments, the 5′-UTR of the mRNA molecule is modified to include the translational silencer, and the translational silencer includes at least one sequence selected from the group consisting of SEQ ID NOs: 4-6.

In some embodiments, the translational enhancer or the translational silence is a translational enhancer or a translational silencer for the translational machinery of a mammal, optionally a human.

In some embodiments, the 5′-UTR of the mRNA molecule is modified to include the translational enhancer, and the translational enhancer includes at least one sequence selected from the group consisting of SEQ ID NOs: 7-18, 29-51 and 56-1342.

In some embodiments, the 5′-UTR of the mRNA molecule is modified to include the translational silencer, and wherein the translational silencer includes at least one sequence selected from the group consisting of SEQ ID NOs: 19-28.

In some embodiments, the 5′-UTR of the mRNA molecule is further modified to include a modified nucleobase.

In some embodiments, the modified nucleobase includes N6-methyladenosine, inosine, N1-propylpseudouridine, N1-methoxymethylpseudouridine, N1-ethylpseudouridine, 5-methoxycytidine, 5-hydroxyuridine, 5-carboxyuridine, 5-formyluridine, 5-hydroxycytidine, 5-hyrdoxymethylcytidine, 5-hydroxymethyluridine, 5-formylcytidine, 5-carboxycytidine, N4-methylcytidine, pseudoisocytidine, 2-thiocytidine, 4-thiocytidine, N1-methylpseudouridine, pseudouridine, 5-methyluridine, 5-methoxyuridine, dihydrouridine, 5-methylcytidine, 4-thiouridine, 2-thiouridine, uridine-5′-O-(1-thiophosphate), 5-aminoallyluridine, or 4-acetylcytidine.

In some embodiments, the 5′-UTR of the mRNA molecule is modified to include the translational enhancer, and a ribosome retention score (RRS) of the modified mRNA molecule is 3× or higher than an RRS of the unmodified mRNA molecule.

In some embodiments, the 5′-UTR of the mRNA molecule is modified to include the translational silencer, and wherein a ribosome retention score (RRS) of the modified mRNA molecule is 0.3× or lower than an RRS of the unmodified mRNA molecule.

mRNA Molecule

In some aspects, the present invention is directed to an mRNA molecule.

In some embodiments, the mRNA molecule includes a 5′-UTR, and the 5′-UTR includes: a translational enhancer including at least one sequence selected from the group consisting of SEQ ID NOs: 1-3, 7-18, 29-51 and 56-1342, or a translational silencer including at least one selected from the group consisting of SEQ ID NOs: 4-6 and 19-28; and a modified nucleobase.

In some embodiments, the modified nucleobase includes N6-methyladenosine, inosine, N1-propylpseudouridine, N1-methoxymethylpseudouridine, N1-ethylpseudouridine, 5-methoxycytidine, 5-hydroxyuridine, 5-carboxyuridine, 5-formyluridine, 5-hydroxycytidine, 5-hyrdoxymethylcytidine, 5-hydroxymethyluridine, 5-formylcytidine, 5-carboxycytidine, N4-methylcytidine, pseudoisocytidine, 2-thiocytidine, 4-thiocytidine, N1-methylpseudouridine, pseudouridine, 5-methyluridine, 5-methoxyuridine, dihydrouridine, 5-methylcytidine, 4-thiouridine, 2-thiouridine, uridine-5′-O-(1-thiophosphate), 5-aminoallyluridine, or 4-acetylcytidine.

In some embodiments, the 5′-UTR includes the translational enhancer, and wherein a ribosome retention score (RRS) of the mRNA molecule is 3× or higher than an RRS of an mRNA molecule that does not include the translational enhancer or the modified nucleobase but otherwise has the same sequence.

In some embodiments, the 5′-UTR includes at least one sequences selected from the group consisting of SEQ ID NOs: 29-50.

In some embodiments, the 5′-UTR includes the translational silencer, and wherein an RRS of the mRNA molecule is 0.3× or lower than an RRS of an mRNA molecule that does not include the translational silencer or the modified nucleobase but otherwise has the same sequence.

In some embodiments, the mRNA molecule encodes a therapeutic peptide or a therapeutic protein.

In some embodiments, the therapeutic peptide or the therapeutic protein includes a vaccine.

Method of Constructing an mRNA Molecule

In some aspects, the present invention is directed to a method of constructing an mRNA molecule for producing a therapeutic peptide or a therapeutic protein.

In some embodiments, the method includes a translational enhancer at a 5′ UTR to the 5′ side of a coding region encoding the therapeutic peptide or a therapeutic protein.

In some embodiments the therapeutic peptide or a therapeutic protein includes a vaccine.

### Yeast Cell for Expressing Protein

In some aspects, the present invention is directed to a yeast cell for expressing a protein.

In some embodiments, the yeast cell includes an mRNA molecule or a nucleotide encoding the mRNA molecule.

In some embodiments, the mRNA molecule includes: a 5′-UTR including at least one yeast translational enhancer; and a coding region encoding the protein, or a coding region for introducing a nucleotide sequence for encoding the protein.

In some embodiments, the 5′-UTR includes at least one sequence selected from the group consisting of SEQ ID NOs: 1-3.

In some embodiments, the yeast cell is a Saccharomyces cerevisiae cell, a Pichia pastoris cell, a Hansenula polymorpha cell, a Yarrowia lipolytica cell, an Arxula adeninivorans cell, a Kluyveromyces lactis cell, or a Schizosaccharomyces pombe cell.

### BRIEF DESCRIPTION OF THE DRAWINGS

The following detailed description of exemplary embodiments will be better understood when read in conjunction with the appended drawings. For the purpose of illustrating, non-limiting embodiments are shown in the drawings. It should be understood, however, that the instant specification is not limited to the precise arrangements and instrumentalities of the embodiments shown in the drawings.

FIGS. 1A-1D demonstrates that DART quantifies 5′-UTR-mediated translational control over a 1,000-fold range, in accordance with some embodiments. FIG. 1A: Different endogenous yeast 5′ UTRs added to a common luciferase coding sequence and 3′ UTR result in large differences in translational output per mRNA in cell lysates. Colored bars represent 5′ UTR isoforms from a single gene. FIG. 1B: Schematic of DNA pool design. All sequences begin with the T7 promoter, followed by ≤122 nts of 5′ UTR sequence, then a minimum of 24 nts of coding sequence, followed by a 10 nt identifier barcode, and finally an RT handle for library preparation. Endogenous 5′ UTR sequences were derived from RNA-seq data. FIG. 1C: DART sequencing overview. FIG. 1D: DART reproducibly measures ribosome recruitment over a 1,000-fold range. Comparison of three DART replicates.

FIGS. 2A-2E demonstrates that the inhibitory RNA features have highly context-dependent effects on initiation, in accordance with some embodiments. FIG. 2A: Design of SL constructs. Two sets of SLs were designed to span a range of folding energies, with the second set scrambling the stem sequences of the first. Stems were inserted into 20 endogenous unstructured 5′ UTRs and positioned every 3 nts. FIG. 2B: Engineered SLs repress RRSs. All stems repress recruitment relative to sequences without an inserted stem, and the strongest stem shows the greatest level of repression, followed by the intermediate stem and the weakest stem. The p value was calculated using Fisher's test, comparing sequences with introduced stems to those without. FIG. 2C: Individual 5′ UTRs show distinct effects of SL insertion. The relative recruitment for the YER003C 5′ UTR with the −12 kcal/mol stem inserted is shown. In this case, the stem is most repressive near the cap and at the start codon. FIG. 2D: An example 5′ UTR from YDR135C showing reduced ribosome recruitment upon mutation of preferred AUGi context nucleotides (−3A, +4G, and +5C). FIG. 2D: Mutations in AUGi context nucleotides cause variable effects on ribosome recruitment, with changes of the −3A being generally the most deleterious. The density plot shows the probability distribution of the relative RRS at each position.

FIGS. 3A-3C demonstrate that eIF4G binding motifs promote translation initiation, in accordance with some embodiments. FIG. 3A: Schematic of eIF4G1 binding to designed 5′ UTR pool including both wild-type oligo(U) motifs and U->CA mutants. FIG. 3B: Violin plots of RRS distributions for 5′ UTRs containing U37, and the matched controls, for three independent replicates. The p values were calculated using Fisher's test. FIG. 3C: Ranked changes in RRSs between oligo(U) 5′ UTRs and matched U->CA mutants. Dots indicate the mean of all replicates that met read coverage thresholds. The minimum and maximum value across the replicates is also depicted. 5′ UTRs with a statistically significant (Bonferroni-corrected two-tailed t test p<0.05) difference between wild-type and mutant variants are shown.

FIGS. 4A-4E demonstrate that DART identifies translational silencers and enhancers within alternative 5′ UTRs, in accordance with some embodiments. FIG. 4A: 5′ UTR isoforms confound the interpretation of averaged ribosome densities observed in coding sequences by ribosome profiling. FIG. 4B: Most alternative 5′ UTR isoforms tested affect ribosome recruitment (RRS). 2,653 isoform pairs differed significantly by >2-fold, 145 differed significantly, but by <2-fold, and 1,332 did not confer significant differences in RRS. The p values were calculated from a Bonferroni-corrected t test. FIG. 4C: Example genes with alternative 5′ UTR isoforms differing in RRS. The long (5′ extended) isoforms of YDL201W, YDR337W, YBR138C, and YLR440C contain putative silencer elements, and the long isoforms of YGL095C, YLR091W, YDR063W, and YGR196C contain putative enhancers. FIG. 4D Violin plots of RRS values from isoform pairs showing differential ribosome recruitment (p<0.05). The additional 5′ sequences included in longer isoforms were used as inputs to identify enriched motifs using DREME. The top two silencing (above) and enhancing (below) motifs are shown. FIG. 4E: ACCAC motifs identified by DART analysis are sufficient to repress translation of full-length mRNAs in vitro (left) and in vivo (right). Translation activity in vivo is normalized to Fluc mRNA levels and total protein.

FIG. 5 illustrates certain aspects of the DART analysis, in accordance with some embodiments.

FIG. 6 lists some resources used in the first study, in accordance with some embodiments.

FIG. 7 shows some exemplary S. cerevisiae 5′ UTR translational enhancers identified with the DART analysis, in accordance with some embodiments.

FIG. 8 shows some exemplary S. cerevisiae 5′ UTR translational silencers identified with the DART analysis, in accordance with some embodiments.

FIGS. 9A-9B demonstrate that ribosome recruitment scores of the human 5′ UTR sequences obtained using the DART analysis are highly reproducible, in accordance with some embodiments. FIG. 9A: 5′ UTRs with unmodified nucleotides. FIG. 9B: 5′ UTRs modified with N1-methylpseudouridine (m1Ψ).

FIGS. 10A-10B demonstrate that the incorporation of N1-methylpseudouridine (m1Psi) into human 5′UTRs alters ribosome recruitment, in accordance with some embodiments.

FIGS. 11A-11B shows some exemplary human translational silencer and enhancer sequences, in accordance with some embodiments. FIG. 11A: Enhancers. FIG. 11B: Silencers.

FIGS. 12A-12B show that the screening method according to some embodiments herein identified thousands of short 5′-UTRs that outperform current class (e.g., those used by Moderna or Pfizer in mRNA-based vaccines). FIG. 12A: pool 1, 100 nucleotides to 230 nucleotides. FIG. 12B: pool 2, 10 nucleotides to 100 nucleotides.

FIG. 13 demonstrates that the m1Ψ sensitivity of translational efficiency is driven by the 5′-UTR, in accordance with some embodiments. The data show that the observed effects of m1Ψ on ribosome recruitment are driven by incorporation into the 5′ UTR and not the coding sequence. The same 5′-UTR sequence (in this case, the Moderna sequence) was testing with 3 different coding sequence segments in DART. Regardless of the coding sequence used, m1Ψ increases recruitment to this RNA.

FIG. 14 shows that that the screening method according to some embodiments herein identified thousands of short 5′-UTRs that outperform current class (e.g., those used by Moderna or Pfizer in mRNA-based vaccines).

FIG. 15: non-limiting examples of modified nucleotides that are compatible with the screening method herein. The present study validated that the modifications shown in FIG. 15 are compatible with the screening method herein. Top: N1-methylpseudouridine, pseudouridine, 5-methyluridine, 5-methoxyuridine, dihydrouridine, 5-methylcytidine, Bottom: 4-thiouridine, 2-thiouridine, uridine-5′-O-(1-thiophosphate), 5-aminoallyluridine, 4-acetylcytidine.

FIG. 16: systematic identification of regulatory elements, in accordance with some embodiments. FIG. 16 describes a variation of the screening method herein in a systematic manner. Here, the present study took the 100 best and 100 worst sequences and generated variants with scanning, non-overlapping 6 nucleotide deletions across the UTR sequence (over 6,000 variants). This pool was then subjected to the screening method herein. Putative regulatory sequences (enhancers or repressors) were identified by comparing the ribosome recruitment scores of the deletion variants to that of the parent UTR sequence.

FIG. 17 shows an example of the screening method according to FIG. 16. The present study identified a possible translation-enhancing element in an individual 5′ UTR. The top graph shows the log 2RRS for each variant, bottom shows the fold change in RRS compared to the parental sequence. Deletion of nucleotides 1-6 and 7-12 causes a significant decrease in ribosome recruitment to this sequence.

FIGS. 18A-18C: a non-limiting screen method according to some embodiments (DART) quantifies 200-fold differences in 5′ UTR-mediated ribosome recruitment that predicts protein synthesis in vitro and in cells. FIG. 18A: Schematic of the DART workflow. The sequences in the DNA pool begin with a T7 promoter followed by >27nt of common GFP coding sequence and an RT handle for library preparation. Endogenous 5′ UTR sequences were derived from Ensembl annotations. FIG. 18B: DART reproducibly measures ribosome recruitment of a 200-fold range. Comparison of three DART replicates. FIG. 18C: Ribosome recruitment scores predict 5′ UTR-driven translational activity in full-length mRNAs in vitro (Hela cytoplasmic lysate, middle) and in cells (Hela cell transfection, lower).

FIGS. 19A-19H: a non-limiting screen method according to some embodiments (DART) quantifies the regulatory impact of 5′ UTR length, secondary structure, Kozak context, and alternative isoform usage. FIG. 19A: 5′ UTR length is significantly anticorrelated with ribosome recruitment. Correlation plot of UTR length versus ribosome recruitment scores. FIG. 19B: The correlation of 5′ UTR length on activity is driven by sequences shorter than 80 nucleotides. 5′ UTRs were combined into increasing 10 nucleotide-length bins and plotted against RRS. RRS steadily decreases as 5′ UTR length increases until leveling off above 80 nucleotides. FIG. 19C: DART analysis of 11,000 5′ UTRs ranging from 100-230 nucleotides long demonstrates negligible correlation between UTR length and ribosome recruitment. FIG. 19D: Secondary structure has a moderate activity on ribosome recruitment activity. In silico predicted free energy of folding for each UTR is plotted against its RRS score. FIG. 19E: Top- and bottom-scoring 5′ UTRs show no meaningful difference in their Kozak sequence composition. Plots show the nucleotide composition of ten nucleotides upstream of the start codon for the top 25% (left, n=4937) and bottom 25% (right, n=4937) 5′ UTR sequences. FIG. 19F: Kozak sequence optimality has no impact on ribosome recruitment activity. Per-sequence Kozak scores were determined using TIS-Predictor. UTRs with the most optimal Kozak sequence (“Top 25%”) show no significant difference compared to the middle 50% or worst 25%. FIG. 19G: The majority of 5′ UTR isoform pairs exhibit significantly different ribosome recruitment activity. 8,109 isoform pairs differed significantly by >2-fold (red), 9,788 differed significantly, but by <2-fold, and 5,058 did not exhibit significant differences in RRS. FIG. 19H: Example genes with alternative 5′ UTR isoforms with large differences in RRS. The shorter isoforms of RBPJ and SYNGR3 show significantly higher activity than the longer isoforms, while the longer isoforms of SEPTIN9 and SLC12A9 are more active than their shorter isoforms.

FIGS. 20A-20D: N1-methylpseudouridine incorporation into 5′ UTRs significantly alters ribosome recruitment, in accordance with some embodiments. FIG. 20A: Replicates of uridine (left)- and N1-methypseuoduridine (m1Ψ, right)-containing 5′ UTRs exhibit strong correlation, while comparing identical sequences containing uridine versus m1Ψ (middle) reveals significant differences. The RRS scores plotted are an average of six replicates (error bars=S.D.). FIG. 20B: The impact of m1Ψ on ribosome recruitment is context dependent. 1,404 5′ UTRs exhibit significantly reduced ribosome recruitment when uridine is replaced with m1Y (right side; fold change >2, Benjamini-Hochberg adjusted p<0.01), while 2,600 5′ UTRs show significantly increased ribosome recruitment with m1Y (left side; fold change >2, Benjamini-Hochberg adjusted p<0.01). FIGS. 20C-20D: Single substitutions of uridine with m1Ψ can significantly impact ribosome recruitment activity both negatively (FIG. 20C, 6.8-fold decrease, p<0.0001), and positively (FIG. 20D, 4.24-fold increase, p<0.0001).

FIGS. 21A-21G: CCC motifs are a novel translational repressor, in accordance with some embodiments. FIG. 21A: Increasing cytidine content significantly represses ribosome recruitment. 5′ UTRs were binned according to their percent cytidine. FIG. 21B: C-rich sequences are enriched in poor-recruiting 5′ UTRs. The poorest 10% of recruiters were used as inputs for motif enrichment using DREME. 3 out of the top 5 motifs contain a CCC trinucleotide element. FIG. 21C: The presence of CCC motifs correlates with decreased ribosome recruitment. 5′ UTRs containing 30-35% cytidine were selected to hold overall cytidine constant. These UTRs were then binned based on the number of CCC motifs they contain. The data are represented as mean RRS for each bin (error bars=S.D.). FIGS. 21D-21E: CCC motifs are sufficient to repress ribosome recruitment in a dose-dependent manner. CCC sequences were iteratively added to 5′ UTRs and these UTRs were then subjected to DART analysis. The dose-dependent repressive effect is displayed for the 5′ UTR of ZFPL1 (FIG. 21D) and across all UTRs tested (FIG. 21E, n=225 parent 5′ UTRs, 1350 total variants). FIG. 21F: CCC motifs identified by DART are sufficient to repress translation in full-length mRNAs. FIGS. 21G-21H Removal of CCC motifs from human 5′ UTRs increases ribosome recruitment. CCC motifs were iteratively deleted from 5′ UTRs and these were subjected to DART analysis. The effect of successive CCC removal is demonstrated for the 5′ UTR of POC1B (FIG. 21G) and across all UTRs tested (FIG. 21H, n=225 parent 5′ UTRs, 1350 total variants).

### DETAILED DESCRIPTION

The following disclosure provides many different embodiments, or examples, for implementing different features of the provided subject matter. Specific examples of components and arrangements are described below to simplify the present disclosure. These are, of course, merely examples and are not intended to be limiting. For example, the formation of a first feature over or on a second feature in the description that follows may include embodiments in which the first and second features are formed in direct contact, and may also include embodiments in which additional features may be formed between the first and second features, such that the first and second features may not be in direct contact. In addition, the present disclosure may repeat reference numerals and/or letters in the various examples. This repetition is for the purpose of simplicity and clarity and does not in itself dictate a relationship between the various embodiments and/or configurations discussed.

In the first study described herein (“the first study”), a method capable of identifying translational enhancers and translational silencers in the 5′ untranslated regions (5′ UTRs) of mRNA molecules was developed. Specifically, in the first study, a library of mRNA molecules having similar coding regions but different 5′ UTR sequences were mixed with 80s ribosomes to form complexes. The complexes in the mixture were stabilized with cycloheximide and the mixture were then subjected to density gradient fractionation to isolate the ribosome-mRNA complexes and the mRNA. The ribosomal recruitment efficiencies of the 5′ untranslated regions were determined according to the relative abundances of the mRNA molecules in the complex.

In the second study described herein (“the second study”), using the method developed in the first study, translational enhancers and translational silencers in the 5′ UTR were determined in the budding yeast.

In the third study described herein (“the third study”), using the method developed in the first study, translational enhancers and translational silencers in the 5′ UTR were determined in humans.

Accordingly, in some aspects, the present invention is directed to a method of modulating a translational efficiency of a messenger RNA (mRNA) molecule.

In some aspects, the present invention is directed to a non-naturally occurring mRNA molecule. In some embodiments, the non-naturally occurring mRNA molecule has increased or decreased translational efficiencies in comparison to similar naturally occurring mRNA molecules.

In some embodiments, the present invention is directed to a method of constructing mRNA molecules for producing therapeutic proteins/peptide.

### Definitions

As used herein, each of the following terms has the meaning associated with it in this section. Unless defined otherwise, all technical and scientific terms used herein generally have the same meaning as commonly understood by one of ordinary skill in the art to which this disclosure belongs. Generally, the nomenclature used herein and the laboratory procedures in animal pharmacology, pharmaceutical science, peptide chemistry, and organic chemistry are those well-known and commonly employed in the art. It should be understood that the order of steps or order for performing certain actions is immaterial, so long as the present teachings remain operable. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the application, where an element or component is said to be included in and/or selected from a list of recited elements or components, it should be understood that the element or component can be any one of the recited elements or components and can be selected from a group consisting of two or more of the recited elements or components.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.”

“About” as used herein when referring to a measurable value such as an amount, a temporal duration, and the like, is meant to encompass variations of 20% or +10%, in certain embodiments ±5%, in certain embodiments 10%, in certain embodiments ±0.1% from the specified value, as such variations are appropriate to perform the disclosed methods.

Method of Identifying Translational Enhancers and/or Translational Silencers in 5′ UTRs of mRNA Molecules

In some embodiments, the instant specification is directed to a method of identifying translational enhancers and/or translational silencers in 5′ untranslated regions (5′ UTRs) of mRNA molecules.

In some embodiments, the method includes: preparing a plurality of mRNA molecules, wherein each mRNA molecules including a 5′ UTR and a coding region to the 3′ end of the 5′ UTR; contacting the plurality of mRNA molecules with a ribosome; isolating the ribosome; and
determining sequences of 5′ UTRs of mRNA molecules in complex with the isolated ribosomes.

In some embodiments, the 5′ UTRs of an mRNA molecule includes a translational enhancer if the mRNA molecule is more likely to be found in complex with the isolated ribosomes than average.

In some embodiments, the 5′ UTRs of an mRNA molecule includes a translational silencer if the mRNA molecule is less likely to be found in complex with the isolated ribosomes than average.

In some embodiments, the method further includes identifying one or more translational enhancers by determining one or more sequence motifs commonly found in 5′ UTRs of mRNA molecules that are more likely to be found in complex with the isolated ribosomes than average; or identifying one or more translational silencers by determining one or more sequence motifs commonly found in 5′ UTRs of mRNA molecules that are less likely to be found in complex with the isolated ribosomes than average.

In some embodiments, the plurality of mRNA molecules has the same sequence in the coding region. In some embodiments, the coding regions of the plurality of mRNA molecules have similar sequences. In some embodiments, the coding regions of the plurality of mRNA molecules affect translational efficiency at similar levels.

In some embodiments, isolating the ribosomes includes isolation according to the sedimentation coefficient of the ribosome.

In some embodiments, the ribosomes are isolated by density gradient fractionation.

In some embodiments, contacting the plurality of mRNA molecules with the ribosomes includes stabilizing complexes formed by mRNA molecules and ribosomes.

In some embodiments, stabilizing the complexes includes contacting the complexes with cycloheximide.

In some embodiments, the method includes calculating a ribosome retention score based on the relative abundance of the mRNA ribosome complex. As used herein, the term ribosome retention score or RRS refers to a numeric descriptor of the ability of a particular mRNA 5′ UTR to promote ribosome binding and thus translation of the 3′ coding region of the mRNA. The attached appendices describe non-limiting examples of methods for determining an RRS.

In some embodiments, the ribosome retention score mRNA molecules in complex with the isolated ribosomes is calculated based on the relative abundance of the mRNA molecule isolated in complex with ribosomes in comparison to an abundance of the mRNA molecule in the input pool.

In some embodiments, the ribosome retention score of an mRNA molecule in complex with the isolated ribosomes is calculated according to a relative abundance the mRNA molecule isolated in complex with ribosome in comparison to a sum of an abundance of the mRNA molecule isolated in complex with ribosome and an abundance of the mRNA molecule isolated without forming complex with ribosome.

In some embodiments, determining the sequences of 5′ UTRs of mRNA molecules includes sequencing the 5′ UTRs.

In some embodiments, the plurality of mRNA molecules further includes barcoding sequences.

In some embodiments, the barcoding sequences correspond to the sequences of the 5′ UTRs.

In some embodiments, determining the sequences of 5′ UTRs of mRNA molecules includes identifying the barcoding sequences of the mRNA molecules.

In some embodiments, the method further includes verifying the identified 5′ UTR translational enhancers and/or translational silencers.

In some embodiments, verifying the identified 5′ UTR translational enhancers and/or translational silencers includes constructing a first mRNA molecule including a first 5′ UTR and a coding region encoding a reporter protein.

In some embodiments, the first 5′ UTR includes the identified translational enhancers and/or translational silencers; constructing a second mRNA molecule including a second 5′ UTR and the coding region encoding the reporter protein.

In some embodiments, the second 5′ UTR does not include the identified translational enhancers and/or translational silencers, but is otherwise the same as the first 5′ UTR.

In some embodiments, verifying the identified 5′ UTR translational enhancers and/or translational silencers further includes contacting the first mRNA molecule and the second mRNA molecule with ribosome.

In some embodiments, verifying the identified 5′ UTR translational enhancers and/or translational silencers further includes measuring a first level of reporter signal from the translation of the first mRNA molecule.

In some embodiments, verifying the identified 5′ UTR translational enhancers and/or translational silencers further includes measuring a second level of reporter signal from the translation of the second mRNA molecule.

In some embodiments, verifying the identified 5′ UTR translational enhancers and/or translational silencers further includes comparing the first level and the second level.

Method of Modulating Translational Efficiency of mRNA Molecules

In some aspects, the present invention is directed to a method of modulating a translational efficiency of an mRNA molecule.

In some embodiments, the method includes modifying a 5′-UTR of the mRNA molecule to include a translational enhancer or a translational silencer.

In some embodiments, modifying the 5′-UTR of the mRNA molecule includes modifying a sequence of a DNA molecule encoding the mRNA molecule.

In some embodiments, the translational enhancer or the translational silence is a translational enhancer or a translational silencer for the translational machinery of yeasts. It is worth noting that, although the working examples described herein were tested in Saccharomyces cerevisiae, since the translation machinery is highly conserved among yeast species, the translational enhancers or translational silencers identified in Saccharomyces cerevisiae are expected to work in yeast in general. In some embodiments, the translational enhancer or the translational silence is a translational enhancer or a translational silencer for the translational machinery of Saccharomyces cerevisiae, Pichia pastoris, Hansenula polymorpha, Yarrowia lipolytica, Arxula adeninivorans, Kluyveromyces lactis, or Schizosaccharomyces pombe.

In some embodiments, the 5′-UTR of the mRNA molecule is modified to include the translational enhancer, and wherein the translational enhancer includes at least one sequence selected from the group consisting of SEQ ID NOs: 1-3.

In some embodiments, the 5′-UTR of the mRNA molecule is modified to include the translational silencer, and wherein the translational silencer includes at least one sequence selected from the group consisting of SEQ ID NOs: 4-6.

In some embodiments, the translational enhancer or the translational silence is a translational enhancer or a translational silencer for the translational machinery of a mammal, such as a human. It is worth noting that, although the working examples described herein were tested in human cells, the translational machinery among mammals are conserved. As such, translational enhancer and translational silencers identified for humans are expected to work in other mammals.

In some embodiments, the 5′-UTR of the mRNA molecule is modified to include the translational enhancer, and wherein the translational enhancer includes at least one sequence selected from the group consisting of SEQ ID NOs: 7-18, 29-51, and 56-1342.

In some embodiments, the 5′-UTR of the mRNA molecule is modified to include the translational silencer, and wherein the translational silencer includes at least one sequence selected from the group consisting of SEQ ID NOs: 19-28.

In some embodiments, the 5′-UTR of the mRNA molecule is further modified to include a modified nucleobase. In some embodiments, the modified nucleobase includes N6-methyladenosine, inosine, N1-propylpseudouridine, N1-methoxymethylpseudouridine, N1-ethylpseudouridine, 5-methoxycytidine, 5-hydroxyuridine, 5-carboxyuridine, 5-formyluridine, 5-hydroxycytidine, 5-hyrdoxymethylcytidine, 5-hydroxymethyluridine, 5-formylcytidine, 5-carboxycytidine, N4-methylcytidine, pseudoisocytidine, 2-thiocytidine, 4-thiocytidine, N1-methylpseudouridine, pseudouridine, 5-methyluridine, 5-methoxyuridine, dihydrouridine, 5-methylcytidine, 4-thiouridine, 2-thiouridine, uridine-5′-O-(1-thiophosphate), 5-aminoallyluridine, or 4-acetylcytidine.

In some embodiments, the 5′-UTR of the mRNA molecule is modified to include the translational enhancer, and wherein a ribosome retention score (RRS) of the modified mRNA molecule is 2× or higher, such as 3× or higher, 10× or higher, 100× or higher or 1000× or higher, than an RRS of the unmodified mRNA molecule.

In some embodiments, the 5′-UTR of the mRNA molecule is modified to include the translational silencer, and wherein a ribosome retention score (RRS) of the modified mRNA molecule is 0.5× or lower, such 0.3× or lower, 0.1× or lower, 0.0 × or lower or 0.00 × or lower, than an RRS of the unmodified mRNA molecule.

In some embodiments, the 5′-UTR is modified to include the translational enhancer and a modified nucleobase that is able to enhance translation. In some embodiments, the 5′-UTR is modified to include the translational silencer and a modified nucleobase that is able to suppress translation. Whether a modified nucleobase in the 5′-UTR enhances or suppresses translation of an mRNA molecule is context dependent (i.e., a modified nucleobase that enhances translation in one position of one 5′UTR sequence might suppress translation if placed in another position, or in another 5′UTR sequence).

mRNA Molecule

In some embodiments, the present invention is directed to an mRNA molecule.

In some embodiments, the mRNA molecule does not exist in the nature. In some embodiments, the mRNA molecule does not exist in the nature because the mRNA molecule includes a modified nucleobase. As used herein, the term “modified nucleobase” refers to nucleobases other than the four common nucleobases adenine (A), cytosine (C), uracil (U), and guanine (G). The term “modified nucleobase” does not exclude naturally occurring nucleobases that are found in non-mRNA molecules. For example, nucleobases like pseudouridine, 5-methylcytosine, N1-methylpseudouridine and 2′-O-methylated exist in nature. These nucleobases are considered to be examples of modified nucleobases herein.

In some embodiments, the mRNA molecule includes a 5′-UTR.

In some embodiments, the 5′-UTR includes:
a translational enhancer including at least one sequence selected from the group consisting of SEQ ID NOs: 1-3, 7-18, 29-51, and 56-1342, or a translational silencer including at least one selected from the group consisting of SEQ ID NOs: 4-6 and 19-28; and
a modified nucleobase.

In some embodiments, the modified nucleobase is the nucleobases of N6-methyladenosine, inosine, N1-propylpseudouridine, N1-methoxymethylpseudouridine, N1-ethylpseudouridine, 5-methoxycytidine, 5-hydroxyuridine, 5-carboxyuridine, 5-formyluridine, 5-hydroxycytidine, 5-hyrdoxymethylcytidine, 5-hydroxymethyluridine, 5-formylcytidine, 5-carboxycytidine, N4-methylcytidine, pseudoisocytidine, 2-thiocytidine, 4-thiocytidine, N1-methylpseudouridine, pseudouridine, 5-methyluridine, 5-methoxyuridine, dihydrouridine, 5-methylcytidine, 4-thiouridine, 2-thiouridine, uridine-5′-O-(1-thiophosphate), 5-aminoallyluridine, or 4-acetylcytidine.

In some embodiments, the 5′-UTR includes the translational enhancer, and the modified nucleobase is able to enhance translation. In some embodiments, the 5′-UTR includes the translational silencer, and the modified nucleobase is able to suppress translation.

In some embodiments, the 5′-UTR includes the translational enhancer. In some embodiments, a ribosome retention score (RRS) of the mRNA molecule is 2× or higher, such as 3× or higher, 10× or higher, 100× or higher or 1000× or higher, than an RRS of an mRNA molecule that does not include the translational enhancer or the modified nucleobase but otherwise has the same sequence.

In some embodiments, the 5′-UTR includes the translational silencer. In some embodiments, an RRS of the mRNA molecule is 0.5× or lower, such as 0.3× or lower, 0.1× or lower, 0.01× or lower or 0.001× or lower, than an RRS of an mRNA molecule that does not include the translational silencer or the modified nucleobase but otherwise has the same sequence.

In some embodiments, the mRNA molecule encodes a therapeutic peptide or a therapeutic protein.

In some embodiments, the therapeutic peptide or the therapeutic protein includes a vaccine.

Method of Constructing mRNA Molecules for Producing Therapeutic Protein/Peptide

In some embodiments, the present invention is directed to a method of constructing an mRNA molecule for producing a therapeutic peptide or a therapeutic protein.

In some embodiments, the method includes including a translational enhancer at a 5′ UTR to the 5′ side of a coding region encoding the therapeutic peptide or a therapeutic protein.

In some embodiments, the therapeutic peptide or a therapeutic protein includes a vaccine.

### Yeast Cell for Expressing Protein

In some embodiments, the present invention is directed to a yeast cell, such as a yeast cell for expressing a protein. In some embodiments, the yeast cell is a bioengineered yeast cell. In some embodiments, the protein to be expressed by the yeast cell is a protein exogenous or endogenous to the yeast cell. In some embodiments, the protein is a secreted protein.

In some embodiments, the yeast cell is a recombinant yeast cell having a sequence encoding a 5′-UTR translational enhancer engineered into either the genomic DNA or a plasmid/expression vector.

In some embodiments, the nucleic acid encoding the protein to be expressed is already in the yeast cell and is located downstream of the 5′-UTR translational enhancer such that the expression of the protein is enhanced by the 5′-UTR translational enhancer. In some embodiments, the nucleic acid encoding the protein to be expressed has not been engineered into the yeast cell, such that any desired nucleic acid sequences for encoding a protein can be placed downstream of the 5′-UTR translational enhancer.

In some embodiments, the 5′-UTR translational enhancers for yeasts are the same as or similar to those as detailed elsewhere herein.

In some embodiments, the yeast cell is Saccharomyces cerevisiae, Pichia pastoris, Hansenula polymorpha, Yarrowia lipolytica, Arxula adeninivorans, Kluyveromyces lactis, or Schizosaccharomyces pombe.

### EXAMPLES

The instant specification further describes in detail by reference to the following experimental examples. These examples are provided for purposes of illustration only, and are not intended to be limiting unless so specified. Thus, the instant specification should in no way be construed as being limited to the following examples, but rather, should be construed to encompass any and all variations which become evident as a result of the teaching provided herein.

### Example 1

In the study described in Example 1 (“the first study”), a method to quantify translation initiation on thousands of 5′ UTRs in parallel was developed. The first study uncovered sequence-specific motifs that control translation by varied mechanisms and establish a platform for systematic interrogation of 5′ UTR variants that can be used to engineer mRNAs for optimized protein output.

Translational control shapes the proteome in normal and pathophysiological conditions. Current high-throughput approaches reveal large differences in mRNA-specific translation activity but cannot identify the causative mRNA features. The first study developed direct analysis of ribosome targeting (DART) and used it to dissect regulatory elements within 5′ untranslated regions that confer 1,000-fold differences in ribosome recruitment in biochemically accessible cell lysates. Using DART, the first study determined a functional role for most alternative 5′ UTR isoforms expressed in yeast, revealed a general mode of increased translation via direct binding to a core translation factor, and identified numerous translational control elements including C-rich silencers that are sufficient to repress translation both in vitro and in vivo. DART enables systematic assessment of the translational regulatory potential of 5′ UTR variants, whether native or disease-associated, and will facilitate engineering of mRNAs for optimized protein production in various systems.

### Example 1-1: Related Information

Translation initiation is an important step in eukaryotic gene expression, the dysregulation of which is linked to heritable human diseases and cancer. Systematic characterization has shown that mRNA-specific translational activity varies by orders of magnitude under normal growth conditions and is extensively regulated in response to a wide range of physiological signals. Despite growing interest in the mRNA features responsible for this widespread translational control, current high-throughput methods, which rely on quantification of the average number of translating ribosomes per transcript, have revealed only partial correlations (R=0.3-0.8) that leave many differences in translation activity unexplained. In contrast, in-depth genetic and biochemical analysis has revealed detailed regulatory mechanisms for certain mRNAs.

Genome-wide association studies (GWASs) show diverse disease phenotypes associated with non-coding single-nucleotide polymorphisms (SNPs) found within 5′ UTRs. However, current efforts to identify causal variants, which are potential targets for new therapies, are largely restricted to non-synonymous changes within protein coding sequences. A richer understanding of mRNA sequence-specific translation mechanisms is needed to predict which 5′ UTR variants are likely to do damage by dysregulating protein levels. The first study developed direct analysis of ribosome targeting (DART) technology as a bridge between high-throughput, but mechanistically difficult to parse, in vivo approaches and mechanistically precise, but low-throughput, in vitro translation reconstitution assays. In DART, thousands of synthetic mRNAs initiate translation in vitro. Differential abundance analysis of ribosome-bound and input mRNA reveals differences in translation activity. By massively parallel testing of defined sequences, both endogenous and mutated, the approach moves beyond correlation analysis to enable rapid, hypothesis-driven dissection of the causative role of specific 5′ UTR elements to determine their mechanisms of action.

Here, the first study demonstrates the broad power of DART to illuminate the mechanisms underlying translational control. Overall, the first study tested 4,354 full-length alternative mRNA isoforms from 2,064 yeast genes, which revealed widespread translational control by alternative 5′ UTRs. The first study also exploited the throughput of the DART approach to systematically interrogate the effects of RNA secondary structure, start codon context, and protein binding motifs on translation initiation. The results establish a broad stimulatory role for eIF4G1 binding sequences and also demonstrate highly context-dependent control by inhibitory 5′ UTR structures. This illustrates the potential of DART to illuminate the function of putative RNA regulatory elements identified by other high-throughput approaches. The first study further leveraged the alternative 5′ UTR isoform comparisons to discover hundreds of previously uncharacterized translational enhancers and silencers, short sequence elements that are sufficient to promote or repress ribosome recruitment. The study validated several C-rich silencers as sufficient to repress translation both in vitro and in vivo. Together, the results reveal thousands of previously unidentified functional elements within 5′ UTRs that substantially affect translation. This study establishes DART as a powerful new high-throughput method that can be broadly applied to both discover and interrogate regulatory features within 5′ UTRs.

### Example 1-2: Development of DART for Quantitative Comparison of Translation Initiation

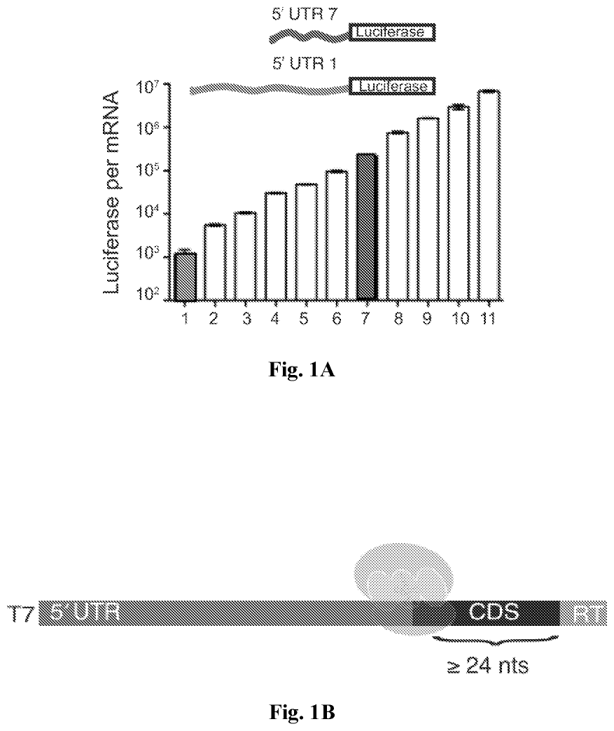

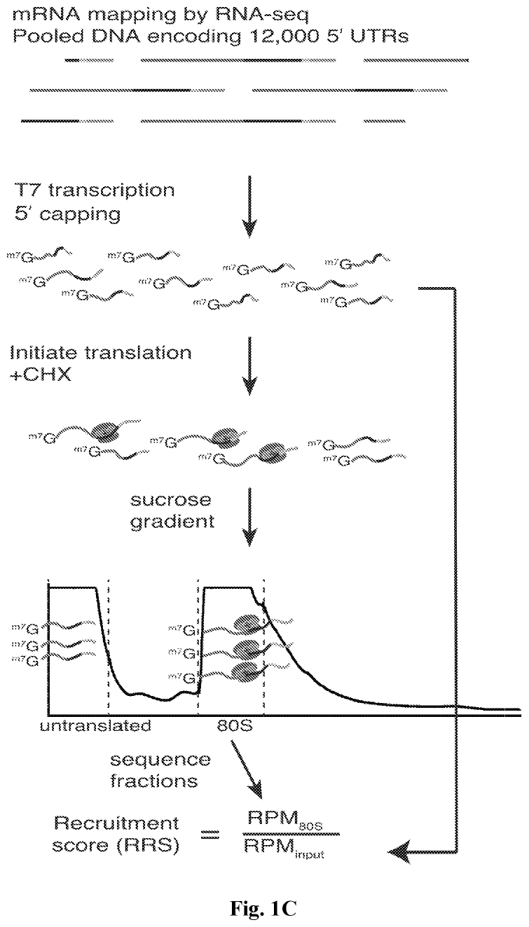

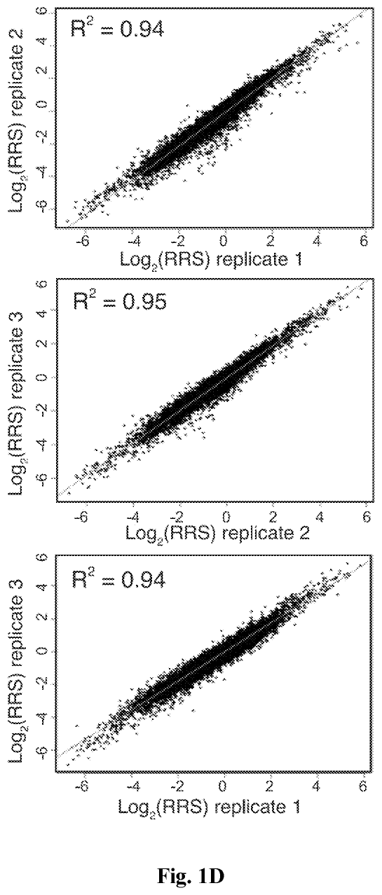

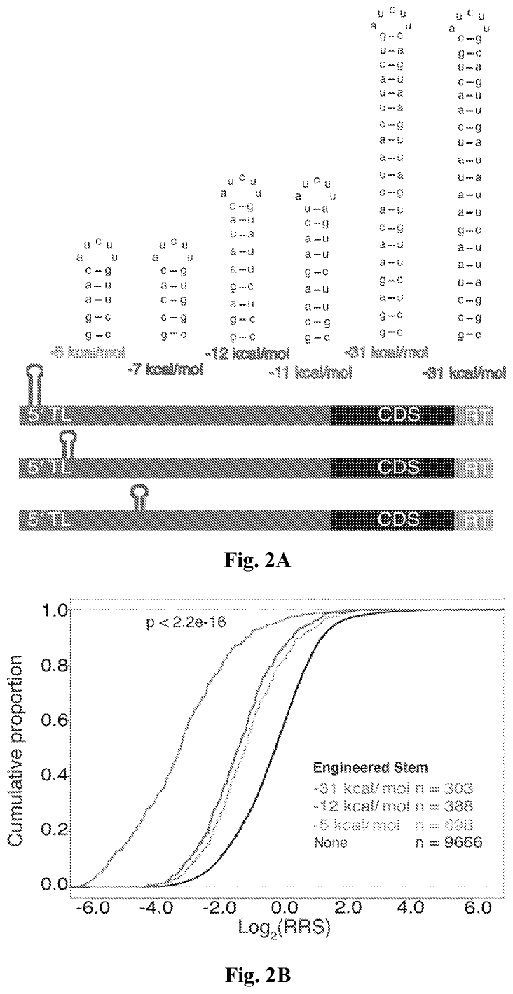

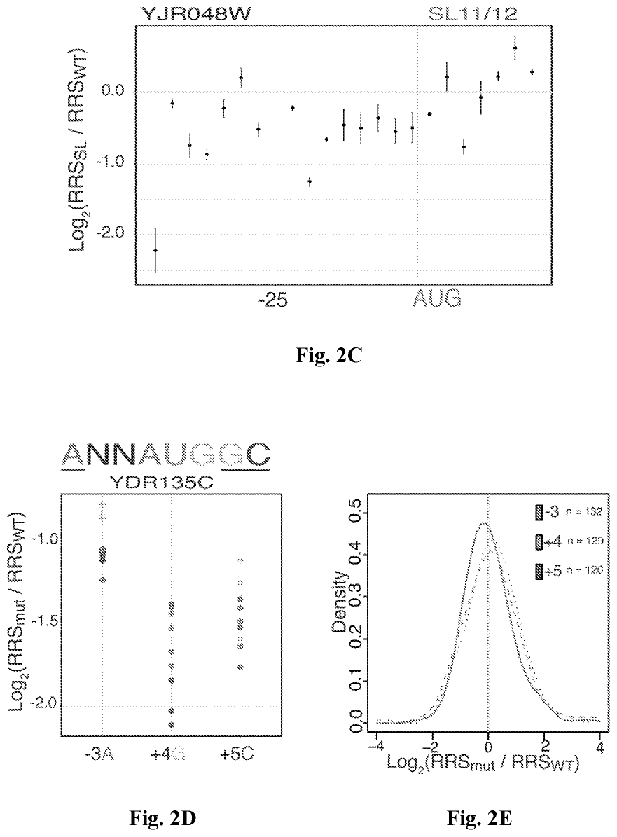

5′ UTRs directly contact the translation initiation machinery and can strongly influence translation activity (FIG. 1A), but the features that distinguish efficiently translated mRNAs are largely unknown. The first study developed DART as a high-throughput method to enable rapid determination of the role of individual regulatory elements within 5′ UTRs. The first study designed a library of DNA oligonucleotides (oligos), based on deep sequencing of yeast mRNA isoforms (Pelechano et al., 2014) (STAR Methods), that included 12,000 sequences corresponding to the full-length 5′ UTR sequences from 4,252 genes. This pool design focuses on identifying functional elements within endogenous 5′ UTRs, in contrast to complementary studies that interrogate random sequences. The library covers 68% of genes expressed during exponential growth and includes alternative mRNA isoforms for many genes. A common (5′) T7 promoter and a (3′) priming site for reverse transcription flanked each unique mRNA sequence. The 5′ UTR sequence was extended to include at least 24 nucleotides of endogenous coding sequence to preserve potentially important downstream sequences that are protected by initiating ribosomes. Each oligo also included a unique 10 nt barcode to distinguish closely related sequences (FIG. 1B).

Next, the first study used these designed pools to generate dsDNA templates for in vitro transcription, followed by enzymatic mRNA capping to produce the pooled RNA substrates for in vitro translation initiation. RNAs were enzymatically biotinylated at their 3′ ends to facilitate quantitative recovery. RNA pools were incubated in yeast translation extracts (Gilbert et al., 2007; Hodgman and Jewett, 2013; Iizuka et al., 1994) for 30 min, a time point that maximizes quantitative differences in ribosome recruitment between 5′ UTRs while yielding enough initiated mRNA for robust library preparation. Cycloheximide was included in the translation reaction to freeze recruited ribosomes at initiation codons and prevent ribosome run-off during ultracentrifugation to separate 80S ribosome-mRNA complexes from untranslated mRNAs. Ribosome-bound mRNA was isolated and sequenced, and the relative abundance of each sequence was compared to its abundance in the input pool to determine a ribosome recruitment score (RRS) for each 5′ UTR (FIG. 1C). DART revealed up to 1,000-fold differences in ribosome recruitment that were highly reproducible (R2=0.94-0.95) among three independent replicates (FIG. 1D). Six 5′ UTRs were selected for low-throughput validation and observed good agreement with DART measurements. Thus, the first study established a robust method to determine the translation initiation activity of defined 5′ UTR sequences.

A caveat to the 80S/input calculation used to assess translation initiation activity is that sequences that destabilize 5′ UTRs in extracts could also lead to a reduction in 80S-bound RNA compared to the input pool. The first study therefore tested an alternative method of calculating RRSs by sequencing the untranslated mRNA from the top of the gradient in addition to the 80S fraction, using the formula RRS0=80S/(80S+mRNP). These two metrics, RRS and RRS0, produced correlated, but not identical, results (R=0.61; p<2.2e-16). Notably, each of the main conclusions reported below is consistent between calculation methods. Because changes in translation initiation can indirectly affect mRNA levels, the 80S/input calculation (RRS) is preferred.

### Example 1-3: A Broad Stimulatory Role for Initiation Factor Binding Motifs in 5′ UTRs

It was hypothesized that 5′ UTR elements that bind preferentially to eukaryotic initiation factors (eIFs) may function as translational enhancers. Consistent with this model, diverse viruses rely on high-affinity interactions between their 5′ UTRs and cellular initiation factors and/or ribosomes for efficient translation. The eIF4G subunit of the cap binding complex is a prime candidate to mediate the activity of translational enhancer sequences. eIF4G contains three RNA binding domains that directly interact with mRNA and are essential for yeast growth, although specific functional interactions between eIF4G and cellular mRNAs had not been characterized. A high-throughput approach, RNA Bind-n-Seq (RBNS), was used to interrogate the RNA binding specificity of eIF4G1. Using a library of random 20-mer RNA to test ˜87,380 distinct RNA 7-mer motifs for binding to different concentrations of recombinant eIF4G1, the first study uncovered a preference for RNA sequences containing oligo-uridine (U). Consistently, it was found that inserting oligo(U) into an unstructured RNA increased binding to eIF4G1 by 20-fold.

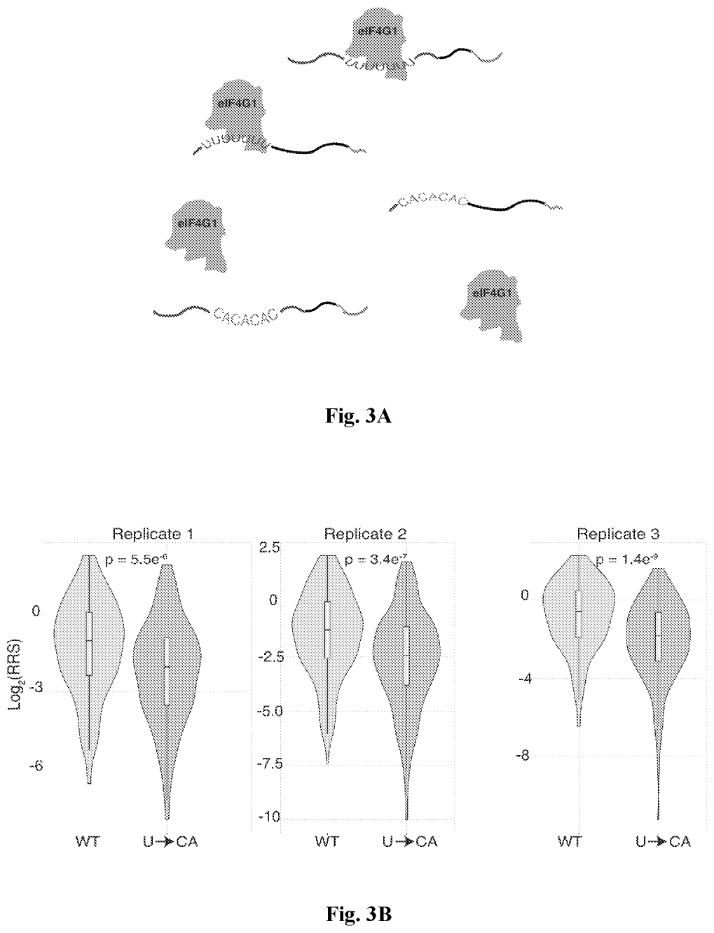

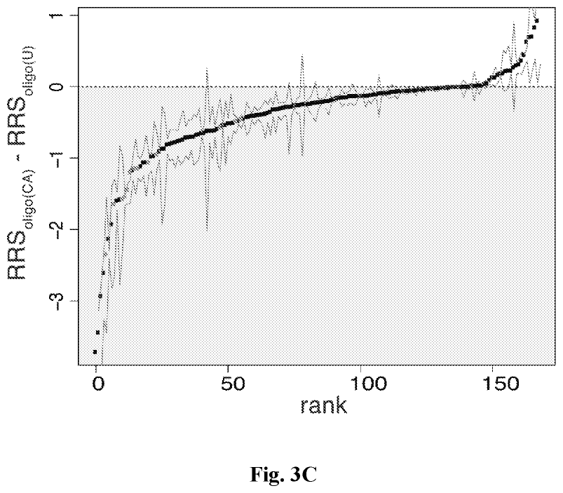

Here, the first study sought to comprehensively test the impact of endogenous oligo(U) motifs on translation initiation. Hundreds of native yeast 5′ UTRs contain oligo(U) sequences, which are evolutionarily conserved among budding yeast species and enriched in genes with regulatory roles. The first synthesized a pool of capped mRNAs consisting of all yeast 5′ UTR sequences ≤94 nt long that contain U³7 (168 in total), together with their start codons and some coding sequence. The pool included matched controls for each 5′ UTR in which the oligo(U) motif was replaced with a CA repeat of equal length (FIG. 3A). The first study performed DART on these native 5′ UTR sequences and compared their activity with and without their endogenous oligo(U) motifs. In all three replicates, wild-type oligo(U)-containing mRNAs were recruited more efficiently to the ribosomal fraction than their matched mutant counterparts (FIG. 3B). Of the 5′ UTR sequences whose ribosome recruitment differed significantly (p<0.05 by two-tailed paired t test) between the wild-type and mutant variants, all were recruited better with the oligo(U) motif present (FIG. 3C). These results are consistent with and substantially extend previous low-throughput experiments that showed endogenous 5′ UTR oligo(U) motifs stimulated translation of full-length mRNA in yeast lysates. The results reveal a broad stimulatory role for eIF4G binding motifs within yeast 5′ UTRs and illustrate the power of DART to assess the function of putative RNA regulatory elements identified by other high-throughput approaches.

### Example 1-4: Production of Alternative 5′ UTR Isoforms Generally Affects Translation Activity

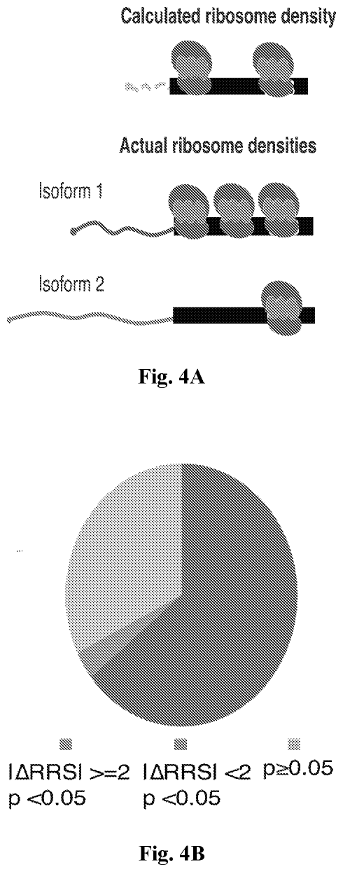

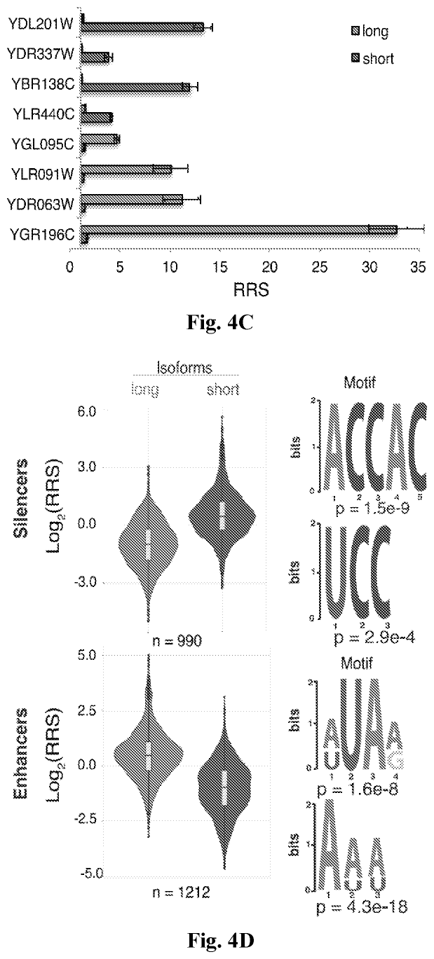

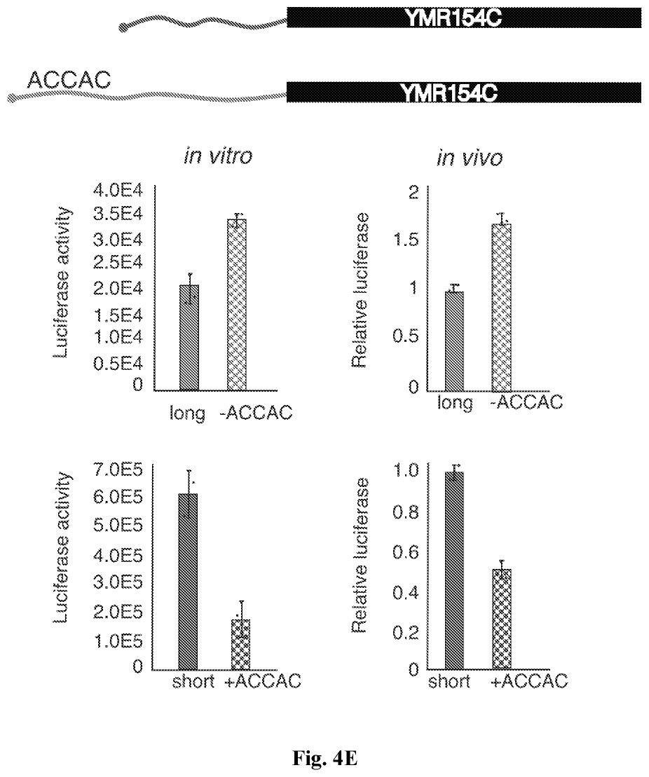

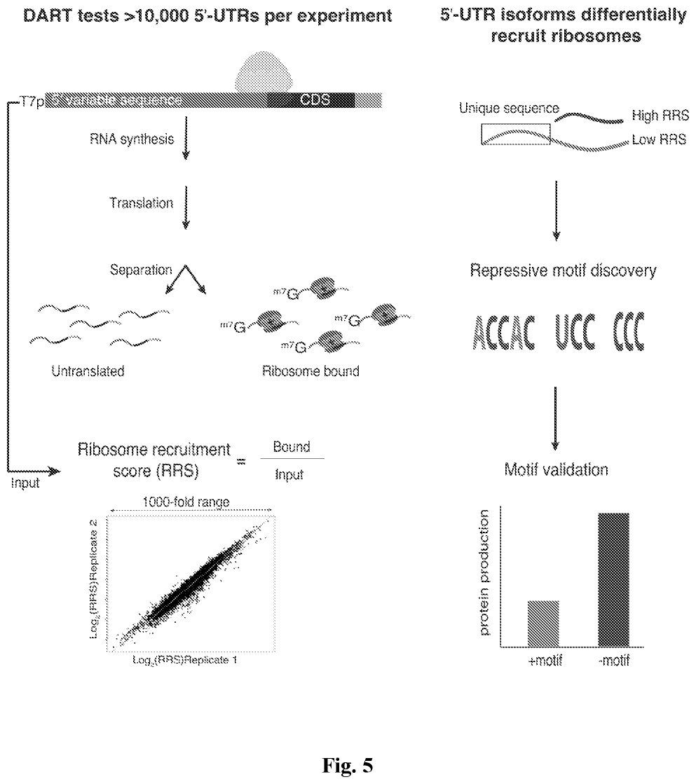

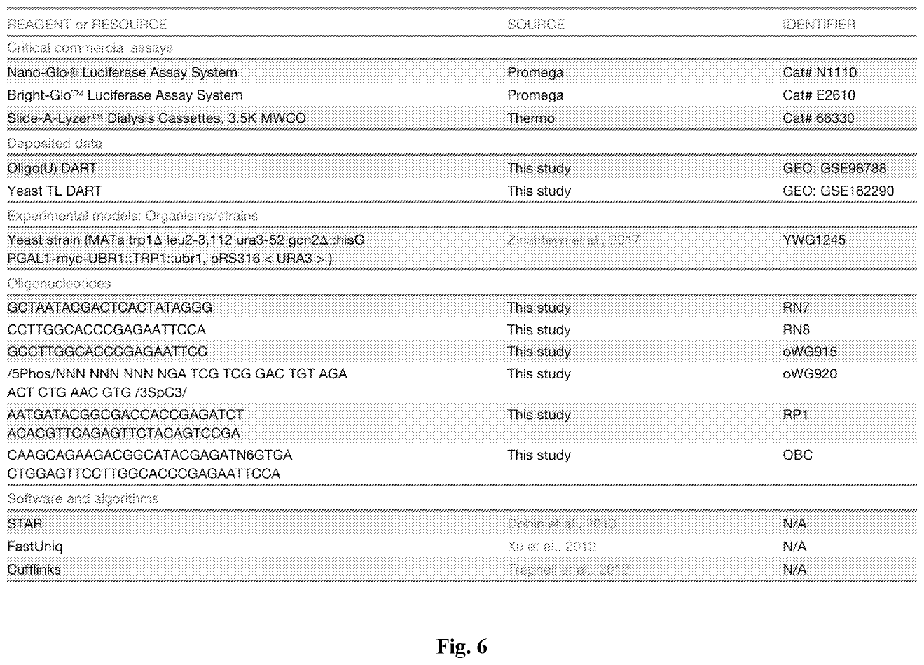

Many eukaryotic genes express multiple mRNA isoforms that differ in their 5′ UTRs, which complicates the analysis of ribosome profiling data to identify translational control elements. Because ribosome-protected footprints within coding sequences cannot be correctly assigned to translation initiation by specific 5 UTRs, the mRNA-specific translational efficiency inferred from ribosome profiling data may not accurately reflect the actual translation activity of any mRNA isoform (FIG. 4A). DART was therefore used to directly compare the activity of expressed alternative mRNA isoforms whose 5′ UTRs differ by at least 10 nucleotides.

The first study analyzed RNA sequencing (RNA-seq) data from wild-type yeast (STAR Methods) and identified 4,354 alternative mRNA isoforms expressed from 2,064 genes that we tested for isoform-specific translation activity by using DART. Notably, 1,639 isoform pairs with reproducible differences in ribosome recruitment were identified (p<0.05, Bonferroni corrected two-tailed t test), of which 843 differed by more than 3-fold. These results provide significantly evidence that alternative 5′ UTRs differ in ribosome association in vivo. DART establishes the direct contribution of differences in 5′ UTR sequences to differences in translation initiation and eliminates potentially confounding effects of co-occurring alternative 3′-UTR sequences.

### Example 1-5. C-Rich Sequences Silence Translation

These mRNA isoforms were leveraged comparisons to identify previously undetected translational control elements within 5′ UTRs. Enhancer and silencer elements were operationally defined as sequences present in a longer 5′ UTR variant that showed a higher RRS than a shorter alternative 5′ UTR of the same gene (enhancer) or lower RRS than the corresponding shorter alternative 5′ UTR (silencer). 658 enhancer regions and 541 silencer regions were identified by the criteria that their inclusion reproducibly changed RRS more than 2-fold (p<0.001, n=3 replicates). Of these, 72 enhancers and 67 silencers changed RRS by more than 10-fold (FIG. 4C). Remarkably, sequence differences of only ten nucleotides were sufficient to significantly alter translation initiation in many cases. Thus, DART reveals relatively short RNA sequences (10-13 nucleotides) that are sufficient to significantly alter ribosome recruitment when present near the 5′ end of the mRNA. Overall, it was found that 68% of genes with alternative 5′ UTRs showed significant differences in translation initiation between isoforms.

To illuminate the mechanisms of translational control, the first study looked for overrepresented motifs within enhancers and silencers using DREME (Bailey et al., 2015). Long 5′ UTR isoforms with higher ribosome recruitment activity were enriched for AU-rich sequences, which are distinct from the previously identified eIF4G-binding U7 motif. Intriguingly, the silencer regions were enriched in CCH motifs. The first study observed similar C-rich motifs in the bottom 10% of all 5′ UTRs (FIG. 4D), which suggests a common mechanism of translational repression.

The first study verified the repressive effects of C-rich silencer motifs on the translational output of full-length mRNAs by using luciferase reporters. For selected genes containing the motif of interest in the longer 5′ UTR isoform, mutations were introduced to disrupt the putative silencer in the long isoform, thereby testing its necessity for translation repression. In parallel, the motif was added to the short isoform, thereby testing its sufficiency. In each case, disruption of the motif from the longer isoform resulted in an increase in protein production whereas addition of the motif reduced luciferase activity from the short isoform (FIG. 4E). Similar effects with these constructs expressed in vivo were observed (FIG. 4E), and somewhat smaller differences in translation between 5′ UTRs in vivo and in vitro were noted. Together, these results validate the C-rich sequence motifs uncovered by DART analysis as translational control elements that repress ribosome recruitment in vitro and protein production in cells.

### Example 1-6

Here DART is presented as a new approach for high-throughput functional testing of endogenous and engineered 5′ UTR variants. The first study applied DART to more than 8,000 endogenous 5′ UTRs from the model eukaryote S. cerevisiae and identified C-rich motifs as translational silencers present within hundreds of 5′ UTRs. The study also used DART to systematically probe the effect of oligo(U) motifs and thereby established a general stimulatory role for this 5′ UTR element in endogenous mRNAs. The results illustrate the power of the DART method to uncover the regulatory elements underlying mRNA-specific differences in protein output, which are anticipated will be broadly applicable to the study of translation initiation, including by human mRNAs.

′ UTR sequences that increased or decreased ribosome recruitment by more than 1,000-fold in cell lysates were identified. Specific sequences, such as C-rich motifs, that affected ribosome recruitment as measured by DART similarly affected protein synthesis from luciferase reporters in cells (e.g., FIGS. 4D and 4E). However, the magnitude of 5′ UTR differences was larger in vitro.

### Example 1-7: Materials and Methods

The yeast strain YWG1245 (MATa trplDleu2-3,112 ura3-52 gcn2D::hisG PGAL1-myc-UBR1::TRP1::ubr1, pRS316 <URA3>) used in this assay was cultured in liquid YPAD (1% yeast extract, 2% peptone, 2% glucose, 0.01% adenine hemisulfate) at 30° C. with constant shaking.

### 5′ UTR Pool Design and Synthesis

Each sequence in the pool consisted of approximately 122 nucleotides of 5′ UTR sequence followed by at least 24 nucleotides of coding sequence followed by a randomized 10 nucleotide unique identifier barcode and an adaptor sequence used for priming reverse transcription and Illumina sequencing (FIG. 1A). For 5′ UTR annotations, 5′ UTR abundances were calculated from deep RNA sequencing of wild type yeast (Pelechano et al., 2013, 2014). Each 5′ UTR was merged with its most abundant neighbor within 10 nts in either direction such that no two 5′ UTRs were within 10 nts of each other. We then imposed the following requirements for inclusion in the pool: (1) 5′ UTRs must be expressed within 25% of the mode abundance for a given 5′ UTR, and (2) 5′ UTRs must make up at least 5% of the total abundance for that ORF, unless the mode was <5% of the total, in which case we used the mode. Upstream AUGs within 761 (6.3% of all) 5′ UTR sequences were mutated to AGT such that the first AUG encountered by a scanning pre-initiation complex moving 50 to 30 would be the annotated AUGi. We included a parallel set of constructs testing the ribosome recruitment activity of each uAUG individually, in which case the downstream (canonical) AUG was mutated to AGT. In total, the study measured recruitment to 1334 uAUGs. Designed oligos were purchased as a pool (Twist Bioscience) and PCR amplified with RN7 and RN8. RNAs were produced by runoff T7 transcription from gel-purified DNA template. RNAs were 5′ capped using the Vaccinia Capping System (NEB M2080S) and 3′ biotinylated using the Pierce RNA 3′ end biotinylation kit (Thermo Scientific 20160).

### Translation Assays

Yeast translation extracts were made as described (Rojas-Duran and Gilbert, 2012) from YWG1245 (MATa trp1Dleu2-3,112 ura3-52 gcn2D::hisG PGAL1-myc-UBR1::TRP1::ubr1, pRS316 <URA3>) cultured in liquid YPAD (1% yeast extract, 2% peptone, 2% glucose, 0.01% adenine hemisulfate). Cultures were grown to mid-log phase, harvested and then washed two times in mannitol buffer (30 mM HEPES pH 7.4, 100 mM KOAc, 2 mM Mg(OAc)₂, 2 mM DTT, 0.1 mM PMSF, 8.5% mannitol). Cells were then resuspended in 1.5× volume of mannitol buffer and subsequently lysed by bead beating with 5× pellet volume of glass beads. Six 1 min rounds of bead beating were performed with intermittent one minute cooling periods on ice. Beads were pelleted and the supernatant transferred to a fresh tube. Cell debris was pelleted by a 30 min spin at 16,000 rcf Supernatant was removed and dialyzed using Slide-A-lyzer, 3-12 mL, 3,500 MWCO (Pierce #66110) for four h (30 mM HEPES pH 7.4, 100 mM KOAc, 2 mM Mg(OAc)₂, 2 mM DTT), changing the buffer once. Approximately 40 pmoles of mRNA were added per 2 mL in vitro translation reaction (22 mM HEPES pH 7.4, 120 mM KOGln, 3 mM MgOGln, 3.75 mM ATP, 0.5 mM GTP, 25 mM creatine phosphate, 1.6 mM DTT, 400U RNaseIn, 2 mM PMSF, 650 mg creatine phosphokinase, 1× cOmplete EDTA-free protease inhibitor) containing 50% yeast extract. Reactions were incubated with 0.5 mg/mL cycloheximide at 26° C. for 30 min in a shaking thermomixer.

To measure translation activity in vivo YWG1245 was transformed with pGAL-5′ UTR-Fluc plasmids and grown to log phase in media containing 2% raffinose. 5′ UTR sequences were generated by gBlock (Twist Bioscience) mRNA expression was induced by addition of galactose to a final concentration of 2%. Whole-cell lysates were prepared by vortexing with glass beads in 1×PBS with protease inhibitors (2 mM phenylmethanesulfonyl fluoride, and 1 3protease inhibitor cocktail (Roche). Luciferase activity was measured using the Luciferase Bright-Glo Assay System (Promega) on a Berthold Centro XS Luminometer. Luciferase values were normalized to both FLUC mRNA levels and total protein. Fluc mRNA levels were determined by Northern blot on total cellular RNA isolated from whole-cell lysates by hot phenol extraction and normalized to U1 RNA levels. Fluc mRNA levels were determined by Northern blot on total cellular RNA isolated from whole-cell lysates by hot phenol extraction and normalized to U1 RNA levels. Probes made using radiolabeled PCR products from the following primer pairs: 5′-AAGGCGTGTTTGCTGACGTTTC-3′ (SEQ ID NO: 52), 5′-CACCCGTTCCTACCAAGACC-30 (U1) (SEQ ID NO: 53); 5′-TGGGCGCGTTATTTATCGGAGTTGC-3′ (SEQ ID NO: 54), 5′-GAGCCCATATCCTTGCCTGATACC-3′ (Fluc) (SEQ ID NO: 55).

### 80S Isolation

Translation reactions were loaded onto 10%-50% sucrose gradients in polysome lysis buffer (20 mM HEPES-KOH pH 7.4, 2 mM magnesium acetate, 0.1 M potassium acetate, 0.1 mg/mL cycloheximide, 1% Triton X-100) and centrifuged at 27,000×g in a Beckman SW28 rotor for 3 h. Gradients were fractionated from the top down using a Biocomp Gradient Station (Biocomp Instruments) with continual monitoring of absorbance at 254 nm. Fractions corresponding to the 80S peak were pooled and RNA extracted using phenol/chloroform followed by isopropanol precipitation.

### Library Preparation

RNA pellets from the input pool, total lysate, and gradient fractions were resuspended in binding buffer (0.5M NaCl, 20 mM Tris-HCl pH 7.5, 1 mM EDTA) and biotinylated 5′ UTRs were recovered using Hydrophilic Streptavidin Beads (NEB S1421S). Isolated RNA was reverse transcribed using the barcoded primer OWG921 and Superscript III (Invitrogen 18080093). Gel-purified cDNA products were ligated to the adaptor OWG920 using T4 RNA ligase 1 (NEB M0437M). cDNA cleanup was performed using 10 ml MyOne Silane beads (Thermo Scientific 37002D) per sample. Libraries were then PCR amplified with primers RP1 and OBC and sequenced on a HiSeq 2500.

### Example 2: Identification of Additional Yeast Translational Enhancers and Translational Silencers

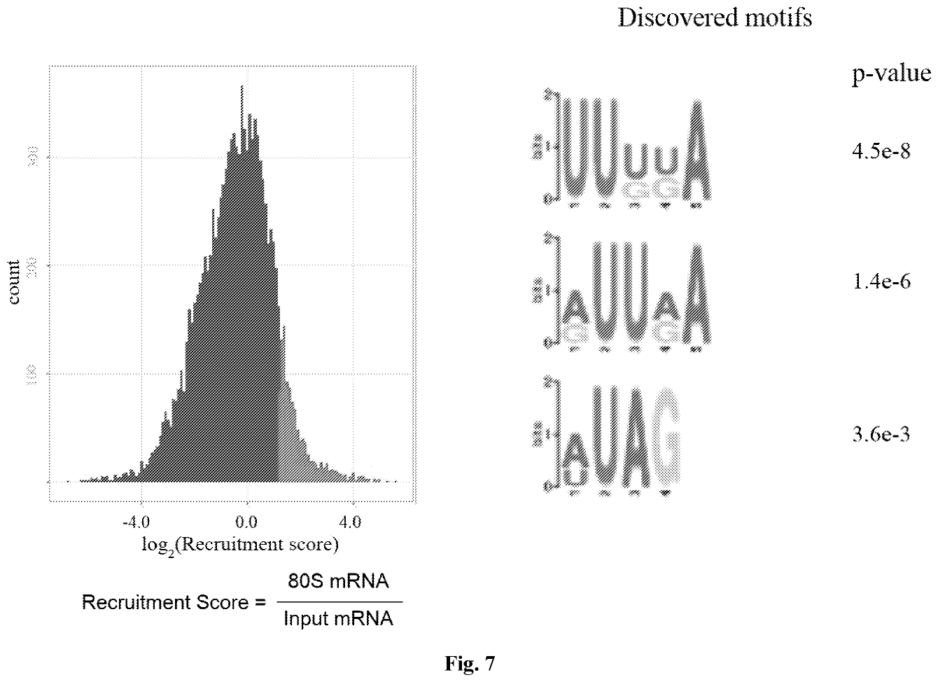

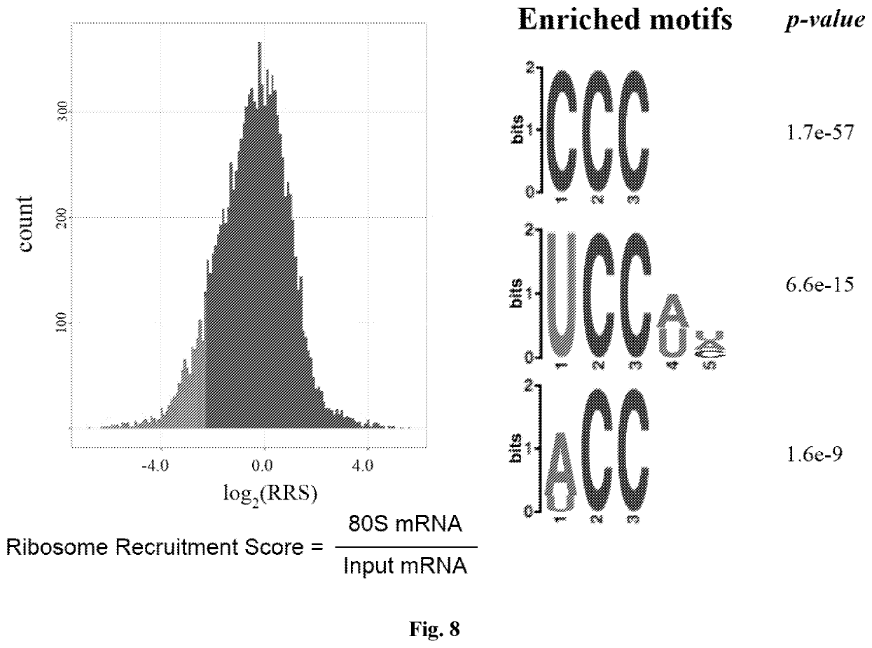

In the second study described herein (“the second study”), using the method developed in the first study, translational enhancers and translational silencers in the 5′ UTR were determined in the budding yeast. These enhancers and silencers are shown in FIGS. 7 and 8 and listed in Table 1 below:

TABLE 1
Identified Saccharomyces cerevisiae translational
enhancers and translational silencers

| | |
|---|---|
| Translational Enhancers | UUKKA (SEQ ID NO: 1) |
| | RUURA (SEQ ID NO: 2) |
| | WUAG (SEQ ID NO: 3) |
| Translational Silencers | CCC (SEQ ID NO: 4) |
| | UCCWH (SEQ ID NO: 5) |
| | WCC (SEQ ID NO: 6) |

### Example 3: Identification of Additional Human Translational Enhancers and Translational Silencers

In the third study described herein (“the third study”), using the method developed in the first study, translational enhancers and translational silencers in the 5′ UTR were determined in humans.

Specifically, a human 5′ UTR pool was designed and synthesized. In this 5′-UTR pool, each sequence in the human 5′ UTR pool consisted of a T7 promoter followed by between 10-230 nucleotides of 5′ UTR sequence followed by at least 24 nucleotides of GFP coding sequence followed by an adaptor sequence used for priming reverse transcription and Illumina sequencing. 5′ UTR sequences were derived from Ensembl annotations. Designed oligos were purchased as pools (Twist Bioscience) and PCR amplified. RNAs were produced by runoff T7 transcription from gel-purified DNA template. RNAs were 5′ capped using the Vaccinia Capping System (NEB M2080S).

In the human translation assays, 20 pmoles of pool RNA were added per 500 microliter translation reaction (16 mM HEPES KOH pH 7.4, 40 mM KOGln, 2 mM MGOGln, 0.8 mM ATP, 0.1 mM GTP, 20 mM creatine phosphate, 0.1 mM spermidine, 1.6 mM DTT, 2 mM PMSF, 140U RNasin Plus, 165 mg creatine phosphokinase, 1× cOmplete EDTA-free protease inhibitor) containing 50% Hela cytoplasmic extract (Ipracell). Reactions were incubated with 0.5 mg/mL cycloheximide at 37° C. for 30 min in a shaking thermomixer.

The human 80S was isolated according to the following: translation reactions were loaded onto 10%-50% sucrose gradients in polysome lysis buffer (20 mM HEPES-KOH pH 7.4, 2 mM magnesium glutamate, 0.1 M potassium glutamate, 0.5 mg/mL cycloheximide, 10% Triton X-100) and centrifuged at 35,000×g in a Beckman SW41 rotor for 3 h. Gradients were fractionated from the top down using a Biocomp Gradient Station (Biocomp Instruments) with continual monitoring of absorbance at 254 nm. Fractions corresponding to the 80S peak were pooled and RNA extracted using phenol/chloroform followed by isopropanol precipitation.

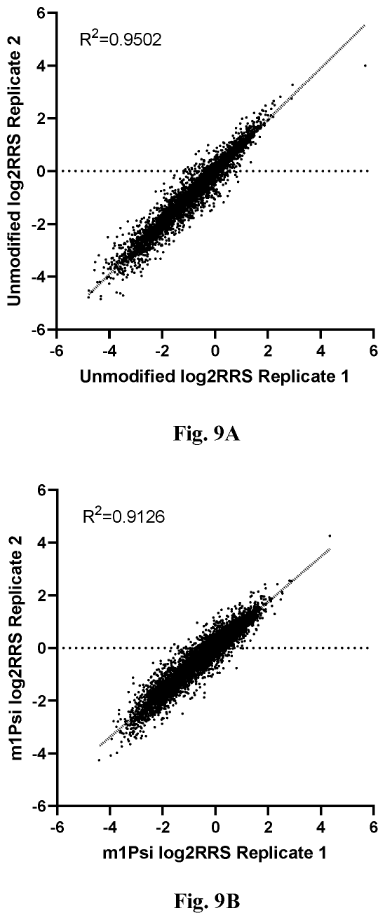

The study confirmed that the DART analysis developed in the first study is highly reproduceable (FIGS. 9A-9B).

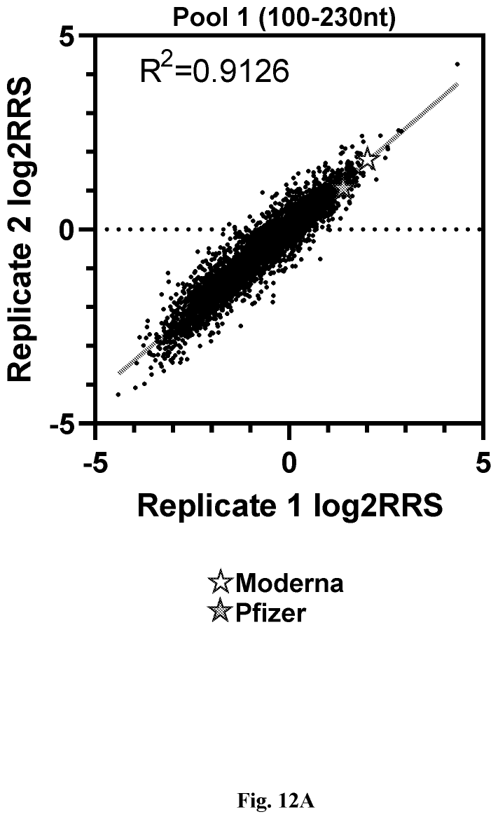

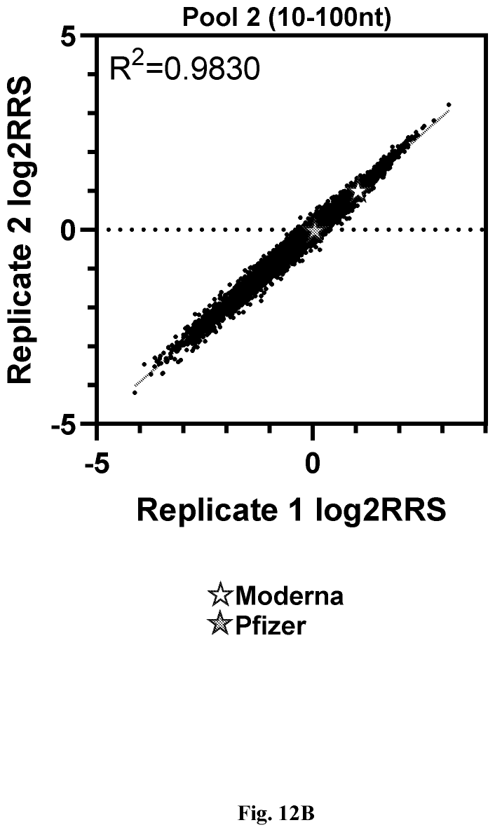

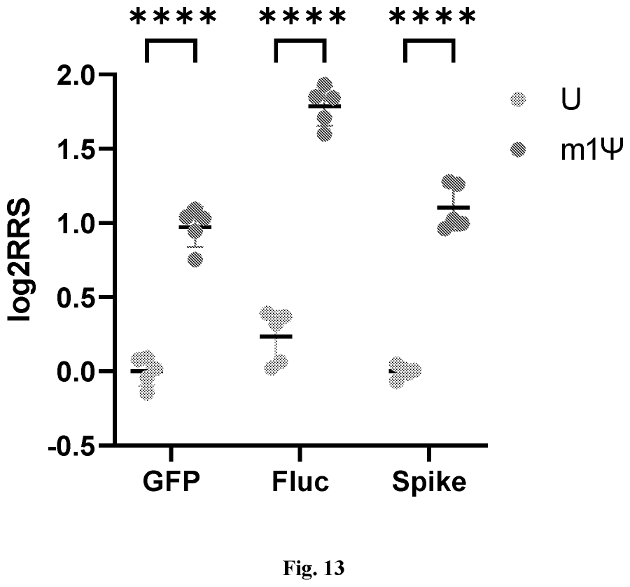

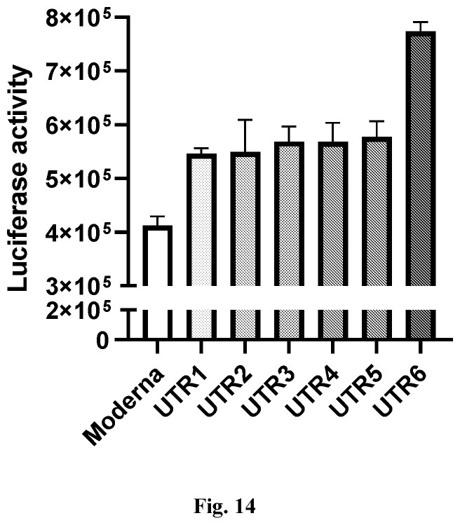

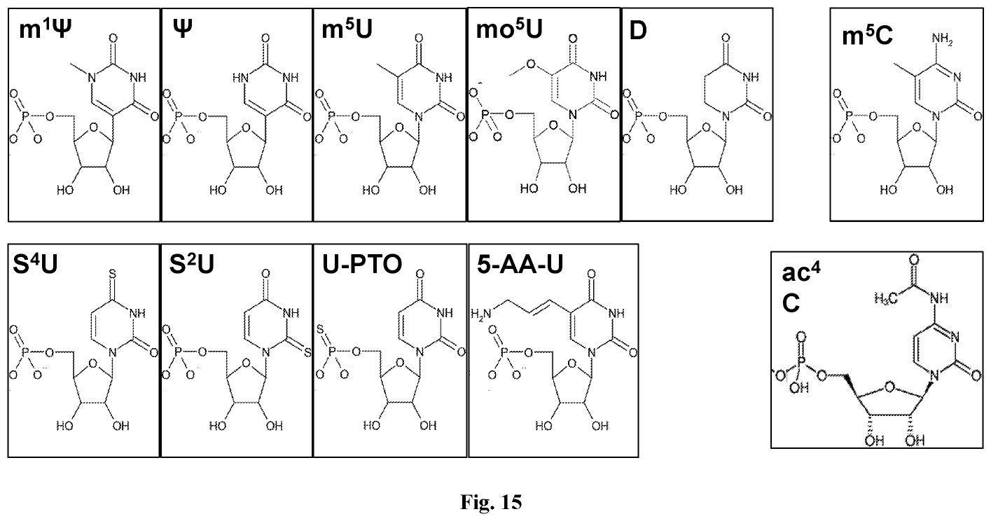

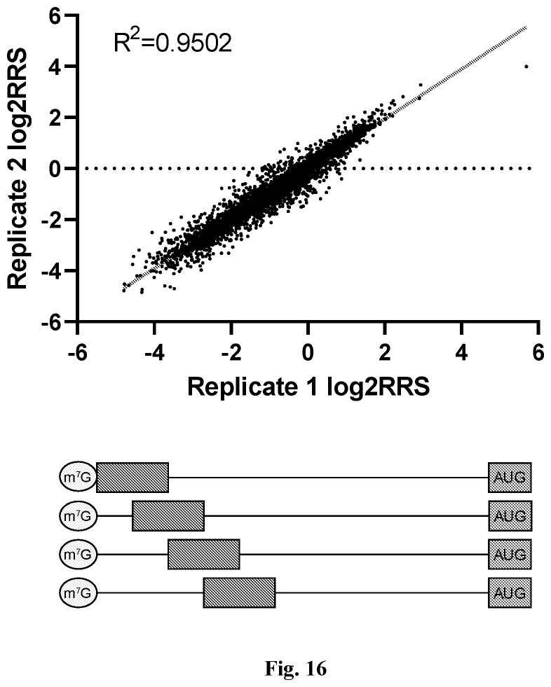

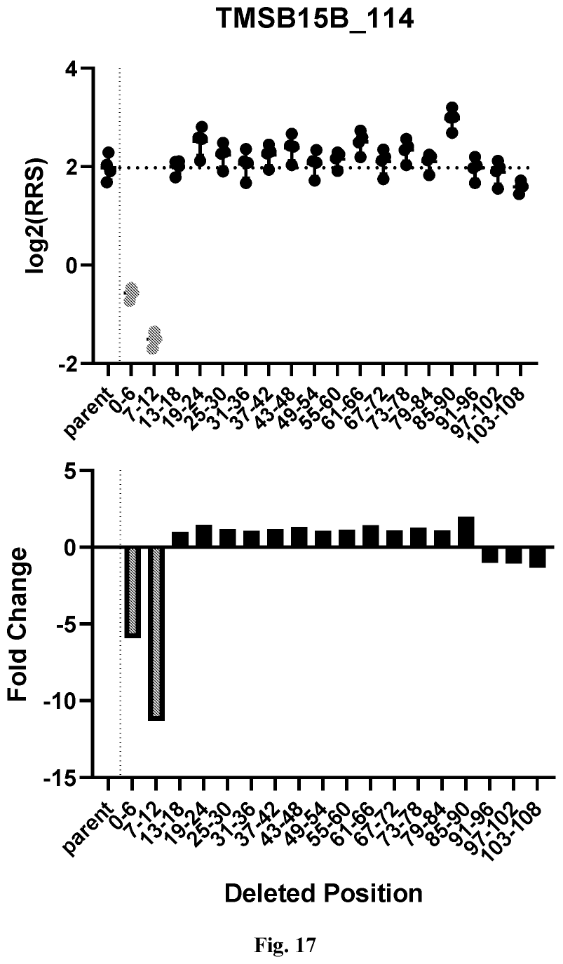

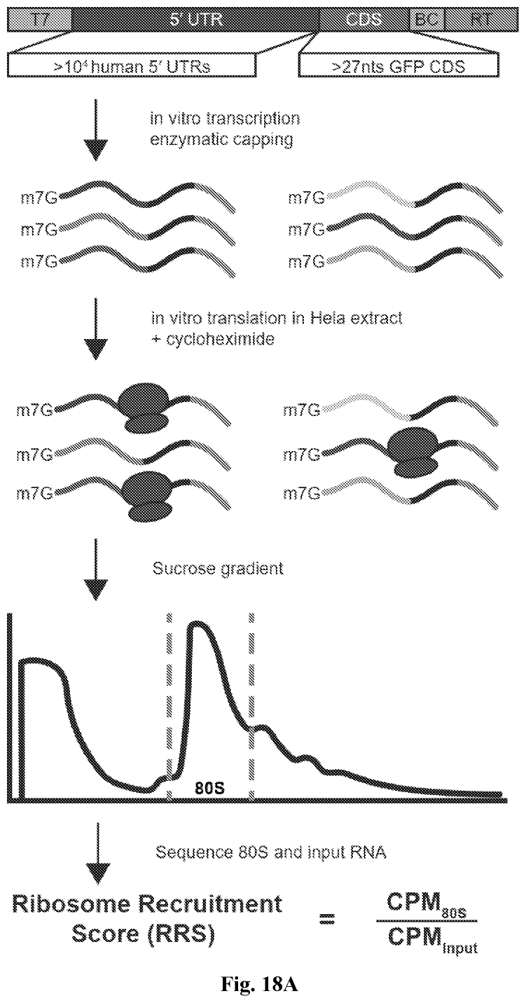

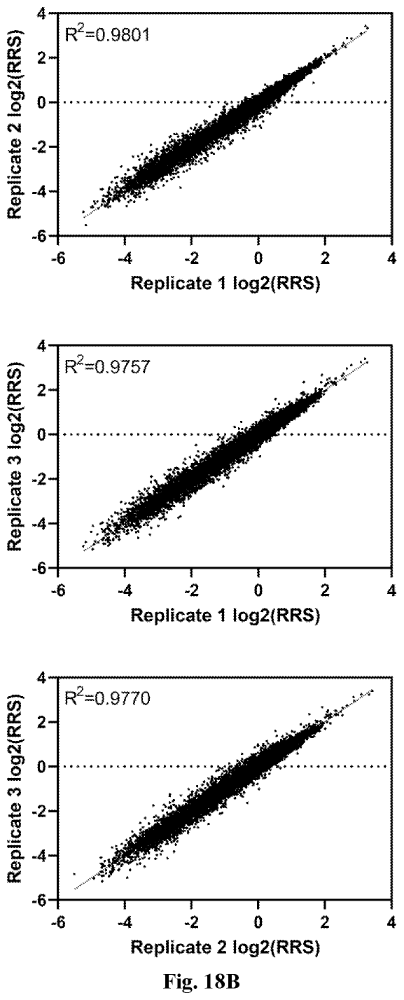

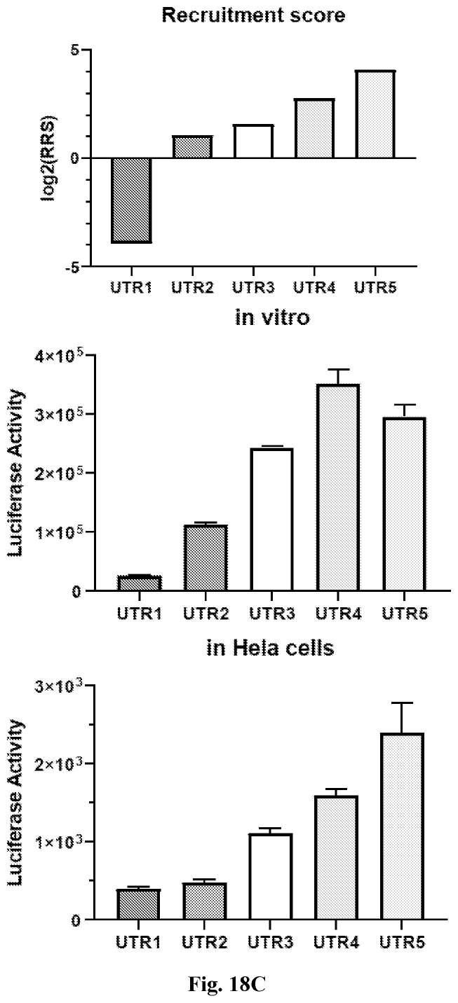

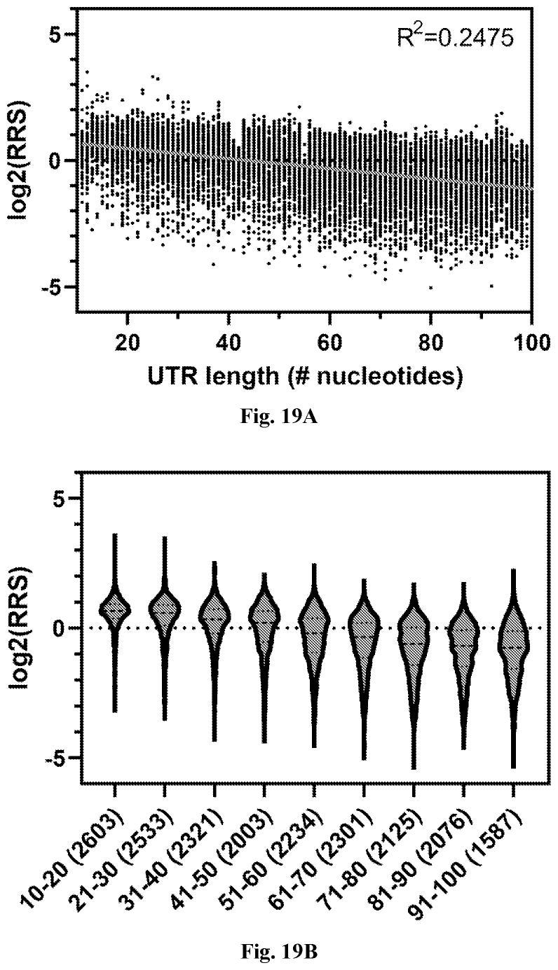

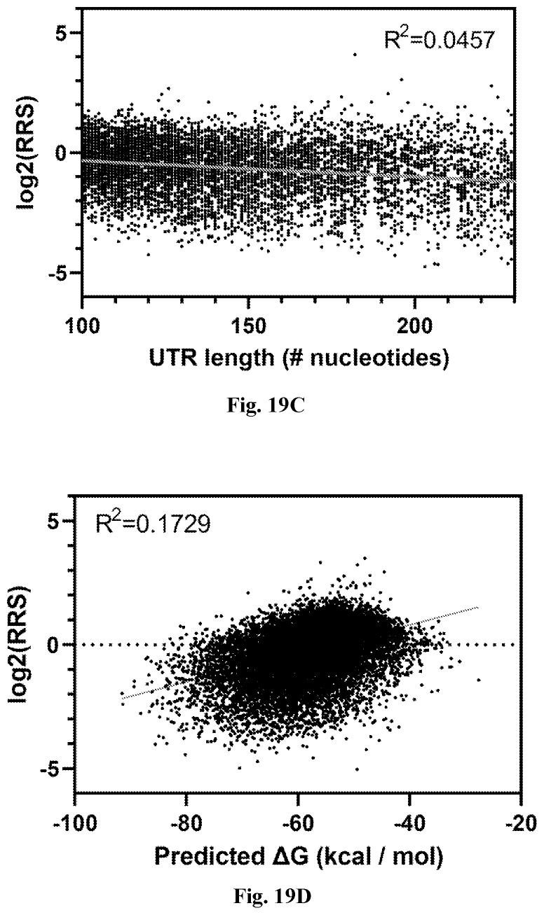

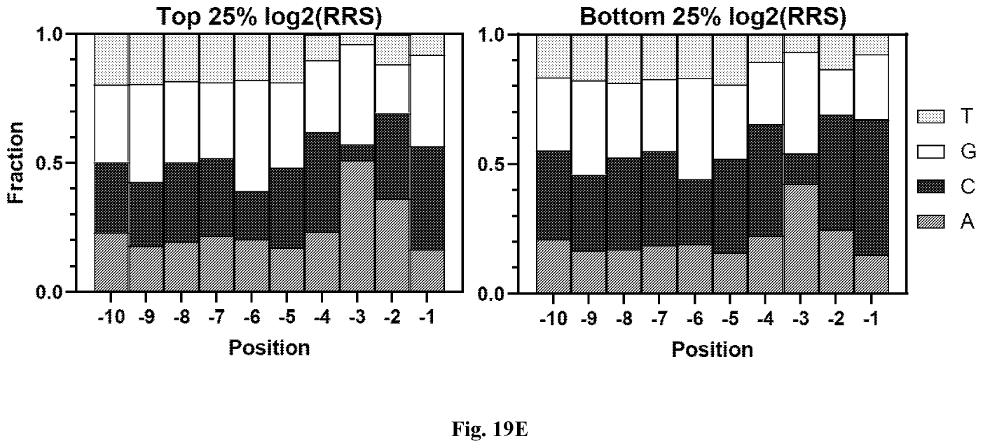

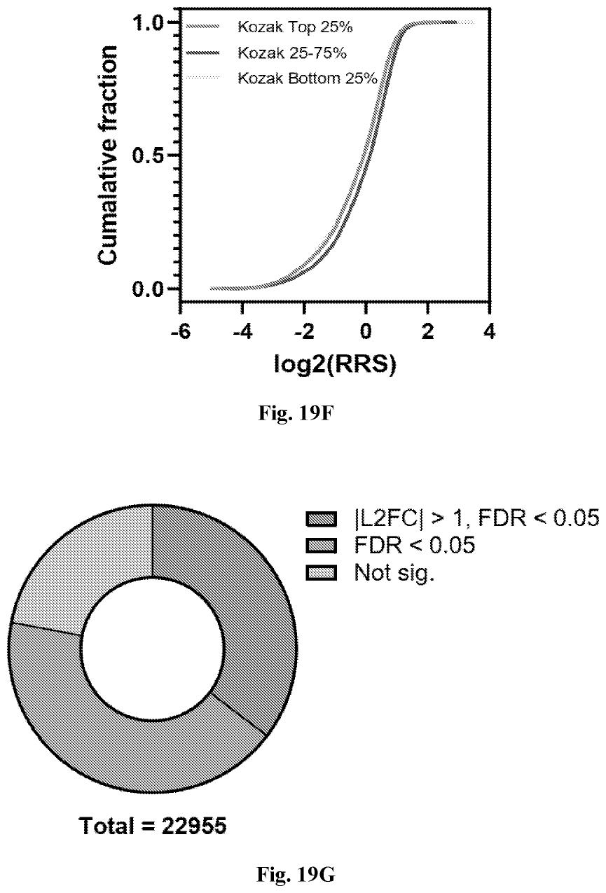

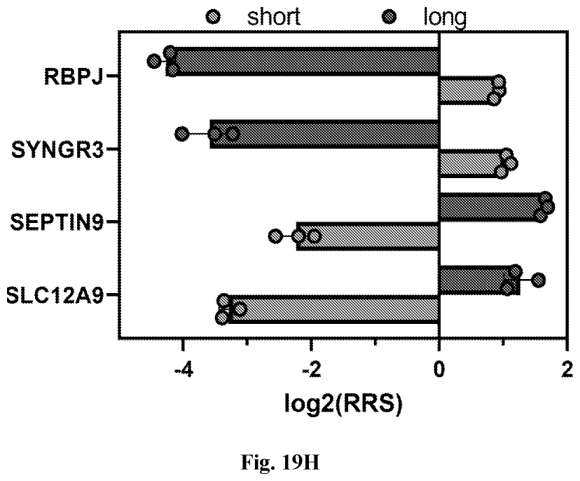

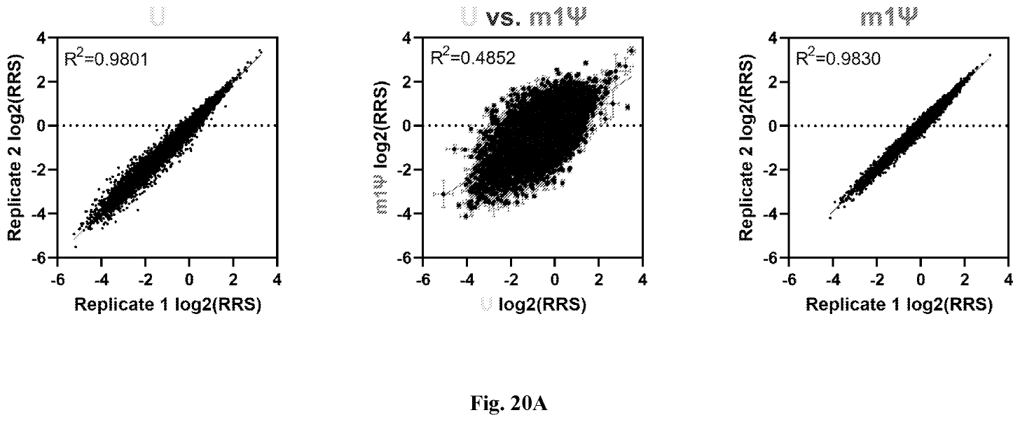

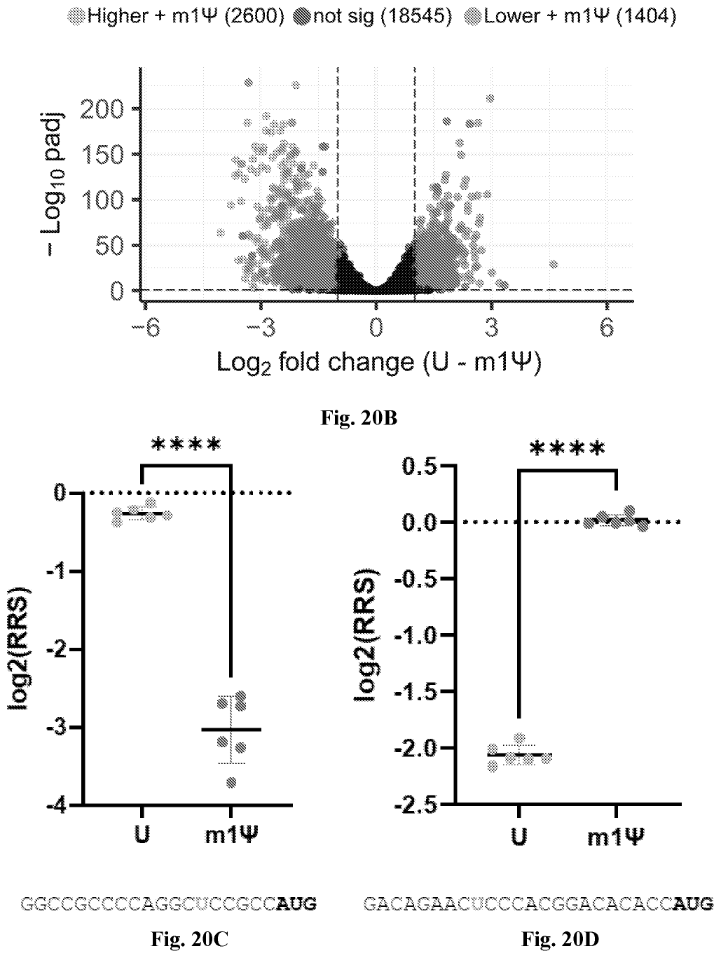

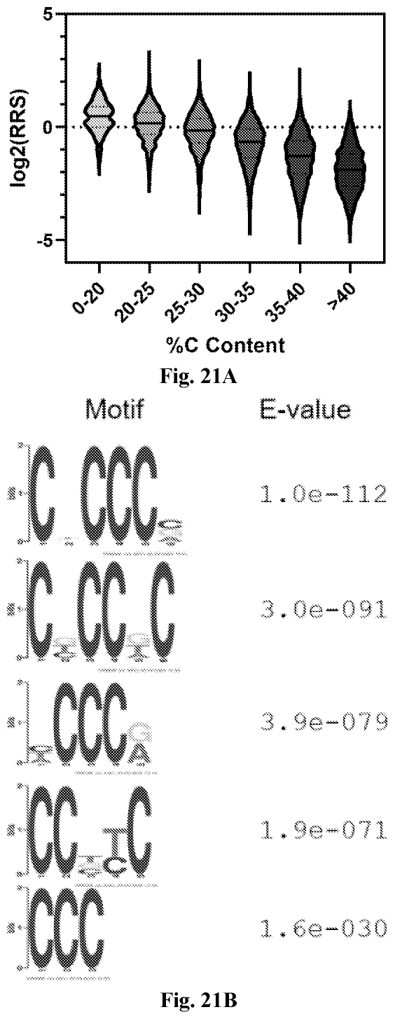

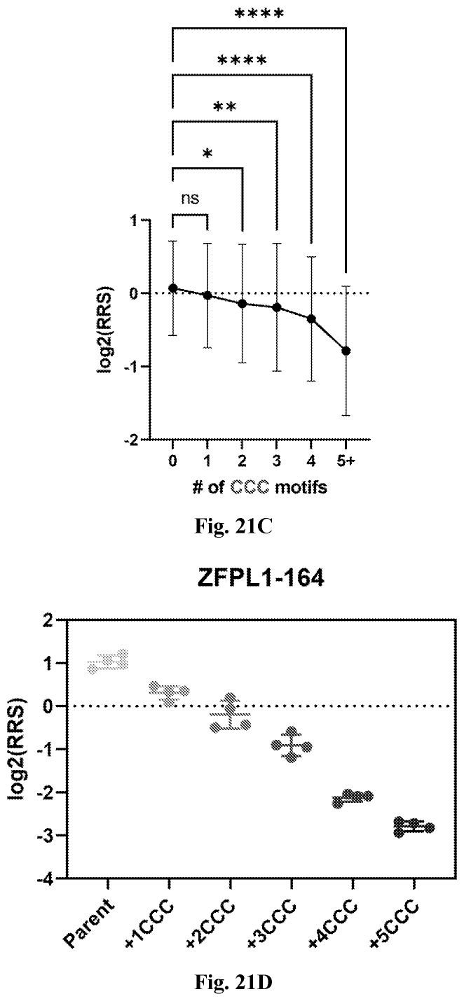

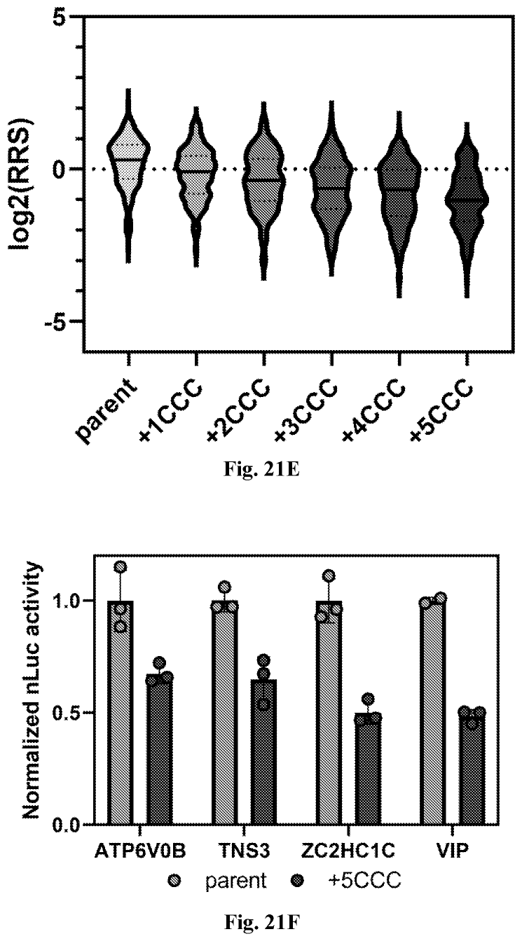

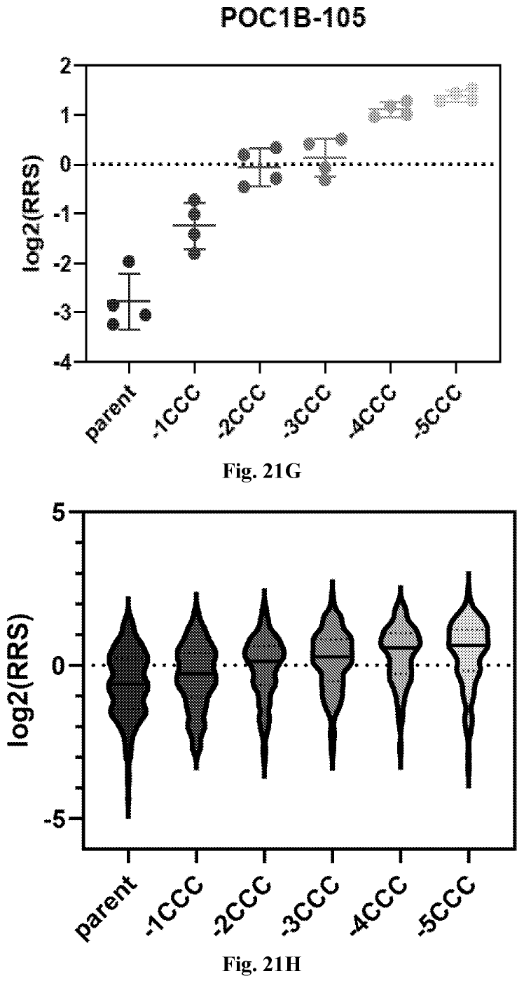

One batch of identified enhancers and silencers are shown in FIGS. 11A-11B, and listed in Table 2 below:

TABLE 2
Identified human translational enhancers
and translational silencers

| | |
|---|---|
| Translational Enhancers | DUUUB (SEQ ID NO: 7) |
| | AADAW (SEQ ID NO: 8) |
| | UUSWD (SEQ ID NO: 9) |
| | UAYW (SEQ ID NO: 10) |
| | WKWA (SEQ ID NO: 11) |
| | WGGWA (SEQ ID NO: 12) |
| | AWW (SEQ ID NO: 13) |
| | CAACUGCW (SEQ ID NO: 14) |
| | USUU (SEQ ID NO: 15) |
| | CUUMCCAU (SEQ ID NO: 16) |
| | AAGACCCM (SEQ ID NO: 17) |
| | GAGACGAA (SEQ ID NO: 18) |
| Translational Silencers | CNCCCV (SEQ ID NO: 19) |
| | CBCCDC (SEQ ID NO: 20) |
| | CCBYC (SEQ ID NO: 21) |
| | HCCCR (SEQ ID NO: 22) |
| | CCC (SEQ ID NO: 23) |
| | CWCC (SEQ ID NO: 24) |
| | CKCGC (SEQ ID NO: 25) |
| | GCC (SEQ ID NO: 26) |
| | YGUCC (SEQ ID NO: 27) |
| | CGR (SEQ ID NO: 28) |

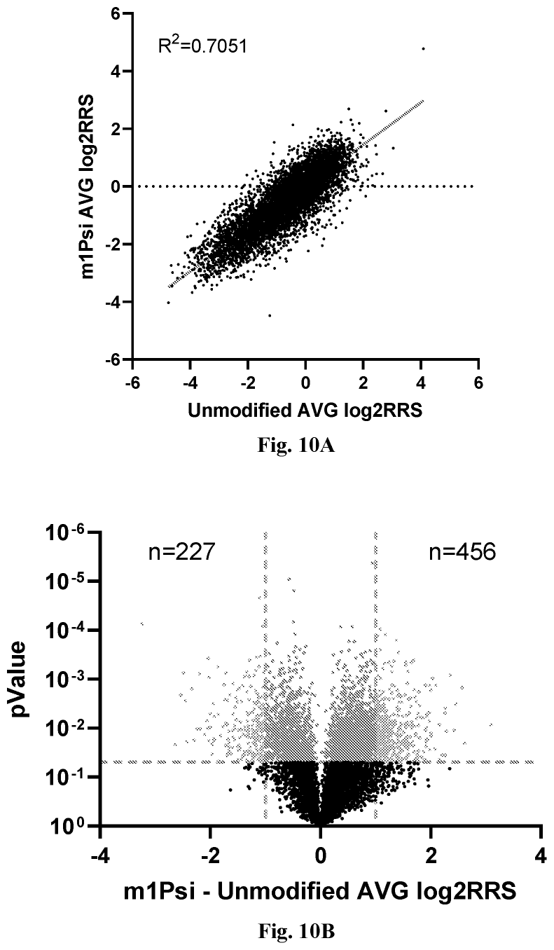

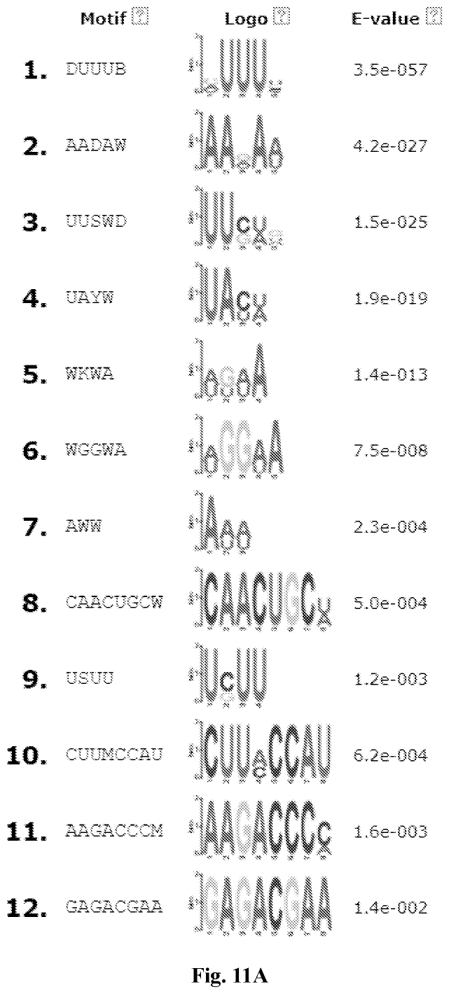

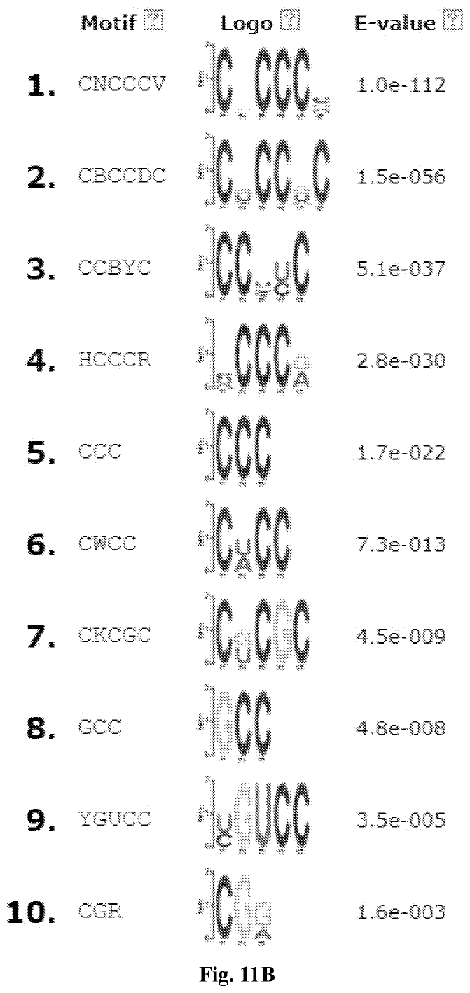

The study further shows that the incorporation of modified nucleobases, such as the NJ-methylpseudouridine, in the 5′ UTRs resulted in in the change of translational efficiencies (FIGS. 10A-10B). The most efficiency human 5′ UTR sequences with N1-methylpseudouridine identified in this batch are listed below in Table 3:

TABLE 3

| | log2RRS | |
|---|---|---|
| Identifier | (m1Psi) | Sequence |
| ENSG00000187514- | 4.778818414 | CCCCCACUGGCUGCUCUGAAAAGCCAUCUUUGCAUUG |
| ENST00000409115- | | UUCCUCAUCCGCCUCCUUGCUCGCCGCAGCCGCCUCCG |
| 182 | | CCGCGCGCCUCCUCCGCCGCCGCGGACUCCGGCAGCUU |
| | | UAUCGCCAGAGUCCCUGAACUCUCGCUUUCUUUUUAA |
| | | UCCCCUGCAUCGGAUCACCGGCGUGCCCCACC |
| | | (SEQ ID NO: 29) |
| ENSG00000206069- | 2.68842281071736 | AUUGUUCUACAAGCUAAAUUAUUCCUUCAAAGGAG |
| ENST00000640159- | | AGGAGAAUUGUAUAAGGUCACAGACGUGGAUCCAA |
| 140 | | AGCCGGAGCCUGAGCAGAGGUGGCCACGCUGGAGG |
| | | GUCUUGACCUAAAGUGUGGUCAGCGAUACUGCCUA |
| | | (SEQ ID NO: 30) |
| ENSG00000184515- | 2.616826934 | AGUUGCAGUCUGGUAGUUGUCGCUGGCCGUGUGACGG |
| ENST00000333643- | | CUCGCUGUUGCCCUGAAGGCAGGCGAGCCAGCUGCCC |
| 223 | | AGGAAAGGUGGAAAGUGGUAGAAGCUGACCCCUGAGC |
| | | CCUGGCAGGUCUUUAAGUGCGUUUGUGCAGCCGAUUU |
| | | CAAGGCUAAGAGAGAAAGACUGCCUCUGAUCCCUGAA |
| | | GGAAGAAAAAAAAAAAAAAAACAGGAAAAAAACUCA |
| | | AC (SEQ ID NO: 31) |
| ENSG00000154305- | 2.187493918 | GACGUGUUUUAUAGGCGGCAUAAAGCGAAAGUGCAGG |
| ENST00000340535- | | CAGCUGUGGAAGGCGAAGUUCAAUCCCAGAGUCCGCC |
| 173 | | CCCUGAAUUGGGGCCUUUCCGGAGGAGGAAGCUCUGA |
| | | AAAACAGGGGGCCCAGUGCCAUUCCGCAGGGAAUUGU |
| | | CGCUUGCGUUCAGCUGUUCUACACA |
| | | (SEQ ID NO: 32) |
| ENSG00000145244- | 1.947769934 | AGUUCAGUUUGUCUCUCUUGAGCUUGGAGAAAAUCAU |
| ENST00000502252- | | CCGUAGUGCCUCCCCGGGGGACACGUAGAGGAGAGAA |
| 123 | | AAGCGACCAAGAUAAAAGUGGACAGAAGAAUAAGCGA |
| | | GACUUUUUAUCC (SEQ ID NO: 33) |
| ENSG00000143799- | 1.934885519 | AGGCAUCAGCAAUCUAUCAGGGAACGGCGGUGGCCGG |
| ENST00000677091- | | UGCGGCGUGUUCGGUGGCGGCUCUGGCCGCUCAGGCG |
| 171 | | CCUGCGGCUGGGUGAGCGCACGCGAGGCGGCGAGGCG |
| | | GCAGCGUGUUUCUAGGUCGUGGCGUCGGGCUUCCGGA |
| | | GCUUUGGCGGCAGCUAGGGGAGG (SEQ ID NO: 34) |
| ENSG00000155380- | 1.872300924 | AAAACAGAAACCAUAAGGCCUACCUCCCAGGGUUGCU |
| ENST00000679803- | | GUGAGAAUUGAGGGGGAGGAUAUUUGGAUUUGACCU |
| 100 | | GCAUUUUGGAAUUUAUCUACACUUAAA |
| | | (SEQ ID NO: 35) |
| ENSG00000115159- | 1.85654596 | AUUAGUACAGCACCCAGAGGUCUUCGGAGCAAAGCUG |
| ENST00000415049- | | UUAAAGAAGUGGGGUCCAUUUGAGCCCCAACUUUGCC |
| 128 | | UUCCUUAGGUCCCCUUGGGCUUGUCUAGCCCUAGGAG |
| | | UUCCAACAAGCUAAGAA (SEQ ID NO: 36) |
| ENSG00000124374- | 1.844203412 | AGUGUGCAGUAAGAGAUAACCUGGCAAGUGGUAGUGG |
| ENST00000244221- | | CGCUUCUCGGGUGCUGUGCUUCACGUUUUGGUACUAA |
| 187 | | AGGCCGAGACUGUUGUGGCGACGGCGACCUCUACGGC |
| | | AACGGCUUAAGCUCUCGGAGGAGUGGCAGAGUACGAU |
| | | CUGAAGGAGGGGCUUCUGGUUAGCCCAGGUUCCAUCA |
| | | UA (SEQ ID NO: 37) |
| ENSG00000154760- | 1.798983187 | GGAAACGCGGAGUGUCCUGGAGCUUGACGCGCUGGAG |
| ENST00000285013- | | CUUGAGAGGUCUUUCAGGACUGAUUUUGGUGGAAGAA |
| 205 | | GACAGCAGGCGGUGGGCAUUCACUGUGCUGGUGGGAA |
| | | GUCUGGUUACAUUAAGAAAGGAAUUCUAGCAAAGUUA |
| | | UUCUGUAAAUACAACAGAAUCAGUUGCUUUUGUUAAA |
| | | UAUAUUGGUGCAAGUUCAAC (SEQ ID NO: 38) |
| ENSG00000176102- | 1.778455315 | AAUUCAUAUUUGAGUGGGGGUGGCGAUUGGUGUUG |
| ENST00000323959- | | GCGGUCUGGCUCAGCUGGGCAGGGGGUAACUUUACUG |
| 155 | | AUUUGGGGGUGGUUUUUAGUUUAAUUUUUCUUUUCU |
| | | AGCUUCCCAUCGACGGUCAGUGCGCACGUUGUAAUCA |
| | | GCUGAGGCC (SEQ ID NO: 39) |
| ENSG00000175294- | 1.769363289 | AGUCUGCAUUGAGCUUGGCUCAGGAAAGAAGGGAAAU |
| ENST00000312106- | | CCAGGCGGGGCCUGUUGGGCCCAGGUCUUGAGCUCUU |
| 100Fluc | | UUGGCUCCAGAGUUCCCAGCACAGUC |
| | | (SEQ ID NO: 40) |
| ENSG00000170290- | 1.743646862 | AGACAGCCUGGGAGGGGAGAAGGAGUUGGAGCUCAAG |
| ENST00000305991- | | UUGGAGACAGCGAGGAGAAACCUGCCAUAGCCAGGGU |
| 169 | | GUGUCUUUGAUCCUCUUCAGGAGGUGAGGAGAAGCCA |
| | | GAGGUCCUUGGUGUGCCCUCAGAAAUCUGCCUGCAGU |
| | | UCUCACCAAGCCGCUGUGAAA (SEQ ID NO: 41) |
| ENSG00000138468- | 1.740036667 | UUUUUUUUUUUUUUUUUUUUUUUUUUUUCCUUUCCCC |
| ENST00000314261- | | UCCCCCUCCCUCUCCAAGCCGGAGGGGUCCUGAGGUG |
| 111 | | ACAGCGCCUGCAACUGAAAUUUCAGCAGCGGGAGAAG |
| | | (SEQ ID NO: 42) |
| ENSG00000171657- | 1.708240385 | ACACUGACUAAAGAUAUUGGAAGAGUGAAGAUCAGAG |
| ENST00000302548- | | AAUUUUAAGUCUGAAAUUUGGCAUCACUGCCCUGAAC |
| 214 | | AAUAUAACCUUAGUUGGCAUAAACUACUCAUACAGAC |
| | | AAAGGCAUUAUCCAUCACAAUAAGUAACUUUUUGUCU |
| | | UUAUUUCAACCGGACAAUCGUGAUUAGAAAAGCUCCU |
| | | GUGACAAAAUUCAAGAAAACCUGACAUAA |
| | | (SEQ ID NO: 43) |
| ENSG00000154760- | 1.687013216 | AAACGCGGAGUGUCCUGGAGCUUGACGCGCUGGAGCU |
| ENST00000534689- | | UGAGAGGUCUUUCAGGACUGAUUUUGGUGGAAGAAG |
| 203 | | ACAGCAGGCGGUGGGCAUUCACUGUGCUGGUGGGAAG |
| | | UCUGGUUACAUUAAGAAAGGAAUUCUAGCAAAGUUAU |
| | | UCUGUAAAUACAACAGAAUCAGUUGCUUUUGUUAAAU |
| | | AUAUUGGUGCAAGUUCAAC (SEQ ID NO: 44) |
| ENSG00000105357- | 1.665273017 | GAGGCUGCAGCAGCACAUACAGGAGCUAGAGGCCC |
| ENST00000262269- | | ACCUUGAGGCUGAGGAGGGUGCGCGGCAGAAGCUG |
| 100 | | CAGCUGGAGAAGGUGACGACAGAGGCAAAA (SEQ |
| | | ID NO: 45) |
| ENSG00000164919- | 1.661077612 | AUUCUGCGCCUGCGCGCGGCUACAGCACGGUUCGUUU |
| ENST00000520468- | | UUCCUUUAGUCAGGAAGGACGUUGGUGUUGAGGUUAG |
| 100 | | CAUACGUAUCAAGGACAGUAACUACC |
| | | (SEQ ID NO: 46) |
| ENSG00000163762- | 1.65902399510468 | GAGAGAGAGAGAGAAAAAAAAGGUCAGGUAGGAAGA |
| ENST00000470080- | | AGAAAAAUAGACUUUCUGUGACUGUAUACUCUUGAAA |
| 100 | | UCUCCCGCAGGUUCUAAGCAGGGCAAA |
| | | (SEQ ID NO: 47) |
| ENSG00000061492- | 1.636603322 | CAGAAUUUUCUCACAUAAAUACUGAGGAAGACCCUGC |
| ENST0000a0361560- | | CCUCUCCUCACUUCUCUGGACUUGGCCCUGAGCUGGA |
| 100 | | CCUGGUCCACUGGGGUAGGCAGGGCG |
| | | (SEQ ID NO: 48) |
| ENSG00000138311- | 1.616348731 | AGUCUGAUUUACGGCUCUGCUGAAAACCGCUUCGCUC |
| ENST00000395254- | | CCGCAGCAUCAGCACCGGAGGCGGCGGCUGCAGCAGC |
| 103 | | AGCAGCAACAAGUCGGGACUUUUAGAGUA |
| | | (SEQ ID NO: 49) |
| ENSG00000117308- | 1.612147772 | AUUAGUCACUGCGCCCAGGAGCCAGUUUACACCUAGC |
| ENST00000445705- | | GAAGGCGGAGGCAUUGGACAAGCCUGGGUCUGAGCAG |
| 210 | | ACAGAACCCCUUCCUGCGACCCUGGAGUCCCAAUCGU |
| | | GCUGCCAUUUUCCUUCUGCCCAGGACUCUCCAGUCCU |
| | | CAGUCACCUUGGACAAAGAAGUGUGGAUCCUCAGAUU |
| | | CCAUCUUUUCCAACUCCAAGGUGCC |
| | | (SEQ ID NO: 50) |
| ENSG00000078369- | 1.610202699 | CUGGAAGGUCUGGGGGACUGAGGGAAGCGAAGAGAAA |
| ENST00000437146- | | AGCAAGAGCAAUUAAAUAUCUCACAUUCUUGAAGGUG |
| 100 | | GCAUUGAAGAGCACUAAGAUCGGAAG |
| | | (SEQ ID NO: 51) |

A further screening identified more than a thousand short 5′-UTR sequences that, when combined with N1-methylpseudouridine, resulted in higher translational efficiency than the 5′-UTR used in Moderna mRNA-based vaccine. The short 5′-UTR sequences in this batch are listed below in Table 4 below:

TABLE 4

| | m1Psi_ | SEQ | |
|---|---|---|---|
| | AVG_log2 | ID | |
| ID | RRS | NO: | Sequence |
| ENSG000 | 1.169790902 | SEQ | CUGCUUGGCUUUGAGGAAGAGUGGCAGUACUGCCU |
| 00000457- | | ID | CACUGCAUAAGGG |
| ENST000 | | NO: | |
| 00367770- | | 56 | |
| 48 | | | |
| ENSG000 | 1.254321398 | SEQ | GAAGCUCUCCUGUUUGACGAAAGU |
| 00000460- | | ID | |
| ENST000 | | NO: | |
| 00459772- | | 57 | |
| 24 | | | |
| ENSG000 | 1.42504224 | SEQ | AUAGCCUAGAUCGAGCUCCCUGUGUGCACCGCGCGC |
| 00002016- | | ID | UGCCCGAGAGGUCAACCAGAAUCAAG |
| ENST000 | | NO: | |
| 00544742- | | 58 | |
| 62 | | | |
| ENSG000 | 1.641692433 | SEQ | UAGAAGAGUGAUAUUUGUGUGAGAGCCUGGGUCAG |
| 00003096- | | ID | GCUUAUUUUGAGACGUGGGCCCCACUGAGGUCAUU |
| ENST000 | | NO: | AUCCAACUUUUAGAGGUG |
| 00541812- | | 59 | |
| 88 | | | |
| ENSG000 | 1.198841244 | SEQ | AGGACUGAGUUCUGAAUUAACUAAAGUAACUUGUC |
| 00003402- | | ID | UGGUUUGCAGAGUGCUG |
| ENST000 | | NO: | |
| 00479953- | | 60 | |
| 52 | | | |
| ENSG000 | 1.386556667 | SEQ | UCAGAAACUGUUAGUUGGCAAAGGAGUUACCAUCU |
| 00003402- | | ID | CCUAUUAAACGGGCAAGGACAAGUUCCCUUUUCUG |
| ENST000 | | NO: | AGAGUGCUG |
| 00494258- | | 61 | |
| 79 | | | |
| ENSG000 | 1.108285028 | SEQ | ACUUCGGCGGAC |
| 00004455- | | ID | |
| ENST000 | | NO: | |
| 00548033- | | 62 | |
| 12 | | | |
| ENSG000 | 1.39092934 | SEQ | ACUAGUGCUUCCUAGUGUCAGGGCCAGCUGUGUAG |
| 00004766- | | ID | UGGCUCGGUGUGAUUUGUUAGCUCUUUGAGGCAGG |
| ENST000 | | NO: | GUACCCUCCUCAGGAUUUCGAU |
| 00436177- | | 63 | |
| 92 | | | |
| ENSG000 | 1.351422107 | SEQ | ACAACAAAGCUUCCAGAGGGAGAGGAAGG |
| 00005981- | | ID | |
| ENST000 | | NO: | |
| 00325885- | | 64 | |
| 29 | | | |
| ENSG000 | 1.473564565 | SEQ | UGGGAAAUAAAGUCAUUUGUAGUUUUUUCUCUCUU |
| 00006576- | | ID | GCACAGCCUAAAAGUACCAAGUUGUGAUUUCAGUG |
| ENST000 | | NO: | GAAUAA |
| 00248550- | | 65 | |
| 76 | | | |
| ENSG000 | 1.538217907 | SEQ | ACAGAAGACUGGACACAAUUCCGAAGAGCUGCCCA |
| 00006659- | | ID | GAAGGAGAGAACA |
| ENST000 | | NO: | |
| 00392052- | | 66 | |
| 48 | | | |
| ENSG000 | 1.117959259 | SEQ | UGCUGCUCUCGGUUUAGUCCAAGAUCAGCGAUAUC |
| 00006747- | | ID | ACGCGUCCCCCGGAGCAUCGCGUGCAGGAGCC |
| ENST000 | | NO: | |
| 00297029- | | 67 | |
| 67 | | | |
| ENSG000 | 1.244066478 | SEQ | GCUGCUCUCGGUUUAGUCCAAGAUCAGCGAUAUCA |
| 00006747- | | ID | CGCGUCCCCCGGAGCAUCGCGUGCAGGAGCC |
| ENST000 | | NO: | |
| 00436854- | | 68 | |
| 66 | | | |
| ENSG000 | 1.154217639 | SEQ | AGCUCCUGGCGCCUGCGAUAGCGGCAAG |
| 00007341- | | ID | |
| ENST000 | | NO: | |
| 00358039- | | 69 | |
| 28 | | | |
| ENSG000 | 1.193659185 | SEQ | CUGCCUUGCUCUGCCAGGAACC |
| 00007384- | | ID | |
| ENST000 | | NO: | |
| 00417043- | | 70 | |
| 22 | | | |
| ENSG000 | 1.105464088 | SEQ | GUGAAAGGUUGCGAAG |
| 00007923- | | ID | |
| ENST000 | | NO: | |
| 00377577- | | 71 | |
| 16 | | | |
| ENSG000 | 1.108125958 | SEQ | AAGAAGCGGAGAUUUAACGGGGACGUGCGGCCAGA |
| 00010704- | | ID | GCUGGGGAA |
| ENST000 | | NO: | |
| 00336625- | | 72 | |
| 44 | | | |
| ENSG000 | 1.66013041 | SEQ | UCUCUUACUUCAAAGACUGAACAUUCUUCACGAUC |
| 00011258- | | ID | UGAAAGGAAAAGAAGAGAUUCUUUCGGG |
| ENST000 | | NO: | |
| 00415868- | | 73 | |
| 63 | | | |
| ENSG000 | 1.1337154 | SEQ | UCGGGCAUUAAAAAAUAUUUAAUCAUUCAGUUGUU |
| 00011454- | | ID | GAGACUCAUUCUUGAGUU |
| ENST000 | | NO: | |
| 00426918- | | 74 | |
| 53 | | | |
| ENSG000 | 1.502675649 | SEQ | AUUCUACAGUGCAAGUAGCUUUGAUUGUCAGGGUU |
| 00011465- | | ID | CAGGUUCCCUGGUUGUGAAAAUACAGUAGAUAAAU |
| ENST000 | | NO: | C |
| 00550563- | | 75 | |
| 71 | | | |
| ENSG000 | 1.182322223 | SEQ | GUUGCUUGGGCGCUUCUCCGCUGCGUGUAGGUGAA |
| 00013375- | | ID | GGGGGCUUCCUGACCGAGAC |
| ENST000 | | NO: | |
| 00512866- | | 76 | |
| 55 | | | |
| ENSG000 | 1.353206743 | SEQ | UACAGUUUGAAGACAGUAAUACCUGGAAUUGAGCA |
| 00013441- | | ID | UCAGUUCUGUCAUUCUUAAAUUUGC |
| ENST000 | | NO: | |
| 00409769- | | 77 | |
| 60 | | | |
| ENSG000 | 1.222017984 | SEQ | AAUCCCUUAAUUAAAUAGCUUCCCCUCUACAGGCUU |
| 00014257 | | ID | UUGAAGUGGUAGCAGUUCCUCCUAACUCCUGCCAG |
| ENST000 | | NO: | AAACAGCUCUCCUCAAC |
| 00495911- | | 78 | |
| 88 | | | |
| ENSG000 | 1.393481764 | SEQ | AGACCACAGAGCCCUGGGUUGUGGAAGAGGUGGCU |
| 00014919- | | ID | GUUCCCUGUCAUCAGU |
| ENST000 | | NO: | |
| 00370483- | | 79 | |
| 51 | | | |
| ENSG000 | 1.447526496 | SEQ | GUUUAAAAUUAUCCAACUGCCAUAGAGCUAAAUUC |
| 00018236- | | ID | UUUUUUGGAAAAUUGAACCGAACUUCUACUGAAUA |
| ENST000 | | NO: | CAAG |
| 00348761- | | 80 | |
| 74 | | | |
| ENSG000 | 1.650397087 | SEQ | GAAAGUUAGUUGCUUUCUUAACCUCAUCAAAUUCA |
| 00021826- | | ID | GUAAGAUUUAGCCGAGGCCCAGUCCACAAAUCAUC |
| ENST000 | | NO: | AAA |
| 00673711- | | 81 | |
| 73 | | | |
| ENSG000 | 1.309628047 | SEQ | GUUAAGCUAGUCCAGAGCCGAGUCCAAAUUUUAUU |
| 00022267- | | ID | CCGGUACAGGG |
| ENST000 | | NO: | |
| 00539015- | | 82 | |
| 46 | | | |
| ENSG000 | 1.148156641 | SEQ | AGAAUUCAGAACUUUGCUUCUAAAAAGGUCCCUCC |
| 00022267- | | ID | AGCUACAAGGUGGGCACC |
| ENST000 | | NO: | |
| 00628032- | | 83 | |
| 53 | | | |
| ENSG000 | 1.321011671 | SEQ | AGAGUUAGCCGAGGCCGCCAUAUUGAAUAAGCGAC |
| 00023228- | | ID | CCGGCCUCCUAGGGGGUCGUCGUGGUCCAGACAGUU |
| ENST000 | | NO: | UAGCAGAACAGCCUCCGCGGCUCCGGGGAGAAGCAA |
| 00233190- | | 84 | U |
| 108 | | | |
| ENSG000 | 1.406012596 | SEQ | AUAUUGAAUAAGCGACCCGGCCUCCUAGGGGGUCG |
| 00023228- | | ID | UCGUGGUCCAGACAGUUUAGCAGAACAGCCUCCGCG |
| ENST000 | | NO: | GCUCCGGGGAGAAGCAAU |
| 00440274- | | 85 | |
| 89 | | | |
| ENSG000 | 1.459698608 | SEQ | UAUUAAUAGAAGAGUCUUCGUUCCAGACGCAGUCC |
| 00023839- | | ID | AGGAAUC |
| ENST000 | | NO: | |
| 00649493- | | 86 | |
| 42 | | | |
| ENSG000 | 1.23653664 | SEQ | CUUCAUUUCCCAUCGUGCUGAGGCGGGUGGCAGUG |
| 00028203- | | ID | CGGAGAAGG |
| ENST000 | | NO: | |
| 00436874- | | 87 | |
| 44 | | | |
| ENSG000 | 1.452349547 | SEQ | GCAGAUCAGCGGAAGUGCUAAAGUAAGGUGCUAUA |
| 00031081- | | ID | CUUUACUUAAAAGGUGAAA |
| ENST000 | | NO: | |
| 00482743- | | 88 | |
| 54 | | | |
| ENSG000 | 1.222568403 | SEQ | AACAGUUGCCUUUUCCUCUCACAGAAGGAAGAUUU |
| 00035720- | | ID | CAUUUUGUUUGAGACGAGAAACCAAACCACACACC |
| ENST000 | | NO: | AAAGAGAGGGGU |
| 00396225- | | 89 | |
| 82 | | | |
| ENSG000 | 1.785440513 | SEQ | ACGUGGACGUCAACAGCA |
| 00036054- | | ID | |
| ENST000 | | NO: | |
| 00344949- | | 90 | |
| 18 | | | |
| ENSG000 | 1.223366542 | SEQ | AAUUGUAAAGAGAGAGAAGUGGAUAAAUCAGUGCU |
| 00038945- | | ID | GCUUUCUUUAGGACGAAAGAAGU |
| ENST000 | | NO: | |
| 00262101- | | 91 | |
| 58 | | | |
| ENSG000 | 1.219486289 | SEQ | UAGGUUUCAAUUGUAAAGAGAGAGAAGUGGAUAAA |
| 00038945- | | ID | UCAGUGCUGCUUUCUUUAGGACGAAAGAAGU |
| ENST000 | | NO: | |
| 00381998- | | 92 | |
| 66 | | | |
| ENSG000 | 1.334322058 | SEQ | AGAUUUGCUCUCACUGCUCCCAAAA |
| 00039123- | | ID | |
| ENST000 | | NO: | |
| 00230640- | | 93 | |
| 25 | | | |
| ENSG000 | 1.24977856 | SEQ | AGUUUUGAGCAUACUUCUGAACUGGCUUUUGUUGA |
| 00048740- | | ID | GACUAUCAGUAUAGAAGC |
| ENST000 | | NO: | |
| 00633200- | | 94 | |
| 53 | | | |
| ENSG000 | 1.183441512 | SEQ | GAGACAGUUGAAAGUUCUCUACAACAGAAGCCAAG |
| 00049247- | | ID | AAGGAAGCCGUCUAUCUUGUGGCGAUC |
| ENST000 | | NO: | |
| 00361696- | | 95 | |
| 62 | | | |
| ENSG000 | 1.305957441 | SEQ | UUUCUCCCCGAG |
| 00049540- | | ID | |
| ENST000 | | NO: | |
| 00428787- | | 96 | |
| 12 | | | |
| ENSG000 | 1.606022945 | SEQ | GAGAAUAAAAACAGUGCUUUUGAGUAAAAAGUCAU |
| 00050730- | | ID | UUAACAGAUUCCAGUUGGAGUGCUC |
| ENST000 | | NO: | |
| 00507879- | | 97 | |
| 60 | | | |
| ENSG000 | 1.135865322 | SEQ | AUAUUGCAGGACGCUGCAUC |
| 00050748- | | ID | |
| ENST000 | | NO: | |
| 00397072- | | 98 | |
| 20 | | | |
| ENSG000 | 1.147992378 | SEQ | AGUCAAUUAGUUAAAGACUCUGACAUCUGCUAGGC |
| 00053702- | | ID | GGGAGGCAAGAGACCUAACAAGCAGGGGCC |
| ENST000 | | NO: | |
| 00542990- | | 99 | |
| 65 | | | |
| ENSG000 | 1.126727287 | SEQ | UUCACUUCUUCAACUGAGAGUGACAGUAUAUAAUC |
| 00054654 | | ID | UCCAUUGAGUCAAAGA |
| ENST000 | | NO: | |
| 00554584- | | 100 | |
| 51 | | | |
| ENSG000 | 1.194826351 | SEQ | AGACACACAUAGCCUCUCUGCCCACCUCUGCUUCCU |
| 00057149- | | ID | CUAGGAACACAGGAGUUCCAGAUCACAUCGAGUUC |
| ENST000 | | NO: | ACC |
| 00283752- | | 101 | |
| 74 | | | |
| ENSG000 | 1.289797178 | SEQ | GUCAGUCCCAGUGGGAAGUUCAACAGGCAGGGGCA |
| 00057593- | | ID | GCACUGCAGAGAUUUCAUC |
| ENST000 | | NO: | |
| 00541084- | | 102 | |
| 54 | | | |
| ENSG000 | 1.251738427 | SEQ | ACGCUUUGCGGCUGUCGUCGGAGAGGCAUCUGGGU |
| 00058729- | | ID | UCGGACUGGGGCCGCC |
| ENST000 | | NO: | |
| 00510384- | | 103 | |
| 51 | | | |
| ENSG000 | 1.098136979 | SEQ | GUCCUCGCAGCC |
| 00061794- | | ID | |
| ENST000 | | NO: | |
| 00081029- | | 104 | |
| 12 | | | |
| ENSG000 | 1.438331965 | SEQ | ACCUGGCCGAGCAGGAGGCGCCAUC |
| 00063177- | | ID | |
| ENST000 | | NO: | |
| 00547897- | | 105 | |
| 25 | | | |
| ENSG000 | 1.361048302 | SEQ | CAAAGCUGGCUCUGCAGGGGAC |
| 00064205- | | ID | |
| ENST000 | | NO: | |
| 00190983- | | 106 | |
| 22 | | | |
| ENSG000 | 1.1348426 | SEQ | CUGCUCUGUUAAAAGCGAAA |
| 00064933- | | ID | |
| ENST000 | | NO: | |
| 00424307- | | 107 | |
| 20 | | | |
| ENSG000 | 1.097381246 | SEQ | CCCUCCGGGAAG |
| 00065427- | | ID | |
| ENST000 | | NO | |
| 00562875- | | 108 | |
| 12 | | | |
| ENSG000 | 1.287728744 | SEQ | AGAGAGAGGGGGAUUUAUUCAAAGUUGUUUCCAAU |
| 00065618- | | ID | UCCUUCAAAACCUCAAACCAGGUGGCUAGUGU |
| ENST000 | | NO: | |
| 00393211- | | 109 | |
| 67 | | | |
| ENSG000 | 1.147916239 | SEQ | UUGGCUCCAGACAAAUAAAC |
| 00066427- | | ID | |
| ENST000 | | NO: | |
| 00557030- | | 110 | |
| 20 | | | |
| ENSG000 | 1.336270833 | SEQ | CGAUAGACGAGGACGCCAACAGCAGCGGAGAAACG |
| 00066654- | | ID | UUUCUCUUUCCUCUCAGUUUGCGCACACC |
| ENST000 | | NO: | |
| 00565248- | | 111 | |
| 64 | | | |
| ENSG000 | 1.295319418 | SEQ | GAGAAAGCUGUCUAGGUAGAGUUCAGUCAGCCCAG |
| 00067225- | | ID | UGACCUCAGCAGCC |
| ENST000 | | NO: | |
| 00564178- | | 112 | |
| 49 | | | |
| ENSG000 | 1.256881067 | SEQ | UGACAAGACCACAAUCUGAUCCCAAAG |
| 00068781- | | ID | |
| ENST000 | | NO: | |
| 00394751- | | 113 | |
| 27 | | | |
| ENSG000 | 1.261796623 | SEQ | GAUUGUCUGUGCUCUGAGCAGGCUGAAGUGACUGG |
| 00069702- | | ID | ACGAGACGCACUGUUGGAGAAAUAAAA |
| ENST000 | | NO: | |
| 00525962- | | 114 | |
| 62 | | | |
| ENSG000 | 1.816127806 | SEQ | AUUUUCUUCAAUCACAUCUGAAUAAAUCACUUGAA |
| 00070669- | | ID | GAAAGCUUAUAGCUUCAUUGCACC |
| ENST000 | | NO: | |
| 00454046- | | 115 | |
| 59 | | | |
| ENSG000 | 1.137119928 | SEQ | AGUUGAAAACUUACAGAGUCCUGAGGCUUUCAGAC |
| 00070718- | | ID | UGAAAAAGGCUUUCUUCUGUCACUGACAAUCGCCA |
| ENST000 | | NO: | CC |
| 00517922- | | 116 | |
| 72 | | | |
| ENSG000 | 1.77062527 | SEQ | CGCUGAGAGCAGCAAGGCCAGAGUUGAAAACUUAC |
| 00070718- | | ID | AGAGUCCUGAGGCUUUCAGACUGAAAAAGGCUUUC |
| ENST000 | | NO: | UUCUGUCACUGACAAUCGCCACC |
| 00530375- | | 117 | |
| 93 | | | |
| ENSG000 | 1.319758271 | SEQ | UCAUCGUUUUGAAGAGUUCUGCGUUUUGCCAGUCA |
| 00071051- | | ID | CCUCUCAACUGUGUGCCAAAGGACUCCAGUAAAG |
| ENST000 | | NO: | |
| 00522586- | | 118 | |
| 69 | | | |
| ENSG000 | 1.829424723 | SEQ | CACAGAGCGACAGAGACAUUUAUUGUUAUUUGUUU |
| 00071054- | | ID | UUUGGUGGCAAAAAGGGAAA |
| ENST000 | | NO: | |
| 00413150- | | 119 | |
| 55 | | | |
| ENSG000 | 1.170950597 | SEQ | AGCCGCACUCGCCAGCGACUCAUCUCUCCAGCGGGU |
| 00071575- | | ID | UUUUUUUUGUUUGUCGUGUGCGAUCCUCACACUC |
| ENST000 | | NO: | |
| 00405331- | | 120 | |
| 70 | | | |
| ENSG000 | 1.118018921 | SEQ | AGUGUUUGAUAAAGACAGCUCCUCUUAGGAAGAAC |
| 00072080- | | ID | UGUCAUCCCCAAACACAUAGAGAGACACUCUCUGUC |
| ENST000 | | NO: | UCUCGAUUACAAUC |
| 00168148- | | 121 | |
| 85 | | | |
| ENSG000 | 1.451902319 | SEQ | AAUCCGCAUUCAGUUGUCGAGGAGUGCCAGUCACC |
| 00072571- | | ID | UUCAGUUUCUGGAGCUGGCCGUCAAC |
| ENST000 | | NO: | |
| 00393915- | | 122 | |
| 61 | | | |
| ENSG000 | 1.447852479 | SEQ | CUUUUCGCGCGAAACCUGGUUGUUGCUGUAGUGGC |
| 00073111- | | ID | GGAGAGGAUCGUGGUACUGCU |
| ENST000 | | NO: | |
| 00265056- | | 123 | |
| 56 | | | |
| ENSG000 | 1.358968597 | SEQ | ACUUUUCGCGCGAAACCUGGUUGUUGCUGUAGUGG |
| 00073111- | | ID | CGGAGAGGAUCGUGGUACUGCU |
| ENST000 | | NO: | |
| 00474964- | | 124 | |
| 57 | | | |
| ENSG000 | 1.410113142 | SEQ | AAAGGGAAGCGAAGCCGGAGCUGCGGGCGCUUUUU |
| 00073584- | | ID | CUGCCCGCGGUGUCUCAGAUUCAUUCUUAAGGAAC |
| ENST000 | | NO: | UGAGAACUUAAUCUUCCAAA |
| 00580419- | | 125 | |
| 90 | | | |
| ENSG000 | 1.460575417 | SEQ | AGAAAGUUUAAAGUGCUGAGUGAUUUGCACUAGCU |
| 00073969- | | ID | GUAUAGAUCUUUGGUGGAGGAGGAAAGUGGAAAAU |
| ENST000 | | NO: | UAUUAAUUUCAGAGC |
| 00571172 | | 126 | |
| 85- | | | |
| ENSG000 | 1.115293315 | SEQ | ACCGAGCUGCCUGCC |
| 00074660- | | ID | |
| ENST000 | | NO: | |
| 00573852- | | 127 | |
| 15 | | | |
| ENSG000 | 1.113548171 | SEQ | CCGAUACUCAGCCCUCUGCGACC |
| 00075239- | | ID | |
| ENST000 | | NO: | |
| 00531813- | | 128 | |
| 23 | | | |
| ENSG000 | 1.192169419 | SEQ | UAGAGUUGAAGAACAGACUCAAACCAGGUUUUUAU |
| 00075303- | | ID | UUAAUUAGUUAAAAAU |
| ENST000 | | NO: | |
| 00429674- | | 129 | |
| 51 | | | |
| ENSG000 | 1.146633395 | SEQ | AGCAGACACUGGAAGUUGAAGUCAUUGGGAGGUGA |
| 00075420- | | ID | GCCCUUAAGGAGAGCCCAA |
| ENST000 | | NO: | |
| 00443501- | | 130 | |
| 54 | | | |
| ENSG000 | 1.316791348 | SEQ | UAUUUUUAUUUACAGUUUUUGUUCAACUAGGCAAC |
| 00075711- | | ID | UUAAAAUAGUUUUGCUUUAGUUAUAAAAUAUUGUA |
| ENST000 | | NO: | AGUGUAGAGUA |
| 00658155- | | 131 | |
| 81 | | | |
| ENSG000 | 1.304083668 | SEQ | UCUCUCUCGCUCGUUCCCUAACAUUAAAGAGAAA |
| 00076067- | | ID | |
| ENST000 | | NO: | |
| 00552916- | | 132 | |
| 34 | | | |
| ENSG000 | 1.094285381 | SEQ | CUAGACGUUUCCUUGGCUCUUCUGGCGCCAAA |
| 00076242- | | ID | |
| ENST000 | | NO: | |
| 00457004- | | 133 | |
| 32 | | | |
| ENSG000 | 1.247415306 | SEQ | UCUAGACGUUUCCUUGGCUCUUCUGGCGCCAAA |
| 00076242- | | ID | |
| ENST000 | | NO: | |
| 00673972- | | 134 | |
| 33 | | | |
| ENSG000 | 1.264737114 | SEQ | AGAGACACGAAUAGUUUUCAGCCGCAACAGGCUGC |
| 00077044- | | ID | GUUUCAGCCGGAAGAGUGAAAGGGCACCUUGAAAA |
| ENST000 | | NO: | CGCAAGUUU |
| 00409813- | | 135 | |
| 79 | | | |
| ENSG000 | 1.360777028 | SEQ | AGAUCGUAAACAAAACCCAGGACGCCAGGCAGAUU |
| 00077063- | | ID | UCUGUGUUAACAAGAAAAAAGAGUUUGAGUUGGAU |
| ENST000 | | NO: | ACUCUCAGUAAAUCCGAGCUGCGG |
| 00434890- | | 136 | |
| 94 | | | |
| ENSG000 | 1.123328923 | SEQ | CGCUUUUUCCGCGGGCGCUUGAUAACGCGGGUGAG |
| 00077935- | | ID | GCGUGGAGGGCGGCGCC |
| ENST000 | | NO: | |
| 00404354- | | 137 | |
| 52 | | | |
| ENSG000 | 1.381292769 | SEQ | UUAUAUUUAAGCAGUGAGAGUAUUUACGUCAAGAU |
| 00078142- | | ID | CAACUUAUUCUUCAAAUCAUUUCACUC |
| ENST000 | | NO: | |
| 00589056- | | 138 | |
| 62 | | | |
| ENSG000 | 1.118461149 | SEQ | CUGGUUUCUUGCCUUAAGGAGCCCAUUGCCUUUCCC |
| 00078304- | | ID | GCUGAAGUCUAG |
| ENST000 | | NO: | |
| 00334743- | | 139 | |
| 48 | | | |
| ENSG000 | 1.718228618 | SEQ | AGCAGUUUCUGAGCUGCAAAAACUAGUUUCUAAAC |
| 00078674- | | ID | AGAGAGUUAAUUGUUAAAUCCAGU |
| ENST000 | | NO: | |
| 00524226- | | 140 | |
| 59 | | | |
| ENSG000 | 1.167182176 | SEQ | ACUUUCUUUUGUAAAGUUGGUUUCUACAAAG |
| 00079557- | | ID | |
| ENST000 | | NO: | |
| 00226355- | | 141 | |
| 31 | | | |
| ENSG000 | 1.358393990 | SEQ | AAAAACAGCAACUCUAAACUGGCUCUGGAUAGUUC |
| 00079819- | | ID | AUAAGCUGUGGCC |
| ENST000 | | NO: | |
| 00529709- | | 142 | |
| 48 | | | |
| ENSG000 | 1.232028523 | SEQ | AGCCUUCGGGCCUUGUAGUAGGUAGCAGUAAACAG |
| 00080503- | | ID | CAGAUUUGAGUAUCCACAAUCCUUUCAGUAUUAAA |
| ENST000 | | NO: | UUUUCAGUAAAAUAAAAUUACUUGUAU |
| 00634343- | | 143 | |
| 97 | | | |
| ENSG000 | 1.347728768 | SEQ | AAAAUAACUAGCAACC |
| 00081051- | | ID | |
| ENST000 | | NO: | |
| 00226359- | | 144 | |
| 16 | | | |
| ENSG000 | 1.220437906 | SEQ | AUUGUGCUUCCACCACUGCCAAUAACAAAAUAACU |
| 00081051- | | ID | AGCAACC |
| ENST000 | | NO: | |
| 00395792- | | 145 | |
| 42 | | | |
| ENSG000 | 1.428014134 | SEQ | AAUUUGAAGGGUUUCUUGUAGAGAGAAAGUCACAU |
| 00082153- | | ID | UUUGACGUACAUUUGAUUCAAGUGCAGGGUGUCUU |
| ENST000 | | NO: | UU |
| 00419090- | | 146 | |
| 72 | | | |
| ENSG000 | 1.701310135 | SEQ | CAGAGGUGCUGUUAUCAGAAUAGUCUUCUGGGUAU |
| 00082269- | | ID | AAAUUAAAA |
| ENST000 | | NO: | |
| 00194672- | | 147 | |
| 44 | | | |
| ENSG000 | 1.114153277 | SEQ | GAGAAUCCCGGUGGUGAGCGGGG |
| 00083123- | | ID | |
| ENST000 | | NO: | |
| 00320393- | | 148 | |
| 23 | | | |
| ENSG000 | 1.232812987 | SEQ | UUUGUUUCUGUCAGGCUGUCCCUGGCCGGGGUUCU |
| 00083814- | | ID | GUAACGCUUGUGUGGGCCGCAGGUGGAGGUGUUGG |
| ENST000 | | NO: | GAAAGCGCGGAGGAG |
| 00600125- | | 149 | |
| 85 | | | |
| ENSG000 | 1.18233773 | SEQ | CCCCUUUGGAGCUACUUCCUC |
| 00084092- | | ID | |
| ENST000 | | NO: | |
| 00264230- | | 150 | |
| 21 | | | |
| ENSG000 | 1.759674184 | SEQ | GUUUUUCUUUUUUUAAGUAACAAGUAAACCAUACA |
| 00084463- | | ID | AAUUGUCAAC |
| ENST000 | | NO: | |
| 00535638- | | 151 | |
| 45 | | | |
| ENSG000 | 1.155467285 | SEQ | GCUGUCCUCUUCAGCUCAAG |
| 00084754- | | ID | |
| ENST000 | | NO: | |
| 00645274- | | 152 | |
| 20 | | | |
| ENSG000 | 1.283122656 | SEQ | CUGGAAGAGGAUCUUGGGCGCCGCCAGGUUCUGUG |
| 00085063- | | ID | GACAAUCACA |
| ENST000 | | NO: | |
| 00533403- | | 153 | |
| 45 | | | |
| ENSG000 | 2.109716508 | SEQ | AUUAUACUUCUUACAGUGAGGAGCCAGAGCUUCCA |
| 00085063- | | ID | GGUUCUGUGGACAAUCACA |
| ENST000 | | NO: | |
| 00652678- | | 154 | |
| 54 | | | |
| ENSG000 | 1.848653664 | SEQ | GUGAGAGGACAUUGUGAUAAGUGAAGUGAAAGAGC |
| 00085117- | | ID | UUUGUCAGCUGCACAGGUGA |
| ENST000 | | NO: | |
| 00526958- | | 155 | |
| 55 | | | |
| ENSG000 | 1.611121882 | SEQ | ACAGAAAGUAGCUCAUUCCUCCUAACUGGGUAAUA |
| 00085721- | | ID | GACAGUGGUGGGGCCUGGAAAAGAUUUCAAAU |
| ENST000 | | NO: | |
| 00327307- | | 156 | |
| 67 | | | |
| ENSG000 | 1.12826237 | SEQ | AGCUACAGCUACCUCCGGACGCUCUCAAG |
| 00086589- | | ID | |
| ENST000 | | NO: | |
| 00518917- | | 157 | |
| 29 | | | |
| ENSG000 | 1.107517003 | SEQ | CAGAUACUUUGGUGUUGAACAUUUGGGAUAGAAGA |
| 00086666- | | ID | GAGAAAUUUAAAAUUAAAAAUUUUCCAUAUUUCUA |
| ENST000 | | NO: | UCACAAAUAUUGGUGUGCAACUGAGGAAC |
| 00558087- | | 158 | |
| 99 | | | |
| ENSG000 | 1.599711439 | SEQ | AGCUUCAAGUGAAGUUAAUCUGAGGUGAAGCAAAC |
| 00086666- | | ID | AGAAAAUUGUGGAGGUUUUGUUGGUGGAGAUAAGU |
| ENST000 | | NO: | AAUUAACAUUUCGAAACAGGAA |
| 00558390- | | 159 | |
| 92 | | | |
| ENSG000 | 1.284347896 | SEQ | GAAGAGAGAAAUUUAAAAUUAAAAAUUUUCCAUAU |
| 00086666 | | ID | UUCUAUCACAAAUAUUGGUAAUUUGACAACAGUUA |
| ENST000 | | NO: | AUUUGAUUGUCGUCUUAAUAAAAUUUA |
| 00558724- | | 160 | |
| 97 | | | |
| ENSG000 | 1.216464705 | SEQ | ACAGCUUCAAGUGAAGUUAAUCUGAGGUGAAGCAA |
| 00086666- | | ID | ACAGAAAAUUGUGGAGGUUUUGUUGGUGGAGAUAA |
| ENST000 | | NO: | GUAAUUAACAUUUCGAAACAGGAA |
| 00560976- | | 161 | |
| 94 | | | |
| ENSG000 | 1.471574188 | SEQ | CAGAUUUGUUUUCCUCUGAGUGGUUUUCAGCUUGC |
| 00086991- | | ID | UGGUUAAUUGAAA |
| ENST000 | | NO: | |
| 00528341- | | 162 | |
| 48 | | | |
| ENSG000 | 2.371610223 | SEQ | AAGCGGCGAGUCUCC |
| 00087157- | | ID | |
| ENST000 | | NO: | |
| 00589689- | | 163 | |
| 15 | | | |
| ENSG000 | 1.108833409 | SEQ | AUAUACGGGCUCCAGGCUGAACGGCUCGGGCCACUU |
| 00087237- | | ID | ACACACCACUGCCUGAUAACC |
| ENST000 | | NO: | |
| 00379780- | | 164 | |
| 57 | | | |
| ENSG000 | 1.362579746 | SEQ | UUCAGCAACCCGUCUGAACAGAUCUGGCGUCCUGGA |
| 00087250- | | ID | GAAUUCUAGA |
| ENST000 | | NO: | |
| 00561640- | | 165 | |
| 46 | | | |
| ENSG000 | 1.18848933 | SEQ | CGCGCCUUCCUGCGGCUAAG |
| 00087365- | | ID | |
| ENST000 | | NO: | |
| 00524627- | | 166 | |
| 20 | | | |
| ENSG000 | 1.595928126 | SEQ | ACAAUCUGCCUCUGAAGCUGGAGAUACUAGCUGCA |
| 00087589- | | ID | GAGCUCAGGGGAGCUGCUCCACAUCACCGAC |
| ENST000 | | NO: | |
| 00679529- | | 167 | |
| 66 | | | |
| ENSG000 | 1.129908239 | SEQ | CCUUUUAUAAGUAACCUACUACUCCUGCAAUAACA |
| 00087884- | | ID | GCAUAAUCUAUUC |
| ENST000 | | NO: | |
| 00525034- | | 168 | |
| 48 | | | |
| ENSG000 | 1.409121561 | SEQ | UUCUGGAGGCUGGGAAGUCCAAGACCAGUUGGCUC |
| 00087884- | | ID | GCAUCUGACUUCAGUGAAGUUCCUU |
| ENST000 | | NO: | |
| 00533193- | | 169 | |
| 60 | | | |
| ENSG000 | 1.355750854 | SEQ | CCCGCUUCCAAA |
| 00088682- | | ID | |
| ENST000 | | NO: | |
| 00567072- | | 170 | |
| 12 | | | |
| ENSG000 | 1.531612167 | SEQ | CAGGAAGCUAGCAGAGUUUGCUUCUGAGACGAAUU |
| 00088756- | | ID | CUGUUACAGUACAGUGCAUUUAGUAGGAUCAAGUU |
| ENST000 | | NO: | AAUU |
| 00532996- | | 171 | |
| 74 | | | |
| ENSG000 | 1.504027167 | SEQ | AUUGCAGCAAGUAAGCCAACAAGGUCUUUUCAGG |
| 00088926- | | ID | |
| ENST000 | | NO: | |
| 00264692- | | 172 | |
| 34 | | | |
| ENSG000 | 1.313077944 | SEQ | AGAACAGACGUUUGAACAGAGCAGGCUUCUGAGGU |
| 00089012 | | ID | CUCCAAA |
| ENST000 | | NO: | |
| 00344103- | | 173 | |
| 42 | | | |
| ENSG000 | 1.138740766 | SEQ | GUUGCCACUCUCUCUCCUGUCA |
| 00089127- | | ID | |
| ENST000 | | NO: | |
| 00550689- | | 174 | |
| 22 | | | |
| ENSG000 | 1.110939792 | SEQ | ACUAUCGCUUACCUACCUCCCUCUGCAGGAACCCGG |
| 00089248- | | ID | CGAU |
| ENST000 | | NO: | |
| 00261735- | | 175 | |
| 40 | | | |
| ENSG000 | 1.294803529 | SEQ | GCUAAGUUUUUGGCCGCUUCAAG |
| 00090316- | | ID | |
| ENST000 | | NO: | |
| 00264750- | | 176 | |
| 23 | | | |
| ENSG000 | 1.175153364 | SEQ | AGCACUCUGACCUAGCAGUCAAC |
| 00090382- | | ID | |
| ENST000 | | NO: | |
| 00548839- | | 177 | |
| 23 | | | |
| ENSG000 | 1.26417695 | SEQ | CUAGCACUCUGACCUAGCAGUCAAC |
| 00090382- | | ID | |
| ENST000 | | NO: | |
| 00549690- | | 178 | |
| 25 | | | |
| ENSG000 | 1.291714013 | SEQ | UUAUUUUGGCAGCCUUAUCCAAGUCUGGUACAACA |
| 00090402- | | ID | UAGCAAAGAGAACAGGCUAGUAAAUAAG |
| ENST000 | | NO: | |
| 00476593- | | 179 | |
| 63 | | | |
| ENSG000 | 1.276220722 | SEQ | ACAAGCCAGCCAGUCCCUGCAGCUCCACAAACUGAC |
| 00090512- | | ID | CCAUCCUGGGCCUUGUUCUCCACAGA |
| ENST000 | | NO: | |
| 00265029- | | 180 | |
| 62 | | | |
| ENSG000 | 1.113273781 | SEQ | CCAACUGGGUCCUGAAGUAGCUGAA |
| 00091844- | | ID | |
| ENST000 | | NO: | |
| 00367225- | | 181 | |
| 25 | | | |
| ENSG000 | 1.703707313 | SEQ | AUUGCAUCACUGCACUCCAGCUUGUAGUACAGUGA |
| 00092199- | | ID | GACAGUGUCUUAAGGAAAG |
| ENST000 | | NO: | |
| 00557157- | | 182 | |
| 54 | | | |
| ENSG000 | 1.476660275 | SEQ | AAGGAGUUCAAGUUGCUUGCCAGACUAACUUACUU |
| 00092871- | | ID | UUAAAAGUAAGCAGCCUUCCAAGUUAAGAGAGUAU |
| ENST000 | | NO: | UUUAAAUUUCAGAUUUUAUC |
| 00413582- | | 183 | |
| 90 | | | |
| ENSG000 | 1.230131751 | SEQ | CAGCCCAUCCACAACCUGCUC |
| 00093010- | | ID | |
| ENST000 | | NO: | |
| 00677397- | | 184 | |
| 21 | | | |
| ENSG000 | 1.796093904 | SEQ | UUUUUCCAGUGAAACAAAACGUAAGAAUCUGAGUU |
| 00093134- | | ID | UGUUUUUCAAAGAUCACUAAAUUUUAGUU |
| ENST000 | | NO: | |
| 00414302- | | 185 | |
| 64 | | | |
| ENSG000 | 1.10489472 | SEQ | UUCUUGGCCUUGGCAAACUUUGGAGAUUGGGGACA |
| 00094841- | | ID | GCCAGUAGAGGUGACUUC |
| ENST000 | | NO: | |
| 00530743- | | 186 | |
| 53 | | | |
| ENSG000 | 1.391468642 | SEQ | GCGUCUGAGCAUUGUUAGUCGUUAUUUUCAAGCUU |
| 00095777- | | ID | UGCUAACGGUUUUUAAACCUUUGAG |
| ENST000 | | NO: | |
| 00543632- | | 187 | |
| 60 | | | |
| ENSG000 | 1.308773902 | SEQ | UGCAGUUUUUAAGAAGAAAAAGAAGGCCCUGAGUC |
| 00095951- | | ID | AAAGAAG |
| ENST000 | | NO: | |
| 00484210- | | 188 | |
| 42 | | | |
| ENSG000 | 1.627930576 | SEQ | AAAACAGGAAAUAGGUGUUUCAUAUAUACGGCUCU |
| 00096006- | | ID | AACCUUCUCUCUCUGCACCUUCCUUCUGUCAAUAG |
| ENST000 | | NO: | |
| 00354620- | | 189 | |
| 70 | | | |
| ENSG000 | 1.424151377 | SEQ | ACCUUCCUUCUGUCAAUAG |
| 00096006- | | ID | |
| ENST000 | | NO: | |
| 00371159- | | 190 | |
| 19 | | | |
| ENSG000 | 1.423812077 | SEQ | AUCUUGCUUGUAGUCCUCUUUCCUUUUCACCUUUCC |
| 00099364- | | ID | AUUGUUCCUGACGAGUAGAA |
| ENST000 | | NO: | |
| 00562798- | | 191 | |
| 56 | | | |
| ENSG000 | 1.107438314 | SEQ | ACAGCAGACGUACCCUCCCUCGCUGCCUGCCUGCAG |
| 00099769- | | ID | CCUGCCCUGCAGUCAGG |
| ENST000 | | NO: | |
| 00215539- | | 192 | |
| 53 | | | |
| ENSG000 | 1.488372727 | SEQ | ACCAGCCCAGCCUUUCAGUGCAGGCUCCAGCCCUCC |
| 00100362- | | ID | ACCCCCACCCGAGUUGCAGG |
| ENST000 | | NO: | |
| 00216200- | | 193 | |
| 56 | | | |
| ENSG000 | 1.734491128 | SEQ | UUAUUACCUCCCUUUUAAAGAGUAGGAAGCUCUGA |
| 00100439- | | ID | CACAGAAAAGCUAAAUA |
| ENST000 | | NO: | |
| 00545034- | | 194 | |
| 52 | | | |
| ENSG000 | 1.217321134 | SEQ | AAGAAAUAGAACAGAAUUCAGCC |
| 00100592- | | ID | |
| ENST000 | | NO: | |
| 00395125- | | 195 | |
| 23 | | | |
| ENSG000 | 1.442046225 | SEQ | AUAUAGUAGUCAGCAACCGGAGGAGUCACGUUGGG |
| 00100625- | | ID | GGAACGGCAGAAAGCAGCUAUCCGUUUACACUACU |
| ENST000 | | NO: | UUGCUCUAACAGCUAUCUUA |
| 00556952- | | 196 | |
| 90 | | | |
| ENSG000 | 1.181338232 | SEQ | GCGGAGGGAGUGGAACCUCGAUAUUGGUGGUGUCC |
| 00100714- | | ID | AUCGUGGGCAGCGGACUAAUAAAGGCC |
| ENST000 | | NO: | |
| 00651537- | | 197 | |
| 62 | | | |
| ENSG000 | 1.166937751 | SEQ | CCAGCGAAGACAGGCUGAG |
| 00100767- | | ID | |
| ENST000 | | NO: | |
| 00216658- | | 198 | |
| 19 | | | |
| ENSG000 | 1.637845248 | SEQ | AGAACCGUUCGCGGAGGAAAGGCGAACUAGUGUUG |
| 00100983- | | ID | GG |
| ENST000 | | NO: | |
| 00643443- | | 199 | |
| 37 | | | |
| ENSG000 | 1.15349591 | SEQ | ACAGUUGUGUUGGGCUCACACCAGUGAACCGAAGC |
| 00101162- | | ID | UCUGGAUUCUGAGAGUCUGAGGAUUCCGUGAAGAU |
| ENST000 | | NO: | CUCAGACUUGGGCUCAGAGCAAGG |
| 00217133- | | 200 | |
| 94 | | | |
| ENSG000 | 1.464676374 | SEQ | AGCCUCGUGACCUUUUACCCCAAC |
| 00101452- | | ID | |
| ENST000 | | NO: | |
| 00252011- | | 201 | |
| 24 | | | |
| ENSG000 | 1.152621177 | SEQ | ACUAGGGCAAUCUGCCGGAAGAGUCAGGUUCUGUG |
| 00101473- | | ID | UAGUUCUCCGCGUCUUCCGCGGAGCGGGUGUGCAG |
| ENST000 | | NO: | GGCCUGCAGCAUUGAACUAG |
| 00481938- | | 202 | |
| 90 | | | |
| ENSG000 | 1.286831533 | SEQ | CCUGGCAAUCCC |
| 00101542- | | ID | |
| ENST000 | | NO: | |
| 00536675- | | 203 | |
| 12 | | | |
| ENSG000 | 1.287310106 | SEQ | AGUGUUGGUUCAGUAAGUUUUACCAUCAAUUCAAG |
| 00101654- | | ID | UAAUCAUAA |
| ENST000 | | NO: | |
| 00262173- | | 204 | |
| 44 | | | |
| ENSG000 | 1.447930373 | SEQ | AAGUAGCGCUGCGGUUGGCAGCGGCGGG |
| 00101751- | | ID | |
| ENST000 | | NO: | |
| 00406285- | | 205 | |
| 28 | | | |
| ENSG000 | 1.155649303 | SEQ | ACUCUCUGUUCUUGCUAUCUAAGUGCCGCUGCUUCC |
| 00101812- | | ID | GCC |
| ENST000 | | NO: | |
| 00355016- | | 206 | |
| 39 | | | |
| ENSG000 | 1.368781976 | SEQ | AAUUUGAGACUCAGCCUAGUCCAGGCAAGCUACUG |
| 00101842- | | ID | GCACCUGCUGCUCUCAACUAACCUCCACACA |
| ENST000 | | NO: | |
| 00217957- | | 207 | |
| 66 | | | |
| ENSG000 | 1.294937074 | SEQ | GGAAUCGGGAGAUUCGGGACC |
| 00101868- | | ID | |
| ENST000 | | NO: | |
| 00677890- | | 208 | |
| 21 | | | |
| ENSG000 | 1.520470786 | SEQ | AGACUGUUCCAAG |
| 00101935- | | ID | |
| ENST000 | | NO: | |
| 00372059- | | 209 | |
| 13 | | | |
| ENSG000 | 1.201193262 | SEQ | UUACAAGGAUCACAGGGGUUGGUACAAGUUGCUUG |
| 00101974- | | ID | AUAAUCUGCAGCAUUCUCUCUGAGAUUUAGCUGCC |
| ENST000 | | NO: | AUUGUGUUGCCAAAUUUCUGCAGUUCAGU |
| 00327569- | | 210 | |
| 99 | | | |
| ENSG000 | 1.253705718 | SEQ | ACACGCGCCCUACCUCGGAGUGUGUGGCGCCAGUAG |
| 00102021- | | ID | UCAGGGAAG |
| ENST000 | | NO: | |
| 00371920- | | 211 | |
| 45 | | | |
| ENSG000 | 1.924927925 | SEQ | GUUAGUUCUACAGAAGAGGCAGAAAAACAAGAGAU |
| 00102271- | | ID | AACAAAGGCUCCGUUUCCUUUCUGUGAGAGAAGGC |
| ENST000 | | NO: | UUUUGUCUUUCCUCCUGCUACG |
| 00373119- | | 212 | |
| 92 | | | |
| ENSG000 | 1.180478522 | SEQ | ACGAGGUGGGCC |
| 00102387- | | ID | |
| ENST000 | | NO: | |
| 00372907- | | 213 | |
| 12 | | | |
| ENSG000 | 1.126298746 | SEQ | CAGACUGUGGUUUUGUGCUUGCUCACCAAAGCUAA |
| 00102452- | | ID | CCUCAGC |
| ENST000 | | NO: | |
| 00675594- | | 214 | |
| 42 | | | |
| ENSG000 | 1.335722854 | SEQ | GGCAGCGGGACGCACCAUUUCAGUUGUGUUCUUGG |
| 00102738- | | ID | UUCAUUUCGUGUCUCGGCG |
| ENST000 | | NO: | |
| 00323563- | | 215 | |
| 54 | | | |
| ENSG000 | 1.716041113 | SEQ | AAAGGGGCCUUCAAGAAGAGAGUUCUCAGAGCAGA |
| 00102780- | | ID | CUCAGCUUUAGGUGCUGAAAGUAAUUUCUCAAGAU |
| ENST000 | | NO: | CAUCACUCCAUUCAGAAGGCUA |
| 00626247- | | 216 | |
| 92 | | | |
| ENSG000 | 1.340489447 | SEQ | ACUCAGCCUCCCUUCCCCAGCCGUGACAGCACUGGA |
| 00102891- | | ID | GCCUUUCGGACACCUGGACC |
| ENST000 | | NO: | |
| 00219162- | | 217 | |
| 56 | | | |
| ENSG000 | 1.563641032 | SEQ | UUCCUUACCUGCUUUGAGUCUGUGGAAGGAGACAG |
| 00102900- | | ID | GAAUUCUGGAUCUGCAUCUCCA |
| ENST000 | | NO: | |
| 00568656- | | 218 | |
| 57 | | | |
| ENSG000 | 1.663197806 | SEQ | CUUGGACCUUGGCCCUCGCUUUCCAGG |
| 00103269- | | ID | |
| ENST000 | | NO: | |
| 00219551- | | 219 | |
| 27 | | | |
| ENSG000 | 1.189560838 | SEQ | GAGAAUCAGAUUGUGCCCACAAGGCGGAAAGUUAC |
| 00103274- | | ID | GACAGCGGGUUCCGGUGACCACGAAGGCGGCAAAG |
| ENST000 | | NO: | GCGACGGA |
| 00574334- | | 220 | |
| 78 | | | |
| ENSG000 | 1.267516878 | SEQ | AUUCCAUCCCAC |
| 00103356- | | ID | |
| ENST000 | | NO: | |
| 00563499- | | 221 | |
| 12 | | | |
| ENSG000 | 1.242641463 | SEQ | ACGUGUGCUAUUCCAUCCCAC |
| 00103356- | | ID | |
| ENST000 | | NO: | |
| 00564668- | | 222 | |
| 21 | | | |
| ENSG000 | 1.185296402 | SEQ | GCAGCAGUUUUCUACGUCUGAAAUUUUUUAGUUCU |
| 00103429- | | ID | CUGGAACCCAGAAUUUGCUAAGAG |
| ENST000 | | NO: | |
| 00566710- | | 223 | |
| 59 | | | |
| ENSG000 | 1.377359015 | SEQ | ACAUUAAAAAUAACAUUUUUCUCUCUUGUAGUGUG |
| 00103540- | | ID | ACUGUGGGAAG |
| ENST000 | | NO: | |
| 00396208- | | 224 | |
| 46 | | | |
| ENSG000 | 1.215285335 | SEQ | GCUGUCCUAGGAUUGCUGGCUUUUGUUUUUGCUGC |
| 00104081- | | ID | UGAGCCUGGAGCUCAGCUCAGACUGAGAGGAGAG |
| ENST000 | | NO: | |
| 00561360- | | 225 | |
| 69 | | | |
| ENSG000 | 1.224564945 | SEQ | GUAGUACGAAUCCGUCAGGCCGGAACC |
| 00104129- | | ID | |
| ENST000 | | NO: | |
| 00220496- | | 226 | |
| 27 | | | |
| ENSG000 | 1.564981531 | SEQ | UUAAAAAAACAGUUUCCUGCUAGUGCCCUGAAGAU |
| 00104205- | | ID | UCCUGCCAAGAGAAUAUUUGGUGAUAAUUUUGAUC |
| ENST000 | | NO: | CAGUCCAGAGUUCAGAGCAUUCCUUCAA |
| 00519396- | | 227 | |
| 98 | | | |
| ENSG000 | 1.46221118 | SEQ | UGUUGCAGACCCAGGAGUCUCUGAAGGCUUCCGCG |
| 00104221- | | ID | |
| ENST000 | | NO: | GCUUUGGGGCCGGUUGUGAG |
| 00520601- | | 228 | |
| 55 | | | |
| ENSG000 | 1.32565235 | SEQ | GAAGACGCUUCCUGGGUUUGAGGAGUUCAGUGACU |
| 00104299- | | ID | GCUAUUGAACCACCAAAAGUCCAUU |
| ENST000 | | NO: | |
| 00522363- | | 229 | |
| 60 | | | |
| ENSG000 | 1.370427866 | SEQ | GUGCAAACAUCUCAAGCCAGUUCAGAGUUUGCUGU |
| 00104313- | | ID | UUCCUCAAGUUGCAGGUCU |
| ENST000 | | NO: | |
| 00419131- | | 230 | |
| 54 | | | |
| ENSG000 | 1.16477741 | SEQ | CUUUUCUUUGGCAAG |
| 00104408- | | ID | |
| ENST000 | | NO: | |
| 00220849- | | 231 | |
| 15 | | | |
| ENSG000 | 1.790039456 | SEQ | ACAGACUCCCUUUUCUUUGGCAAG |
| 00104408- | | ID | |
| ENST000 | | NO: | |
| 00519627- | | 232 | |
| 24 | | | |
| ENSG000 | 1.361434181 | SEQ | AGACUCCCUUUUCUUUGGCAAG |
| 00104408- | | ID | |
| ENST000 | | NO: | |
| 00678042- | | 233 | |
| 22 | | | |
| ENSG000 | 1.169183735 | SEQ | CUUCCGCGUGC |
| 00104687- | | ID | |
| ENST000 | | NO: | |
| 00221130- | | 234 | |
| 11 | | | |
| ENSG000 | 2.021321532 | SEQ | UUGGCUGCUAGAGCG |
| 00104763- | | ID | |
| ENST000 | | NO: | |
| 00518087- | | 235 | |
| 15 | | | |
| ENSG000 | 1.260127344 | SEQ | AGUGGCGUUGGCUGCUAGAGCG |
| 00104763- | | ID | |
| ENST000 | | NO: | |
| 00637603- | | 236 | |
| 22 | | | |
| ENSG000 | 1.189060489 | SEQ | UGGCGUUGGCUGCUAGAGCG |
| 00104763- | | ID | |
| ENST000 | | NO: | |
| 00637638- | | 237 | |
| 20 | | | |
| ENSG000 | 1.346252571 | SEQ | ACCCGCAAACCAGAUCGCCGCUCCGGAGCCCCCAGC |
| 00104833- | | ID | CCACUGGGAGGGCUCGCGGUUACUGCAAGGAUUCU |
| ENST000 | | NO: | GGCGAAGUAGGUCUAAAUAGA |
| 00598635- | | 238 | |
| 92 | | | |
| ENSG000 | 2.107429577 | SEQ | ACCCGGAGCGGGAAG |
| 00104853- | | ID | |
| ENST000 | | NO: | |
| 00591304- | | 239 | |
| 15 | | | |
| ENSG000 | 1.137899399 | SEQ | GGUCCUAAUUCGGGAAACGUGCACCUUGACUAAUU |
| 00104907- | | ID | CGGUUUUCUGUGGCUACGAGAGCAGGCUUGGCGGG |
| ENST000 | | NO: | CGGAGGCGCCAGCGGAGUUCUC |
| 00592814- | | 240 | |
| 92 | | | |
| ENSG000 | 1.383285769 | SEQ | ACACGAUUCUCCAAC |
| 00104980- | | ID | |
| ENST000 | | NO: | |
| 00270538- | | 241 | |
| 15 | | | |
| ENSG000 | 1.094498183 | SEQ | UCUAGUCCACGCUCGGAGCC |
| 00105193- | | ID | |
| ENST000 | | NO: | |
| 00599539- | | 242 | |
| 20 | | | |
| ENSG000 | 1.408944673 | SEQ | AACACCCUCCUGGAGG |
| 00105219- | | ID | |
| ENST000 | | NO: | |
| 00221818- | | 243 | |
| 16 | | | |
| ENSG000 | 1.419375769 | SEQ | GGAAAAGCUCGAUAAUUACCCAGCCUAACCAUUUC |
| 00105248- | | ID | UCAGGUGCUUGCGAGGUGAUCAGAAGGCAAAG |
| ENST000 | | NO: | |
| 00262962- | | 244 | |
| 67 | | | |
| ENSG000 | 3.405465052 | SEQ | AAGACGCCAAG |
| 00105278- | | ID | |
| ENST000 | | NO: | |
| 00438164- | | 245 | |
| 11 | | | |
| ENSG000 | 1.471425722 | SEQ | UAAGACCGGCCCCUUCCGGUUACGAAACCUUAGCAA |
| 00105341- | | ID | G |
| ENST000 | | NO: | |
| 00592922- | | 246 | |
| 37 | | | |
| ENSG000 | 1.252950824 | SEQ | CUUCCUUUGACAAG |
| 00105373- | | ID | |
| ENST000 | | NO: | |
| 00246802- | | 247 | |
| 14 | | | |
| ENSG000 | 2.147015895 | SEQ | ACUCCCUUCCUCUUUUCUGCUCACACAGGAAGCCCU |
| 00105383- | | ID | GGAAGCUGCUUCCUCAGAC |
| ENST000 | | NO: | |
| 00421133- | | 248 | |
| 55 | | | |
| ENSG000 | 1.161684562 | SEQ | AGCCUCCAGCGGUGGCUACAGUUGAAACCCUCACAC |
| 00105398- | | ID | CACGCAGGAAGAGGUCAUCAUC |
| ENST000 | | NO: | |
| 00222002- | | 249 | |
| 58 | | | |
| ENSG000 | 1.28954586 | SEQ | ACAGCAGCUCUACCCCUCACGACGCAGAC |
| 00105404- | | ID | |
| ENST000 | | NO: | |
| 00222008- | | 250 | |
| 29 | | | |
| ENSG000 | 1.196237228 | SEQ | GGGUUUGGUGGCAGAAGGAGAGUGUGGGGGAGGCA |
| 00105467- | | ID | GGGGUCUUCUCCCUGAUCGUCUUCUCCUCCCUGCUG |
| ENST000 | | NO: | ACCGACGGCUACCAGAACAAG |
| 00600863- | | 251 | |
| 92 | | | |
| ENSG000 | 1.127089717 | SEQ | CUUCGACUCGCU |
| 00105583- | | ID | |
| ENST000 | | NO: | |
| 00596731- | | 252 | |
| 12 | | | |
| ENSG000 | 1.153290946 | SEQ | CCCCUGCCUCUGAGG |
| 00105605- | | ID | |
| ENST000 | | NO: | |
| 00391766- | | 253 | |
| 15 | | | |
| ENSG000 | 1.209464013 | SEQ | AGAGGACACUUCCUUUUGCGGGUGGCGGCGAACGC |
| 00105640- | | ID | GGAGAGCACGCC |
| ENST000 | | NO: | |
| 00222247- | | 254 | |
| 47 | | | |
| ENSG000 | 2.004472198 | SEQ | AAGAGGAGCUGGUGAGAAGACAGCGAA |
| 00105669- | | ID | |
| ENST000 | | NO: | |
| 00351079- | | 255 | |
| 27 | | | |
| ENSG000 | 1.312591204 | SEQ | AUUGUGGGAUUGG |
| 00105705- | | ID | |
| ENST000 | | NO: | |
| 00247001- | | 256 | |
| 13 | | | |
| ENSG000 | 1.168007566 | SEQ | CCUGUUGCAGCC |
| 00105793- | | ID | |
| ENST000 | | NO: | |
| 00439832- | | 257 | |
| 12 | | | |
| ENSG000 | 1.248962899 | SEQ | GUGGGAACCUUGACAAGCAGC |
| 00105849- | | ID | |
| ENST000 | | NO: | |
| 00222567- | | 258 | |
| 21 | | | |
| ENSG000 | 1.220285281 | SEQ | ACUGUGCUCUGCGAGCCGAAUC |
| 00105879- | | ID | |
| ENST000 | | NO: | |
| 00222597- | | 259 | |
| 22 | | | |
| ENSG000 | 1.329508674 | SEQ | AUAAACAUUGGGCUGCACAUAGAGACUUAAUUUUA |
| 00105954- | | ID | GAUUUAGACAAA |
| ENST000 | | NO: | |
| 00222674- | | 260 | |
| 47 | | | |
| ENSG000 | 1.200722879 | SEQ | AUAAAUUGUGCAAGGGUGCUAUAGACGCACAAACG |
| 00106004- | | ID | ACCGCGAGCCACAAAUCAAGCACACAUAUCAAAAAA |
| ENST000 | | NO: | CAA |
| 00222726- | | 261 | |
| 74 | | | |
| ENSG000 | 1.71541121 | SEQ | UAGAUCGUCAGGAAAAGCCUAAAGAUUAGACUGUA |
| 00106028- | | ID | AGAAAAGAAAAUAGAAGCC |
| ENST000 | | NO: | |
| 00465582- | | 262 | |
| 54 | | | |
| ENSG000 | 1.160270956 | SEQ | AUCUUUGCAGAUUAUCCUGUGGAAGGAAA |
| 00106245- | | ID | |
| ENST000 | | NO: | |
| 00456893- | | 263 | |
| 29 | | | |
| ENSG000 | 1.865183436 | SEQ | UACAGCUUUGAUUUUAUAACAUCCUGCUAAUUUUU |
| 00106392- | | ID | GUUCUUACAGAAAUACACUUUCGGGAA |
| ENST000 | | NO: | |
| 00223122- | | 264 | |
| 62 | | | |
| ENSG000 | 1.124579959 | SEQ | AAAAUCAAUUCUCACACGCUGGG |
| 00106484- | | ID | |
| ENST000 | | NO: | |
| 00458161- | | 265 | |
| 23 | | | |
| ENSG000 | 1.341577069 | SEQ | UCAUUGCGGCUGGCGUUCCCAAUACAGACGCAUCGU |
| 00106524- | | ID | UUCUUUUUUAAUACUCCCUAAGAAAGGGAAUAACC |
| ENST000 | | NO: | UUCAAGCUGGCGGGAGCA |
| 00453623- | | 266 | |
| 89 | | | |
| ENSG000 | 1.112496496 | SEQ | CUGGUUCCUCUCGGAGCGGAGACGGCAA |
| 00106682- | | ID | |
| ENST000 | | NO: | |
| 00265753- | | 267 | |
| 28 | | | |
| ENSG000 | 1.243776181 | SEQ | CAAUUCCUGUCUGGAUUAUAUUGCUUUUUCAUUUA |
| 00106771- | | ID | GCAUCCCUGGCUGGCUCAGUGAGAAUUCCAGUAUU |
| ENST000 | | NO: | CCUGGUUAUUGUUUUCCUG |
| 00491854- | | 268 | |
| 89 | | | |
| ENSG000 | 1.116866223 | SEQ | GAGCCGGAGGAGGUGGAGAGUA |
| 00106785- | | ID | |
| ENST000 | | NO: | |
| 00375098- | | 269 | |
| 22 | | | |
| ENSG000 | 1.356659093 | SEQ | ACAGCCUGCGGGGAACGUAGCUAGCUGCAAGCAGA |
| 00107201- | | ID | GGCCGGC |
| ENST000 | | NO | |
| 00679771- | | 270 | |
| 42 | | | |
| ENSG000 | 1.456823895 | SEQ | CUUCUUGGUGAAUUUUUGGAGUAAGCCAUUAAAUU |
| 00107643- | | ID | AAUUGCUUGCCAUC |
| ENST000 | | NO: | |
| 00360332- | | 271 | |
| 49 | | | |
| ENSG000 | 1.45184235 | SEQ | AGUGGUGUGGUACCGGGUACCCGGAGACGUGUAUC |
| 00107651- | | ID | GGACGGUGGGCCGCAGCC |
| ENST000 | | NO: | |
| 00369075- | | 272 | |
| 53 | | | |
| ENSG000 | 1.160501833 | SEQ | AAGCUCUUACUGCAGGCAGAUUGAAGCGUCUUACU |
| 00107771- | | ID | UUCCGAAAAUUGGAGUGUGACAAU |
| ENST000 | | NO: | |
| 00493409- | | 273 | |
| 59 | | | |
| ENSG000 | 1.155153638 | SEQ | GUGAGCGGCAAC |
| 00107949- | | ID | |
| ENST000 | | NO: | |
| 00278100- | | 274 | |
| 12 | | | |
| ENSG000 | 1.114402864 | SEQ | AUUUCAACACUACACUUGCACA |
| 00108094- | | ID | |
| ENST000 | | NO: | |
| 00374746- | | 275 | |
| 22 | | | |
| ENSG000 | 1.183590449 | SEQ | AGUUUGCAGUUGAGCCCAAACUGAAGUCUGUCUGU |
| 00108094- | | ID | AGAAGGAAACAACAAACUUUGUACUUU |
| ENST000 | | NO: | |
| 00421317- | | 276 | |
| 62 | | | |
| ENSG000 | 1.098761062 | SEQ | GUUCUCCGUAAG |
| 00108349- | | ID | |
| ENST000 | | NO: | |
| 00264645- | | 277 | |
| 12 | | | |
| ENSG000 | 1.323960217 | SEQ | AUUGUGCCCUACCAGAGAGGACAGAGUGGCACCGCC |
| 00108352- | | ID | UCCAGUGGCA |
| ENST000 | | NO: | |
| 00456989- | | 278 | |
| 46 | | | |
| ENSG000 | 1.567342301 | SEQ | UGCAUUUGGUAUUAGCUAGUGGAAGCAGUAGUUAG |
| 00108375- | | ID | UGUUGAAGUGCAUUGCUGCAGCUGGUAGC |
| ENST000 | | NO: | |
| 00583753- | | 279 | |
| 64 | | | |
| ENSG000 | 1.711817659 | SEQ | AGCCGGAGCCGUGGCCUGCGGGGCCGGCGAC |
| 00108433- | | ID | |
| ENST000 | | NO: | |
| 00638219- | | 280 | |
| 31 | | | |
| ENSG000 | 1.348128084 | SEQ | CUUUAUUAGAGAGAUACUUGUUGACCGACGAAGUC |
| 00108852- | | ID | AGUGGGGCAGCCGCUAAGAAUA |
| ENST000 | | NO: | |
| 00518766- | | 281 | |
| 57 | | | |
| ENSG000 | 1.535761109 | SEQ | AUUGGGAGCCUUUGGGGGUUUAUUCUCUUCCCUUC |
| 00108932- | | ID | UAACUUGAUUAAGA |
| ENST000 | | NO: | |
| 00580666- | | 282 | |
| 49 | | | |
| ENSG000 | 1.214309141 | SEQ | CACAAUCUACACAGCAGCCUUGAAGGAAAAGACGCC |
| 00109113- | | ID | AGACUUCAGACGUCUCUCUCCUCGGGCGCCCUGCAA |
| ENST000 | | NO: | GGCCGCAGGCAGG |
| 00412625- | | 283 | |
| 85 | | | |
| ENSG000 | 1.649774593 | SEQ | AAAAUAUCAGAGUCACCAGAGACACAAGGCUUGAA |
| 00109163- | | ID | GCUCUGUCCUGGGAAAAU |
| ENST000 | | NO: | |
| 00226413- | | 284 | |
| 53 | | | |
| ENSG000 | 1.585246933 | SEQ | AAAAGAAUUAUCACAUUGCACAAGG |
| 00109181- | | ID | |
| ENST000 | | NO: | |
| 00458688- | | 285 | |
| 25 | | | |
| ENSG000 | 1.24414818 | SEQ | UUAUAAAGCUAAAGAUCACAACUUAUUUUCUAGAU |
| 00109205- | | ID | AUAUCAUACGAAA |
| ENST000 | | NO: | |
| 00396094- | | 286 | |
| 48 | | | |
| ENSG000 | 1.199283504 | SEQ | AGAGCUGAGAGAGGAAAAGAACACAGAUCUCGCAG |
| 00109205- | | ID | UGUUCAGAUUUUUCUUUUUAGGUCCAGGAGUAAGA |
| ENST000 | | NO: | UAUAUCAUACGAAA |
| 00683306- | | 287 | |
| 84 | | | |
| ENSG000 | 1.137668917 | SEQ | CCGAGGAGCUGAGGAAGCCUAGAGCUCUCAGAAGC |
| 00109475- | | ID | AGUCCUUUGAGCUGGUGUAGGGGCACUCAGA |
| ENST000 | | NO: | |
| 00394668- | | 288 | |
| 66 | | | |
| ENSG000 | 1.177103964 | SEQ | UCCUAUUGGGUUAACACUGGAGGCUCUAAG |
| 00109911- | | ID | |
| ENST000 | | NO: | |
| 00639878- | | 289 | |
| 30 | | | |
| ENSG000 | 1.556481021 | SEQ | ACAGGGCAGACCUGUAGUGUUCGUUUAUUCCUGGG |
| 00110060- | | ID | GUUGUCAUAUC |
| ENST000 | | NO: | |
| 00530811- | | 290 | |
| 46 | | | |
| ENSG000 | 1.620457185 | SEQ | ACCGGCUGAGCC |
| 00110104- | | ID | |
| ENST000 | | NO: | |
| 00227520- | | 291 | |
| 12 | | | |
| ENSG000 | 1.441213556 | SEQ | GGCUCAGAGAGGUUAAGUGACUCAGCCAAGGUCAA |
| 00110200- | | ID | ACAGCUAUCUAGGAGCC |
| ENST000 | | NO: | |
| 00438939- | | 292 | |
| 52 | | | |
| ENSG000 | 1.692791453 | SEQ | UAGUAAGUGGUGGAGCCAGGACUCAAAGCCAGUCU |
| 00110200- | | ID | AGGAGCC |
| ENST000 | | NO: | |
| 00543050- | | 293 | |
| 42 | | | |
| ENSG000 | 1.329757167 | SEQ | AUUAUUCUUUUGAAGAUUCUUCGUUGUCAAGCCGC |
| 00110321- | | ID | CAAA |
| ENST000 | | NO: | |
| 00531647- | | 294 | |
| 39 | | | |
| ENSG000 | 2.178657666 | SEQ | ACAGCGGCUUCCUUGAUCCUUGCCACCCGCGACUGA |
| 00110484- | | ID | ACACCGACAGCAGCAGCCUCACC |
| ENST000 | | NO: | |
| 00227918- | | 295 | |
| 59 | | | |
| ENSG000 | 1.323790599 | SEQ | GUCACUUUCAGUAAUUCAAAGGCAAUUUACCAGUG |
| 00110693- | | ID | AUUUCUGGGUGCUGGGGCUGAUAUUUUUUGUGCAU |
| ENST000 | | NO: | AUUUAAGA |
| 00396356- | | 296 | |
| 78 | | | |
| ENSG000 | 1.146761652 | SEQ | CUUUCGUUGCCUGAUCGCCGCCAUC |
| 00110700- | | ID | |
| ENST000 | | NO: | |
| 00228140- | | 297 | |
| 25 | | | |
| ENSG000 | 1.132076128 | SEQ | UCCCGAUUGACUGGCCUGCUUGGCAAGGCAAGUAG |
| 00110717- | | ID | CGGCGGCGCUUCAAG |
| ENST000 | | NO: | |
| 00526446- | | 298 | |
| 50 | | | |
| ENSG000 | 1.250931727 | SEQ | GUUGUCACUCGACUACCAAG |
| 00110871- | | ID | |
| ENST000 | | NO: | |
| 00445328- | | 299 | |
| 20 | | | |
| ENSG000 | 1.331264455 | SEQ | ACUAGGCGGUUUGGUAACACG |
| 00110880- | | ID | |
| ENST000 | | NO: | |
| 00547294- | | 300 | |
| 21 | | | |
| ENSG000 | 1.254153987 | SEQ | CCGGAAUAGUGAGCCCUGGGCGCGGGGGCGGCGUG |
| 00110911- | | ID | UCAGGUGGUUGCGGAGCUGAAUCAUAUUCUAAGAA |
| ENST000 | | NO: | CUCAGCCACUCAGGUAUCCACC |
| 00546636- | | 301 | |
| 92 | | | |
| ENSG000 | 1.503396046 | SEQ | GCGUGUCAGGUGGUUGCGGAGCUGAAUCAUAUUCU |
| 00110911- | | ID | AAGAACUCAGCCACUCAGGUAUCCACC |
| ENST000 | | NO: | |
| 00547198- | | 302 | |
| 62 | | | |
| ENSG000 | 1.261552878 | SEQ | ACUGUGGUUCUGCGCUUGUCAUC |
| 00111300- | | ID | |
| ENST000 | | NO: | |
| 00549711- | | 303 | |
| 23 | | | |
| ENSG000 | 1.095459425 | SEQ | ACAGGGCAGUGUAGUUCCAGAAAAUAGGACUGACC |
| 00111404- | | ID | AAGAAGCAGAAAAGCAAG |
| ENST000 | | NO: | |
| 00538724- | | 304 | |
| 53 | | | |
| ENSG000 | 1.489070215 | SEQ | AUUAGUAUCUCUUGGGCUGCUGUGAGUGACACACG |
| 00111536- | | ID | CUGAGUGGGGUGAAGGGAA |
| ENST000 | | NO: | |
| 00229134- | | 305 | |
| 54 | | | |
| ENSG000 | 1.613651694 | SEQ | AGGUGAUCAUUUCAAACCAAGCAUCAGCAACAAUU |
| 00111700- | | ID | AAAAAUAUUCACUUGGUAUCUGUAGUUUAAUA |
| ENST000 | | NO: | |
| 00261196- | | 306 | |
| 67 | | | |
| ENSG000 | 1.146171964 | SEQ | CUUGGGGACCCUGGACCACUAUC |
| 00111752- | | ID | |
| ENST000 | | NO: | |
| 00541181- | | 307 | |
| 23 | | | |
| ENSG000 | 1.132594155 | SEQ | GUCCAUCUCAUAGCAGGCACAAACUCAUCCAUCCCC |
| 00112115- | | ID | AGUUGAUUGGAAGAAACAACG |
| ENST000 | | NO: | |
| 00648244- | | 308 | |
| 57 | | | |
| ENSG000 | 1.314688872 | SEQ | GAAGAAGAAGAAGCUUUUGAUUGGACAGUCGCUAG |
| 00112118- | | ID | UCCGACCUCAGAGAACUCAGCCAAUCAUA |
| ENST000 | | NO: | |
| 00229854- | | 309 | |
| 64 | | | |
| ENSG000 | 1.438278383 | SEQ | AACGUCAUCUAGGAGCACCGAGCAGCUUGGCUAAA |
| 00112651- | | ID | AGUAAGGGUGUCGUGCUG |
| ENST000 | | NO: | |
| 00388752- | | 310 | |
| 53 | | | |
| ENSG000 | 1.185669251 | SEQ | AUUAUUUCAUCAUUUGGGCUUGCCCUUUCAGUUGG |
| 00112655- | | ID | AGCACUGUGAUUGGACCCAAGUUGGCAAGAGUGGA |
| ENST000 | | NO: | AGACCAGGGGACAGAACAGAAAUCCCC |
| 00473339- | | 311 | |
| 97 | | | |
| ENSG000 | 1.365249769 | SEQ | CAAACGUGUUUGCGGAAAGGAGUUUGGGUUCCAUC |
| 00112742- | | ID | UUUUCAUUUCCCCAGCGCAGCUUUCUGUAGAA |
| ENST000 | | NO: | |
| 00369798- | | 312 | |
| 67 | | | |
| ENSG000 | 1.184779413 | SEQ | AGUUAAAGUAGGAGUUAGUACUGUUCAGUUUUUUU |
| 00112742- | | ID | CUUAGAA |
| ENST000 | | NO: | |
| 00504040- | | 313 | |
| 42 | | | |
| ENSG000 | 1.476021554 | SEQ | AGAAGUUUGGCUCAAUUAAGAAACAUCAGGGAGAU |
| 00112851- | | ID | AAAUUCAACCCAGUGUGUCUAAAA |
| ENST000 | | NO: | |
| 00508515- | | 314 | |
| 59 | | | |
| ENSG000 | 1.205223684 | SEQ | AAGACUGCAUCCGGCUCCAGGAAAAGCGAGUGGGA |
| 00112874- | | ID | UAUCCCAAUCUUUGGACUGCAUCCUGGUUGCCUCUA |
| ENST000 | | NO: | CUGUGGUCACCUUUGGGAAGAA |
| 00230792- | | 315 | |
| 93 | | | |
| ENSG000 | 1.359402443 | SEQ | CAAGUAUAUUAGCUAGUACAGCACGUAGUAGCCUG |
| 00112964- | | ID | GGGUCCUACAGGU |
| ENST000 | | NO: | |
| 00537449- | | 316 | |
| 48 | | | |
| ENSG000 | 1.281458891 | SEQ | ACUUCGGGGCCAAG |
| 00112983- | | ID | |
| ENST000 | | NO: | |
| 00450756- | | 317 | |
| 14 | | | |
| ENSG000 | 1.119058459 | SEQ | CCUUUUGGUACGCUCCAAG |
| 00113048- | | ID | |
| ENST000 | | NO: | |
| 00261413- | | 318 | |
| 19 | | | |
| ENSG000 | 1.273308197 | SEQ | GUGAGGCCGACAGCUCCAAACUCCGUCCCCAGUCCU |
| 00113141- | | ID | CACUCCUACUCCAAGUG |
| ENST000 | | NO: | |
| 00502899- | | 319 | |
| 53 | | | |
| ENSG000 | 1.593984875 | SEQ | GGAAGACGGCAGUUUGGCGACAUUUCUCGGCCGAA |
| 00113240- | | ID | GGGCCAUUUGCUUUUGCGGAG |
| ENST000 | | NO: | |
| 00611575- | | 320 | |
| 56 | | | |
| ENSG000 | 1.094820619 | SEQ | CCUUUCCUUUCCGCGUGUAGA |
| 00113272- | | ID | |
| ENST000 | | NO: | |
| 00231198- | | 321 | |
| 21 | | | |
| ENSG000 | 1.520153816 | SEQ | ACUUCCGGUUCUCUGUCAGUCGCGAGCGAACGACCA |
| 00113387- | | ID | AGAGGGUGUUCGACUGCUAGAGCCGAGCGAAGCG |
| ENST000 | | NO: | |
| 00265073- | | 322 | |
| 70 | | | |
| ENSG000 | 1.17560556 | SEQ | UGCAUCGUUAGCUUCUCCUGAUAAACUAAUUGCCU |
| 00113520 | | ID | CACAUUGUCACUGCAAAUCGACACCUAUUA |
| ENST000 | | NO: | |
| 00622422 | | 323 | |
| 65- | | | |
| ENSG000 | 1.140745863 | SEQ | AGUCACUUUCUUUGCCAAAGGCAAACGCAGAACGU |
| 00113525- | | ID | UUCAGAGCC |
| ENST000 | | NO: | |
| 00231454- | | 324 | |
| 44 | | | |
| ENSG000 | 1.147296502 | SEQ | AUUGGAACUGUCACCCGGAGGACAGGCUGUCAGAG |
| 00113721- | | ID | CUGGACACC |
| ENST000 | | NO: | |
| 00517957- | | 325 | |
| 44 | | | |
| ENSG000 | 1.633358985 | SEQ | GUUAAAUCAAUUUCUGUUUGCUUUCUUCUCUUCUC |
| 00113916- | | ID | UGUAGGUUUUGAGCAAAAUUUUGGACUGUGAAGCA |
| ENST000 | | NO: | AGGCAUUGGUGAAGACAAA |
| 00430339- | | 326 | |
| 89 | | | |
| ENSG000 | 1.256566246 | SEQ | AAAGUGCUGAGUUAAAGUACAUUUCUGGAUUUGGG |
| 00113924- | | ID | A |
| ENST000 | | NO: | |
| 00476082- | | 327 | |
| 36 | | | |
| ENSG000 | 1.257047904 | SEQ | GUAUUUGAGUUCUACCGUCAGUCCUGGCAUUAUUU |
| 00114013- | | ID | CUCUCUCUACAAGGAGCCUUAGGAGCACU |
| ENST000 | | NO: | |
| 00482356- | | 328 | |
| 64 | | | |
| ENSG000 | 1.242432585 | SEQ | CUUUCUUUUCGCCAUCUUUUGUCUUUCCGUGGAGC |
| 00114391- | | ID | UGUCGCC |
| ENST000 | | NO: | |
| 00394077- | | 329 | |
| 42 | | | |
| ENSG000 | 1.52300545 | SEQ | UUUCCGUGGAGCUGUCGCC |
| 00114391- | | ID | |
| ENST000 | | NO: | |
| 00469605- | | 330 | |
| 19 | | | |
| ENSG000 | 1.231832202 | SEQ | GUGUAGUGUUUUGAGUCCGGCUUCCAUUUAGCGUA |
| 00114416 | | ID | GAAGCAAUUUAAUUUUAAUUAGUUUUUUUCCAAUU |
| ENST000 | | NO: | UAGAAGAUAAUAC |
| 00480918- | | 331 | |
| 83 | | | |
| ENSG000 | 1.253039015 | SEQ | AAUUUGCGCGCUCUCUUUCUGCUGCUCCCCAGCUCU |
| 00114942- | | ID | CGGAUACAGCCGACACC |
| ENST000 | | NO: | |
| 00415904- | | 332 | |
| 53 | | | |
| ENSG000 | 1.178219002 | SEQ | AGUAAGGGUCGUUGGUGGGAAAG |
| 00114988 | | ID | |
| ENST000 | | NO: | |
| 00434865- | | 333 | |
| 23 | | | |
| ENSG000 | 1.444686796 | SEQ | AGAAUAUAACAGCACUCCCAAAGAACUGGGUACUC |
| 00115009- | | ID | AACACUGAGCAGAUCUGUUCUUUGAGCUAAAAACC |
| ENST000 | | NO: | |
| 00358813- | | 334 | |
| 70 | | | |
| ENSG000 | 1.505063683 | SEQ | UAAAAUAGCCCUUCUGUUACUGUCCGGGGCUGCGG |
| 00115365- | | ID | GGUGAGAGGCCAGGGCUUGCUUCCGGCGUC |
| ENST000 | | NO: | |
| 00441020- | | 335 | |
| 65 | | | |
| ENSG000 | 1.534506324 | SEQ | AGAAGCCAACUCAGACUCAGCCAACAGAGAUUGUU |
| 00115386- | | ID | GAUUUGCCUCUUAAGCAAGAGAUUCAUUGCAGCUC |
| ENST000 | | NO: | AGC |
| 00233735- | | 336 | |
| 73 | | | |
| ENSG000 | 1.253091384 | SEQ | GCGAGUCUCUUUGCGGCGCUACACUAGAGCAGAGU |
| 00115541- | | ID | ACGAGUCUGAGGCGGAGGGAGUA |
| ENST000 | | NO: | |
| 00233893- | | 337 | |
| 58 | | | |
| ENSG000 | 1.802128923 | SEQ | AUUUUGUUUUUGUGUUUUUGCAGGGAGGAGCUCUU |
| 00115548- | | ID | CCUGCAGGCGUGGAAACC |
| ENST000 | | NO: | |
| 00427678- | | 338 | |
| 53 | | | |
| ENSG000 | 1.19044608 | SEQ | GACAGCUCCGCGAGCCCCCUACAACUCGUUUCCUUC |
| 00115649- | | ID | CGUUCACCUUCGCAGGGCGGCGACUGGCGGCGCG |
| ENST000 | | NO: | |
| 00451647- | | 339 | |
| 70 | | | |
| ENSG000 | 1.165056902 | SEQ | AACAGUUCCUAGAAA |
| 00115850- | | ID | |
| ENST000 | | NO: | |
| 00264162- | | 340 | |
| 15 | | | |
| ENSG000 | 1.782780655 | SEQ | AUUGCAGGGUCUUUCUGAGUUGAUUUCUUAUAGCA |
| 00115896- | | ID | CCUCUCAAGAAA |
| ENST000 | | NO: | |
| 00487695- | | 341 | |
| 47 | | | |
| ENSG000 | 1.389940816 | SEQ | AAGUAGAAGAAGCCCUUAGCCUAAGUAAGCGAUAA |
| 00115935- | | ID | GAUUUAUCAGCAAGACUGUUGAACGCAUAACUGCC |
| ENST000 | | NO: | CAAG |
| 00436221- | | 342 | |
| 74 | | | |
| ENSG000 | 1.791769809 | SEQ | AUUGCAAUUCCAAAAGUUGUAAGACGGGAUAUUCU |
| 00116044- | | ID | CUUCUGUGCUGUCAAGGGAC |
| ENST000 | | NO: | |
| 00423513- | | 343 | |
| 55 | | | |
| ENSG000 | 1.144305482 | SEQ | AGAAGCGGCAGGAGCAGCGUUGGCACCGGCGAACC |
| 00116106- | | ID | |
| ENST000 | | NO: | |
| 00409854- | | 344 | |
| 35 | | | |
| ENSG000 | 1.202555624 | SEQ | AAGAGAACCCUCGGCGGCAAG |
| 00116151- | | ID | |
| ENST000 | | NO: | |
| 00378531- | | 345 | |
| 21 | | | |
| ENSG000 | 1.168149072 | SEQ | CUCUGCAGCAUAAUUUAGGCAUUGGAGGAGCUGUG |
| 00116171- | | ID | GUUGUAACACUCUACAAG |
| ENST000 | | NO: | |
| 00435345- | | 346 | |
| 53 | | | |
| ENSG000 | 1.756620821 | SEQ | AGAAAGAGUCUGGUUUCGAGUUUUGGAGAUCCUUG |
| 00116539- | | ID | UUUUUUAGUGAACACAGUUCUGUAAAAUUUUCAUA |
| ENST000 | | NO: | AGAUUCCUUGGCAAUAACAUACGCUUGUG |
| 00677042- | | 347 | |
| 99 | | | |
| ENSG000 | 1.186342555 | SEQ | AUUUUGCCUUUUUGUUAGGUUUCCUAAAGACAAAA |
| 00116747- | | ID | AAAA |
| ENST000 | | NO: | |
| 00367441- | | 348 | |
| 39 | | | |
| ENSG000 | 1.533309333 | SEQ | AGUCUCACUUCAGUUCCUUUUGCAGUAAGAGCUCA |
| 00116824- | | ID | GAAUCAAAAGAGGAAACCAACCCCUAAG |
| ENST000 | | NO: | |
| 00369478- | | 349 | |
| 63 | | | |
| ENSG000 | 1.198622228 | SEQ | CUUUUUCUAGCCAGGCUCUCAACUGUCUCCUGCGUU |
| 00117091- | | ID | GCUGGGAAGUUCUGGAAGGAAGC |
| ENST000 | | NO: | |
| 00368046- | | 350 | |
| 59 | | | |
| ENSG000 | 1.198255056 | SEQ | AGAGCUCCCUGUACUUUCCCAACAGUAUCGCUCUUU |
| 00117118- | | ID | AUAUUCAUCGUGA |
| ENST000 | | NO: | |
| 00491274- | | 351 | |
| 49 | | | |
| ENSG000 | 1.681815645 | SEQ | AGAAGGUUUCCGGUUCCGGUGUAACGUUCGGGCUC |
| 00117360- | | ID | CGUCUCAGGGGCUGAAGUUUGUGAGGUGUAGUAUU |
| ENST000 | | NO: | GAGUCCUGUUUGAGCUAUUGUUCUCUUUUUCCUGA |
| 00324862- | | 352 | AAA |
| 108 | | | |
| ENSG000 | 1.599803688 | SEQ | ACCCCGUGGAGCACGCCGAU |
| 00117481- | | ID | |
| ENST000 | | NO: | |
| 00474844- | | 353 | |
| 20 | | | |
| ENSG000 | 1.342415184 | SEQ | AUUACCUGGCUGGUGUUUGCUUGUUCUGGAGUGAU |
| 00117682- | | ID | CUUCUGACUGGAAAAGAACU |
| ENST000 | | NO: | |
| 00430232- | | 354 | |
| 55 | | | |
| ENSG000 | 1.149980607 | SEQ | CUUGCCUUGCGCUGCGCGCUCACC |
| 00117691- | | ID | |
| ENST000 | | NO: | |
| 00366988- | | 355 | |
| 24 | | | |
| ENSG000 | 1.766812098 | SEQ | AGAGCAGAGACC |
| 00118194- | | ID | |
| ENST000 | | NO: | |
| 00367315- | | 356 | |
| 12 | | | |
| ENSG000 | 1.132807482 | SEQ | UCUGUGCUUGAAAUUCUGUGACUCUUCCCUGUAUC |
| 00118260- | | ID | U |
| ENST000 | | NO: | |
| 00457101- | | 357 | |
| 36 | | | |
| ENSG000 | 1.314243435 | SEQ | AGCCUGUGCAAGUGAUUUGGGAGAGGAAGGGGAGA |
| 00118263- | | ID | GUAGUUAACUGGUUCGGC |
| ENST000 | | NO: | |
| 00412414- | | 358 | |
| 53 | | | |
| ENSG000 | 1.398087945 | SEQ | GAGACGCAGAGGCGGACAAG |
| 00118363- | | ID | |
| ENST000 | | NO: | |
| 00263672- | | 359 | |
| 20 | | | |
| ENSG000 | 1.101739656 | SEQ | ACAGUGGUGGGGACAACCUGAGUGCUCAGUCGUAA |
| 00118514- | | ID | AGAGGAAAGGCAGAAUUUUUCCUUGCU |
| ENST000 | | NO: | |
| 00367845- | | 360 | |
| 62 | | | |
| ENSG000 | 1.370569545 | SEQ | UAGCAGAAGCUCUACAAGUAGUAUUCAUUCAGUUU |
| 00118520- | | ID | CUUACUCUGUUGUCCAGGCUAGAGUACAGUGGCAC |
| ENST000 | | NO: | GAUC |
| 00672233- | | 361 | |
| 74 | | | |
| ENSG000 | 1.267324767 | SEQ | CUUCCUCGCUUUCUCUGUCUCUCUGUCUCUCUCUCU |
| 00118526- | | ID | CUCUCUCCCUCGUCCACUCCCCCAAAC |
| ENST000 | | NO: | |
| 00237316- | | 362 | |
| 63 | | | |
| ENSG000 | 1.256610665 | SEQ | GAGGAAGCCGACUAGGCGAAUUCACUUACUGACCG |
| 00118640- | | ID | GCCUGGGCUGCUCUGAGAC |
| ENST000 | | NO: | |
| 00409760- | | 363 | |
| 54 | | | |
| ENSG000 | 1.279088258 | SEQ | CAAGUUUUAAGCAAGAAGAAGGAAAAACUGACAGA |
| 00119397- | | ID | AGAGCUUCAGAAACUACAGAAAGACAUAGAG |
| ENST000 | | NO: | |
| 00373844- | | 364 | |
| 66 | | | |
| ENSG000 | 1.428293972 | SEQ | ACCAGUAAGUCGUGUAUUUAGCAUUCAUUCAUCAA |
| 00119403- | | ID | AGCCUCCGGAGUCCUCCUACGUGCCCUGCACU |
| ENST000 | | NO: | |
| 00616568- | | 365 | |
| 67 | | | |
| ENSG000 | 1.296681277 | SEQ | GUAGGUGAGCUCGUGAAACAAUAGUAAGAGGAGAA |
| 00119487- | | ID | AAUAGCCUUUUAAGGAAAUUGGCCCACAGAAAGG |
| ENST000 | | NO: | |
| 00373498- | | 366 | |
| 69 | | | |
| ENSG000 | 1.145538231 | SEQ | CAGGCCCUCAUCACCUUUUUUCAAGUCAAGAUUUCA |
| 00119508- | | ID | UCCCAUAC |
| ENST000 | | NO: | |
| 00330847- | | 367 | |
| 44 | | | |
| ENSG000 | 1.168101099 | SEQ | UUCUUUCCCGAGCUUGGAACUUCGUUAUCCGCG |
| 00119655- | | ID | |
| ENST000 | | NO: | |
| 00434013- | | 368 | |
| 33 | | | |
| ENSG000 | 1.176659639 | SEQ | UACGCCUCUUCAUUCCCGUCC |
| 00119950- | | ID | |
| ENST000 | | NO: | |
| 00650752- | | 369 | |
| 21 | | | |
| ENSG000 | 1.143186536 | SEQ | AUAAGUUCCUAGUUAGACACAGGACCUGCUGGGCC |
| 00120160- | | ID | ACAGAAAGGAGGCUCUGGGUAGACGCACUAGAUUA |
| ENST000 | | NO: | CUGGAUAAAUCACUUCAAUUUCCCA |
| 00380032- | | 370 | |
| 95 | | | |
| ENSG000 | 1.354852912 | SEQ | UAGCUCAGUGUUGACAGCUCAGAGAGAGAAAGAUC |
| 00120868- | | ID | UGAGGGAAG |
| ENST000 | | NO: | |
| 00552268- | | 371 | |
| 44 | | | |
| ENSG000 | 1.166143999 | SEQ | CGCUGCACUGUCUCUCCGACGCAAGACUGUCCCGGC |
| 00120963- | | ID | CCGGAU |
| ENST000 | | NO: | |
| 00523146- | | 372 | |
| 42 | | | |
| ENSG000 | 1.408580833 | SEQ | ACAGAAGUUAGCCACCUUUCUUAACGGUCCGGGAA |
| 00121207- | | ID | ACCAGAGGUCUCUCCAGCUACUCAGGG |
| ENST000 | | NO: | |
| 00507827- | | 373 | |
| 62 | | | |
| ENSG000 | 1.55909511 | SEQ | GUGUAUUUGAAUAAACCAGGUUGGCAAAUCAUACU |
| 00121270- | | ID | AUAGCUGAAAGAAUUGGCAGGAACUGAAA |
| ENST000 | | NO: | |
| 00353782- | | 374 | |
| 64 | | | |
| ENSG000 | 2.05704384 | SEQ | GUCUAAGAGGCAGACAGAUACAUUGUUCUUCACUG |
| 00121381- | | ID | GUCAGCUGAAAGUCAAUUCAUCAUCAGUGAUAUUC |
| ENST000 | | NO: | AAAAAUCACAAGUCUUUGCUGGC |
| 00240691- | | 375 | |
| 93 | | | |
| ENSG000 | 1.219648877 | SEQ | AUUGCUGCAGACGCUCACCCCAGACACUCACUGCAC |
| 00121410- | | ID | CGGAGUGAGCGCGACCAUC |
| ENST000 | | NO: | |
| 00263100- | | 376 | |
| 55 | | | |
| ENSG000 | 1.257256177 | SEQ | GUUGUUGUAGUUCCGAGAGACACGUGAGGUUCUGC |
| 00121749- | | ID | UACGUCAUUACCAGGCACGCGCAGGAAAC |
| ENST000 | | NO: | |
| 00550746- | | 377 | |
| 64 | | | |
| ENSG000 | 1.400484744 | SEQ | ACAAAUCAAUCCACGGGUACAGAAGUCUGACUUCCC |
| 00121871- | | ID | GACACUCAAUUUCACAGGGUGUCUCUUGAAGAGCU |
| ENST000 | | NO | GCCAGUUUUGUAAUCAACCACGAACG |
| 00497724- | | 378 | |
| 97 | | | |
| ENSG000 | 1.129313299 | SEQ | UAAUUCGCCAAA |
| 00122026- | | ID | |
| ENST000 | | NO: | |
| 00483765- | | 379 | |
| 12 | | | |
| ENSG000 | 1.123234948 | SEQ | AGCCUUGUUCAGACGCCCAGUGACCUGCCGAGGUCG |
| 00122136- | | ID | GCAGCACAGAGCUCUGGAG |
| ENST000 | | NO: | |
| 00371776- | | 380 | |
| 55 | | | |
| ENSG000 | 1.374253728 | SEQ | UAUAUUGAGAGUGUCCACGUCUCCUCGCUGAACCU |
| 00122257- | | ID | UAGGAAUCCCUUGGCACC |
| ENST000 | | NO: | |
| 00646282- | | 381 | |
| 53 | | | |
| ENSG000 | 1.362705352 | SEQ | AGGCGGUGGGGCCCACAGCC |
| 00122481- | | ID | |
| ENST000 | | NO: | |
| 00263893- | | 382 | |
| 20 | | | |
| ENSG000 | 1.297662757 | SEQ | GUCUGGCUAAGUUUGACAAUAGUGGAAAGUGACAA |
| 00122507- | | ID | GAUAAAAUAAUUGUGGGUAGCUUU |
| ENST000 | | NO: | |
| 00425508- | | 383 | |
| 59 | | | |
| ENSG000 | 1.485480999 | SEQ | CUGGUGUAGCUCGUCCGGUUACCUUCAGGAAGAUU |
| 00122643 | | ID | GGAUAACCAAGAA |
| ENST000 | | NO: | |
| 00643244- | | 384 | |
| 48 | | | |
| ENSG000 | 1.14985107 | SEQ | AGUUUUCUUGGAGGCUUUUUUGGUCCAAUUUGUGA |
| 00122958- | | ID | GAUCGAUAUUGUUCUUAAGUAGUGGGAAACCAGGA |
| ENST000 | | NO: | AA |
| 00489794- | | 385 | |
| 72 | | | |
| ENSG000 | 1.659647385 | SEQ | UAAAUAUUUUUUGCUUUAGUUUUUCUUAUAAGGCU |
| 00123219- | | ID | AAAAAUUCACAAAGCAUAUAUCA |
| ENST000 | | NO: | |
| 00242872- | | 386 | |
| 58 | | | |
| ENSG000 | 1.097159879 | SEQ | GAGAGUCAGGUGGAUUCUUGGGCAUUUGCUAUUAA |
| 00123561- | | ID | CUUCCUUCCAAA |
| ENST000 | | NO: | |
| 00372563- | | 387 | |
| 47 | | | |
| ENSG000 | 1.754687177 | SEQ | ACACGCCUUUCUUAUUGGAUACCGGCUUUUCAGUG |
| 00124155 | | ID | GGCCGAGCGCCGCUGGGUAGGCGGAAGUAGCCGCA |
| ENST000 | | NO: | GGC |
| 00279035- | | 388 | |
| 73 | | | |
| ENSG000 | 2.111689527 | SEQ | AAGUAGCCGCAGGC |
| 00124155- | | ID | |
| ENST000 | | NO: | |
| 00638953- | | 389 | |
| 14 | | | |
| ENSG000 | 1.283329662 | SEQ | CUUUCCUGCCUCUGAUUCCGGGCUGUC |
| 00124194- | | ID | |
| ENST000 | | NO: | |
| 00342560- | | 390 | |
| 27 | | | |
| ENSG000 | 1.310536821 | SEQ | ACGACAACUUCCGGAAGAGGUCGCC |
| 00124217- | | ID | |
| ENST000 | | NO: | |
| 00244051- | | 391 | |
| 25 | | | |
| ENSG000 | 1.565674628 | SEQ | UGAGUUUGGGUCAAUCAGUUUCUCAAGUUAUUUAA |
| 00124279- | | ID | CCAGUUGUUUUUAAAUUUUUUAAUUUAGUUAUCAA |
| ENST000 | | NO: | AUUGAGAAUCUGAGUGU |
| 00507036- | | 392 | |
| 87 | | | |
| ENSG000 | 1.12182243 | SEQ | AACUGAGUCAGUUGAAUUACAUAUCUCCCUGAUAG |
| 00124564- | | ID | GUGAAAACCAUUCACUAGGAGAGGAGAAACACA |
| ENST000 | | NO: | |
| 00449356- | | 393 | |
| 68 | | | |
| ENSG000 | 1.402410839 | SEQ | AGUCUGUUCUGACAGUUUGAGAUUACUUAUUGUCU |
| 00124575- | | ID | UUUCUGGGAAGACAAAAAC |
| ENST000 | | NO: | |
| 00244534- | | 394 | |
| 54 | | | |
| ENSG000 | 1.567524235 | SEQ | GCAUCCUGCUUCGUCAGGUUUAUACCACUUUAUUU |
| 00124610- | | ID | GGUGUGCUGUGUUAGUCACC |
| ENST000 | | NO: | |
| 00244573- | | 395 | |
| 55 | | | |
| ENSG000 | 1.384338821 | SEQ | CCGGACCCUGCAGCCGCAGAG |
| 00124614- | | ID | |
| ENST000 | | NO: | |
| 00344700- | | 396 | |
| 21 | | | |
| ENSG000 | 1.234780887 | SEQ | ACGCUGUUUUUCCUUUUCGUUGGCGCUUUAUAGCU |
| 00124635- | | ID | ACACAGUGCU |
| ENST000 | | NO: | |
| 00339812- | | 397 | |
| 45 | | | |
| ENSG000 | 1.097694872 | SEQ | GAGACAGAAGGUUGUGUUGGGGAACUGAAGGAGUU |
| 00124641- | | ID | UCUGUGGGCGGACAGGGAACCCUGCGUUUCUACUG |
| ENST000 | | NO: | UGUGAUUCUGCCACCUUCCUGGCCCGACGCC |
| 00409312- | | 398 | |
| 101 | | | |
| ENSG000 | 1.449876957 | SEQ | AACAGUUUGAGCUCUGGCAUAGAAGAGGCUGGUGG |
| 00124762- | | ID | CUAUUUUGUCCUUGGGCUGCCUGUUUUCAGGCGCC |
| ENST000 | | NO: | |
| 00615513- | | 399 | |
| 70 | | | |
| ENSG000 | 1.173610821 | SEQ | CGGAGCCGCCGAGAGCCUUAGCCGACGGAAACUGGA |
| 00124783- | | ID | CACUGGACCGGCAGCGCC |
| ENST000 | | NO: | |
| 00489567- | | 400 | |
| 54 | | | |
| ENSG000 | 1.23925685 | SEQ | UAGUAAAGUGUUCAAGCUGGCUUAUUCAGGUCUGC |
| 00124818- | | ID | UGAGAUCUCUACUGGUUUGCUUUUCAGAUCCCUCG |
| ENST000 | | NO: | UGGUUCGAGAACAGA |
| 00489301- | | 401 | |
| 85 | | | |
| ENSG000 | 1.855733836 | SEQ | ACAGCAACUUCCUUGAUCCCUGCCACGCACGACUGA |
| 00124939- | | ID | ACACAGACAGCAGCCGCCUCGCC |
| ENST000 | | NO: | |
| 00244930- | | 402 | |
| 59 | | | |
| ENSG000 | 1.58350479 | SEQ | AAAGGAUCAAGCAUCCCUGAGUUUCAAACAGAAAC |
| 00125363- | | ID | UUGCACUGAAUACAUUCAAAGAACCAUCAAGAA |
| ENST000 | | NO: | |
| 00348912- | | 403 | |
| 68 | | | |
| ENSG000 | 1.692324353 | SEQ | UUACUUUCUUACUCUUGGGUCAGUUCCUGCUACAG |
| 00125434- | | ID | CUAUCCCACC |
| ENST000 | | NO: | |
| 00380067- | | 404 | |
| 45 | | | |
| ENSG000 | 1.428100736 | SEQ | CUCAACAGCAGCACUUCCGGGUUGGGAGAAAGGUG |
| 00125482- | | ID | GCGGCGCUUUCGGAGGGAAUAAA |
| ENST000 | | NO: | |
| 00334270- | | 405 | |
| 58 | | | |
| ENSG000 | 1.302825379 | SEQ | AACAACAUCCUGGGAUUGGGACACACUUUCUGGGC |
| 00125551- | | ID | ACUGCUGGCCAGUCCCAAA |
| ENST000 | | NO: | |
| 00410086- | | 406 | |
| 54 | | | |
| ENSG000 | 1.294196952 | SEQ | CUUCAUUCCAUUUUCUGUUGAGUAAUAAACUCAAC |
| 00125571- | | ID | GUUGAAA |
| ENST000 | | NO: | |
| 00349806- | | 407 | |
| 42 | | | |
| ENSG000 | 1.552818327 | SEQ | ACACAUCCGGGCUUCUGCUACUAGUGAGAGGAAG |
| 00125676- | | ID | |
| ENST000 | | NO: | |
| 00355725- | | 408 | |
| 34 | | | |
| ENSG000 | 1.129692343 | SEQ | CCGGCGUUCAAG |
| 00125691- | | ID | |
| ENST000 | | NO: | |
| 00394332- | | 409 | |
| 12 | | | |
| ENSG000 | 1.135389126 | SEQ | CCUUUUUUCUUUUUUCCGGCGUUCAAG |
| 00125691- | | ID | |
| ENST000 | | NO: | |
| 00577407- | | 410 | |
| 27 | | | |
| ENSG000 | 1.125753565 | SEQ | CUCAACACUGGGUCAGAAGUCACGGCCAGACAAGA |
| 00125775- | | ID | AACGGGGUGUUCUGAGAAUCAGCC |
| ENST000 | | NO: | |
| 00339987- | | 411 | |
| 59 | | | |
| ENSG000 | 1.10603883 | SEQ | CUGAGGAGUACAGAGAACACUCUUCCUGUUCCUCU |
| 00125831 | | ID | GCAGCUGAAGG |
| ENST000 | | NO: | |
| 00377007- | | 412 | |
| 46 | | | |
| ENSG000 | 1.606487715 | SEQ | AGAUUCAAGGCUUCCUCUCUGGCACCCAACC |
| 00125903- | | ID | |
| ENST000 | | NO: | |
| 00246105- | | 413 | |
| 31 | | | |
| ENSG000 | 1.191481023 | SEQ | UCCGCUGUGGUCAUUUAGGAAAUCGUAAAUCAGUU |
| 00126091- | | ID | GAAG |
| ENST000 | | NO: | |
| 00645165- | | 414 | |
| 39 | | | |
| ENSG000 | 1.101837922 | SEQ | UUUAGGAAAUCGUAAAUCAGUUGAAG |
| 00126091- | | ID | |
| ENST000 | | NO: | |
| 00646686- | | 415 | |
| 26 | | | |
| ENSG000 | 1.271196805 | SEQ | ACAGUAGGUAGGAAGUCUCAGAGGAAGGAGCCCCU |
| 00126467 | | ID | CCCUGGGAGCCAGACCCCCACACC |
| ENST000 | | NO: | |
| 00246801- | | 416 | |
| 59 | | | |
| ENSG000 | 1.827283611 | SEQ | ACAUUGUGUCUAUUGUAUCCCUUGGCUGGUGUAUU |
| 00126970- | | ID | UGUACAUCUCUCGGGACGUGAAAUUGACAGUGAAA |
| ENST000 | | NO: | AGU |
| 00374839- | | 417 | |
| 73 | | | |
| ENSG000 | 1.756976608 | SEQ | AAGUGAAUAACCAAGUUUAACCUGUUUUAAUUGAA |
| 00127328- | | ID | UUGCAUUUUGACGAAUUAUCAAUAAAA |
| ENST000 | | NO: | |
| 00550847- | | 418 | |
| 62 | | | |
| ENSG000 | 1.829718954 | SEQ | UCAGUGAGGAGAUUCUAGUUAUCAACAGAAAGAAC |
| 00127362- | | ID | AAAGAUCAGGGCUGCCUAAUUGCUGAC |
| ENST000 | | NO: | |
| 00247879- | | 419 | |
| 62 | | | |
| ENSG000 | 1.281544561 | SEQ | AGUGGUUGAUUCUUCACCACACUGAAACCAUUAGG |
| 00127483- | | ID | AAAAAUCCUUGUGGUUAACAGCAGAGGCUUCAGAG |
| ENST000 | | NO: | UGUAACCUGUACUCGGGCCUAGAAAUUAUUUAAA |
| 00312239- | | 420 | |
| 104 | | | |
| ENSG000 | 1.441516297 | SEQ | UAUAGCCUUGCAGAACUUCUACUUGCCUGCCUCCCU |
| 00128218- | | ID | GCCUCUGGCC |
| ENST000 | | NO: | |
| 00398465- | | 421 | |
| 46 | | | |
| ENSG000 | 1.271370506 | SEQ | AUUGCAGCCUCCCUGAUUCCAGAAGGUACCUUGGU |
| 00128242- | | ID | GUCUGAG |
| ENST000 | | NO: | |
| 00427899- | | 422 | |
| 42 | | | |
| ENSG000 | 1.343106118 | SEQ | AUACUGCCUUCCUGUA |
| 00128245- | | ID | |
| ENST000 | | NO: | |
| 00420430- | | 423 | |
| 16 | | | |
| ENSG000 | 1.322318192 | SEQ | AUAAAUACAGCUUGACUCAGCCACUGUAGUACUGA |
| 00128510- | | ID | CUCCCCGGGGAC |
| ENST000 | | NO: | |
| 00445470- | | 424 | |
| 47 | | | |
| ENSG000 | 1.198048305 | SEQ | UCUCUACCUGGAAAUUGGUACAGAGAUUCAGAGUC |
| 00128519- | | ID | UUUGUCCAGGAAGACACUUUGGAGUAGAAGA |
| ENST000 | | NO: | |
| 00249284- | | 425 | |
| 66 | | | |
| ENSG000 | 1.22100676 | SEQ | ACGCACAGUUGUGACCAUCAAGU |
| 00128536- | | ID | |
| ENST000 | | NO: | |
| 00496633- | | 426 | |
| 23 | | | |
| ENSG000 | 1.499517508 | SEQ | UUGAAACCGGGAAGUUUGCUCUAACAUUUCCAGAG |
| 00128573- | | ID | AAGGUAUUAAGUC |
| ENST000 | | NO: | |
| 00393489- | | 427 | |
| 48 | | | |
| ENSG000 | 1.541931367 | SEQ | GUGAGUCUGGGAUUGAAUUUUCCUAGGACUCCGUU |
| 00128573- | | ID | UCAAGGUUAAUUCGAAAGUCUUGUGGAAGAUCUGG |
| ENST000 | | NO: | AAGAAACAGGUAUUAAGUC |
| 00634623- | | 428 | |
| 89 | | | |
| ENSG000 | 1.400013537 | SEQ | CUAGAGAGUUCAGCCUACAAAGGACACAAUCUCUC |
| 00128710- | | ID | UUCUUCAAAUUCUUCCCCAAA |
| ENST000 | | NO: | |
| 00249501- | | 429 | |
| 56 | | | |
| ENSG000 | 1.285459599 | SEQ | CUGAACGGAGUCCACACAGGCAGACUUGACCUUGAC |
| 00129170- | | ID | CAGAUAGUCUUCAAG |
| ENST000 | | NO: | |
| 00647990- | | 430 | |
| 51 | | | |
| ENSG000 | 1.234745603 | SEQ | CCUUUCUCCUGCAGCC |
| 00129221- | | ID | |
| ENST000 | | NO: | |
| 00571740- | | 431 | |
| 16 | | | |
| ENSG000 | 1.989152396 | SEQ | ACAGCGUUGUGCAAAGUAAGUCCUUCCACUGAACC |
| 00129317- | | ID | GAGGCACUGUUAUAGAAGA |
| ENST000 | | NO: | |
| 00553166- | | 432 | |
| 54 | | | |
| ENSG000 | 1.236668508 | SEQ | ACAGAACCGCC |
| 00129347- | | ID | |
| ENST000 | | NO: | |
| 00312962- | | 433 | |
| 11 | | | |
| ENSG000 | 1.501828781 | SEQ | AAGGAACUCUAUCACAAUUUGAAAAGAUAAGCAAA |
| 00129351- | | ID | AGUUUGAUUUCCAGACACUACAGAAGAAGUAAAA |
| ENST000 | | NO: | |
| 00250241- | | 434 | |
| 69 | | | |
| ENSG000 | 1.644735004 | SEQ | AUAGAGUUGAAGUAUUGAUAACACCAAGGAACUCU |
| 00129351- | | ID | AUCACAAUUUGAAAAGAUAAGCAAAAGUUUGAUUU |
| ENST000 | | NO: | CCAGACACUACAGAAGAAGUAAAA |
| 00587505- | | 435 | |
| 94 | | | |
| ENSG000 | 1.38430594 | SEQ | CUCUUCCGUCGCAGAGUUUCGCC |
| 00129824- | | ID | |
| ENST000 | | NO: | |
| 00250784- | | 436 | |
| 23 | | | |
| ENSG000 | 1.601091851 | SEQ | AUACCUUAGACCCUCAGUC |
| 00130173- | | ID | |
| ENST000 | | NO: | |
| 00252453- | | 437 | |
| 19 | | | |
| ENSG000 | 1.325354514 | SEQ | ACACUCGCGGCACAGGCUGACAGUAUUGUCGGGGG |
| 00130224- | | ID | CUAUUCUCUCUCCCAGGAAC |
| ENST000 | | NO: | |
| 00317135- | | 438 | |
| 55 | | | |
| ENSG000 | 1.227032478 | SEQ | AGUCUAGGGAAAGUCAUUCAGUGGAGUUGAUCUUG |
| 00130234- | | ID | GCUCACAGGGGACG |
| ENST000 | | NO: | |
| 00252519- | | 439 | |
| 49 | | | |
| ENSG000 | 1.639319664 | SEQ | ACACAGCUUCUUGGUGCUCCAAAGACGGGCGGUAG |
| 00130592- | | ID | GAGGAAGAUCAAAGAAGCGAGAAAGUG |
| ENST000 | | NO: | |
| 00457279- | | 440 | |
| 62 | | | |
| ENSG000 | 1.706803609 | SEQ | AUUUUAGGCAGCUAUUGCUUCAGAAGUCACAGUCU |
| 00130638- | | ID | GUUCCACCAGCCUUUGCCAAGG |
| ENST000 | | NO: | |
| 00402380- | | 441 | |
| 57 | | | |
| ENSG000 | 1.16505064 | SEQ | GUUGCUGCCGCG |
| 00130731- | | ID | |
| ENST000 | | NO: | |
| 00397666- | | 442 | |
| 12 | | | |
| ENSG000 | 1.153743825 | SEQ | AGCUUCUCCUGGGCCGGCGCC |
| 00130943- | | ID | |
| ENST000 | | NO: | |
| 00253255- | | 443 | |
| 21 | | | |
| ENSG000 | 1.3496519 | SEQ | AUUCAUCUUCCAGGCUCUCCUUCCAUCAAGUCUCUC |
| 00131015- | | ID | AUCCCUAGCGCUCUGGGUCCUUA |
| ENST000 | | NO: | |
| 00367351- | | 444 | |
| 59 | | | |
| ENSG000 | 1.61688911 | SEQ | GAAGAGUAACGCUACUACUUCUGUUUCCUUUUGAG |
| 00131018- | | ID | UCUGCUCUGACACCCCGGUGGGGGAA |
| ENST000 | | NO: | |
| 00537033- | | 445 | |
| 61 | | | |
| ENSG000 | 1.367433332 | SEQ | CCGAACACGAGCACCACAGGCGCCCGAAGGCCGGAA |
| 00131051- | | ID | CAGGCGUUUAGAGAAA |
| ENST000 | | NO: | |
| 00416529- | | 446 | |
| 52 | | | |
| ENSG000 | 1.117929139 | SEQ | AGCAGAGCCAGAGCAGG |
| 00131095- | | ID | |
| ENST000 | | NO: | |
| 00588037- | | 447 | |
| 17 | | | |
| ENSG000 | 1.472465384 | SEQ | AGUCUCUGAAAACUCUUCAGACACUGAGGGGCACC |
| 00131203- | | ID | AGAGGAGCAGACUACAAGA |
| ENST000 | | NO: | |
| 00253513- | | 448 | |
| 54 | | | |
| ENSG000 | 1.196423496 | SEQ | AGCUUCCGGGUUUCCUGGGCUACUACG |
| 00131475- | | ID | |
| ENST000 | | NO: | |
| 00253794- | | 449 | |
| 27 | | | |
| ENSG000 | 1.289764047 | SEQ | CUAGCGGCUCUCCCCGCGUCCAAG |
| 00131558- | | ID | |
| ENST000 | | NO: | |
| 00253861- | | 450 | |
| 24 | | | |
| ENSG000 | 2.021349727 | SEQ | GCGGGGCCUCUACCGGCCCG |
| 00131778- | | ID | |
| ENST000 | | NO: | |
| 00369258- | | 451 | |
| 20 | | | |
| ENSG000 | 1.856306094 | SEQ | AAACCAAAUACUCAGCUGCGUUAGGAAUUCCUUCU |
| 00131981- | | ID | GAUUCUUGCUGCAGGAAA |
| ENST000 | | NO: | |
| 00553493- | | 452 | |
| 53 | | | |
| ENSG000 | 1.500074403 | SEQ | GCAUUGCCGUUUUUGCUCAGAAUCGCGACUAAGGC |
| 00132141- | | ID | UUUUUUUUUUUCUCCCUCUGAACGGUUAGGCU |
| ENST000 | | NO: | |
| 00314144- | | 453 | |
| 67 | | | |
| ENSG000 | 1.568651568 | SEQ | AUUGCCGUUUUUGCUCAGAAUCGCGACUAAGGCUU |
| 00132141- | | ID | UUUUUUUUUCUCCCUCUGAACGGUUAGGCU |
| ENST000 | | NO: | |
| 00436961- | | 454 | |
| 65 | | | |
| ENSG000 | 1.405876675 | SEQ | ACUUCAAGUCUUUUUCUUUUAACGGAUUGAUCUUU |
| 00132170- | | ID | UGCUAGAUAGAGACAAAAUAUCAGUGUGAAUUACA |
| ENST000 | | NO: | GCAAACCCCUAUUCCAGUCUGUU |
| 00683699- | | 455 | |
| 93 | | | |
| ENSG000 | 1.533415588 | SEQ | GUUUCAGUUUGAAGAAAAUCAGUGUUGGGGUUGCA |
| 00132185- | | ID | GGAGACCUAAACACAGUCACC |
| ENST000 | | NO: | |
| 00367957- | | 456 | |
| 56 | | | |
| ENSG000 | 1.274076278 | SEQ | ACAGUCUCUGCAGAGAAAUCAAAG |
| 00132300- | | ID | |
| ENST000 | | NO: | |
| 00409783- | | 457 | |
| 24 | | | |
| ENSG000 | 1.421294695 | SEQ | AUCUUUCCAGCCUCAGUCGGACGGGCGCGGAGACGC |
| 00132341- | | ID | UUCUGGAAGGAACGCCGCG |
| ENST000 | | NO: | |
| 00541679- | | 458 | |
| 55 | | | |
| ENSG000 | 1.129460196 | SEQ | AAAAAAGCUCUGUGCUGGCUGGAGCCCCCUCAGUG |
| 00132386- | | ID | UGCAGGCUUAGAGGGACUAGGCUGGGUGUGGAGCU |
| ENST000 | | NO: | GCAGCGUAUCCACAGGCCCCAGG |
| 00571360- | | 459 | |
| 93 | | | |
| ENSG000 | 1.119292049 | SEQ | AUCAGCAAGCUCCAGUGCUACGUGUCCCUGGCAUUU |
| 00132432- | | ID | UAGGUGUCGGUUGGGUAGGCAGUC |
| ENST000 | | NO: | |
| 00352861- | | 460 | |
| 60 | | | |
| ENSG000 | 1.219214634 | SEQ | GAGUUCUGAUUUGAAGACUAAUAGUAAUAUAUCUA |
| 00132465- | | ID | GUUUUUCAGAAGAAGUGAAGUCAAG |
| ENST000 | | NO: | |
| 00510614- | | 461 | |
| 60 | | | |
| ENSG000 | 1.403220563 | SEQ | UUGGAGAGACU |
| 00132471- | | ID | |
| ENST000 | | NO: | |
| 00591831- | | 462 | |
| 11 | | | |
| ENSG000 | 1.376167926 | SEQ | CCGAAAGCCUGCAGAGAGCAGAAC |
| 00132530- | | ID | |
| ENST000 | | NO: | |
| 00576724- | | 463 | |
| 24 | | | |
| ENSG000 | 1.197074799 | SEQ | AAAAGAGCUUAAAAAGUAAAACUGUGCUUUGUCCU |
| 00132604- | | ID | AGACUGUUCCUUUGAU |
| ENST000 | | NO: | |
| 00566257- | | 464 | |
| 51 | | | |
| ENSG000 | 1.146047515 | SEQ | AAAGCGUGGCAGCGCAGGCGCAAGCGCAGAGAGCG |
| 00132716- | | ID | GAGGCGGUGGUGGUGGCGGCCGCUGGCCAGUGCUG |
| ENST000 | | NO: | GCUACAGCAAACAUCAUUCAAG |
| 00447377- | | 465 | |
| 92 | | | |
| ENSG000 | 1.236696574 | SEQ | CAGCGCACCAGGUGUCUUUAAGAGUGAUUGAAGAG |
| 00132849- | | ID | AAUAAUUCAAA |
| ENST000 | | NO: | |
| 00635023- | | 466 | |
| 46 | | | |
| ENSG000 | 1.334296863 | SEQ | GGCGAGGAGAGGCACACCACUAUUGUUUUAAUUUG |
| 00133027- | | ID | CUUUUACCUGAUUCCUGGCAGACUUCUGCGUU |
| ENST000 | | NO: | |
| 00395782- | | 467 | |
| 67 | | | |
| ENSG000 | 1.194767908 | SEQ | UCCUAUUCAUUCUCUUGAUUUUUUUUAAAGAAAAC |
| 00133067- | | ID | UUCUUUUGAAAAUUUCCAUCCAAUUCCAAACAAGC |
| ENST000 | | NO: | UAGGACGAACAAAGGGACCUCAGC |
| 00423542- | | 468 | |
| 94 | | | |
| ENSG000 | 2.706005771 | SEQ | AGCGAUCUCCGAGCGAGGCGGCAAG |
| 00133226- | | ID | |
| ENST000 | | NO: | |
| 00323848- | | 469 | |
| 25 | | | |
| ENSG000 | 1.405632638 | SEQ | AAUUUAAGGCUGGACACAGAGUCAGCAACACACAA |
| 00133328- | | ID | GACAUCAUCUUGAAGGAAGG |
| ENST000 | | NO: | |
| 00255695- | | 470 | |
| 55 | | | |
| ENSG000 | 1.229968723 | SEQ | AGUAAAGGCAAAGUACUUUUCAAACAUUCUGCAGC |
| 00133424- | | ID | UGGGAGUACUUCUUUUUUUAAUUUUUUAAAAUAGU |
| ENST000 | | NO: | UCAAGGUGAUUUGUAAUCCACAGCAAA |
| 00674780- | | 471 | |
| 97 | | | |
| ENSG000 | 1.954195587 | SEQ | ACAGAGCUAGCCGGACGUCCUCCGACUCAAGAUAUU |
| 00133731- | | ID | UGUCAAAUAUUUUCAGAAG |
| ENST000 | | NO: | |
| 00519816- | | 472 | |
| 55 | | | |
| ENSG000 | 1.437882414 | SEQ | AUAUUCACCCUUCAGUAGGAGUAGCACAGAGCAAG |
| 00134042- | | ID | AAGCUAGCCACGCUGGGCUCCUCAUUGCCUAGCUCA |
| ENST000 | | NO: | GUUCC |
| 00588444- | | 473 | |
| 76 | | | |
| ENSG000 | 1.289162858 | SEQ | UUUCCAGGUUAACUAAGGUCUCAGCUGUAAACCAA |
| 00134121- | | ID | AAGUGAGAGGAGACAUUAAGAUUUUCAUUCUUACC |
| ENST000 | | NO: | GGGUUGUCUUCUUCCUGAAGAGCA |
| 00620033- | | 474 | |
| 94 | | | |
| ENSG000 | 1.356759582 | SEQ | AAAGCAGUGGUGUUUUACCACUCUGCAGAGAAAGC |
| 00134215- | | ID | UAAGGUUGCUAACCAAACAAAAUAACUUUACACUG |
| ENST000 | | NO: | ACA |
| 00415432- | | 475 | |
| 73 | | | |
| ENSG000 | 1.347501055 | SEQ | ACUCAACCUCUGAAGUUUAGUGACACUAUUGGGAC |
| 00134256- | | ID | GAAAAAGGACUGUGCUGGCCCAA |
| ENST000 | | NO: | |
| 00682167- | | 476 | |
| 58 | | | |
| ENSG000 | 1.148492689 | SEQ | GGAAAAAGCAGUACAUUUAGAAGUAGAAGACUUAG |
| 00134352- | | ID | CUUCAAAUCCCUACUCCUUCACUUACUAAUUUUGUG |
| ENST000 | | NO: | AUUUGGAAAUAUCCGCGCAAG |
| 00522633- | | 477 | |
| 92 | | | |
| ENSG000 | 1.438008154 | SEQ | CAGUGCAACUGAAACUUUUGCAUUACUAUACUACU |
| 00134365- | | ID | GAGAAUAUCUAAC |
| ENST000 | | NO: | |
| 00367418- | | 478 | |
| 48 | | | |
| ENSG000 | 1.150004902 | SEQ | UUUUGUCUUCCUUGAACAGCCACA |
| 00134419- | | ID | |
| ENST000 | | NO: | |
| 00576436- | | 479 | |
| 24 | | | |
| ENSG000 | 2.277302108 | SEQ | GCAAAUACUAGUAGAAAAGGUUAUUGGAAUUCUGU |
| 00134480- | | ID | UCGGUGUUUAAGCCAGCA |
| ENST000 | | NO: | |
| 00508855- | | 480 | |
| 53 | | | |
| ENSG000 | 1.994580395 | SEQ | ACACAUUUUAGGUAGUUGAUUAGAAAACAUACUUG |
| 00134489- | | ID | UCAGAAUUGUCUGGCUGGAUUAAUUUGCUAAUUUG |
| ENST000 | | NO: | ACCUUCUUCAUCAUUUGAGUUG |
| 00256906- | | 481 | |
| 92 | | | |
| ENSG000 | 1.777013002 | SEQ | CUUCAAGAUUAUCAAUAAUCGGAGAUACGUAUAUU |
| 00134588- | | ID | UUAUUUGUAAAGAAAAC |
| ENST000 | | NO: | |
| 00370832- | | 482 | |
| 52 | | | |
| ENSG000 | 1.25325593 | SEQ | CAGGGCCCGUGUUGUGCUUUUGGCCCAGGUAGGUG |
| 00134668- | | ID | GACAGAC |
| ENST000 | | NO: | |
| 00533231- | | 483 | |
| 42 | | | |
| ENSG000 | 1.882459719 | SEQ | UUCAAAGAAUAUAGAGUGUUUUGAAAAGUUCAGUC |
| 00134769- | | ID | UGUCCCUUCAUUGAAUUUUAGA |
| ENST000 | | NO: | |
| 00598142- | | 484 | |
| 57 | | | |
| ENSG000 | 1.102219776 | SEQ | AUUCUCAGCGCUGAUUUUGAG |
| 00135052- | | ID | |
| ENST000 | | NO: | |
| 00470762- | | 485 | |
| 21 | | | |
| ENSG000 | 1.283815746 | SEQ | ACAUAUCCCAGGAGGCAAGUCGCAGGGGAGCACGG |
| 00135205- | | ID | AAUCGUGAAAA |
| ENST000 | | NO: | |
| 00415750- | | 486 | |
| 46 | | | |
| ENSG000 | 1.496409599 | SEQ | GGAGACAGAGAUCCCAGGACAAAGUUGCAGCAGGC |
| 00135248- | | ID | CCCUGAGCAGUUGUAGAA |
| ENST000 | | NO: | |
| 00315184- | | 487 | |
| 53 | | | |
| ENSG000 | 1.178726176 | SEQ | CAGAUAGAUUUUUCUCCCAGC |
| 00135297- | | ID | |
| ENST000 | | NO: | |
| 00680758- | | 488 | |
| 21 | | | |
| ENSG000 | 1.400766672 | SEQ | GUUGCUUGGACUUGGCCCUAUUCUGUAUCAGGUCG |
| 00135314- | | ID | GAGCAGAGC |
| ENST000 | | NO: | |
| 00610330- | | 489 | |
| 44 | | | |
| ENSG000 | 1.964019989 | SEQ | UUCUCACCCUGCCUUUGUUACUGGGAAACCGAACGC |
| 00135373- | | ID | GGCGCUGUGGCUUUCAGCUUGGAUC |
| ENST000 | | NO: | |
| 00529527- | | 490 | |
| 61 | | | |
| ENSG000 | 1.690695667 | SEQ | CCCUGCCUUUGUUACUGGGAAACCGAACGCGGCGCU |
| 00135373- | | ID | GUGGCUUUCAGCUUGGAUC |
| ENST000 | | NO: | |
| 00531728- | | 491 | |
| 55 | | | |
| ENSG000 | 1.931349561 | SEQ | GAUAUUUGGUCUUUGCCUGGCUGAGGGACACAAAG |
| 00135525- | | ID | CAAGAAGAGGGAGACAGUCAGAUAGUUUGCAGGAU |
| ENST000 | | NO: | AUUCAAGAAAGAAGGAUUUUAGC |
| 00544465- | | 492 | |
| 93 | | | |
| ENSG000 | 1.296682686 | SEQ | AAUACUGGGAUCUUCAGUGGCAGGAGGAGUAAUCA |
| 00135605- | | ID | GAAGACGGAG |
| ENST000 | | NO: | |
| 00505452- | | 493 | |
| 45 | | | |
| ENSG000 | 1.225110078 | SEQ | AUUUCCCUUUUUACCAAGAAGAAAGAUUUGUUCAG |
| 00135905- | | ID | CCUGGUGACGCUUCAGAAGAGAAGAGUUUAACGAC |
| ENST000 | | NO: | AUUGAUAUUUUGAAAUAUUUUGUGACUCAAAUUAU |
| 00409592- | | 494 | AGCAAUACG |
| 114 | | | |
| ENSG000 | 1.663519082 | SEQ | ACUGUUUUGCAAACAUAGCAUCAACAUAGAAGUUA |
| 00135905- | | ID | UUUUGUAGGCUCCAGGAGUCAGCG |
| ENST000 | | NO: | |
| 00644695- | | 495 | |
| 59 | | | |
| ENSG000 | 1.388582632 | SEQ | AACGCUUCCUUCGCGGGGCUUUGUGGGUAGCCGAC |
| 00135974- | | ID | UGGGGUCUCCUGGCGACGACC |
| ENST000 | | NO: | |
| 00410049- | | 496 | |
| 56 | | | |
| ENSG000 | 1.196904506 | SEQ | UAGAGAGAGGAA |
| 00136002- | | ID | |
| ENST000 | | NO: | |
| 00355771- | | 497 | |
| 12 | | | |
| ENSG000 | 1.445754515 | SEQ | CAGAGAGUUCGGCUUAUUUAUAGGAAUUGCUUGAA |
| 00136144- | | ID | GCCAGAGUC |
| ENST000 | | NO: | |
| 00258646- | | 498 | |
| 44 | | | |
| ENSG000 | 1.171193993 | SEQ | CAGUCAUUCCCUUGUAGGGACAACUAGAAGAAAUU |
| 00136261- | | ID | UU |
| ENST000 | | NO: | |
| 00446596- | | 499 | |
| 37 | | | |
| ENSG000 | 1.312722487 | SEQ | AGAAAUUCUGCAAAAUAAGUUGGUCUCUAGGAUAC |
| 00136273- | | ID | AUAAGUUGCAUUUUAGUAAUUCCUUAUUUUAACCC |
| ENST000 | | NO: | CUGCUUUAGGAAUCAGUAAC |
| 00433977- | | 500 | |
| 90 | | | |
| ENSG000 | 1.274652444 | SEQ | AGUCGGUGAAGGGCGAGCGGCGCGGCGGCUGCG |
| 00136404- | | ID | |
| ENST000 | | NO: | |
| 00379384- | | 501 | |
| 33 | | | |
| ENSG000 | 1.497033428 | SEQ | CUCUUCCUGAGAAACGAGCAAACCUGAAAGCUACUC |
| 00136514- | | ID | UCUCAGCUUCAGAGGGAAAAA |
| ENST000 | | NO: | |
| 00259030- | | 502 | |
| 57 | | | |
| ENSG000 | 1.420321563 | SEQ | AUUUACUCCAUCCUUUAUAGUGAGUCUACAGGACG |
| 00136560- | | ID | AAGAGGA |
| ENST000 | | NO: | |
| 00403609- | | 503 | |
| 42 | | | |
| ENSG000 | 1.600646987 | SEQ | AUCACUGACCUUGGAAGCUGCUGGAGCCACGAUUC |
| 00136688- | | ID | AGUCCCCUGGACUGUAGAUAAAGACCCUUUCUUGCC |
| ENST000 | | NO: | AGGUGCUGAGACAACCACACU |
| 00259205- | | 504 | |
| 92 | | | |
| ENSG000 | 1.123375448 | SEQ | ACCAACGCUGUCGCCACCUCUGUAGCC |
| 00136861- | | ID | |
| ENST000 | | NO: | |
| 00473282- | | 505 | |
| 27 | | | |
| ENSG000 | 1.120416332 | SEQ | ACUGCUUUCUUGGGAAG |
| 00136930- | | ID | |
| ENST000 | | NO: | |
| 00259457- | | 506 | |
| 17 | | | |
| ENSG000 | 1.440814772 | SEQ | AGACAGUUCCUGCAGCGCUUACCGCCUGGCCUCUCG |
| 00136937- | | ID | GUUCCGCGGCGCACCGGAGGGCAGC |
| ENST000 | | NO: | |
| 00375147- | | 507 | |
| 61 | | | |
| ENSG000 | 1.188196376 | SEQ | ACACGCUGCAAAAGGCUUUCCAAGAAUCCUCGAC |
| 00136960- | | ID | |
| ENST000 | | NO: | |
| 00520066- | | 508 | |
| 34 | | | |
| ENSG000 | 1.221018744 | SEQ | ACAGAAUACUGAAAA |
| 00137033- | | ID | |
| ENST000 | | NO: | |
| 00611532- | | 509 | |
| 15 | | | |
| ENSG000 | 1.409150199 | SEQ | ACAGAGCUGCAGCUCUUCAGGGAAGAAAUCAAAAC |
| 00137033- | | ID | AAGAUCACAAGAAUACUGAAAA |
| ENST000 | | NO: | |
| 00682010- | | 510 | |
| 57 | | | |
| ENSG000 | 1.213289572 | SEQ | CGCCUCUUCUCUUUUUGGGAACGUGGCGAGCAAAA |
| 00137074- | | ID | AAGUCUUGAGCCAGAGUG |
| ENST000 | | NO: | |
| 00467331- | | 511 | |
| 53 | | | |
| ENSG000 | 1.375070072 | SEQ | UUCAAGGUUACCCAUCUCAAGUAGCCUAGCAAUAU |
| 00137080- | | ID | UGGCAACAUCCCA |
| ENST000 | | NO: | |
| 00380225- | | 512 | |
| 48 | | | |
| ENSG000 | 1.149603089 | SEQ | AGUGUCGAUCCCUCAGCCAGGGC |
| 00137135- | | ID | |
| ENST000 | | NO: | |
| 00475323- | | 513 | |
| 23 | | | |
| ENSG000 | 1.31471218 | SEQ | CCGGGCGAGCGCAGCCAAC |
| 00137177- | | ID | |
| ENST000 | | NO: | |
| 00378826- | | 514 | |
| 19 | | | |
| ENSG000 | 1.37316859 | SEQ | AUUGAGAUCACAUAGCCAACUCUG |
| 00137204- | | ID | |
| ENST000 | | NO: | |
| 00436107- | | 515 | |
| 24 | | | |
| ENSG000 | 2.196827289 | SEQ | AUUACACAUUCAAAGUAUCCAUUUGUUCUCAAGAG |
| 00137397- | | ID | GUAGCAAGAAACAGUAUCCACAGUGGACUCCGGGG |
| ENST000 | | NO: | CUCCUACAGACUUGGCACAGCUUCCUACAGUCUUGA |
| 00357569- | | 516 | AACAGCCCUGUUGUUCUGUC |
| 126 | | | |
| ENSG000 | 1.220664428 | SEQ | AUUUCUCGCC |
| 00137404- | | ID | |
| ENST000 | | NO: | |
| 00376421- | | 517 | |
| 10 | | | |
| ENSG000 | 1.286952453 | SEQ | ACACUACGCCAGAACAAG |
| 00137413- | | ID | |
| ENST000 | | NO: | |
| 00372977- | | 518 | |
| 18 | | | |
| ENSG000 | 1.30072718 | SEQ | AACGAAGAGGCACACAGGAGGUCGGAGAACACAAU |
| 00137491- | | ID | AUCAACAGGGCCAGCGGGUGAGGUACCCCAGGUACC |
| ENST000 | | NO: | AGACAAGGAAACCAAAGCCACA |
| 00530556- | | 519 | |
| 93 | | | |
| ENSG000 | 1.874881202 | SEQ | AGAAGAGGACGUUGUCACAGAUAAAGAGCCAGGCU |
| 00137496- | | ID | CACCAGCUCCUGACGCAGUCAUC |
| ENST000 | | NO: | |
| 00393707- | | 520 | |
| 58 | | | |
| ENSG000 | 1.250896159 | SEQ | GUGAUCAGCUC |
| 00137642- | | ID | |
| ENST000 | | NO: | |
| 00534286- | | 521 | |
| 11 | | | |
| ENSG000 | 2.329684846 | SEQ | AUUAUUUUCUAAAUUCUGUGCUAACAGAACCGGCU |
| 00137675- | | ID | UCAGCUGAAGAAAGAGAGGA |
| ENST000 | | NO: | |
| 00260229- | | 522 | |
| 55 | | | |
| ENSG000 | 1.524938971 | SEQ | AUACUUUCAGUUUCAGUCACACAAGAAGGGAGGAG |
| 00137752- | | ID | AGAAAAGCC |
| ENST000 | | NO: | |
| 00353247- | | 523 | |
| 44 | | | |
| ENSG000 | 1.096978726 | SEQ | CAGUAAAUAAAAACACAACUAUUGAUUCAGAGAAA |
| 00137871- | | ID | GGAAAAUUGAUC |
| ENST000 | | NO: | |
| 00558067- | | 524 | |
| 47 | | | |
| ENSG000 | 1.349626178 | SEQ | CCUUUCCCUGUUAGAC |
| 00137955- | | ID | |
| ENST000 | | NO: | |
| 00319942- | | 525 | |
| 16 | | | |
| ENSG000 | 1.443191362 | SEQ | AACAGUUAAUCCUCAAUUUAAGCCUGAUCUAACCCC |
| 00137959- | | ID | UAGAAACAGUAUAGGAUAUAUUCUGUUAAAGAGUG |
| ENST000 | | NO: | AAAAAAGUGAAAAUCUCUGCCAUUU |
| 00680295- | | 526 | |
| 96 | | | |
| ENSG000 | 1.522146348 | SEQ | ACUCAGAGGCCGUCCAAGACACUGGCAAGCCGCAGA |
| 00138039- | | ID | AGCCCAGUUCGCCGGCC |
| ENST000 | | NO: | |
| 00294954- | | 527 | |
| 53 | | | |
| ENSG000 | 1.197719658 | SEQ | ACAGAGCAGGUUCUUUUACAGGGAGCCACC |
| 00138068- | | ID | |
| ENST000 | | NO: | |
| 00535679- | | 528 | |
| 30 | | | |
| ENSG000 | 1.181668687 | SEQ | GUCACCUCUGACAAAAUUCUGGGGACGCUGGGAAC |
| 00138162- | | ID | ACUGAAUCAAC |
| ENST000 | | NO: | |
| 00334433- | | 529 | |
| 46 | | | |
| ENSG000 | 1.342668752 | SEQ | AUUGUUUGAAUUUGAAAACGGUAACAUCGCAGUGC |
| 00138182- | | ID | UGCUCGCGGGUCUGGCUAGUCAGGCGAAGUUUGCA |
| ENST000 | | NO: | GA |
| 00260753- | | 530 | |
| 72 | | | |
| ENSG000 | 1.291351298 | SEQ | GCAGGAGUCUACACCCAGGCGGUGGCUAUACUUAU |
| 00138231- | | ID | UAAAGUUGGCGGGAGCUGAGCGAUCAGUCGGUCCU |
| ENST000 | | NO: | GGGCUGGCUGUGGUGUAGGGCGUUGCAGGCAGCGC |
| 00260803- | | 531 | UCACCUCCUCAGGCCGGCAGA |
| 126 | | | |
| ENSG000 | 1.680490531 | SEQ | GCAUUUAUAUAGCAGAGUAAGAAUCAGGAAAGAAG |
| 00138592- | | ID | CACUUGUAAGGAAAUAUAGCAUCCAUUGUGAAAGU |
| ENST000 | | NO: | GGAAAAGUAAAGAUAAUUCAUC |
| 00558091- | | 532 | |
| 92 | | | |
| ENSG000 | 1.599779967 | SEQ | CUCAGUCAAGCUGAAGUGAAAACGAGACCAAGGUC |
| 00138684- | | ID | UAGCUCUACUGUUGGUACUU |
| ENST000 | | NO: | |
| 00648588- | | 533 | |
| 55 | | | |
| ENSG000 | 1.392715227 | SEQ | GAAAUCUUUUAGGCCUUUAGAGUUUGUUCUGGAAA |
| 00138688- | | ID | AACUUUAGUA |
| ENST000 | | NO: | |
| 00388738- | | 534 | |
| 45 | | | |
| ENSG000 | 1.354008715 | SEQ | ACAUUGACUGCUCUGCUGCUGCA |
| 00138696- | | ID | |
| ENST000 | | NO: | |
| 00440890- | | 535 | |
| 23 | | | |
| ENSG000 | 1.271036129 | SEQ | GGGCCCUGACAGUUGAAAGGAAGGAAGUUGCCCUA |
| 00138741- | | ID | AUAUUCUACAGUUGUUUUAUCGUUGCUACUGAUUA |
| ENST000 | | NO: | GGUCC |
| 00506449- | | 536 | |
| 75 | | | |
| ENSG000 | 1.408945716 | SEQ | AAAGAAUUUCUCAGGCUCAAAAUCCAAUACAGGAG |
| 00138755- | | ID | UGACUUGGAACUCCAUUCUAUCACU |
| ENST000 | | NO: | |
| 00264888- | | 537 | |
| 60 | | | |
| ENSG000 | 1.413788622 | SEQ | AUUGUGGGAAAAG |
| 00139180- | | ID | |
| ENST000 | | NO: | |
| 00266544- | | 538 | |
| 13 | | | |
| ENSG000 | 1.222487091 | SEQ | AAGUCUGUUCAGCUGCCUGUUUGAAGAAAGUUUAU |
| 00139220- | | ID | UUUUUAAAAACUAGUUUUGCAGUUGGCUGAAGAGA |
| ENST000 | | NO: | GACAGUGAAAUAUUGAAGAACGU |
| 00551461- | | 539 | |
| 93 | | | |
| ENSG000 | 1.311768333 | SEQ | ACUUCCGGGCUCGGCAGCUAGACUAGCGGGGGCAGC |
| 00139631- | | ID | CUCAGAGCGGCUACCUGUGCACCCUGCCUCCGGCUC |
| ENST000 | | NO: | UCCUGAGCAGAGAGAUCCUG |
| 00437073- | | 540 | |
| 92 | | | |
| ENSG000 | 1.144898413 | SEQ | UAGGGCCGCUGUGG |
| 00139746- | | ID | |
| ENST000 | | NO: | |
| 00267229- | | 541 | |
| 14 | | | |
| ENSG000 | 1.140503021 | SEQ | GUCUUGGAGAGAGGCUGUGUGCAUUAGCUACAAAU |
| 00140262- | | ID | AGUAACAUUUUCAAAAUCAAC |
| ENST000 | | NO: | |
| 00343827- | | 542 | |
| 56 | | | |
| ENSG000 | 1.699983351 | SEQ | ACUGAAGACUUACUCCCUUUGCCUGUGUGGAUAUA |
| 00140262- | | ID | GUUCUUGGAGAGAGGCUGUGUGCAUUAGCUACAAA |
| ENST000 | | NO: | UAGUAACAUUUUCAAAAUCAAC |
| 00543579- | | 543 | |
| 92 | | | |
| ENSG000 | 1.390016553 | SEQ | GAGUCAUACUCCAGUGGUUUAGUUGAUAAAGUAUC |
| 00140386- | | ID | UUGGCAUAGAAGAGAAUUUAAAGA |
| ENST000 | | NO: | |
| 00324767- | | 544 | |
| 59 | | | |
| ENSG000 | 1.159919678 | SEQ | AGUCUCCUUUAAGGAGAGCUUGUAGGCUUCUAAUU |
| 00140403- | | ID | UCACAUUUCAGCAAGCUGGCUAAAGAC |
| ENST000 | | NO: | |
| 00446172- | | 545 | |
| 62 | | | |
| ENSG000 | 1.237967637 | SEQ | CCGCUGAAGUGGAGCAGGUACAGUCACAGCUGUGG |
| 00140459- | | ID | GGACAGC |
| ENST000 | | NO: | |
| 00435365- | | 546 | |
| 42 | | | |
| ENSG000 | 1.223971881 | SEQ | GUGAGGCCGAGGUGACUGCAGAGCGGCUCGCGAGU |
| 00140525- | | ID | UCUGUGAUAGUAGCAACA |
| ENST000 | | NO: | |
| 00563250- | | 547 | |
| 53 | | | |
| ENSG000 | 1.113367469 | SEQ | UCCACGUUUCUUUAGCGGGCAGUGGUAAAAAGAGU |
| 00140718- | | ID | AUUUAAGAUCGGUCGAGUUUGAGUGGCUGAGGCAG |
| ENST000 | | NO: | UUUUGGUUUCAAGGCAAUCGAUACAGAAAGUGCAC |
| 00460382- | | 548 | UGACUGGUGGUGUCAACCC |
| 124 | | | |
| ENSG000 | 1.379159616 | SEQ | GUGGUUUCCUAGAAACAGUAUUGUUUAUUGGCAUU |
| 00141027- | | ID | GAUCUCACAGUCUGGUGAGGACUUCUUUACUGAUA |
| ENST000 | | NO: | |
| 00395848- | | 549 | |
| 70 | | | |
| ENSG000 | 1.134255567 | SEQ | GCCUAAAGUAAAAUUCAGCUCGAAGUACAGCAGUA |
| 00141255- | | ID | UAAAGACAG |
| ENST000 | | NO: | |
| 00574051- | | 550 | |
| 44 | | | |
| ENSG000 | 1.329912052 | SEQ | CAAUAAAGUUUUAUAUUUAUUUUGUGAAGGUGCC |
| 00141279- | | ID | |
| ENST000 | | NO: | |
| 00530514- | | 551 | |
| 34 | | | |
| ENSG000 | 1.796554333 | SEQ | ACUUGCCGAUAGUGGUGACGGCGGCAAC |
| 00141380- | | ID | |
| ENST000 | | NO: | |
| 00582792- | | 552 | |
| 28 | | | |
| ENSG000 | 1.230257795 | SEQ | AGCGAUAUCCGGACACAGAUCACAGCGUUCUUUCU |
| 00141404- | | ID | GUUUGUUUGCAGGGGCUGGUGAGUCUGGGAAAAGC |
| ENST000 | | NO: | ACUAUCGUCAAACAG |
| 00585642- | | 553 | |
| 85 | | | |
| ENSG000 | 1.191594015 | SEQ | UCAGAACCAUCAAGUAGAGUCAAAC |
| 00141431- | | ID | |
| ENST000 | | NO: | |
| 00592288- | | 554 | |
| 25 | | | |
| ENSG000 | 1.398730258 | SEQ | CAACCCUGAAGUUCAUAGUUAGCUACUUUCAACUG |
| 00141434- | | ID | GAAGCUACAAC |
| ENST000 | | NO: | |
| 00581447- | | 555 | |
| 46 | | | |
| ENSG000 | 1.513906113 | SEQ | ACUCGCCUCAUUUUCUCUAAGCAGAGCAACUUCGCG |
| 00141668- | | ID | GACAGCGGAGCUCGCCCAGCAGUG |
| ENST000 | | NO: | |
| 00584764- | | 556 | |
| 60 | | | |
| ENSG000 | 1.212522073 | SEQ | ACUGGGCGGACCGCGGCGGCAGC |
| 00141744- | | ID | |
| ENST000 | | NO: | |
| 00269582- | | 557 | |
| 23 | | | |
| ENSG000 | 1.259900848 | SEQ | ACUUUCACUCACCGCCUGUCCUUCCUGACACCUCAC |
| 00142484- | | ID | C |
| ENST000 | | NO: | |
| 00270560- | | 558 | |
| 37 | | | |
| ENSG000 | 1.395323206 | SEQ | GUGACAGAGCGCUUUACGACAGUUGCUUUGAGGCA |
| 00142507- | | ID | GUACCGGAGGAGAAAG |
| ENST000 | | NO: | |
| 00614486- | | 559 | |
| 51 | | | |
| ENSG000 | 1.24945238 | SEQ | CUGGGAGUCGUUAGUGAGG |
| 00142546- | | ID | |
| ENST000 | | NO: | |
| 00594932- | | 560 | |
| 19 | | | |
| ENSG000 | 1.254143024 | SEQ | ACAUUUGCGGGAACGCAGAGCGGAGCGUGGAGAGC |
| 00142552- | | ID | GGAGCGAAGCUGGAUAACAGGGGACCG |
| ENST000 | | NO: | |
| 00270645- | | 561 | |
| 62 | | | |
| ENSG000 | 1.128512499 | SEQ | AGUGUGUGGCUGCUUCUGGACUCAAGGAGGAGGAG |
| 00142611- | | ID | AGAGAUUCCGCGAGCCGACACC |
| ENST000 | | NO: | |
| 00270722- | | 562 | |
| 57 | | | |
| ENSG000 | 1.379157796 | SEQ | CUCUUCCUGCUCUCCAUC |
| 00142676- | | ID | |
| ENST000 | | NO: | |
| 00643754- | | 563 | |
| 18 | | | |
| ENSG000 | 1.359498231 | SEQ | AGUCCUUGGCGUUUCCUUUGGCGGAUUUUCUGUUU |
| 00142856- | | ID | UCGGAAGUUGCUGGGUUCGUUUUAUUCAGCGGCAG |
| ENST000 | | NO: | UGGUGCUUUCCCGAAUCUCAGA |
| 00271002- | | 564 | |
| 92 | | | |
| ENSG000 | 1.688343768 | SEQ | GUUUUCGGAAGUUGCUGGGUUCGUUUUAUUCAGCG |
| 00142856- | | ID | GCAGUGGUGCUUUCCCGAAUCUCAGA |
| ENST000 | | NO: | |
| 00371092- | | 565 | |
| 61 | | | |
| ENSG000 | 1.357553822 | SEQ | ACAGUCAUAGCUCUUAGCUUCUGUGUAAGAAGUUG |
| 00142875- | | ID | UGAGCUCCUUCUGGAAACAUUUGCAGUUACAUUAA |
| ENST000 | | NO: | GUAAAGUGUAAAGUCACAGUA |
| 00370685- | | 566 | |
| 91 | | | |
| ENSG000 | 1.148814753 | SEQ | GUUAUUUUGAGCAAUAGUUUUUGGAAAGGUUGGUU |
| 00142875- | | ID | UUCAUC |
| ENST000 | | NO: | |
| 00394839- | | 567 | |
| 41 | | | |
| ENSG000 | 1.130408224 | SEQ | ACGAGCCUUUCCGGUGCU |
| 00143183- | | ID | |
| ENST000 | | NO: | |
| 00612311- | | 568 | |
| 18 | | | |
| ENSG000 | 1.229689439 | SEQ | CUCUUGCGCCCUGGUCCAAG |
| 00143222- | | ID | |
| ENST000 | | NO: | |
| 00368003- | | 569 | |
| 20 | | | |
| ENSG000 | 1.144374529 | SEQ | ACAGUGCUGGG |
| 00143226- | | ID | |
| ENST000 | | NO: | |
| 00271450- | | 570 | |
| 11 | | | |
| ENSG000 | 1.15445736 | SEQ | AGAAGUGUCUGACAAAACAAAGAAACAAAACCUCA |
| 00143248- | | ID | GUGAAAA |
| ENST000 | | NO: | |
| 00367903- | | 571 | |
| 42 | | | |
| ENSG000 | 1.628278755 | SEQ | CAAAGACUGGCUCUGCUGUUAAGAAGUUGUACUUA |
| 00143248- | | ID | AAGCGGAGGAGCUAAGCCACCUGCCAAA |
| ENST000 | | NO: | |
| 00531476- | | 572 | |
| 63 | | | |
| ENSG000 | 1.22937685 | SEQ | AGAAUUGUUAAAAUCUUUGUGAAGCACACCACUGA |
| 00143278- | | ID | AG |
| ENST000 | | NO: | |
| 00367412- | | 573 | |
| 37 | | | |
| ENSG000 | 1.233402722 | SEQ | AGAACUGAGCUCAUCUUUGCGGGGUUAGCUAGCCG |
| 00143321- | | ID | UUGAGUCCAAGUCUCCUUGGCAAGACAGUAGCUUA |
| ENST000 | | NO: | GAGAAACGAGAUCGA |
| 00368206- | | 574 | |
| 85 | | | |
| ENSG000 | 1.180621821 | SEQ | CUCUUUACACAGAACAGUUUAACUUUUGUGCUUCU |
| 00143322- | | ID | GGGCAGAGGU |
| ENST000 | | NO: | |
| 00344730- | | 575 | |
| 45 | | | |
| ENSG000 | 1.242198062 | SEQ | CUGGGCAGAGGU |
| 00143322- | | ID | |
| ENST000 | | NO: | |
| 00512653- | | 576 | |
| 12 | | | |
| ENSG000 | 1.485125549 | SEQ | AAAAAGAGCCCUUGAAGCUGGAAGACGGGAGAGGA |
| 00143379- | | ID | CAAAAGC |
| ENST000 | | NO: | |
| 00368964- | | 577 | |
| 42 | | | |
| ENSG000 | 1.66529389 | SEQ | AAGAGAGAAUCUCUUAUAGUUGCUGAUACAGGUUU |
| 00143398- | | ID | CAACUUUCUGUGCUGUGGGGUCAGUGGUAAGAGAG |
| ENST000 | | NO: | CAGCAUCUGGAAUCAAGAGACCC |
| 00418435- | | 578 | |
| 93 | | | |
| ENSG000 | 1.11234934 | SEQ | AUUCUGUGCCUGUGAAC |
| 00143436- | | ID | |
| ENST000 | | NO: | |
| 00368830- | | 579 | |
| 17 | | | |
| ENSG000 | 1.134078117 | SEQ | AUUUUCGAGUUGGUGAGACACCGCGGCAGUCGACU |
| 00143507- | | ID | UGGAAGAGUCUCUGGUGGUAGAGGUGGACUUAGUA |
| ENST000 | | NO: | GUUUUAAGCAGAACC |
| 00468085- | | 580 | |
| 85 | | | |
| ENSG000 | 1.192767603 | SEQ | GUGAGAGGAGGCUGCAACGCCGAGCGGAGGAGGCA |
| 00143549- | | ID | GGAACCGGAGCGCGAGCAGUAGCUGGGUGGGCACC |
| ENST000 | | NO: | |
| 00368533- | | 581 | |
| 70 | | | |
| ENSG000 | 1.667823427 | SEQ | ACACAUCUCACUCAUCCUUCUACUCGUGACGCUUCC |
| 00143556- | | ID | CAGCUCUGGCUUUUUGAAAGCAAAG |
| ENST000 | | NO: | |
| 00368723- | | 582 | |
| 61 | | | |
| ENSG000 | 1.175624102 | SEQ | GUCAGUGUGGAGGAGACUGAGUAUUCUACCUUGUA |
| 00143569- | | ID | AAUACUGUUAUUUGUAUAUACUGUAA |
| ENST000 | | NO: | |
| 00428931- | | 583 | |
| 61 | | | |
| ENSG000 | 1.413605476 | SEQ | GGAAGGAAGUGUUUUUAAAACUACGUGAGGCAUCA |
| 00143977- | | ID | GAAUCCGAAAGCCACUUUAGUCUUAGCAA |
| ENST000 | | NO: | |
| 00413456- | | 584 | |
| 64 | | | |
| ENSG000 | 1.267636273 | SEQ | GCCUCCAGGAUC |
| 00144115 | | ID | |
| ENST000 | | NO: | |
| 00324166- | | 585 | |
| 12 | | | |
| ENSG000 | 1.142958067 | SEQ | CCUCACACCAACAACUGCACCUACUUUCUUACAGGG |
| 00144130- | | ID | UUGGAGUCCUAUAAC |
| ENST000 | | NO: | |
| 00327581- | | 586 | |
| 51 | | | |
| ENSG000 | 1.201604128 | SEQ | GUGUAAGACACUGAAGACACUGCAGAGCAAGGUGC |
| 00144290- | | ID | UUAUUCCAGAGGCGUUACAAAAC |
| ENST000 | | NO: | |
| 00421911- | | 587 | |
| 58 | | | |
| ENSG000 | 1.100129357 | SEQ | UAGACUAUAAGCUUUUGGAAAACAGGGACUAGUUC |
| 00144331- | | ID | UUCUUCAACAAGUUGUACACAACAAGUAGCAGGUU |
| ENST000 | | NO: | GUUUGGCACAGUGCAG |
| 00439340- | | 588 | |
| 86 | | | |
| ENSG000 | 1.107466439 | SEQ | ACAGUCUUGAGUCAUUCAUUCUCAAGGACACUUGA |
| 00144410- | | ID | UCCACUGCCAGAGAGGCCCAGAAUUUUCUAACUUAC |
| ENST000 | | NO: | UGUGUGGCAGA |
| 00272852- | | 589 | |
| 82 | | | |
| ENSG000 | 1.145354517 | SEQ | AGUAGCGGGCGGCAGAGCUGGAGUGAAGGGAGCUA |
| 00144591- | | ID | GUGGUAGCAGUCACCAUU |
| ENST000 | | NO: | |
| 00682481- | | 590 | |
| 53 | | | |
| ENSG000 | 1.422725123 | SEQ | UCUUUCCGGGACAAC |
| 00144895- | | ID | |
| ENST000 | | NO: | |
| 00482093- | | 591 | |
| 15 | | | |
| ENSG000 | 1.371132109 | SEQ | AGAGAAGAAG |
| 00145191- | | ID | |
| ENST000 | | NO: | |
| 00648682- | | 592 | |
| 10 | | | |
| ENSG000 | 1.273612863 | SEQ | ACCACUUUUACAGUGUCACCUACAGGAGAGAGGAG |
| 00145321- | | ID | GUGCUGCAAGACUCUCUGGUAGAAAA |
| ENST000 | | NO: | |
| 00273951- | | 593 | |
| 61 | | | |
| ENSG000 | 1.234149722 | SEQ | ACUGUUUUUUCAAAGAGAUUGCUUACUCCACCAGG |
| 00145321- | | ID | AGAAGUAUAUCUACCUAUUCUACCACUGGUGAGUA |
| ENST000 | | NO: | UCUGGACUAGUCCAACUGGUAGGAA |
| 00504199- | | 594 | |
| 95 | | | |
| ENSG000 | 1.510041506 | SEQ | GUACAGGCGGCACGGUUUGAGUGCAGAGAUAUUUU |
| 00145362- | | ID | CUUUCCAAACUGUUCAAA |
| ENST000 | | NO: | |
| 00394537- | | 595 | |
| 53 | | | |
| ENSG000 | 1.86606588 | SEQ | GGUUUAAUUCUUCAGAUCACAUUGAGAGUAAAGGU |
| 00145414- | | ID | AUUAAAAUAAAGGAGACU |
| ENST000 | | NO: | |
| 00509434- | | 596 | |
| 53 | | | |
| ENSG000 | 1.184037397 | SEQ | UAUUUUCAGAAUUUCAGAAAUUGAAAAGGGAUAAG |
| 00145416- | | ID | AGGAAGGAGAAA |
| ENST000 | | NO: | |
| 00274056- | | 597 | |
| 47 | | | |
| ENSG000 | 1.164271099 | SEQ | CUAUAUUCUCUGUUCUGCAUUAUAAGCAAC |
| 00145416- | | ID | |
| ENST000 | | NO: | |
| 00339875- | | 598 | |
| 30 | | | |
| ENSG000 | 1.506110818 | SEQ | ACAAGCGCCUUACGAAAGGCGGCAAAAAGGGAGCC |
| 00145425- | | ID | AAGAAGAAAGGUUGAUCCAUUUUCUAAGAAAGAUU |
| ENST000 | | NO: | GGUAGUAGUUGAAAGCACCUGCU |
| 00507327- | | 599 | |
| 93 | | | |
| ENSG000 | 1.169807393 | SEQ | CUCUUCCGGUCUUUCUGGUCUCGGCCGCAGAAGCGA |
| 00145592- | | ID | G |
| ENST000 | | NO: | |
| 00274242- | | 600 | |
| 37 | | | |
| ENSG000 | 1.654899794 | SEQ | UAUAGGUUUUAAAAGUAAUUCCAAGACACCAUUAC |
| 00145675- | | ID | AAAGAAAGCCGGACUCUUUUCUUAUAACUGAGCUC |
| ENST000 | | NO: | AGCCAAGGAAACUCUUGCACAA |
| 00336483- | | 601 | |
| 92 | | | |
| ENSG000 | 1.228077774 | SEQ | AGCAGCUUGUAUUCUUAGAAACGCAGCAGACAGCA |
| 00145703- | | ID | ACUUUAGCAGGUGAUUUCAAUCAAGUCAGCUGAA |
| ENST000 | | NO: | |
| 00396234- | | 602 | |
| 69 | | | |
| ENSG000 | 1.249023976 | SEQ | ACGAGAGGAAGCUUAAAUCUGUCGUUUGAAUUUAG |
| 00145723- | | ID | GACCACCUCGGUUCACA |
| ENST000 | | NO: | |
| 00512248- | | 603 | |
| 52 | | | |
| ENSG000 | 1.298039977 | SEQ | AGUGUCGAGUCAGCUGUAGUGACAAUCUCAGAGCA |
| 00145879- | | ID | GCUUCUACACCACAGCCAUUUCCAGC |
| ENST000 | | NO: | |
| 00274565- | | 604 | |
| 61 | | | |
| ENSG000 | 1.268497747 | SEQ | CUUUUCUGAAAACAUCAGCAUUCUGCCAUAUCUGG |
| 00145934- | | ID | AAUA |
| ENST000 | | NO: | |
| 00518659- | | 605 | |
| 39 | | | |
| ENSG000 | 1.189230872 | SEQ | ACUUCGCUGUGAAG |
| 00146007- | | ID | |
| ENST000 | | NO: | |
| 00274712- | | 606 | |
| 14 | | | |
| ENSG000 | 1.272249185 | SEQ | UAGUAGUAGGCUCCCUUCCCACCUCAGUCAUUGGUU |
| 00146147- | | ID | UGCUCGCUCCCUUAGUUGAGUAAUCA |
| ENST000 | | NO: | |
| 00503951- | | 607 | |
| 62 | | | |
| ENSG000 | 1.327706632 | SEQ | UCUUUCUCUCUUUCUCAUUCUCUUUCAAU |
| 00146147- | | ID | |
| ENST000 | | NO: | |
| 00509997- | | 608 | |
| 29 | | | |
| ENSG000 | 1.358539776 | SEQ | GGAAACUUAUUUAAGGAGUGCUGACUUACCGUAUC |
| 00146267- | | ID | AGGUGAUAAGAAGUACAUC |
| ENST000 | | NO: | |
| 00538471- | | 609 | |
| 54 | | | |
| ENSG000 | 1.734820535 | SEQ | UUUUAGUGGUGGAGUCAAUUUAUUUCUGAAACGAU |
| 00146592- | | ID | CUCAUUUACCUGAAGUAGGAGCUCAUAUUUAUUUU |
| ENST000 | | NO: | CAGGAUUUAGUAGGAAUCCAAG |
| 00396300- | | 610 | |
| 92 | | | |
| ENSG000 | 1.10489515 | SEQ | ACUCAAGUUCGGAACGUUCCGAAG |
| 00146776- | | ID | |
| ENST000 | | NO: | |
| 00318724- | | 611 | |
| 24 | | | |
| ENSG000 | 1.899199977 | SEQ | GAUCGCUUCAGGUUCCAAAAGUACAACAGUUUUAC |
| 00146776- | | ID | AGUCGUCGUAGCAUCAGAUUAAGUUGGCAGCCUGA |
| ENST000 | | NO: | GUCUCUCUAGAGAAGACACGGUUCA |
| 00472195- | | 612 | |
| 95 | | | |
| ENSG000 | 1.74032445 | SEQ | GUAGAUUAGCUCAACACUCGAGAAUCAUUGCAGCU |
| 00146809- | | ID | UCAAGGA |
| ENST000 | | NO: | |
| 00275699- | | 613 | |
| 42 | | | |
| ENSG000 | 1.121553935 | SEQ | GAGCACGGAACUUAUUCCUGCCCAGAGUUCAGACAC |
| 00147010- | | ID | AGCCAGAAAUUACCAUUUCAUACUUCACAGA |
| ENST000 | | NO: | |
| 00379698- | | 614 | |
| 67 | | | |
| ENSG000 | 1.219039436 | SEQ | AAAAUUAGAAAAUUUAAACUUCUUCAGUAGGACGU |
| 00147044- | | ID | GUUGGAACACCUCAUUUU |
| ENST000 | | NO: | |
| 00643043- | | 615 | |
| 53 | | | |
| ENSG000 | 1.170463811 | SEQ | GCGGCCGAUCAGAGGAGCAGGAACUGGAACCCUGG |
| 00147099- | | ID | CCGAGUCCGAAAAAAGCCAGAUCUGGAAGGUGGCU |
| ENST000 | | NO: | GCGGAACGGUUUUAAGCGGAAG |
| 00373554- | | 616 | |
| 92 | | | |
| ENSG000 | 1.643128391 | SEQ | CCGAGUCCGAAAAAAGCCAGAUCUGGAAGGUGGCU |
| 00147099- | | ID | GCGGAACGGUUUUAAGCGGAAG |
| ENST000 | | NO: | |
| 00648834- | | 617 | |
| 57 | | | |
| ENSG000 | 1.20698 | SEQ | GCAGCUGAGCGUUGGAAGCCAUUUUGGCUGCAACC |
| 00147127- | | ID | UACCUGAAGCG |
| ENST000 | | NO: | |
| 00276066- | | 618 | |
| 46 | | | |
| ENSG000 | 1.248024213 | SEQ | AUUCGGCAGCCAGACUCCUUGAAAUACCCUUUCAGU |
| 00147166- | | ID | AAUCAUUCAACCAACGCUUCC |
| ENST000 | | NO: | |
| 00373829- | | 619 | |
| 57 | | | |
| ENSG000 | 1.4528557 | SEQ | ACAGUACUCAACCUACUUCAAGGGAAGAUUGGGAU |
| 00147437- | | ID | CUUUUUGGCUCUCUGCCUCUAAACAGA |
| ENST000 | | NO: | |
| 00421054- | | 620 | |
| 62 | | | |
| ENSG000 | 1.954172457 | SEQ | AAGAGGACUCCAAGCGCC |
| 00147576- | | ID | |
| ENST000 | | NO: | |
| 00276576- | | 621 | |
| 18 | | | |
| ENSG000 | 1.494732017 | SEQ | ACUCCAAGCGCC |
| 00147576- | | ID | |
| ENST000 | | NO | |
| 00422166- | | 622 | |
| 12 | | | |
| ENSG000 | 1.112748848 | SEQ | CCUCUUUUUCCGGCUGGAACC |
| 00147604- | | ID | |
| ENST000 | | NO: | |
| 00352983- | | 623 | |
| 21 | | | |
| ENSG000 | 1.257158970 | SEQ | UCUUUCUUCCUGUCUGCUUGGAAAG |
| 00147677- | | ID | |
| ENST000 | | NO: | |
| 00518995- | | 624 | |
| 25 | | | |
| ENSG000 | 1.272203789 | SEQ | AAGAGACGCUUCCUUCUGAUC |
| 00147862- | | ID | |
| ENST000 | | NO: | |
| 00636735- | | 625 | |
| 21 | | | |
| ENSG000 | 1.158431422 | SEQ | ACCUCUGGUGCCAAAGGGCGGCGCAGCGGCUGCCGA |
| 00147889- | | ID | GCUCGGCCCUGGAGGCGGCGAGAAC |
| ENST000 | | NO: | |
| 00579755- | | 626 | |
| 61 | | | |
| ENSG000 | 1.322161708 | SEQ | CGGCGUGUUGGUCCCAGCGGUUCAGCUGAGGUAGG |
| 00147996- | | ID | GACGUGCCGUAGGCCGGA |
| ENST000 | | NO: | |
| 00430059- | | 627 | |
| 53 | | | |
| ENSG000 | 1.575979209 | SEQ | GUCUUAUUCUUUUUAGUGGAGUUUUCCAAGGAUUG |
| 00148187- | | ID | UCUUCAGUC |
| ENST000 | | NO: | |
| 00489572- | | 628 | |
| 44 | | | |
| ENSG000 | 1.265236768 | SEQ | CAGUGCUGCAAUAGAACUUUUCAGCAGACUACAAG |
| 00148200- | | ID | AAGCAGUAA |
| ENST000 | | NO: | |
| 00475178- | | 629 | |
| 44 | | | |
| ENSG000 | 1.284654826 | SEQ | GUCUGCCGGGCC |
| 00148803- | | ID | |
| ENST000 | | NO: | |
| 00278025- | | 630 | |
| 12 | | | |
| ENSG000 | 1.27621693 | SEQ | CAUUAUAAGUUCAGUAGUACAACAUUUUGUGAGAG |
| 00148935- | | ID | AAGUCUGUAUAAAAAGCAAUUGCUCUUUUGUUUCC |
| ENST000 | | NO: | AAAACAGGUAUUACAAGUGGAUAAAUA |
| 00532398- | | 631 | |
| 97 | | | |
| ENSG000 | 1.604750689 | SEQ | AUUCACUCGCUCUUUUCAUUCACGAAGGUAGUGAG |
| 00149084- | | ID | GCCUAGUGGAAAGCC |
| ENST000 | | NO: | |
| 00636007- | | 632 | |
| 50 | | | |
| ENSG000 | 1.133062745 | SEQ | AUUCACGAAGGUAGUGAGGCCUAGUGGAAAGCC |
| 00149084- | | ID | |
| ENST000 | | NO: | |
| 00636722- | | 633 | |
| 33 | | | |
| ENSG000 | 1.383974628 | SEQ | AUUCCGUGUUC |
| 00149084- | | ID | |
| ENST000 | | NO: | |
| 00638034- | | 634 | |
| 11 | | | |
| ENSG000 | 2.328080788 | SEQ | AAGCUGAGAGGGCCCGCGGGUAGGC |
| 00149262- | | ID | |
| ENST000 | | NO: | |
| 00534064- | | 635 | |
| 25 | | | |
| ENSG000 | 1.108821914 | SEQ | UUAGGGAUUCCGGUUCACA |
| 00149292- | | ID | |
| ENST000 | | NO: | |
| 00314756- | | 636 | |
| 19 | | | |
| ENSG000 | 1.268076996 | SEQ | GGAAAGUCACAAGCCUCUUGAGUCAUAAAAACAGC |
| 00149300- | | ID | UGGGCUCCCUUGGAGACAGAGCGCC |
| ENST000 | | NO: | |
| 00278601- | | 637 | |
| 60 | | | |
| ENSG000 | 1.465679578 | SEQ | GCAUUCCCUUGGGCAGAGAGUGGAG |
| 00149489- | | ID | |
| ENST000 | | NO: | |
| 00278833- | | 638 | |
| 25 | | | |
| ENSG000 | 1.352591675 | SEQ | UGGCAUCUUCCUUACUUUGUCCAUCCUCCGGACUCG |
| 00149532- | | ID | CGAUCUUCCUUCCGGAGCC |
| ENST000 | | NO: | |
| 00536145- | | 639 | |
| 55 | | | |
| ENSG000 | 1.209434894 | SEQ | ACAAGGAGGCAGGCAAGACAGCAAGGCAUAGAGAC |
| 00149968- | | ID | AACAUAGAGCUAAGUAAAGCCAGUGGAA |
| ENST000 | | NO: | |
| 00299855- | | 640 | |
| 63 | | | |
| ENSG000 | 1.183808331 | SEQ | AAGGAGGCAGGCAAGACAGCAAGGCAUAGAGACAA |
| 00149968- | | ID | CAUAGAGCUAAGUAAAGCCAGUGGAA |
| ENST000 | | NO: | |
| 00524478- | | 641 | |
| 61 | | | |
| ENSG000 | 1.751805475 | SEQ | AUUUCAGUUUAUACUUAAUAAAACAAAACAUACCU |
| 00150045- | | ID | GUAUACACACACAUUCACUCACAUUGAAG |
| ENST000 | | NO: | |
| 00279545- | | 642 | |
| 64 | | | |
| ENSG000 | 1.529361133 | SEQ | AAGUAAAAGUGGUCAAACAACUUAACAGUGUGCCA |
| 00150093- | | ID | GUUUUCCUAAGCGUGCUUGCCACUGAUUGUAAGUC |
| ENST000 | | NO: | UUUUAUUGUUUGGCUUCUUGAUAA |
| 00480226- | | 643 | |
| 94 | | | |
| ENSG000 | 1.367487177 | SEQ | CUUUCUCAGAAAACCAAAU |
| 00150540- | | ID | |
| ENST000 | | NO: | |
| 00280096- | | 644 | |
| 19 | | | |
| ENSG000 | 1.341507935 | SEQ | UGUCUUUAAAAAAUAACUGUAUACUAAGCAAAACU |
| 00150636- | | ID | CAGGUCUUAAAAAUAAAU |
| ENST000 | | NO: | |
| 00581520- | | 645 | |
| 53 | | | |
| ENSG000 | 1.133266124 | SEQ | GGAGUGUGUCUGAGGAAACACAUCCCGGACACCAC |
| 00150722- | | ID | UUAGGGUUAGUCUUUCUGAGCUCUUCACAUCUCUG |
| ENST000 | | NO: | ACUAAUUAUCAUCAUUACC |
| 00409702- | | 646 | |
| 89 | | | |
| ENSG000 | 1.349622591 | SEQ | UUCCUACGACAAAAGAUACAUCAUCAAGACUAUUA |
| 00150867- | | ID | CCAGUGAAGACGUGGCCGAA |
| ENST000 | | NO: | |
| 00323883- | | 647 | |
| 55 | | | |
| ENSG000 | 1.281733641 | SEQ | CUUCCCUGGCCUUGCCCUCUCUCUUUCUGCCCUGUA |
| 00151062- | | ID | GCCGCGGGCGUCCAA |
| ENST000 | | NO: | |
| 00538027- | | 648 | |
| 51 | | | |
| ENSG000 | 1.343539722 | SEQ | GUCUGACGGCACUUAAGGUCUAAAAAAGAAGGAAG |
| 00151093- | | ID | UUGUUUCUGAUCAUCUGAAUCUUAAUC |
| ENST000 | | NO: | |
| 00428266- | | 649 | |
| 62 | | | |
| ENSG000 | 1.141568488 | SEQ | CUGUGGUUUAAGGGACUGUGCUCGGGGAAGACGUG |
| 00151474- | | ID | CUGUCAGCG |
| ENST000 | | NO: | |
| 00649155- | | 650 | |
| 44 | | | |
| ENSG000 | 1.565400149 | SEQ | AGCUGGGGAGUUUGCAGAUCUGAUCUGUCAGCGGG |
| 00151474- | | ID | GACUGAGAGCAAAUCGGGACAUUCAUC |
| ENST000 | | NO: | |
| 00649947- | | 651 | |
| 62 | | | |
| ENSG000 | 1.297593807 | SEQ | UCACAAUCACAUUGAGCCAAAACGCAUCCAGUGUU |
| 00151611- | | ID | UUCUCCAGUUACAAAUAAAACGAAU |
| ENST000 | | NO: | |
| 00647947- | | 652 | |
| 60 | | | |
| ENSG000 | 1.490938996 | SEQ | GAUAAUCUUUUCCUUCUUAUUCAUUGCAGAAGCUU |
| 00151687- | | ID | CAAAAAGGACACACUAGAUUUAAUUAGAA |
| ENST000 | | NO: | |
| 00313581- | | 653 | |
| 64 | | | |
| ENSG000 | 1.392974894 | SEQ | AGUAUCCCGAUAUUUUAAAAGCUGAGAUCUCCACU |
| 00151718- | | ID | ACAAGAUUAAGGU |
| ENST000 | | NO: | |
| 00504005- | | 654 | |
| 48 | | | |
| ENSG000 | 1.243892669 | SEQ | ACGCGCUCUACUCCUGUAACGGAAAGGUCGCGGCUU |
| 00151846- | | ID | GUGUGCCUGCGGGCAGCCGUGCCGAGA |
| ENST000 | | NO: | |
| 00281589- | | 655 | |
| 63 | | | |
| ENSG000 | 1.655756045 | SEQ | UUAUACUUCAGUUGUUCAGUCAGAUUGGACCCUGU |
| 00151923- | | ID | AAAAGCUGUAAA |
| ENST000 | | NO: | |
| 00369086- | | 656 | |
| 47 | | | |
| ENSG000 | 1.18852873 | SEQ | GCACAUUUGGCUAUUUUUCAAAGAGAGCAGUUAGU |
| 00152061- | | ID | UCAAGAAAGUGCUUAUUUUGUUUUUUAUUCUUCUU |
| ENST000 | | NO: | UUUGUGGUCUAUUUAGGAAAACAAA |
| 00347255- | | 657 | |
| 95 | | | |
| ENSG000 | 1.316014396 | SEQ | AGUCUGGUCUGGAGCUGGGACAGCAGCUGCUGCUG |
| 00152061 | | ID | UGGAAAGGCCAGCUGGCAAG |
| ENST000 | | NO: | |
| 00392064- | | 658 | |
| 55 | | | |
| ENSG000 | 1.553255941 | SEQ | ACCUUUCCGCGGGCCGCGGGG |
| 00152082- | | ID | |
| ENST000 | | NO: | |
| 00281871- | | 659 | |
| 21 | | | |
| ENSG000 | 1.214248325 | SEQ | AAAAAUCUCAGU |
| 00152234- | | ID | |
| ENST000 | | NO: | |
| 00585650- | | 660 | |
| 12 | | | |
| ENSG000 | 2.480118118 | SEQ | AGCCGCAGCU |
| 00152240- | | ID | |
| ENST000 | | NO: | |
| 00585518- | | 661 | |
| 10 | | | |
| ENSG000 | 1.460980997 | SEQ | GCAGUCCAAUUCAUCAGUUGUCUUUAGUUUACUCA |
| 00152266- | | ID | GCAUCAGCUACUAACAUACCUGAACGAAGAUCUUG |
| ENST000 | | NO: | UUCUAAGACAUUGUAGUUGAAG |
| 00282091- | | 662 | |
| 92 | | | |
| ENSG000 | 1.418126325 | SEQ | AGACUUGGUAUAGUGGGAGAGCCUGACUGAGAGUG |
| 00152556- | | ID | GAUC |
| ENST000 | | NO: | |
| 00312352- | | 663 | |
| 39 | | | |
| ENSG000 | 1.56883394 | SEQ | AUCUUGAUUCCUGCUGUGUCUUAACUGACCAUUGU |
| 00152556- | | ID | CUUAAAUUCUAGAGUGGAUC |
| ENST000 | | NO: | |
| 00546465- | | 664 | |
| 55 | | | |
| ENSG000 | 1.495932291 | SEQ | AGAAUAAACAUUUCUGGGA |
| 00152670- | | ID | |
| ENST000 | | NO: | |
| 00511853- | | 665 | |
| 19 | | | |
| ENSG000 | 1.267655147 | SEQ | GUGAAGUAGAGUGAGCAUUAGCCUGGCAGACAGCU |
| 00152818- | | ID | AGGGUAAUAGAGUACUUCCAGUGGAAGUCAAAGCA |
| ENST000 | | NO: | CAA |
| 00455022- | | 666 | |
| 73 | | | |
| ENSG000 | 2.319422253 | SEQ | GUGAAUUAUAGUUUAAGUUACUGCAAGUCCUAAAC |
| 00152942- | | ID | UACGGAGUGGAACUAUUACAGUUUAUA |
| ENST000 | | NO: | |
| 00380774- | | 667 | |
| 62 | | | |
| ENSG000 | 1.241287767 | SEQ | CAGGAGUUCCUCCUUGGCCGCGGCUGUAGCUGAGA |
| 00153048- | | ID | GAAGGGACAGGAGGUCAGCC |
| ENST000 | | NO: | |
| 00567554- | | 668 | |
| 55 | | | |
| ENSG000 | 1.347315863 | SEQ | ACUUCCGGUUAAGAAGUCAACACUCAGGUCUGAAA |
| 00153130- | | ID | AUUGAACAAG |
| ENST000 | | NO: | |
| 00338517- | | 669 | |
| 45 | | | |
| ENSG000 | 1.348682671 | SEQ | ACCUUUAAUUUCCUCGAAAACGCCUGUAACUCGGCU |
| 00153234- | | ID | GAAGCC |
| ENST000 | | NO: | |
| 00409108- | | 670 | |
| 42 | | | |
| ENSG000 | 1.120345783 | SEQ | UUACGACACUGUCCACCUUUAAUUUCCUCGAAAACG |
| 00153234- | | ID | CCUGUAACUCGGCUGAAGCUUCAGUACCUUU |
| ENST000 | | NO: | |
| 00429376- | | 671 | |
| 67 | | | |
| ENSG000 | 1.525738722 | SEQ | AUUUGCCUUCUCUUCACUUGGGGAGUAUCAAUUUG |
| 00153250- | | ID | CCACAGCAGUCUCUGGUCCCAGCCCACCCC |
| ENST000 | | NO: | |
| 00409289- | | 672 | |
| 65 | | | |
| ENSG000 | 1.413535488 | SEQ | UAAGGUAAUUCGUAGUCAAGAAGCUACACGUAAUU |
| 00153253- | | ID | AAAGUUGCAGGAGUAAAAG |
| ENST000 | | NO: | |
| 00440431- | | 673 | |
| 54 | | | |
| ENSG000 | 1.184687091 | SEQ | AAUUGACUUUGUGAUCAUUACAGAAAGUCUGGUGU |
| 00153283- | | ID | AAGGUGUUCAGAAGACA |
| ENST000 | | NO: | |
| 00283285- | | 674 | |
| 52 | | | |
| ENSG000 | 1.429514237 | SEQ | CAUCAAUUGACUUUGUGAUCAUUACAGAAAGUCUG |
| 00153283- | | ID | GUGUAAGGUGUUCAGAAGACA |
| ENST000 | | NO: | |
| 00494798- | | 675 | |
| 56 | | | |
| ENSG000 | 1.202250959 | SEQ | ACACUCAGUUUUGAACACACGGUGACACCUGAGAA |
| 00153303- | | ID | GC |
| ENST000 | | NO: | |
| 00336070- | | 676 | |
| 37 | | | |
| ENSG000 | 1.688431066 | SEQ | AUUCAUUUGAGUGGGAAUCUCAAAGCAGUUGAGUA |
| 00153802- | | ID | GGCAGAAAAAAGAACCUCUUCAUUAAGGAUUAAA |
| ENST000 | | NO: | |
| 00283916- | | 677 | |
| 69 | | | |
| ENSG000 | 1.180284691 | SEQ | UAAUCAGCUGUAAGUGCCGUCACUGGAUAGAAGGU |
| 00153814- | | ID | CCGUCCUUGGGUCUGU |
| ENST000 | | NO: | |
| 00430432- | | 678 | |
| 51 | | | |
| ENSG000 | 1.300920619 | SEQ | AUACAAAAACAUCAUAGUUAAGUAAACUCACCAGA |
| 00154165- | | ID | UUUGGCAUCUGCUCUUUGGUG |
| ENST000 | | NO: | |
| 00284311- | | 679 | |
| 56 | | | |
| ENSG000 | 1.534661839 | SEQ | AGUAACUGUGUUUCUAGAUUUUUCCUCCAGCUUUC |
| 00154258- | | ID | CUGAGAGAAACAGGUCAAA |
| ENST000 | | NO: | |
| 00495634- | | 680 | |
| 54 | | | |
| ENSG000 | 1.348255679 | SEQ | AUACAGUUUCUCUUUGCCAGACUAGACUAAAGAAG |
| 00154269- | | ID | GAGCACUAAUUUAUUCUGAUAAAACAGGUCUAGUC |
| ENST000 | | NO: | AGCUACCAGGACA |
| 00357639- | | 681 | |
| 83 | | | |
| ENSG000 | 1.316702052 | SEQ | ACAGUUUCUCUUUGCCAGACUAGACUAAAGAAGGA |
| 00154269- | | ID | GCACUAAUUUAUUCUGAUAAAACAGGUCUAGUCAG |
| ENST000 | | NO: | CUACCAGGACA |
| 00423831- | | 682 | |
| 81 | | | |
| ENSG000 | 1.233629493 | SEQ | AGCUGUUUUUCGUCUUCCCUAGGCUAUUUCUGCCG |
| 00154277- | | ID | GGCGCUCCGCGAAG |
| ENST000 | | NO: | |
| 00284440- | | 683 | |
| 49 | | | |
| ENSG000 | 1.301628321 | SEQ | UAUUGCUAGACCACCUUGCUUGGAAUUAAAAGUUA |
| 00154310- | | ID | CACAGUUAUUUUUCCCAGAACAGUUAUUUUCAGCU |
| ENST000 | | NO: | UAAGGA |
| 00468757- | | 684 | |
| 76 | | | |
| ENSG000 | 1.103263975 | SEQ | ACUUUUCCAGAUCUAGGAGCAUCAGUGACACCUCG |
| 00154479- | | ID | UCAGAGAGUCUGGUACGGUUUGGACGGCGCUGUGG |
| ENST000 | | NO: | ACGGGCGAAGGAAAGUACAGGGG |
| 00421028- | | 685 | |
| 93 | | | |
| ENSG000 | 1.147633269 | SEQ | UUUUCAGGCCAAAAGGUGUGUUUUGCUGACUUCAA |
| 00154645- | | ID | GCAUCCCUGCUACAAA |
| ENST000 | | NO: | |
| 00338326- | | 686 | |
| 51 | | | |
| ENSG000 | 1.486960459 | SEQ | GCGUGGCAGUUGAAAGAGUGGCGGGAGAAGUUGCA |
| 00154920- | | ID | GGGAAUUAUUUGAUAGCACAUACUG |
| ENST000 | | NO: | |
| 00511711- | | 687 | |
| 60 | | | |
| ENSG000 | 1.313618755 | SEQ | UGCCAAUCAUUGUGACAUUUUUCUGAUUUUUUUU |
| 00155249- | | ID | UUUAGGUAACUGAAUAUUGGAUAC |
| ENST000 | | NO: | |
| 00285600- | | 688 | |
| 59 | | | |
| ENSG000 | 1.460542016 | SEQ | AGUUGUUUCUGUAACAGCAGAAAGUGCACUCACUA |
| 00155307- | | ID | GGAGUAGUCAGAAUUCAAA |
| ENST000 | | NO: | |
| 00400566- | | 689 | |
| 54 | | | |
| ENSG000 | 1.212930496 | SEQ | CUUCCGGGCAAA |
| 00155463- | | ID | |
| ENST000 | | NO: | |
| 00442110- | | 690 | |
| 12 | | | |
| ENSG000 | 1.214818394 | SEQ | CUCUUCCGGGCAAA |
| 00155463- | | ID | |
| ENST000 | | NO: | |
| 00612549- | | 691 | |
| 14 | | | |
| ENSG000 | 1.14747866 | SEQ | GUUCGCCCUAAAGGUACCAAU |
| 00155545- | | ID | |
| ENST000 | | NO: | |
| 00381199- | | 692 | |
| 21 | | | |
| ENSG000 | 1.13181364 | SEQ | UCGUUCGCCCUAAAGGUACCAAU |
| 00155545- | | ID | |
| ENST000 | | NO: | |
| 00381213- | | 693 | |
| 23 | | | |
| ENSG000 | 2.132610821 | SEQ | UUCGCCCUAAAGGUACCAAU |
| 00155545- | | ID | |
| ENST000 | | NO: | |
| 00452157- | | 694 | |
| 20 | | | |
| ENSG000 | 1.232923977 | SEQ | UGACAGAAGCGUCAAUUCCUGGGAGUAGUUCGUUG |
| 00155621 | | ID | GUUUUCUUUCCCCUCAUCCUUUUGCCUGCUCCCGGC |
| ENST000 | | NO: | GAGGGGUGGCUUUGAUUUCGGCG |
| 00473252- | | 695 | |
| 94 | | | |
| ENSG000 | 1.094757446 | SEQ | GAAGUUUGAGCCUCUUUGGUAGCAGGAGGCUGGAA |
| 00155659- | | ID | GAAAGGACAGAAGUAGCUCUGGCUGUG |
| ENST000 | | NO: | |
| 00412866- | | 696 | |
| 62 | | | |
| ENSG000 | 1.806665059 | SEQ | AGAGCUGGAAUCUCUGCACGGGCCUUGGAAAACGA |
| 00155827- | | ID | CUGUCUUCUUCUGCCAAA |
| ENST000 | | NO: | |
| 00374819- | | 697 | |
| 53 | | | |
| ENSG000 | 1.25660662 | SEQ | AGUUUGAACUUACAAUACUUAAGUCCUGGCAAGUC |
| 00155833- | | ID | AUAAGUGGGGAAA |
| ENST000 | | NO: | |
| 00374798- | | 698 | |
| 48 | | | |
| ENSG000 | 1.226924038 | SEQ | ACAAUACUUAAGUCCUGGCAAGUCAUAAGUGGGGA |
| 00155833- | | ID | AA |
| ENST000 | | NO: | |
| 00487798- | | 699 | |
| 37 | | | |
| ENSG000 | 1.379887787 | SEQ | GAGUUCACCUUUCAACAUCACCAGUUUGAACUUAC |
| 00155833- | | ID | AAUACUUAAGUCCUGGCAAGUCAUAAGUGGGGAAA |
| ENST000 | | NO: | |
| 00612124- | | 700 | |
| 70 | | | |
| ENSG000 | 1.175313108 | SEQ | CGGCUGCAGAGGGGAAGGCGGCUACCAGUGUAAAG |
| 00155868- | | ID | CCAGAGCUGAGGUUCUUGAUAGUCCACA |
| ENST000 | | NO: | |
| 00286317- | | 701 | |
| 63 | | | |
| ENSG000 | 1.809713411 | SEQ | GACAGUUUCCCGAUUUGAGGUGAAACCAGUAAGAG |
| 00155893- | | ID | AAAAUAGAAUACUUAAUA |
| ENST000 | | NO: | |
| 00637579- | | 702 | |
| 53 | | | |
| ENSG000 | 1.195673929 | SEQ | GCUGUUGCCAUC |
| 00155957- | | ID | |
| ENST000 | | NO: | |
| 00613669- | | 703 | |
| 12 | | | |
| ENSG000 | 1.244232447 | SEQ | ACACUUUGCCACAGAAAGCAGUGAGCAAGA |
| 00155974- | | ID | |
| ENST000 | | NO: | |
| 00545666- | | 704 | |
| 30 | | | |
| ENSG000 | 1.204091313 | SEQ | ACUUUAUUACAGACCUUGGAAGCAAGAGGAUUGCA |
| 00156006- | | ID | UUCAGCCUAGUUCCUGGUUGCUGGCCAAAGGGAUC |
| ENST000 | | NO: | |
| 00286479- | | 705 | |
| 70 | | | |
| ENSG000 | 1.911499754 | SEQ | UUUGUACUUCCUGAGAGAAGGCAGAGAAGAAGAAG |
| 00156026- | | ID | UUUUUAUUACAUAGAGUAAC |
| ENST000 | | NO: | |
| 00604152- | | 706 | |
| 55 | | | |
| ENSG000 | 1.101524448 | SEQ | AGAAAGGAGCAGCAACUGGAAAACAAGCAUUGCAU |
| 00156096- | | ID | UGCAUCAGG |
| ENST000 | | NO: | |
| 00305107- | | 707 | |
| 44 | | | |
| ENSG000 | 1.416432995 | SEQ | ACAGCCUGGACUCAGAGCUCAAGUCUGAACUCUACC |
| 00156234- | | ID | UCCAGACAGA |
| ENST000 | | NO: | |
| 00682537- | | 708 | |
| 46 | | | |
| ENSG000 | 1.381627379 | SEQ | AUACACAAGAAGUUUCAGGAGUCAGAGGAGGGUUG |
| 00156273- | | ID | AGUAUAAUUAGAAGCAGUCUUUCCACUGAACUUCC |
| ENST000 | | NO: | CGACAACAUUUGUUAGUCAGA |
| 00447177- | | 709 | |
| 91 | | | |
| ENSG000 | 1.172091281 | SEQ | UCUCUUUCUGGUCAAA |
| 00156467- | | ID | |
| ENST000 | | NO: | |
| 00521036- | | 710 | |
| 16 | | | |
| ENSG000 | 1.380721414 | SEQ | UUAAAUAAGUAGGGAUUGUUUAAAAAAAAAAGCUG |
| 00156687- | | ID | UAAUAGUUUAUCUGGGGGUGG |
| ENST000 | | NO: | |
| 00453357- | | 711 | |
| 56 | | | |
| ENSG000 | 1.117138641 | SEQ | UCUAGCGAAAUAGUGCUACAGAGAGCCCUGCUACG |
| 00156958- | | ID | CGUCGGGUCCAGGUUACUGAAGCUAAAGGAG |
| ENST000 | | NO: | |
| 00559423- | | 712 | |
| 66 | | | |
| ENSG000 | 1.383482115 | SEQ | AUUGACUUGAGUCAAAACAUCACAAAGGCUCCAUA |
| 00157106- | | ID | UUAUACAGU |
| ENST000 | | NO: | |
| 00565224- | | 713 | |
| 44 | | | |
| ENSG000 | 1.303449037 | SEQ | ACUCCCCUGGCAGACCCCGCCAAGCGCCUCGGAGCG |
| 00157303- | | ID | CGCAGG |
| ENST000 | | NO: | |
| 00375472- | | 714 | |
| 42 | | | |
| ENSG000 | 1.176651374 | SEQ | GGUCAGAGAGAAGCAAUAAUUAUUAUUAACAUUUA |
| 00157554- | | ID | UUAACGAUCAAUAAACUUGAUCGCAUU |
| ENST000 | | NO: | |
| 00398905- | | 715 | |
| 62 | | | |
| ENSG000 | 1.10797867 | SEQ | UUUACUUUUCUUUUAUUAGGUUGUUUGCU |
| 00157578- | | ID | |
| ENST000 | | NO: | |
| 00380671- | | 716 | |
| 29 | | | |
| ENSG000 | 1.125559456 | SEQ | CUUACUUUGCAAAGAAGGAAG |
| 00157601- | | ID | |
| ENST000 | | NO: | |
| 00619682- | | 717 | |
| 21 | | | |
| ENSG000 | 1.530186557 | SEQ | CAGGACGUCUUCCAGAGUGUGUUUUUUGGCUACAG |
| 00157703- | | ID | UGUUUUCAGUAUCCUCUUUGGCCUCCUGGCUGACA |
| ENST000 | | NO: | GAU |
| 00436657- | | 718 | |
| 73 | | | |
| ENSG000 | 1.135989055 | SEQ | GUUUUAUCUCCCUUUCUGUUUUGUUUGUGACCAGG |
| 00157916- | | ID | CAUAAAGCAGUCACCUUCAACAGUUACAGA |
| ENST000 | | NO: | |
| 00443438- | | 719 | |
| 65 | | | |
| ENSG000 | 1.10366532 | SEQ | AGGAGAGUUGCCAAACUGUUAAGAGUGGUUAUUUC |
| 00158055- | | ID | UGAGCAGAAGA |
| ENST000 | | NO: | |
| 00236255- | | 720 | |
| 46 | | | |
| ENSG000 | 1.126411487 | SEQ | ACAGAGCAGUGAUAACUACCUGCCAGUGUCUCUUA |
| 00158125- | | ID | GGAGUGAGGUACCUGGAGUUCGGGGACCCCAACCU |
| ENST000 | | NO: | GUGACA |
| 00379416- | | 721 | |
| 76 | | | |
| ENSG000 | 1.166225024 | SEQ | AGUGUUCUAACUAUUAACGCUACG |
| 00158373- | | ID | |
| ENST000 | | NO: | |
| 00289316- | | 722 | |
| 24 | | | |
| ENSG000 | 1.274062176 | SEQ | CCUGUCAUUCGUUCGUCCUCAGUGCAGGGCAACAGG |
| 00158578- | | ID | ACUUUAGGUUCAAG |
| ENST000 | | NO: | |
| 00493869- | | 723 | |
| 50 | | | |
| ENSG000 | 1.727126472 | SEQ | UUCUUUUUGGAUUGAAGCCUUUUCCCCACGACUCU |
| 00158941- | | ID | GAAAGAGGACAGCGUUCCCA |
| ENST000 | | NO: | |
| 00523349- | | 724 | |
| 55 | | | |
| ENSG000 | 1.482570527 | SEQ | AUUAGAUAUUUCAUCUUACUGCAGAGCAUAGAAUC |
| 00158987- | | ID | AAUAGGAGCUUCUCCUUAGGGAGCUGCUUUGAAAC |
| ENST000 | | NO: | AGCUUCCAGCUGAUCUUACCAAG |
| 00513227- | | 725 | |
| 93 | | | |
| ENSG000 | 1.12745884 | SEQ | GUCCAUUCUUCCGGUGGAG |
| 00159111- | | ID | |
| ENST000 | | NO: | |
| 00421763- | | 726 | |
| 19 | | | |
| ENSG000 | 1.427199471 | SEQ | CUUCCGGUGGAG |
| 00159111- | | ID | |
| ENST000 | | NO: | |
| 00423147- | | 727 | |
| 12 | | | |
| ENSG000 | 1.20729524 | SEQ | AGUCUGGGAGCCUGGAGGACUAGCGAGGAGGAGUU |
| 00159140- | | ID | GAGAGAACGGAGCGGACGCC |
| ENST000 | | NO: | |
| 00356577- | | 728 | |
| 55 | | | |
| ENSG000 | 1.276294002 | SEQ | GCUACCGUGACUAAG |
| 00159377- | | ID | |
| ENST000 | | NO: | |
| 00290541- | | 729 | |
| 15 | | | |
| ENSG000 | 1.126393286 | SEQ | AUCUUCUUGCCAUUCAGUUGUAGAUCUUUGAAGAC |
| 00159516- | | ID | UCUGAUUCUCUGAGACUGAGGAGAG |
| ENST000 | | NO: | |
| 00368748- | | 730 | |
| 60 | | | |
| ENSG000 | 1.422864852 | SEQ | UUUUUCCUCUGCGAAUAGUGUUUUCUGAUUAACUA |
| 00159708- | | ID | CUAGUUAGUUCUAGUCCAAAAAAAGAAAC |
| ENST000 | | NO: | |
| 00435835- | | 731 | |
| 64 | | | |
| ENSG000 | 1.158459245 | SEQ | GCAGGUCCCACCUUGUGGAGG |
| 00160049- | | ID | |
| ENST000 | | NO: | |
| 00476658- | | 732 | |
| 21 | | | |
| ENSG000 | 1.36178054 | SEQ | ACUUCCGGUGUUACCUGUGUCGUUACCGGGAGCUG |
| 00160588- | | ID | UAAACAAGGUGUGCAAGCAUCUGAAGAGCUGCCGG |
| ENST000 | | NO: | G |
| 00278949- | | 733 | |
| 71 | | | |
| ENSG000 | 1.675653398 | SEQ | GUCGUUACCGGGAGCUGUAAACAAGGUGUGCAAGC |
| 00160588- | | ID | AUCUGAAGAGCUGCCGGG |
| ENST000 | | NO: | |
| 00525386- | | 734 | |
| 53 | | | |
| ENSG000 | 1.293935903 | SEQ | CUCAACUUCCCUAGUCAGUGACGCGGCGCCGGCCAG |
| 00161217- | | ID | AAAUCCGACCGGACCGGGCUCGGGGGAGCGUGAGU |
| ENST000 | | NO: | UGCAGUUAAAAGAAG |
| 00441879- | | 735 | |
| 86 | | | |
| ENSG000 | 1.389024992 | SEQ | CUGGCCUUCUCC |
| 00161249- | | ID | |
| ENST000 | | NO: | |
| 00436012- | | 736 | |
| 12 | | | |
| ENSG000 | 1.258751599 | SEQ | CCAGACCUGGAGGGCUUGGGUAAAA |
| 00161265- | | ID | |
| ENST000 | | NO: | |
| 00601236- | | 737 | |
| 25 | | | |
| ENSG000 | 1.194196332 | SEQ | ACUUCCGGAACAAGCGUCGCGUUUCUGAGGAGAAA |
| 00161920- | | ID | CUCUUGGUGAGAAUUCCCAGAGUGAUA |
| ENST000 | | NO: | |
| 00293777- | | 738 | |
| 62 | | | |
| ENSG000 | 1.296649421 | SEQ | CUAGUUUCUAAGGAUC |
| 00161960- | | ID | |
| ENST000 | | NO: | |
| 00578476- | | 739 | |
| 16 | | | |
| ENSG000 | 1.190368102 | SEQ | ACACUGGCCCUUGCUUUGGAGUCAGGAGGCCUCUCU |
| 00162078- | | ID | UCUUCCCACAGAGCCCUGGG |
| ENST000 | | NO: | |
| 00572863- | | 740 | |
| 56 | | | |
| ENSG000 | 1.191410773 | SEQ | CUUUUAUUGCUUUUGUACAAGACCAGACAGGAUCU |
| 00162086- | | ID | CAUUUGUUAAACGUGGUACCAAUUGGGUGUCUUAA |
| ENST000 | | NO: | CACAGGAGCAGAACUUCCUAGAGCAGA |
| 00612186- | | 741 | |
| 97 | | | |
| ENSG000 | 1.595056086 | SEQ | CAGCCAGUCCUGAGCACCUAACC |
| 00162438- | | ID | |
| ENST000 | | NO: | |
| 00375943- | | 742 | |
| 23 | | | |
| ENSG000 | 1.372891159 | SEQ | GUCACCGCUUUCGCUUCUGCUUGAGUAGUCAAGUG |
| 00162600- | | ID | AAAAA |
| ENST000 | | NO: | |
| 00371226- | | 743 | |
| 40 | | | |
| ENSG000 | 1.146783963 | SEQ | UGGAGAAAAGCCAAACAAUUGCAGUUUUCCAUAUU |
| 00162601- | | ID | UUAGAGAAAAUAAAUUAAUCAGUCUCAGUUUUCU |
| ENST000 | | NO: | |
| 00622766- | | 744 | |
| 69 | | | |
| ENSG000 | 1.242603249 | SEQ | UGACAGCAGCCAGCUCAGGAGGCAGAGGACGAGAG |
| 00162641- | | ID | CGAUCGGGCUGCACACAC |
| ENST000 | | NO: | |
| 00369994- | | 745 | |
| 53 | | | |
| ENSG000 | 1.196267171 | SEQ | UUAUCUGCAGAGAUCAGGCCAAAGGGAGCUCUAGA |
| 00162735- | | ID | AGGUUGUUGGGGAGCACUAAACUGGAGAAUAAGAG |
| ENST000 | | NO: | AAGAUAGUUUAAGUCCGUACC |
| 00392220- | | 746 | |
| 91 | | | |
| ENSG000 | 1.27790561 | SEQ | CAGACAGGAGCCGAACGGGGGCUUCCGCUCAGCAGA |
| 00162736- | | ID | GAGGCAAG |
| ENST000 | | NO: | |
| 00437169- | | 747 | |
| 44 | | | |
| ENSG000 | 1.115541037 | SEQ | UCUUCCCUAUCCCUAGUUCCAAAAG |
| 00162929- | | ID | |
| ENST000 | | NO: | |
| 00356719- | | 748 | |
| 25 | | | |
| ENSG000 | 1.662013999 | SEQ | AGUGUCCAGAAGUCUGGAGCGUAGGUGAAAGGACA |
| 00163072- | | ID | AAAGCCAGACACAUUUCAAC |
| ENST000 | | NO: | |
| 00439509- | | 749 | |
| 55 | | | |
| ENSG000 | 1.519405663 | SEQ | AGCAUAUUUCAUUUUCUGUCAUUGGACUUUGAGCC |
| 00163110- | | ID | AUUAGAACC |
| ENST000 | | NO: | |
| 00508216- | | 750 | |
| 44 | | | |
| ENSG000 | 1.390898977 | SEQ | UGAACUGAAAUCUCCUUGUUGCUUUGAAAUCUUAG |
| 00163131- | | ID | AAGAGAGCCCACUAAUUCAAGGACUCUUACUGUGG |
| ENST000 | | NO: | GAGCAACUGCUGGUUCUAUCACA |
| 00680311- | | 751 | |
| 93 | | | |
| ENSG000 | 1.430130946 | SEQ | CAGCACUCCACCAAAGCCUCUGCCUCAGCCUUACUG |
| 00163207- | | ID | UGAGUCUGGUUGACAGUAGCUUCUAAG |
| ENST000 | | NO: | |
| 00368764- | | 752 | |
| 63 | | | |
| ENSG000 | 1.642518385 | SEQ | UAAACUCCUGGUACUCUAGCACCGAUCUGCUUUGG |
| 00163216- | | ID | AGAACCUGAUCCUGAGACUCCAGCAGG |
| ENST000 | | NO: | |
| 00360379- | | 753 | |
| 62 | | | |
| ENSG000 | 1.40718731 | SEQ | UAUAAUUUCGUACAGCAACUUGCGGAGAGAUUUGU |
| 00163297- | | ID | GAGCCCUGAA |
| ENST000 | | NO: | |
| 00449651- | | 754 | |
| 45 | | | |
| ENSG000 | 1.275629366 | SEQ | ACAGCUGGCAUUCAGCCUCCAGAGCACCAGCACUGG |
| 00163331- | | ID | CACUGGCACUGGCACACGCU |
| ENST000 | | NO: | |
| 00309950- | | 755 | |
| 56 | | | |
| ENSG000 | 1.111938635 | SEQ | CUUCCUCGGAACUGUUGCCCAC |
| 00163352- | | ID | |
| ENST000 | | NO: | |
| 00368427- | | 756 | |
| 22 | | | |
| ENSG000 | 1.10833621 | SEQ | AGAAUACGGUGAGAAAUUCCAGGAGCACAGAAGUC |
| 00163354- | | ID | AGCGGGCAUCUCCUGGAGACAGGCAGACAUUUGGU |
| ENST000 | | NO: | CUCAGCAGCC |
| 00368424- | | 757 | |
| 80 | | | |
| ENSG000 | 1.22356696 | SEQ | CUUUCUUGGCCAGAGGCGCCGGUUGGACUCACGGGC |
| 00163463- | | ID | GGGGCAGU |
| ENST000 | | NO: | |
| 00482246- | | 758 | |
| 44 | | | |
| ENSG000 | 1.209765684 | SEQ | GAGAAUUGCUUCCUGAGAAGGUAAGUA |
| 00163468- | | ID | |
| ENST000 | | NO: | |
| 00446905- | | 759 | |
| 27 | | | |
| ENSG000 | 1.220822728 | SEQ | CUCUUCCUGUCUUUGUGGCUCCGGAAAGGCGUUUG |
| 00163479- | | ID | GGAGUCCAACG |
| ENST000 | | NO: | |
| 00496742- | | 760 | |
| 46 | | | |
| ENSG000 | 1.584138237 | SEQ | ACUGUCAGCUUUGAACACUGAACGCGAGGACUGUU |
| 00163600- | | ID | AACUGUUUCUGGCAAAC |
| ENST000 | | NO: | |
| 00316386- | | 761 | |
| 52 | | | |
| ENSG000 | 1.126515178 | SEQ | AGUAUAUUAGUGCUAAUUUCCCUCCGUUUGUCCUA |
| 00163631- | | ID | GCUUUUCUCUUCUGUCAACCCCACACGCCUUUGGCA |
| ENST000 | | NO: | CA |
| 00621628- | | 762 | |
| 73 | | | |
| ENSG000 | 1.156801668 | SEQ | ACAGAAGCCGUUUCUCAUC |
| 00163646- | | ID | |
| ENST000 | | NO: | |
| 00327047- | | 763 | |
| 19 | | | |
| ENSG000 | 1.157314453 | SEQ | AGAAUCCAGCACUCCAAGCACUGCUGUCUUCUCACA |
| 00163687- | | ID | GAGUCUUGAAGCCAGAGCAGCGCCAGG |
| ENST000 | | NO: | |
| 00486455- | | 764 | |
| 63 | | | |
| ENSG000 | 1.141606568 | SEQ | ACCUUCAGGCCAGUCAGCCAGUUUCCGGGAGCCCUA |
| 00163701- | | ID | AUUGCACAGAAGCCC |
| ENST000 | | NO: | |
| 00454992- | | 765 | |
| 51 | | | |
| ENSG000 | 2.057044302 | SEQ | AGGUGGAGGAG |
| 00163743- | | ID | |
| ENST000 | | NO: | |
| 00507014- | | 766 | |
| 11 | | | |
| ENSG000 | 1.222486514 | SEQ | GUAUCUGCCCAUCUUAGAUUCUUCCUAGCAAUAAU |
| 00163746- | | ID | UCUGAGAGACUCUGGAUUGUGGGCCGAAGUGAGA |
| ENST000 | | NO: | |
| 00613069- | | 767 | |
| 69 | | | |
| ENSG000 | 1.196060089 | SEQ | CAAAGAAGAACC |
| 00163751- | | ID | |
| ENST000 | | NO: | |
| 00296046- | | 768 | |
| 12 | | | |
| ENSG000 | 1.371076129 | SEQ | AGAGAUAGAGUCUUCCCUGGCAUUGCAGGAGAGAA |
| 00163810- | | ID | UCUGAAGGG |
| ENST000 | | NO: | |
| 00422219- | | 769 | |
| 44 | | | |
| ENSG000 | 1.421708959 | SEQ | UCAUUCAAGUGAGUAAGUUAAGAAGUGCCAAGGGU |
| 00163885- | | ID | GAGGAUCUCCUUUAGAAGAGGAGAAGCCUUGCAUC |
| ENST000 | | NO: | AACCUCUUGGGCCUCAGCCAAG |
| 00510833- | | 770 | |
| 92 | | | |
| ENSG000 | 1.644096472 | SEQ | AGGUGAAAAAUUAGACGAUUUCAAGAAUUGGAGCU |
| 00164118- | | ID | GAAUCUGAGUUUAAGUA |
| ENST000 | | NO: | |
| 00296519- | | 771 | |
| 52 | | | |
| ENSG000 | 1.221022678 | SEQ | GUAAGUUUUUUUUGGUUGCUGACAAAGUUCUUUUU |
| 00164129- | | ID | AUUCCAAGCAGGACUAUAAU |
| ENST000 | | NO: | |
| 00506953- | | 772 | |
| 55 | | | |
| ENSG000 | 1.538892719 | SEQ | UUACACUAAGAACUCUUUGUUACAUCAGGUAUUGC |
| 00164164- | | ID | ACUCUCAGUCUCGCCAGUUUGAAGUCAGA |
| ENST000 | | NO: | |
| 00509620- | | 773 | |
| 64 | | | |
| ENSG000 | 1.583020697 | SEQ | AUUAUAAACUCAUUUCUGUUUUAUUUUACUGAAGA |
| 00164181- | | ID | ACCCAUUAAAAGAGCCAGUAAAUCCUGUGGAAA |
| ENST000 | | NO: | |
| 00504455- | | 774 | |
| 68 | | | |
| ENSG000 | 1.129415916 | SEQ | AGAAACAUUCAGAUUCUCCAGAGAAAGUUGUAUUU |
| 00164483- | | ID | ACAGGAGACAGCA |
| ENST000 | | NO: | |
| 00529723- | | 775 | |
| 48 | | | |
| ENSG000 | 1.184568011 | SEQ | ACAGCACGCUGCCACGCCGACGCAGACCCCUCUCUG |
| 00164687- | | ID | CACGCCAGCCCGCCCGCACCCACC |
| ENST000 | | NO: | |
| 00297258- | | 776 | |
| 60 | | | |
| ENSG000 | 1.176135501 | SEQ | ACAUAUCCACUCCUGCUCUCCCUCCUGCAGGUGACC |
| 00164816- | | ID | CCAGCC |
| ENST000 | | NO: | |
| 00330590- | | 777 | |
| 42 | | | |
| ENSG000 | 1.525410616 | SEQ | ACUCUGCAUUUCUUUCCCGCCCUCUGCAGUAGUGUU |
| 00164828- | | ID | UGAAGUGGUGAAC |
| ENST000 | | NO: | |
| 00401592- | | 778 | |
| 49 | | | |
| ENSG000 | 1.215169189 | SEQ | AGCACGGUUCGUUUUUCCUUUAGUCAGGAAGGACG |
| 00164919- | | ID | UUGGUGUUGAGCAUACGUAUCAAGGACAGUAACUA |
| ENST000 | | NO: | CC |
| 00297564- | | 779 | |
| 72 | | | |
| ENSG000 | 1.216700317 | SEQ | UUUCCGGCUCCGGUGUC |
| 00165055- | | ID | |
| ENST000 | | NO: | |
| 00497665- | | 780 | |
| 17 | | | |
| ENSG000 | 1.106943014 | SEQ | AGGGCGGAGCGGGCGGCAGACCCGGAGCAGC |
| 00165060- | | ID | |
| ENST000 | | NO: | |
| 00484259- | | 781 | |
| 31 | | | |
| ENSG000 | 1.471090327 | SEQ | AGUCAUCUUUAUCCCUUUAAGCAGUGUUUAUUUUC |
| 00165120- | | ID | UGAGCAAGGAGUCAUC |
| ENST000 | | NO: | |
| 00297819- | | 782 | |
| 51 | | | |
| ENSG000 | 1.528556817 | SEQ | ACAUUCAACCUCUGCCACC |
| 00165168- | | ID | |
| ENST000 | | NO: | |
| 00378588- | | 783 | |
| 19 | | | |
| ENSG000 | 1.428969599 | SEQ | AGGAAGAGAGUCAGGAGGCGAUUUUUACCUCAGAG |
| 00165181- | | ID | UUUGAAAUUUACACUGCUUUGAAAGAAAGAAGAU |
| ENST000 | | NO: | |
| 00683944- | | 784 | |
| 69 | | | |
| ENSG000 | 1.256873376 | SEQ | AGACCUCUCGGGCGAGCGCGGCGCAGCGCAGAUUAA |
| 00165325- | | ID | AAAUCAAGAAAUAUAAACCAGAGUUAGCAGUUUCU |
| ENST000 | | NO: | UGAC |
| 00298050- | | 785 | |
| 75 | | | |
| ENSG000 | 1.431428471 | SEQ | AUUAAAAAUCAAGAAAUAUAAACCAGAGUUAGCAG |
| 00165325- | | ID | UUUCUUGAC |
| ENST000 | | NO: | |
| 00532819- | | 786 | |
| 44 | | | |
| ENSG000 | 1.414595795 | SEQ | GAGAAGUAGGCGAUUUCGGAGGAGUGAGAAAUAGC |
| 00165409- | | ID | CCCGAGUCCCGUGGAAA |
| ENST000 | | NO: | |
| 00555326- | | 787 | |
| 52 | | | |
| ENSG000 | 1.545112709 | SEQ | CUUCCUUGUAAG |
| 00165526- | | ID | |
| ENST000 | | NO: | |
| 00298317- | | 788 | |
| 12 | | | |
| ENSG000 | 1.156547604 | SEQ | UUCCAGGAUCAGAAGUACUGAGGAGCAGAGCCAAU |
| 00165555- | | ID | CUUCUCUGUUUGGAAAUAGUGGCUUGGAA |
| ENST000 | | NO: | |
| 00555603- | | 789 | |
| 64 | | | |
| ENSG000 | 1.241647736 | SEQ | AGUUGUUCUCAGGUGUUUGGGCUUGUUGUUCCGUA |
| 00165630- | | ID | UACUCAGUGGGUUCGCGGCCGCCGGCCCAGUGAGGC |
| ENST000 | | NO: | UGGGUUCGAGGAGCUGGAGCGGGAAACUGGAGCUU |
| 00378572- | | 790 | AAAUUCUGGCGGCGAG |
| 122 | | | |
| ENSG000 | 1.148512534 | SEQ | GCGAGUUUGACGGGGUCCUAAGAGUGCUGCUGGGC |
| 00165669- | | ID | GACCACUUCCUGACAGAAGAAAAG |
| ENST000 | | NO: | |
| 00369172- | | 791 | |
| 59 | | | |
| ENSG000 | 1.10941127 | SEQ | ACUCUUGUUGCAUCAUCAGCGUGCACCUCCACGAGU |
| 00165832- | | ID | AAACAGGUCUGGGCUACAAAAGU |
| ENST000 | | NO: | |
| 00298746- | | 792 | |
| 59 | | | |
| ENSG000 | 1.229538326 | SEQ | GUCAGCUUCACAUUCUCAGGAACUCUCCUUCUUUGG |
| 00165949- | | ID | GUCUGGCUGAAGUUGAGGAUCUCUUACUCUCUAGG |
| ENST000 | | NO: | CCACGGAAUUAACCCGAGCAGGC |
| 00620396- | | 793 | |
| 94 | | | |
| ENSG000 | 1.351093994 | SEQ | CAAACCCAGUCUUGUCAAUAGCGGGUUUCACCCUCC |
| 00165970- | | ID | ACCAGUUCAGUCUGUUGCCUGUGUCAGAC |
| ENST000 | | NO: | |
| 00525748- | | 794 | |
| 65 | | | |
| ENSG000 | 1.409596512 | SEQ | AGAAAAAAGAAAUCCUCUUUUUAGAACAUCUCUUG |
| 00165983- | | ID | GUGGUACCAUCAGAA |
| ENST000 | | NO: | |
| 00298942- | | 795 | |
| 50 | | | |
| ENSG000 | 1.273258764 | SEQ | GUUUCUCUCCGCAAGCGCGCG |
| 00166123- | | ID | |
| ENST000 | | NO: | |
| 00562132- | | 796 | |
| 21 | | | |
| ENSG000 | 1.102281595 | SEQ | GUCAGAGGGCCGGAGCGAGAAG |
| 00166128- | | ID | |
| ENST000 | | NO: | |
| 00559927- | | 797 | |
| 22 | | | |
| ENSG000 | 1.226043733 | SEQ | AAACGUGAAGAAGGUGAAG |
| 00166136- | | ID | |
| ENST000 | | NO: | |
| 00528425- | | 798 | |
| 19 | | | |
| ENSG000 | 1.316731002 | SEQ | AAACAGAUACCAGUGAGUAGUUUGAAACAGGAACA |
| 00166199- | | ID | AAAUCUUCUGAAAGCUCGGAGCAGAAGCCUUUUUG |
| ENST000 | | NO: | GUCAAC |
| 00529366- | | 799 | |
| 76 | | | |
| ENSG000 | 1.152093597 | SEQ | CUCUCUUUGUCCAACGUAGAAGAGACA |
| 00166329- | | ID | |
| ENST000 | | NO: | |
| 00299415- | | 800 | |
| 27 | | | |
| ENSG000 | 1.836787708 | SEQ | GAAAUCUGUCUUUGUUGCUCUGGGAGAGGGGACUC |
| 00166352- | | ID | CUGGAAGUUGUCUGUGAAUAAAGACUAGCCGAAGA |
| ENST000 | | NO: | UACUGUGGCCUCAGUAAUAGGA |
| 00676885- | | 801 | |
| 92 | | | |
| ENSG000 | 1.266002899 | SEQ | UCUUCCCUUCUUUGUAGUUCAGGAUUCGCCCAAA |
| 00166415- | | ID | |
| ENST000 | | NO: | |
| 00559418- | | 802 | |
| 34 | | | |
| ENSG000 | 1.3308146 | SEQ | CUUUUUCGUCUGGGCUGCCAAC |
| 00166441- | | ID | |
| ENST000 | | NO: | |
| 00314138- | | 803 | |
| 22 | | | |
| ENSG000 | 1.411305208 | SEQ | CAGAAGAUACUUGUGUUUUCCUGUAGUUGACAAAA |
| 00166478- | | ID | AACGGUAGAAG |
| ENST000 | | NO: | |
| 00417726- | | 804 | |
| 46 | | | |
| ENSG000 | 1.164322509 | SEQ | AGUGUCUUUAUCCCUCUUUUGCACAGUCAGCUUCU |
| 00166479- | | ID | GCAGCUCUCCCGGGCUAGC |
| ENST000 | | NO: | |
| 00569053- | | 805 | |
| 54 | | | |
| ENSG000 | 1.391687773 | SEQ | AACACUCCUGAGGAGAAAGAAAGAGAGGGAGGGAG |
| 00166523- | | ID | AGAAAAAGAGAGAGAGAGAAACAAAAAACCAAAGA |
| ENST000 | | NO: | GAGAGAAAAA |
| 00446457- | | 806 | |
| 80 | | | |
| ENSG000 | 1.12078269 | SEQ | GGAGGUUUUCUCUUGGGUUUGUUUGGAUUUUUUUU |
| 00166569- | | ID | UUCAGCUUUUGUCUGAAUUUUGGUUUUAUUUUUCU |
| ENST000 | | NO: | GGGGCAGAGAAA |
| 00587244- | | 807 | |
| 82 | | | |
| ENSG000 | 1.305837737 | SEQ | CAGCCCUUGCCAGCAUCCAGCC |
| 00166716- | | ID | |
| ENST000 | | NO: | |
| 00299927- | | 808 | |
| 22 | | | |
| ENSG000 | 1.344619977 | SEQ | GCGGAGCGGCAGGAGUCAGGAGGAGCUGGCGUGGG |
| 00166839- | | ID | AGACCGACGGCCUUUGGG |
| ENST000 | | NO: | |
| 00496660- | | 809 | |
| 53 | | | |
| ENSG000 | 1.313277674 | SEQ | CGGGCCAAGCCUACCGGGGGUCACAAUUUGGGUCU |
| 00167528- | | ID | GCAGCAGAA |
| ENST000 | | NO: | |
| 00549125- | | 810 | |
| 44 | | | |
| ENSG000 | 1.24220233 | SEQ | ACAGUGAAAAAGCAGUCUGGCUCCCGAGGUCCACCC |
| 00167615- | | ID | CUUAUACCCCAAGGUCCAG |
| ENST000 | | NO: | |
| 00610347- | | 811 | |
| 55 | | | |
| ENSG000 | 1.107255084 | SEQ | UUCUUCAGCCUUAACCUAAGGUCUCAUACUCGGAGC |
| 00167634- | | ID | ACU |
| ENST000 | | NO: | |
| 00328092- | | 812 | |
| 39 | | | |
| ENSG000 | 1.26005911 | SEQ | ACAGCCCACCAGUGACCACGAAGGCUGUGCUGCUUG |
| 00167653- | | ID | CCCUGUUG |
| ENST000 | | NO: | |
| 00301258- | | 813 | |
| 44 | | | |
| ENSG000 | 1.416393545 | SEQ | AAACGUGCCCCUUCACCGGAACCGAGCAACGGUAAC |
| 00167693- | | ID | UGUGCCCCUUCACCGGAACCGAGCA |
| ENST000 | | NO: | |
| 00575801- | | 814 | |
| 61 | | | |
| ENSG000 | 1.235074399 | SEQ | UAGUGCAAGUAAGAAAGAAACUGAAUUUGUCAUUU |
| 00167904- | | ID | UCACCUGAAGAAAA |
| ENST000 | | NO: | |
| 00522030- | | 815 | |
| 49 | | | |
| ENSG000 | 1.311760597 | SEQ | AGUGGCAUCCUUCCCUUUCUAAUCAGAGAUUUUCU |
| 00167910- | | ID | UCCUCAGAGAUUUUGGCCUAGAUUUGCAAA |
| ENST000 | | NO: | |
| 00301645- | | 816 | |
| 65 | | | |
| ENSG000 | 1.377414837 | SEQ | ACACUCUUCGUCAUCACCUCUCCUAUUCGCCUGGAC |
| 00167916- | | ID | AAGCUCAGUUUUGCAGGAGCACC |
| ENST000 | | NO: | |
| 00264651- | | 817 | |
| 59 | | | |
| ENSG000 | 1.343453797 | SEQ | GUAAAUCCGCCAUCUUCCUGCGGCGCGUUGCGAC |
| 00167967- | | ID | |
| ENST000 | | NO: | |
| 00565090- | | 818 | |
| 34 | | | |
| ENSG000 | 1.14438112 | SEQ | AGAGACGACCUUAAGUGGACAGAGAUUCUAGAACA |
| 00168070- | | ID | GGAGAAAUAAAACAGCUAUCGUUUGGGAGCA |
| ENST000 | | NO: | |
| 00432175- | | 819 | |
| 66 | | | |
| ENSG000 | 1.117737096 | SEQ | UGCGUAUACAUACAUACUAGUUAGUUGCGUUUAGU |
| 00168385- | | ID | UGUGUCUGUGUGUUUUUUUUUUAACAGACGAAGCU |
| ENST000 | | NO: | UCACAAAAG |
| 00449239- | | 820 | |
| 79 | | | |
| ENSG000 | 2.183440562 | SEQ | AGAGCAUAGCACCUGCAGCAAG |
| 00168484- | | ID | |
| ENST000 | | NO: | |
| 00524255- | | 821 | |
| 22 | | | |
| ENSG000 | 1.13522483 | SEQ | GCUUUAAUAGGGAGAUCAAGAGUAAUUUGAGUUAU |
| 00168502- | | ID | AAUCAGAAUCCUAGAGUGAGGAGGAGCACCUCUAG |
| ENST000 | | NO: | UCCUUGAGGUGGAAAGAACUGGAA |
| 00518815- | | 822 | |
| 94 | | | |
| ENSG000 | 1.102748702 | SEQ | ACACCUUCUGAGGCAGCGGC |
| 00168925- | | ID | |
| ENST000 | | NO: | |
| 00361017- | | 823 | |
| 20 | | | |
| ENSG000 | 1.435108539 | SEQ | UUAAGUCGUUCCCUCUACAAAGGACUUCCUAGUGG |
| 00168961- | | ID | GUGUGAAAGGCAGCGGUGGCCACAGAGGCGGCGGA |
| ENST000 | | NO: | GAG |
| 00577392- | | 824 | |
| 73 | | | |
| ENSG000 | 1.255378288 | SEQ | AAGUUGCUUUGGUUUCUAUUUCUUUGUUAAGUCGU |
| 00168961- | | ID | UCCCUCUACAAAGGACUUCCUAGUGGGUGUGAAAG |
| ENST000 | | NO: | GCAGCGGUGGCCACAGAGGCGGCGGAGAG |
| 00580779- | | 825 | |
| 99 | | | |
| ENSG000 | 1.22432666 | SEQ | GAUUAUUAAGUUCCUGCAACUUAACUGGGAACUGA |
| 00169239- | | ID | UCAAGAUUUCAAGCUAAAG |
| ENST000 | | NO: | |
| 00454127- | | 826 | |
| 54 | | | |
| ENSG000 | 1.380880693 | SEQ | ACAGAGUCUCUUUCCAGUCC |
| 00169282- | | ID | |
| ENST000 | | NO: | |
| 00475456- | | 827 | |
| 20 | | | |
| ENSG000 | 1.264156377 | SEQ | ACAAGCUCAGCG |
| 00169299- | | ID | |
| ENST000 | | NO | |
| 00512556- | | 828 | |
| 12 | | | |
| ENSG000 | 1.109415953 | SEQ | AGAACAACCAGCUGGAUCAGUUCUCACAGGAGCUA |
| 00169385- | | ID | CAGCGCGGAGACUGGGAAAC |
| ENST000 | | NO: | |
| 00304625- | | 829 | |
| 55 | | | |
| ENSG000 | 1.802634697 | SEQ | GUAAUAGUGUUUUAAGAAUAUUUCAGCGUAGUAGA |
| 00169398- | | ID | GGAUUUAAGCAGAUCGCUUUGCCUACUUUAAUUAU |
| ENST000 | | NO: | |
| 00519024- | | 830 | |
| 70 | | | |
| ENSG000 | 1.44833275 | SEQ | GUUUUAAGAAUAUUUCAGCGUAGUAGAGGAUUUAA |
| 00169398- | | ID | GCAGAUCGCUUUGCCUACUUUAAUUAU |
| ENST000 | | NO: | |
| 00520151- | | 831 | |
| 62 | | | |
| ENSG000 | 1.149006677 | SEQ | AAUAGUGUUUUAAGAAUAUUUCAGCGUAGUAGAGG |
| 00169398- | | ID | AUUUAAGCAGAUCGCUUUGCCUACUUUAAUUAU |
| ENST000 | | NO: | |
| 00523670- | | 832 | |
| 68 | | | |
| ENSG000 | 1.393839725 | SEQ | AUAACACCUAGUUUGAGUCAACCUGGUUAAGUACA |
| 00169605- | | ID | AAUAGUAGAAGGCUUCUCAUUCAGGUCC |
| ENST000 | | NO: | |
| 00673932- | | 833 | |
| 63 | | | |
| ENSG000 | 1.137406067 | SEQ | ACUGAAGAGUC |
| 00169607- | | ID | |
| ENST000 | | NO: | |
| 00302450- | | 834 | |
| 11 | | | |
| ENSG000 | 1.3820307 | SEQ | UCAGAGCUCUCAAGCACCCACG |
| 00169756- | | ID | |
| ENST000 | | NO: | |
| 00414829- | | 835 | |
| 22 | | | |
| ENSG000 | 1.321902051 | SEQ | GUUAGUAGUACCGUCGCUUUCCUGGAUAGUGGCUA |
| 00169764- | | ID | UCUUAGCAAAGCA |
| ENST000 | | NO: | |
| 00475462- | | 836 | |
| 48 | | | |
| ENSG000 | 1.220633954 | SEQ | UCCAGACAGGACUGUCCAGAGCAACGGUGUGCCAG |
| 00169783- | | ID | AGAGACUGAGAAGUACCCAGAA |
| ENST000 | | NO: | |
| 00557798- | | 837 | |
| 57 | | | |
| ENSG000 | 1.654364873 | SEQ | AAACUCCUCUUUGAUUCUUCUAGCUGUUUCACUAU |
| 00169906- | | ID | UGGGCAACCAGACACCAGA |
| ENST000 | | NO: | |
| 00380200- | | 838 | |
| 54 | | | |
| ENSG000 | 1.103786962 | SEQ | ACUCUCCUGUAAAACGCUAGAGCGGCGAGUUGUUA |
| 00169976- | | ID | CCUGCGUCCUCUGACCUGAGAGCGAAGGGGAAAGC |
| ENST000 | | NO: | GGCGAG |
| 00367569- | | 839 | |
| 76 | | | |
| ENSG000 | 1.217667466 | SEQ | GCAGUUCCGGUGAGUCCGUGG |
| 00170043- | | ID | |
| ENST000 | | NO: | |
| 00571947- | | 840 | |
| 21 | | | |
| ENSG000 | 1.132252954 | SEQ | AGCUACAACCAAGUAAGCAAGUGUCAGGGCUCACC |
| 00170054- | | ID | AACCAGUCAAGGACAGGGCAGGAGAAGAGGAACCU |
| ENST000 | | NO: | GCAAAGACAUAUUUUGUUCCAAA |
| 00298845- | | 841 | |
| 93 | | | |
| ENSG000 | 1.277076802 | SEQ | AAGUCAUUCCUUCUGCAAAGGACUGCCUGGCAGGU |
| 00170298- | | ID | GUGAAAGGCAGCGGUGGCCACAGAGGCGGUGGAG |
| ENST000 | | NO: | |
| 00423676- | | 842 | |
| 69 | | | |
| ENSG000 | 1.329092069 | SEQ | AGCCCUUGCGCCCUGUUUGUGCCAGCAGGCACCAGC |
| 00170325- | | ID | GAACCAAAAGCAGGUGUCC |
| ENST000 | | NO: | |
| 00526082- | | 843 | |
| 55 | | | |
| ENSG000 | 1.347256771 | SEQ | CCGGAACGUCUUUUUGCGGACGCCCUCGGAGCAGCC |
| 00170390- | | ID | GCG |
| ENST000 | | NO: | |
| 00411937- | | 844 | |
| 39 | | | |
| ENSG000 | 1.106061418 | SEQ | ACACACACAUCUGCACCUCAACCACAGACUACACUU |
| 00170476- | | ID | GCUGAACUGGCUCCUGGGGCC |
| ENST000 | | NO: | |
| 00302125- | | 845 | |
| 57 | | | |
| ENSG000 | 1.173639899 | SEQ | CUGCUUUUCUCCAGCUCUGUCAACUCAACCUUUCCC |
| 00170484- | | ID | ACC |
| ENST000 | | NO: | |
| 00549343- | | 846 | |
| 39 | | | |
| ENSG000 | 1.22328703 | SEQ | ACAGGCCCUUGGUUCCAUCCUCUGCCAUCUACUCCA |
| 00170523- | | ID | CUGUUCAGACACCUCCUAACCUCCGUC |
| ENST000 | | NO: | |
| 00293670- | | 847 | |
| 63 | | | |
| ENSG000 | 1.692965547 | SEQ | ACAGAUCUGCCUCUUCUCCAAGAAACUCAACCACUA |
| 00170545- | | ID | GUGACA |
| ENST000 | | NO: | |
| 00380103- | | 848 | |
| 42 | | | |
| ENSG000 | 1.655208685 | SEQ | GCUGUUUAGAACUAUUGUACCGCUGCCAGUGACUG |
| 00170579- | | ID | UCUGUAUUAGGGUUGGGAGCAGGAAC |
| ENST000 | | NO: | |
| 00515196- | | 849 | |
| 61 | | | |
| ENSG000 | 1.099285922 | SEQ | UAGGGACGGGAAGUCGCGACCAGAGCCAUUGGAGG |
| 00170581- | | ID | GCGCGGGGACUGCAACCCUAAUCAGAGCCCAA |
| ENST000 | | NO: | |
| 00652624- | | 850 | |
| 67 | | | |
| ENSG000 | 1.17560493 | SEQ | AGAAGUCCUGUAGGGAGAGUGGCAGUUACCUUCUC |
| 00170613- | | ID | UCCGUCUCUCUCUCGGGGACUCUAUUUGUGGCUGG |
| ENST000 | | NO: | UGGAGCUGUCUUAAACGAAGAGAACC |
| 00302938- | | 851 | |
| 96 | | | |
| ENSG000 | 1.114255163 | SEQ | CGAGCACUUGGAUUCAGCUUCUUCAUUUCCAAC |
| 00170627- | | ID | |
| ENST000 | | NO: | |
| 00546931- | | 852 | |
| 33 | | | |
| ENSG000 | 1.563860374 | SEQ | CCAGUGAAGUCCUGGGCAGAAGA |
| 00170782- | | ID | |
| ENST000 | | NO: | |
| 00379829- | | 853 | |
| 23 | | | |
| ENSG000 | 1.264633689 | SEQ | CACUUGACCUUCCAUCAGUAAGACGGACUUUUCUA |
| 00170852- | | ID | AUCUGUUCCUGGAGAUAUUAAGUGAAUACAGUC |
| ENST000 | | NO: | |
| 00621876- | | 854 | |
| 68 | | | |
| ENSG000 | 1.112044721 | SEQ | CUCUUUCUCAGUGACCGGGUGGUUUGCUUAGGCGC |
| 00170889- | | ID | AGACGGGGAAGCGGAGCCAAC |
| ENST000 | | NO: | |
| 00302907- | | 855 | |
| 56 | | | |
| ENSG000 | 1.222934026 | SEQ | UAAUUCAGCCACAUCUCCUCCUUCUCACUCAACUCA |
| 00170954- | | ID | GACCUCUCAGGAUAACUUGGUGAA |
| ENST000 | | NO: | |
| 00595174- | | 856 | |
| 60 | | | |
| ENSG000 | 1.159693194 | SEQ | CCUGACCGUAGUGCGGCCAGCAGUUGCAGGCAGACG |
| 00170955- | | ID | GAGCAGAGCGGUCAGGGAUC |
| ENST000 | | NO: | |
| 00530979- | | 857 | |
| 56 | | | |
| ENSG000 | 1.509461646 | SEQ | ACAGCAUUCCUGGAGCCCAGGCUCUUUUCCACAGAG |
| 00170956- | | ID | GAGGAAAGAGCAGGCAGCAGAGACC |
| ENST000 | | NO: | |
| 00415495- | | 858 | |
| 61 | | | |
| ENSG000 | 1.533932631 | SEQ | UUGAGUAAGUUCACUGGUGUGUCAUCAGUUUGCAG |
| 00171109- | | ID | UUCUAUCAUCCACUUUAGUUGGUGCUUUUCUAACU |
| ENST000 | | NO: | UUAUCUCCCUCUAGUAGCAUA |
| 00263969- | | 859 | |
| 91 | | | |
| ENSG000 | 1.373999542 | SEQ | GCAGAGGUCACCGUCCAGGUUGGACAGCAGCACCUU |
| 00171174- | | ID | UGAGCG |
| ENST000 | | NO: | |
| 00302188- | | 860 | |
| 42 | | | |
| ENSG000 | 1.36119849 | SEQ | GCCGUCCGCGGAAAUUUGAA |
| 00171241- | | ID | |
| ENST000 | | NO: | |
| 00303383- | | 861 | |
| 20 | | | |
| ENSG000 | 1.451884879 | SEQ | AAGCCUUCCAAACCCUCCUUCCUGGAAACCCACCCA |
| 00171396- | | ID | GAACCUCCACCCUCUGACACC |
| ENST000 | | NO: | |
| 00390661- | | 862 | |
| 57 | | | |
| ENSG000 | 1.31453917 | SEQ | UGGAAGUGCUCAAGUGUGGUAUACACUCAGGUGCA |
| 00171405- | | ID | UUUUUCCAAUCACAAUAUUCUGGACUGAAAU |
| ENST000 | | NO: | |
| 00351072- | | 863 | |
| 66 | | | |
| ENSG000 | 1.357584551 | SEQ | AAGAUUAUAGAGACUUGCUUUAGAACCACAAGAAG |
| 00171533- | | ID | AAAGAGGAGGCCGGCUUUUCAGCUAGCAUC |
| ENST000 | | NO: | |
| 00434603- | | 864 | |
| 65 | | | |
| ENSG000 | 1.402997763 | SEQ | AAUCCUUUCUUUCAGCUGGAGUGCUCCUCAGGAGCC |
| 00171560- | | ID | AGCCCCACCCUUAGAAAAG |
| ENST000 | | NO: | |
| 00403106- | | 865 | |
| 55 | | | |
| ENSG000 | 1.743207923 | SEQ | UUCUUCUUUUUUUCUCAAGAGAAUAUUUGACCUGA |
| 00171681- | | ID | GUUAAUUCUUGGAUCUCUGAGGUCUUCUACACAGA |
| ENST000 | | NO: | AGUAUAUUUAGAGAGCAAUUCAGA |
| 00534828- | | 866 | |
| 94 | | | |
| ENSG000 | 1.291928251 | SEQ | AAACUAAGAGUAGUGCUGCAGCAAGCUCCCUCUUG |
| 00171681- | | ID | GAUUCAGA |
| ENST000 | | NO: | |
| 00539057- | | 867 | |
| 43 | | | |
| ENSG000 | 1.339719472 | SEQ | AAGUUGCUUUGGUUUCUAUUACUUUGUUAAGUCAU |
| 00171916- | | ID | UCCUUCUGCAAAGGACUGCCUGGCAGGUGUCAAAG |
| ENST000 | | NO: | GCAGUGGUGGCCACAGAGGCGGUGGAGAG |
| 00577691- | | 868 | |
| 99 | | | |
| ENSG000 | 1.276933056 | SEQ | UGGUUUCUAUUACUUUGUUAAGUCAUUCCUUCUGC |
| 00171916- | | ID | AAAGGACUGCCUGGCAGGUGUCAAAGGCAGUGGUG |
| ENST000 | | NO: | GCCACAGAGGCGGUGGAGAG |
| 00582333- | | 869 | |
| 90 | | | |
| ENSG000 | 1.099509523 | SEQ | CAUCGCUCUGUGUUUCCUGCUCCAGGUUUCGCGUCU |
| 00171953- | | ID | CGCAUCUUCCCAGACCCUCGGGCGCG |
| ENST000 | | NO: | |
| 00462733- | | 870 | |
| 62 | | | |
| ENSG000 | 1.387947552 | SEQ | CUGUUUGGAGUUGGAAGCCGAGACCUAAAGUUGGG |
| 00172167- | | ID | GGGUGAUCUCUGAGGAG |
| ENST000 | | NO: | |
| 00305949- | | 871 | |
| 52 | | | |
| ENSG000 | 1.165971712 | SEQ | GUGAGACUUCCAGAUCUUCUCUGGUGAAGUGUGUU |
| 00172179- | | ID | UCCUGCAACGAUCACGAAC |
| ENST000 | | NO: | |
| 00306482- | | 872 | |
| 54 | | | |
| ENSG000 | 1.135030651 | SEQ | ACAUCACAGAAGUAGAGGCAGACCUUGCUUCAGUA |
| 00172215- | | ID | GCAAGCUCAUCUCUGGAACAAACUGGCAAAGCAUC |
| ENST000 | | NO: | UCUGCUGGUGUUCAUCAGAACAGACACC |
| 00304552- | | 873 | |
| 98 | | | |
| ENSG000 | 1.197357555 | SEQ | AGACAGUCAUCUCAGGAGCAGAAAGAAAAGAGCUC |
| 00172243- | | ID | CCAAAGUCUAUAUCUAUUCAGGGGCUCUCAAGAAC |
| ENST000 | | NO: | A |
| 00298523- | | 874 | |
| 71 | | | |
| ENSG000 | 1.411417369 | SEQ | AUUUCCUGCUCUUGAAUAUCUGGUUGAACUACUUA |
| 00172243- | | ID | AGCUUAAUUUGUUAAACUCCGGGGCUCUCAAGAAC |
| ENST000 | | NO: | A |
| 00529761- | | 875 | |
| 71 | | | |
| ENSG000 | 1.14069491 | SEQ | GGAAUAGGAAUC |
| 00172270- | | ID | |
| ENST000 | | NO: | |
| 00614867- | | 876 | |
| 12 | | | |
| ENSG000 | 1.162152591 | SEQ | GCGAGGAAUAGGAAUC |
| 00172270- | | ID | |
| ENST000 | | NO: | |
| 00618006- | | 877 | |
| 16 | | | |
| ENSG000 | 1.37492807 | SEQ | GUAGGACCGGCGAGGAAUAGGAAUC |
| 00172270- | | ID | |
| ENST000 | | NO: | |
| 00680326- | | 878 | |
| 25 | | | |
| ENSG000 | 1.528653385 | SEQ | UUGCUUUAUUUCUCAUUGCUACAGGUCAUCUUAAG |
| 00172365- | | ID | UUUCUCAGAUAAUACUG |
| ENST000 | | NO: | |
| 00302581- | | 879 | |
| 52 | | | |
| ENSG000 | 1.312636872 | SEQ | ACUGGGUUGCUGAUCCAGCU |
| 00172367- | | ID | |
| ENST000 | | NO: | |
| 00355547- | | 880 | |
| 20 | | | |
| ENSG000 | 2.852381712 | SEQ | AGAAUUUGAUUUGACUAUAUUUACCAUAUCAGAUU |
| 00172410- | | ID | CACAUUCAGUCCUCAGCAAA |
| ENST000 | | NO: | |
| 00304526- | | 881 | |
| 55 | | | |
| ENSG000 | 1.225818801 | SEQ | AUUGGAUUCCUGAUCUGAUUGAAGCCCAGAAGUGA |
| 00172421- | | ID | CAGAGUCACUGGCCAC |
| ENST000 | | NO: | |
| 00520404- | | 882 | |
| 51 | | | |
| ENSG000 | 1.151508341 | SEQ | UUGGGAUUUUGGAGUUGAGCACC |
| 00172425- | | ID | |
| ENST000 | | NO: | |
| 00528570- | | 883 | |
| 23 | | | |
| ENSG000 | 1.266011212 | SEQ | UAAGUGUACAGUCUUAGGCCUUCUGAAGCAGCAUU |
| 00172468- | | ID | UGAAGCUGCAGUCCUGAAAACCAGUCAGGCCGGAA |
| ENST000 | | NO: | GAGUAGAUAAAGAAAUAUUUAUUUGAG |
| 00309834- | | 884 | |
| 97 | | | |
| ENSG000 | 1.277180577 | SEQ | UUUGUUUUGUAGAUUGGUUGUUUCAUU |
| 00172775- | | ID | |
| ENST000 | | NO: | |
| 00389447- | | 885 | |
| 27 | | | |
| ENSG000 | 1.123937221 | SEQ | CCCUCCAUUGCU |
| 00173218- | | ID | |
| ENST000 | | NO: | |
| 00369509- | | 886 | |
| 12 | | | |
| ENSG000 | 1.328926193 | SEQ | AGUUUGUUGAAGUUCGUGACUGCUUCACUCUCUCA |
| 00173391- | | ID | UUCUUAGCUUGAAUUUGGAA |
| ENST000 | | NO: | |
| 00309539- | | 887 | |
| 55 | | | |
| ENSG000 | 2.00761585 | SEQ | AUUUUUAGUUUGUUGAAGUUCGUGACUGCUUCACU |
| 00173391- | | ID | CUCUCAUUCUUAGCUUGAAUUUGGAA |
| ENST000 | | NO: | |
| 00544577- | | 888 | |
| 61 | | | |
| ENSG000 | 1.16294317 | SEQ | AGCCCAGAAAGGAGCCACGGGGAGAAGCGGAGUUU |
| 00173452- | | ID | CUUCUUUGUGGCAUUUGUGGAAUAUUGUGUGCCAA |
| ENST000 | | NO: | AAAAAAAUCAGGACUUGUC |
| 00422233- | | 889 | |
| 89 | | | |
| ENSG000 | 1.180531783 | SEQ | UCUUCCUUGCGGAAAUUUGCAUCGUGGGCUUGUAG |
| 00173567- | | ID | GGACUGCUUCACUGAGCC |
| ENST000 | | NO: | |
| 00421160- | | 890 | |
| 53 | | | |
| ENSG000 | 1.793917021 | SEQ | GUCAGUGGAGGCAGCAGCGGUAGAGGCGGCGGCGA |
| 00173599- | | ID | GGACUGGCGACGGCGAGGAGAUAGUGUCUGCCUUC |
| ENST000 | | NO: | UGGAGAGCUGACCAAACACUAAGG |
| 00524491- | | 891 | |
| 94 | | | |
| ENSG000 | 1.411983838 | SEQ | AGUAUAUUCUGUUCUCUUGGGACAUUACAGUUUUG |
| 00173908- | | ID | CAUAGACAAGGUGAACAUUUCUCAAAACACC |
| ENST000 | | NO: | |
| 00306658- | | 892 | |
| 66 | | | |
| ENSG000 | 1.167350608 | SEQ | GCAAGUAGUACCUCAGGGUAAACUGCCAGAAGGCU |
| 00174038- | | ID | AAGGGAA |
| ENST000 | | NO: | |
| 00378745- | | 893 | |
| 42 | | | |
| ENSG000 | 1.099566468 | SEQ | GUCACAGAGGAG |
| 00174175- | | ID | |
| ENST000 | | NO: | |
| 00367795- | | 894 | |
| 12 | | | |
| ENSG000 | 1.906898622 | SEQ | ACUUAGUUAAAUUUCAGUAACUAUUAUAUCCGUUU |
| 00174371- | | ID | UCCUCGGAGUGAGAGAAAACUCUUUUUAGAUAUCA |
| ENST000 | | NO: | UCUGAGAGGUAGUUAAUUUGGCACC |
| 00437497- | | 895 | |
| 95 | | | |
| ENSG000 | 1.578869575 | SEQ | ACACUCAUUUUAGCCAGUGUCCAGGCUAUCAGCAG |
| 00174429- | | ID | AGAAAGACAGGUGGGCAGC |
| ENST000 | | NO: | |
| 00311955- | | 896 | |
| 54 | | | |
| ENSG000 | 1.147621291 | SEQ | GUUGCCAGGACGACGCCUGCGAAG |
| 00174483- | | ID | |
| ENST000 | | NO: | |
| 00525809- | | 897 | |
| 24 | | | |
| ENSG000 | 1.325274445 | SEQ | ACUAUUGGGCGUUACGCGAGGGCGGGGCCGGUUGC |
| 00174483- | | ID | CAGGACGACGCCUGCGAAG |
| ENST000 | | NO: | |
| 00526760- | | 898 | |
| 54 | | | |
| ENSG000 | 2.422909313 | SEQ | AACGCGGUUACCGUGGAAACCGCGGCC |
| 00174628- | | ID | |
| ENST000 | | NO: | |
| 00564186- | | 899 | |
| 27 | | | |
| ENSG000 | 1.442627439 | SEQ | CCCCUCGUCUCCUCCAAG |
| 00174780- | | ID | |
| ENST000 | | NO: | |
| 00504757- | | 900 | |
| 18 | | | |
| ENSG000 | 1.157674769 | SEQ | AGUAGAAAAGUUUCUUUUCUUUGAAGUACAGAACU |
| 00174837- | | ID | ACAGCAUA |
| ENST000 | | NO: | |
| 00250572- | | 901 | |
| 43 | | | |
| ENSG000 | 1.426691301 | SEQ | UCUGCAGAGUGAGAGCAAACUAAAAGAGAAAAAUC |
| 00174970- | | ID | AAAGUUGACUACAAUA |
| ENST000 | | NO: | |
| 00312345- | | 902 | |
| 51 | | | |
| ENSG000 | 1.096097895 | SEQ | CUUUCCUCUCCUGACUAAGUUUCUCUGGCUUCCCUG |
| 00175121- | | ID | AGGCUGCAGGUGUUAAUCUGGGGGGCCCUGGGCCC |
| ENST000 | | NO: | UGAGCCGGCAGCAGAAAU |
| 00372789- | | 903 | |
| 89 | | | |
| ENSG000 | 1.190833023 | SEQ | AGGUUCGAUACAAGAGGCUGUUUUCCUAGCGUGGC |
| 00175387- | | ID | UUGCUGCCUUUGGUAAGAAC |
| ENST000 | | NO: | |
| 00586040- | | 904 | |
| 55 | | | |
| ENSG000 | 1.543381243 | SEQ | AUUUGCCUCUGCAACUUGACUCUCCUCUAGGAAGA |
| 00175787- | | ID | GUACUCCAGAGAGCAGGGAU |
| ENST000 | | NO: | |
| 00480716- | | 905 | |
| 55 | | | |
| ENSG000 | 1.640477609 | SEQ | CUAUCUGACCACAUUGUUAAACUUGGUUAGUUUUG |
| 00176009- | | ID | CAGGUUAAAGAGGAA |
| ENST000 | | NO: | |
| 00531618- | | 906 | |
| 50 | | | |
| ENSG000 | 1.239154308 | SEQ | AUAGUAAAGGUCGGCCCGCGCAAG |
| 00177034- | | ID | |
| ENST000 | | NO: | |
| 00512528- | | 907 | |
| 24 | | | |
| ENSG000 | 1.46662933 | SEQ | CCUCUUUAGUGC |
| 00177103- | | ID | |
| ENST000 | | NO: | |
| 00527706- | | 908 | |
| 12 | | | |
| ENSG000 | 1.10877424 | SEQ | CCGAGCCCGAGCC |
| 00177225- | | ID | |
| ENST000 | | NO: | |
| 00524550- | | 909 | |
| 13 | | | |
| ENSG000 | 1.266570569 | SEQ | ACUCUUGGAGGAAGGCAUCAGUUAAGGACACUGGA |
| 00177669- | | ID | AAAGACAGGCUCUUUUGUUCUGGUUCCUA |
| ENST000 | | NO: | |
| 00320542- | | 910 | |
| 64 | | | |
| ENSG000 | 1.985642212 | SEQ | GAAAGUUCUAUAGUCUCUAUAUUCUGUUAUCAAGU |
| 00177684- | | ID | CUUUCAACAACUUCUUUCUACAGA |
| ENST000 | | NO: | |
| 00322066- | | 911 | |
| 59 | | | |
| ENSG000 | 1.449863416 | SEQ | AGAAGGUCACAAAGCUUGCAGGGUAAGUGACACAA |
| 00177694- | | ID | CUUGAAACUGCUUGGCCCUCUUUAAAAAGAAAUAA |
| ENST000 | | NO: | UAAA |
| 00454872- | | 912 | |
| 74 | | | |
| ENSG000 | 1.295821214 | SEQ | AAGUGAUUUGCUUAAAAUUUCUCACACAAAGCUAC |
| 00177752- | | ID | AUUUACUGGAGGAUCUUUCUAUAAAAAAUAAACAG |
| ENST000 | | NO: | AGG |
| 00332990- | | 913 | |
| 73 | | | |
| ENSG000 | 1.1016613 | SEQ | UGGCCAGGCCCAGGAAAUCC |
| 00178104- | | ID | |
| ENST000 | | NO: | |
| 00585156- | | 914 | |
| 20 | | | |
| ENSG000 | 1.11417304 | SEQ | ACAGAGAACCUCAGCUGGACAAAGCAGCCUUGAUC |
| 00178172- | | ID | UGAGUGAGCUAACUGACACA |
| ENST000 | | NO: | |
| 00325630- | | 915 | |
| 55 | | | |
| ENSG000 | 2.126320054 | SEQ | UCAUAUUUGUGCUCCUUCACGGGAGGGCAGGGAGG |
| 00178287- | | ID | UUCAACGGACCUUAAAAC |
| ENST000 | | NO: | |
| 00326625- | | 916 | |
| 53 | | | |
| ENSG000 | 1.28415615 | SEQ | UCUAGGUUUUCACAUCCUUUCUAGUAGUUUCGCCA |
| 00178460- | | ID | UCUAUAGCUUUUAUCAACAAUUGUGGUUUAAUCAA |
| ENST000 | | NO: | UUAGAAAUAUUAACCAGGAGAAA |
| 00415737- | | 917 | |
| 93 | | | |
| ENSG000 | 1.346267224 | SEQ | AAUUUUAAUCUUCUUUUCUUAGAACUAUCUUGGUU |
| 00178522- | | ID | GGCAUCAUCAGGCCCUGAGAGCACAGUGC |
| ENST000 | | NO: | |
| 00449493- | | 918 | |
| 64 | | | |
| ENSG000 | 1.452521093 | SEQ | CCGCGAGCUUACACCGGCUUCUCUCUGUCCUCAGCC |
| 00178741- | | ID | CGCGCGCCGCCAUCGCCGUC |
| ENST000 | | NO: | |
| 00562233- | | 919 | |
| 56 | | | |
| ENSG000 | 1.945486005 | SEQ | GUGAGUUGCAGUUUGUGUGAGGAUCCCGGGGCCGC |
| 00179262- | | ID | CGCGUCGCUCGGGCCCCGCC |
| ENST000 | | NO: | |
| 00593114- | | 920 | |
| 55 | | | |
| ENSG000 | 1.367184063 | SEQ | UUGCCUUUAGGUUUUCCGAAGGUCAACA |
| 00179626- | | ID | |
| ENST000 | | NO: | |
| 00394256- | | 921 | |
| 28 | | | |
| ENSG000 | 1.238926188 | SEQ | AUUUGUGACUUCUUCAGACCACUUUC |
| 00179915- | | ID | |
| ENST000 | | NO: | |
| 00331040- | | 922 | |
| 26 | | | |
| ENSG000 | 1.249845236 | SEQ | AUUCUCUGCCUCUGAAGGCUCCAGAGGCUGUGGGA |
| 00180287- | | ID | AGCAGCACAUCAGCGACAGCUCCUGGCUGCUGGACU |
| ENST000 | | NO: | CCGCAGGGAGGGAAGGAAGAUUGGUCGCA |
| 00314833- | | 923 | |
| 100 | | | |
| ENSG000 | 1.105668147 | SEQ | AAAAGUAAAACCAGUUGUUGUGGAGAAAUAAGAGG |
| 00180336- | | ID | ACCUGCUCCUCCAAUUUAAAGUCAGUUUGGCCA |
| ENST000 | | NO: | |
| 00432494- | | 924 | |
| 68 | | | |
| ENSG000 | 1.452912777 | SEQ | AUAGUUCUGCAGUCACAAGGGCUUUGUAGUCAGUC |
| 00180347- | | ID | UUAGCUGCUGGAACAUUCAAGGAGACAAAAAGGCA |
| ENST000 | | NO: | CAGAAGAUCUCCACACAUACUUUG |
| 00451887- | | 925 | |
| 94 | | | |
| ENSG000 | 1.104190423 | SEQ | AGAGAGGGAGUCGGUGUGGACGCGAGGAGCCGGGC |
| 00180353- | | ID | GCUUAGAACAGAGGCUUGCACAGGUGGAG |
| ENST000 | | NO: | |
| 00464274- | | 926 | |
| 64 | | | |
| ENSG000 | 1.334429401 | SEQ | ACUGAAGCAGCAGCUCAGUUUCUCACUCCGAAGUG |
| 00180353- | | ID | GCAGCAGCCAGAGAGGGAGUCGGUGUGGACGCGAG |
| ENST000 | | NO: | GAGCCGGGCGCUUAGAACAGAGGCUUGCACAGGUG |
| 00495491- | | 927 | GAG |
| 108 | | | |
| ENSG000 | 1.247964792 | SEQ | AAAGCGGCCAGUUUUUACAUAUUUCUUGAUUUUGU |
| 00180573- | | ID | UUGUUUUCUCGUGAGCUUAGGCCGCUGGUUUUGGU |
| ENST000 | | NO: | GAUUUUUGUCUGAUUGCA |
| 00314088- | | 928 | |
| 88 | | | |
| ENSG000 | 1.296417677 | SEQ | GAUUUUGUUUGUUUUCUCGUGAGCUUAGGCCGCUG |
| 00180573- | | ID | GUUUUGGUGAUUUUUGUCUGAUUGCA |
| ENST000 | | NO: | |
| 00377791- | | 929 | |
| 61 | | | |
| ENSG000 | 1.693230226 | SEQ | CUUGGACCAAUCAGCCUUGUGUUUAUAGGAAUUAU |
| 00180773- | | ID | UUCUGUUCACUGU |
| ENST000 | | NO: | |
| 00534116- | | 930 | |
| 48 | | | |
| ENSG000 | 1.480900202 | SEQ | AACAACAGCUGAGUCAGACAGAGUAUCUGUACACU |
| 00180872- | | ID | GUUUUAUACCAAGAAAUCAUUUACA |
| ENST000 | | NO: | |
| 00651554- | | 931 | |
| 60 | | | |
| ENSG000 | 1.872737891 | SEQ | ACAGGCAGUAACCACGAGUCUCAGCCCCUUAGCUGA |
| 00180919- | | ID | ACUUUUAAACAGAGAUUUCUUCUAUUUCAGACAGA |
| ENST000 | | NO: | CACUGAGACUGAGGCACCCAUUCC |
| 00316529- | | 932 | |
| 95 | | | |
| ENSG000 | 1.282665562 | SEQ | UUCUCUUCAGGAGUAAACUUCUGGAAAAAUAGA |
| 00181009- | | ID | |
| ENST000 | | NO: | |
| 00317093- | | 933 | |
| 33 | | | |
| ENSG000 | 1.205743985 | SEQ | UUCUGUGGUGGUUCCAACCUGUGAUAACUGAGAAC |
| 00181023- | | ID | AAUACAAAUAGAGAUUUGAAAUUCAGUUUGAAUC |
| ENST000 | | NO: | |
| 00317121- | | 934 | |
| 69 | | | |
| ENSG000 | 1.376599392 | SEQ | CUUGUACCUGUCGUUCGCAGUGGCUCACUUUCCCAA |
| 00181322- | | ID | GCC |
| ENST000 | | NO: | |
| 00484930- | | 935 | |
| 39 | | | |
| ENSG000 | 1.30411828 | SEQ | CCCUCUGUCUUUAGGGGCUGAGAUUUAGUCCAUCU |
| 00181616- | | ID | GCCUCUGCC |
| ENST000 | | NO: | |
| 00641796- | | 936 | |
| 44 | | | |
| ENSG000 | 1.341404417 | SEQ | AGUCACUUGCCAUUUCUCAUAACAGCGUCAGAGAG |
| 00181617- | | ID | AAAGAACUGACUGAAACGUUUGAG |
| ENST000 | | NO: | |
| 00317987- | | 937 | |
| 59 | | | |
| ENSG000 | 1.219852565 | SEQ | UCUCAGCAGUUUAAAGCAAUUGAACAUC |
| 00181693- | | ID | |
| ENST000 | | NO: | |
| 00313022- | | 938 | |
| 28 | | | |
| ENSG000 | 1.197577581 | SEQ | UGUUGAUAGCCCAUUGUGCAAAAUAAGUGAUAAAA |
| 00181785- | | ID | UUCAAAGUUCUUGAGUGUGCUGAGGUCCAGUGGGA |
| ENST000 | | NO: | AAAACAAGAAAACUAAG |
| 00641320- | | 939 | |
| 87 | | | |
| ENSG000 | 1.644675067 | SEQ | AAGAAAGUGGGUCAGCAGGAGUUCCAGCAUCUCAG |
| 00181904- | | ID | AAUUGUGGUGAAAACCAAUACAAAAA |
| ENST000 | | NO: | |
| 00338051- | | 940 | |
| 61 | | | |
| ENSG000 | 1.591939527 | SEQ | UUCUUUUUUUAACGUUUCCACUUUUAGUAUUAUUU |
| 00182004- | | ID | CAGAGAUCGCGGAUUCAGGUGUGGCUCUAGUAGCA |
| ENST000 | | NO: | AGUGAAU |
| 00367208- | | 941 | |
| 77 | | | |
| ENSG000 | 1.643557117 | SEQ | CUUUAUUCCGGAAGUUGCUCUCAGAGGCAGCGUGC |
| 00182004- | | ID | GGGUGUGCUCUUUGUGAAAUUCCACC |
| ENST000 | | NO: | |
| 00414487- | | 942 | |
| 61 | | | |
| ENSG000 | 1.769986651 | SEQ | GCACUCGACGGCUGACUGGAGCAGCGGAUAUAAGU |
| 00182134- | | ID | CAAAGAGUACAGCAGAAA |
| ENST000 | | NO: | |
| 00526413- | | 943 | |
| 53 | | | |
| ENSG000 | 1.362800917 | SEQ | GUACAGGUUAUUUCAUUUCUGGCUGCAGUUUUGCA |
| 00182150- | | ID | UAAAAAGGGAACUCGUGAGGAUAUUGAAAAUAAC |
| ENST000 | | NO: | |
| 00402838- | | 944 | |
| 69 | | | |
| ENSG000 | 1.206313264 | SEQ | CUUUUCUUUGCUGGAUCUGGAGGC |
| 00182185- | | ID | |
| ENST000 | | NO: | |
| 00553334- | | 945 | |
| 24 | | | |
| ENSG000 | 1.302329302 | SEQ | GAGUAGUUCUACCAUUCUCUUUUGAGUCAUAUAGA |
| 00182287- | | ID | GAGUGCGCAAGUUAUAGCACAG |
| ENST000 | | NO: | |
| 00673445- | | 946 | |
| 57 | | | |
| ENSG000 | 1.162249211 | SEQ | AAAAUACUAACCAGCCAGAGAAACAGAAUCCUAAC |
| 00182333- | | ID | UAUUUCUGAGGAAACUGCAGGUCCAAA |
| ENST000 | | NO: | |
| 00608620- | | 947 | |
| 62 | | | |
| ENSG000 | 1.163065949 | SEQ | UUAUUAGCAAGCAGCAAAU |
| 00182504- | | ID | |
| ENST000 | | NO: | |
| 00465011- | | 948 | |
| 19 | | | |
| ENSG000 | 1.256476998 | SEQ | UAUUAACAAAAUCAGUUAAGCAGUAGAAAGGUGGG |
| 00182568- | | ID | UUCUUCUGAAGAUAAAGUGAACC |
| ENST000 | | NO: | |
| 00475083- | | 949 | |
| 58 | | | |
| ENSG000 | 1.501244068 | SEQ | CUGCGGAAGUUCAACUAUUCAAC |
| 00182628- | | ID | |
| ENST000 | | NO: | |
| 00330137- | | 950 | |
| 23 | | | |
| ENSG000 | 1.428481425 | SEQ | AAAACACUGACUAGUAAGGAGGAAGACAUUGAAGA |
| 00182732- | | ID | AGGAAAGCCAUCUAUCUCUGUAAGAGGACA |
| ENST000 | | NO: | |
| 00554782- | | 951 | |
| 65 | | | |
| ENSG000 | 1.28907986 | SEQ | ACUGAUAUAAAUCUGCUUAGGAGGCCUGAUUCACA |
| 00182853- | | ID | GACGCUACAGG |
| ENST000 | | NO: | |
| 00416307- | | 952 | |
| 46 | | | |
| ENSG000 | 1.372846253 | SEQ | ACUCGCUUUCCUCGGAUUCCCGGAGUUGGGGGAUU |
| 00182923- | | ID | UUUGACAUCUUGUGAAGCAGAACUACAGGAGCUC |
| ENST000 | | NO: | |
| 00684170- | | 953 | |
| 69 | | | |
| ENSG000 | 2.763412453 | SEQ | AAGGAGAGAAA |
| 00182944- | | ID | |
| ENST000 | | NO: | |
| 00332035- | | 954 | |
| 11 | | | |
| ENSG000 | 1.101929967 | SEQ | AGAGGGAAAGCGAGAGGGAGACGGACGUUGAGAGA |
| 00182944- | | ID | ACGAGGAGGAAGGAGAGAAA |
| ENST000 | | NO: | |
| 00437155- | | 955 | |
| 55 | | | |
| ENSG000 | 1.664718743 | SEQ | AACAUUACCAGUUUCAACUUACUAGUCUCAAGUCC |
| 00183035- | | ID | AGGCAACGUACAGGCAGGGGAA |
| ENST000 | | NO: | |
| 00621735- | | 956 | |
| 57 | | | |
| ENSG000 | 1.208273829 | SEQ | AGCAAGGUUCCUUGGAAGAAG |
| 00183077- | | ID | |
| ENST000 | | NO: | |
| 00586731- | | 957 | |
| 21 | | | |
| ENSG000 | 1.194329717 | SEQ | AAUUGGUUUAGUAUCUGUAUAGUGGCAAUACUCUG |
| 00183230- | | ID | UCCUGCAGUAGGCAGC |
| ENST000 | | NO: | |
| 00683624- | | 958 | |
| 51 | | | |
| ENSG000 | 1.178516795 | SEQ | ACAAACCGGACCCGCAACCACC |
| 00183283- | | ID | |
| ENST000 | | NO: | |
| 00604900- | | 959 | |
| 22 | | | |
| ENSG000 | 1.240103799 | SEQ | CAUUGUGGCUGGCAUAGUAUACAUACUUGGUUAAA |
| 00183389- | | ID | UAUAAGGGAGG |
| ENST000 | | NO: | |
| 00330728- | | 960 | |
| 46 | | | |
| ENSG000 | 1.176566889 | SEQ | AAUAGCAGCAGUACAGCCUUCUGAAUUUGGUACAC |
| 00183389- | | ID | UGGGUCUCCAGAUUGAUCAGGAAACUUUAC |
| ENST000 | | NO: | |
| 00641835- | | 961 | |
| 65 | | | |
| ENSG000 | 1.372175066 | SEQ | AGGCUGACAGAGAAAUUUUUCAUUUUCUUUCUUGU |
| 00183395- | | ID | UUGACUGUAGUCAAACAUCAAACUAAGA |
| ENST000 | | NO: | |
| 00329406- | | 962 | |
| 63 | | | |
| ENSG000 | 1.204264954 | SEQ | UCAUUUCCAUCAAGUGUGCAGCAGUGGUCUCUCUG |
| 00183560- | | ID | UAGCAGGCC |
| ENST000 | | NO: | |
| 00440961- | | 963 | |
| 44 | | | |
| ENSG000 | 1.155796664 | SEQ | ACCACUGGUCUUCUUGUGCUUAUCCGGGCAAGAAC |
| 00183625- | | ID | UUAUCGAAAUACAAUAGAAGUUUUUACUUAGAAGA |
| ENST000 | | NO: | GAUUUUCAGGGAGAAGUGAA |
| 00395940- | | 964 | |
| 90 | | | |
| ENSG000 | 1.511096092 | SEQ | GAGAGACUACAACUCCCAGUAGUCACCGCG |
| 00183632- | | ID | |
| ENST000 | | NO: | |
| 00569420- | | 965 | |
| 30 | | | |
| ENSG000 | 1.432209209 | SEQ | UAUAACAAGAGGAUUGCCUGAUCCAGCCAAG |
| 00183735- | | ID | |
| ENST000 | | NO: | |
| 00676469- | | 966 | |
| 31 | | | |
| ENSG000 | 1.213968303 | SEQ | CAAACAGGGACA |
| 00183765- | | ID | |
| ENST000 | | NO: | |
| 00464581- | | 967 | |
| 12 | | | |
| ENSG000 | 1.382016145 | SEQ | ACUUGCCGAAGC |
| 00183853- | | ID | |
| ENST000 | | NO: | |
| 00368172- | | 968 | |
| 12 | | | |
| ENSG000 | 1.400768934 | SEQ | CUGGCAGAUAAGUGCUCUGGUUGUACAGUGAGG |
| 00183862- | | ID | |
| ENST000 | | NO: | |
| 00329903- | | 969 | |
| 33 | | | |
| ENSG000 | 1.194208509 | SEQ | AGAUCGUCGCCUAUCCUGCUUGCGCAGCCGCUAUCG |
| 00184083- | | ID | CGUCUGCCCACCGACGAAGC |
| ENST000 | | NO: | |
| 00375180- | | 970 | |
| 56 | | | |
| ENSG000 | 1.348401227 | SEQ | CUGCAUUUUGUGAUCCUCUAUUUCAACACUGGUCC |
| 00184140- | | ID | AGUUAGCAGUAGAGUUGUCACAGCCGACAGAGGCA |
| ENST000 | | NO: | |
| 00328882- | | 971 | |
| 70 | | | |
| ENSG000 | 1.669547111 | SEQ | UGCUGGGAUUACAGGCGUGAGCCACCGCGUCUGGCC |
| 00184209- | | ID | UCAGCCAAGGUUUUUAAGUAACAUAUUUCAGCAUU |
| ENST000 | | NO: | GGCUCUACAGCGUUGCAGAAC |
| 00350887- | | 972 | |
| 92 | | | |
| ENSG000 | 1.921123475 | SEQ | ACACAUCUCACUCAUCCUUCUACUCGUGACACUUCC |
| 00184330- | | ID | CAGUUCUGGCUUUUUGAAAGCAAAG |
| ENST000 | | NO: | |
| 00368729- | | 973 | |
| 61 | | | |
| ENSG000 | 2.014335888 | SEQ | AACGCAAAACCUGCUCUUUAGAUUUCGAGCUUAUU |
| 00184357- | | ID | CUCUUCUAGCAGUUUCUUGCCACC |
| ENST000 | | NO: | |
| 00331442- | | 974 | |
| 59 | | | |
| ENSG000 | 1.120565441 | SEQ | CUUUCCCGGAGGCUGGCAG |
| 00184428- | | ID | |
| ENST000 | | NO: | |
| 00329245- | | 975 | |
| 19 | | | |
| ENSG000 | 1.797996465 | SEQ | AUACGAGUUACUUUUUUUAAGUCCGUUGAAACAGA |
| 00184434- | | ID | GUUAAUUUCCUUUAGCACACAAGUCUUAGAGACAA |
| ENST000 | | NO: | AAGAAAAAAAGGUCUGCAAC |
| 00380055- | | 976 | |
| 90 | | | |
| ENSG000 | 1.09960343 | SEQ | AGAAACCUGUGUAGCCAGAG |
| 00184451- | | ID | |
| ENST000 | | NO: | |
| 00332438- | | 977 | |
| 20 | | | |
| ENSG000 | 1.196399177 | SEQ | CUGAGCGCAGUUGGAGUUCGGAGGAAAAGGGCUCA |
| 00184465- | | ID | GAUUGAAG |
| ENST000 | | NO: | |
| 00423258- | | 978 | |
| 43 | | | |
| ENSG000 | 1.462206433 | SEQ | AGGACACAUUUAGUCAGAGUAGCUUAUAAGAGACC |
| 00184588- | | ID | GUUCCCUCCGCCUUCUUCCUCAGAGGAAGUUUCUUG |
| ENST000 | | NO: | AAGAAUCUUAUCAAAAAUUAGCA |
| 00526197- | | 979 | |
| 94 | | | |
| ENSG000 | 1.854117048 | SEQ | AGAAGGAUCCAUCUAACAUUUACUGAAAUAAGGAA |
| 00184613- | | ID | GAGUCUUUAUUCAAUCUAAAAGUGAAAAAAGAACC |
| ENST000 | | NO: | UUUUAGACAUUAUUAUUAUAAA |
| 00333837- | | 980 | |
| 92 | | | |
| ENSG000 | 1.343693352 | SEQ | CUGUGUCUUACC |
| 00184678- | | ID | |
| ENST000 | | NO: | |
| 00369155- | | 981 | |
| 12 | | | |
| ENSG000 | 1.456029376 | SEQ | ACUCGGCCUAGGAAUUUCCCUUAUCUCCUUCGCAGU |
| 00184697- | | ID | GCAGCUCCUUCAACCUCGCC |
| ENST000 | | NO: | |
| 00328796- | | 982 | |
| 56 | | | |
| ENSG000 | 1.646705337 | SEQ | ACCUGCCCAGUGCCACUUCCUUCUCUCUGUCCCUGU |
| 00184925- | | ID | GGGCCCAGCAGCUGCCAGG |
| ENST000 | | NO: | |
| 00371633- | | 983 | |
| 55 | | | |
| ENSG000 | 1.559106462 | SEQ | UUUUGCUCUCUGGGAUUGGUUUCUUAAAGAAUCUG |
| 00185008- | | ID | GAUCCUUUUUAAUAGUUCAAA |
| ENST000 | | NO: | |
| 00332191- | | 984 | |
| 56 | | | |
| ENSG000 | 1.126932167 | SEQ | GAGAAAGUUGUCUGCGUCUCGAGGCGAGUUGGCGG |
| 00185043- | | ID | AGCUGUGCGCGCGGCGGGGCG |
| ENST000 | | NO: | |
| 00328649- | | 985 | |
| 56 | | | |
| ENSG000 | 1.497642295 | SEQ | AUCCCUCUCAGCUCUUGCUAAGUGAUCACUAGCUUG |
| 00185069- | | ID | CCUCCAAGCUCUCUCACU |
| ENST000 | | NO: | |
| 00332411- | | 986 | |
| 54 | | | |
| ENSG000 | 1.360169832 | SEQ | UUGGUUUUGCCACUAUUGUUUCAUU |
| 00185130- | | ID | |
| ENST000 | | NO: | |
| 00377401- | | 987 | |
| 25 | | | |
| ENSG000 | 1.105239705 | SEQ | AUCUGUUUUUUUCCCUUCUGAGCA |
| 00185432- | | ID | |
| ENST000 | | NO: | |
| 00332160- | | 988 | |
| 24 | | | |
| ENSG000 | 1.59079098 | SEQ | UGCAAUUAAAGGAGUCGGGUCUCUAACUGUUGAUC |
| 00185432- | | ID | UGUUUUUUUCCCUUCUGAGCA |
| ENST000 | | NO: | |
| 00547104- | | 989 | |
| 56 | | | |
| ENSG000 | 1.313763959 | SEQ | ACAUUUAAGAAAUACAUUGGUUCAGUCCAGAAAGG |
| 00185432- | | ID | CAGGACAACCCGACGCGAGGCAGGGAGCUUCCAGGU |
| ENST000 | | NO: | UAUAGCA |
| 00550502- | | 990 | |
| 78 | | | |
| ENSG000 | 1.131954047 | SEQ | CCCCCGGCAGCCCGGAGGAGC |
| 00185532- | | ID | |
| ENST000 | | NO: | |
| 00373976- | | 991 | |
| 21 | | | |
| ENSG000 | 1.468037556 | SEQ | CCCCGCCUGAUCCGUGUCCGAC |
| 00185624- | | ID | |
| ENST000 | | NO: | |
| 00680226- | | 992 | |
| 22 | | | |
| ENSG000 | 1.51099076 | SEQ | CUUCCUGCUGUC |
| 00185627- | | ID | |
| ENST000 | | NO: | |
| 00382671- | | 993 | |
| 12 | | | |
| ENSG000 | 1.703911723 | SEQ | AAAAGUAGUCUACCCUGAAGACCCCAGAUUUUUGA |
| 00185737- | | ID | UCUGCUCUUUUGAGUCAGUUGAUAGAAUA |
| ENST000 | | NO: | |
| 00602794- | | 994 | |
| 64 | | | |
| ENSG000 | 1.478052558 | SEQ | ACACCUCAGUUCACAAUCCUGGGGCGAU |
| 00185792- | | ID | |
| ENST000 | | NO: | |
| 00332836- | | 995 | |
| 28 | | | |
| ENSG000 | 1.519311248 | SEQ | UUAAAGUACUUGGAGUUUUUAUAGGUUGGCACUCA |
| 00185864- | | ID | UCAGUAGCCCCUGUUCUCAUUCUGCAA |
| ENST000 | | NO: | |
| 00529969- | | 996 | |
| 62 | | | |
| ENSG000 | 1.387501218 | SEQ | AUUUCAAGGAUUCUACCAUCAGAAAAGAGGCCAAA |
| 00186007- | | ID | CUUCUAUCAUC |
| ENST000 | | NO: | |
| 00367149- | | 997 | |
| 46 | | | |
| ENSG000 | 1.385222169 | SEQ | CCCAGAUCUCUUCAGUUUUUAGUCCUCAUUCUGUG |
| 00186092- | | ID | AAAAUUGCUGUAGUCUCUUCCAGUU |
| ENST000 | | NO: | |
| 00641515- | | 998 | |
| 60 | | | |
| ENSG000 | 1.245881817 | SEQ | ACUGUGCUGGGGUGUACAUCUACACUAGACACCUU |
| 00186335- | | ID | CCUGCUUCCCUCCUUCCAGAGCAGACCUCUUUGUCA |
| ENST000 | | NO: | CCCCGAGCUCCUUGUUUCUUAAGCAGUC |
| 00335244- | | 999 | |
| 99 | | | |
| ENSG000 | 1.18259916 | SEQ | GUUUUUCUCUCUGCUUUCUCAACAGCCAGAUCUGCC |
| 00186469- | | ID | AGUGAGCCUCAGGCUUUAGGAACUGAAGAGUGUUU |
| ENST000 | | NO: | CUGAAAGAUCUAUCCAGCACUCCG |
| 00556752- | | 1000 | |
| 95 | | | |
| ENSG000 | 1.103341541 | SEQ | UCAGGGCAGUAGAACAUAUUUUAAUCAAGUCCCCU |
| 00186572- | | ID | UGAUAAAAGAGCUUGAUUCCAGCUACUGCACUUCC |
| ENST000 | | NO: | UUCAUCAGCUCUCCACUG |
| 00335021- | | 1001 | |
| 88 | | | |
| ENSG000 | 1.998127013 | SEQ | CCUUGCCAGUCCUUCAGAGCCAUUGUAGCAGGAGGC |
| 00186635- | | ID | AGAGUACAGAGUG |
| ENST000 | | NO: | |
| 00455638- | | 1002 | |
| 49 | | | |
| ENSG000 | 1.135427481 | SEQ | GGCUUUGGGAAGAUCUAAAGACCCAGGAAGGUCUC |
| 00186652- | | ID | UGGGUGGGAUAAAGCCAAG |
| ENST000 | | NO: | |
| 00533605- | | 1003 | |
| 54 | | | |
| ENSG000 | 1.450039966 | SEQ | CAAGGUUAUCCAUCUCAAGUAGCCUAGCAAUAUUU |
| 00186803- | | ID | GCAACAUCCCA |
| ENST000 | | NO: | |
| 00357374- | | 1004 | |
| 46 | | | |
| ENSG000 | 1.129244458 | SEQ | CUGCUAUACAACUGAGCCUACCUUUCUUGAAACC |
| 00186924- | | ID | |
| ENST000 | | NO: | |
| 00334680- | | 1005 | |
| 34 | | | |
| ENSG000 | 1.466846422 | SEQ | AGAAUCUUCUUAGGUACACUCAGCUGAACUCCCAUC |
| 00186971- | | ID | UCUCAUCAGC |
| ENST000 | | NO: | |
| 00334068- | | 1006 | |
| 46 | | | |
| ENSG000 | 1.14098428 | SEQ | AAGUGGGGCAGC |
| 00187024- | | ID | |
| ENST000 | | NO: | |
| 00416214- | | 1007 | |
| 12 | | | |
| ENSG000 | 1.538600768 | SEQ | AGUCACACUUCAGAAAUAUCCUCCUACACCUCAACU |
| 00187026- | | ID | GAAAUCUAACCUCCUGAAAUC |
| ENST000 | | NO: | |
| 00333892- | | 1008 | |
| 57 | | | |
| ENSG000 | 1.102818675 | SEQ | CUUUGCCAGUCCAUCUUCAAAUUGGAAUUAUAGAA |
| 00187098- | | ID | AGUAGAGGGAGGGAUAGUCUACCGUCUCUCACUGG |
| ENST000 | | NO: | AUUGGUGCCACCUAAAACAUUGUU |
| 00394348- | | 1009 | |
| 94 | | | |
| ENSG000 | 1.247153321 | SEQ | UCUUUGCCAGUCCAUCUUCAAAUUGGAAUUAUAGA |
| 00187098- | | ID | AAGUAGAGGGAGGGAUAGUCUACCGUCUCUCACUG |
| ENST000 | | NO: | GAUUGGUGCCACCUAAAACAUUGUU |
| 00478490- | | 1010 | |
| 95 | | | |
| ENSG000 | 1.906298135 | SEQ | AGAACCAGUAAGCAAAUUAAGUGUAUCGAUUGCAA |
| 00187391- | | ID | UUUUGUUGGACUAACCCUGCUUUUUUUUUCUUAAG |
| ENST000 | | NO: | GAAUAAACUUGAUUCCUUUUAGU |
| 00636178- | | 1011 | |
| 93 | | | |
| ENSG000 | 1.346087887 | SEQ | AGUCUUUUCGCUGGUUAUUACAUCUUGCGUUUCUC |
| 00187475- | | ID | UGUUGUU |
| ENST000 | | NO: | |
| 00338379- | | 1012 | |
| 42 | | | |
| ENSG000 | 1.286957338 | SEQ | GCUGUCUGUGUCUUCAGGUCAUC |
| 00187492- | | ID | |
| ENST000 | | NO: | |
| 00343366- | | 1013 | |
| 23 | | | |
| ENSG000 | 1.262401764 | SEQ | AUUUGUGACUUGUCUC |
| 00187627- | | ID | |
| ENST000 | | NO: | |
| 00409776- | | 1014 | |
| 16 | | | |
| ENSG000 | 1.35268997 | SEQ | ACCAGUUCGCGCUCUCCUCCGGCAGAAGUAGCAGCU |
| 00187690- | | ID | GCGCUCUUGCUCUCUGGGGGAGAACCUGGCGUU |
| ENST000 | | NO: | |
| 00342995- | | 1015 | |
| 69 | | | |
| ENSG000 | 1.512590793 | SEQ | UGAACAAUUGCAGUUACUACCUUAGUUCUGUAAGG |
| 00187701- | | ID | UUUCUCUCUAAAUAUUCAGAUCAUCGUGUUUGGGG |
| ENST000 | | NO: | AAGAUACUGUGUAAGAGCU |
| 00460972- | | 1016 | |
| 89 | | | |
| ENSG000 | 1.933200485 | SEQ | AAAAGUUGCUUCUUACAGGAAUAUAAAUAGUUUCU |
| 00187733- | | ID | GGAAAGGACACUGACAACUUCAAAGCAAA |
| ENST000 | | NO: | |
| 00370079- | | 1017 | |
| 64 | | | |
| ENSG000 | 2.222280021 | SEQ | AGCCGCCGCCGGGGCUGUAGGCGCCAAGGCC |
| 00187741- | | ID | |
| ENST000 | | NO: | |
| 00568369- | | 1018 | |
| 31 | | | |
| ENSG000 | 1.302585382 | SEQ | GGAAUUAGAGAAUAAAAUCCCAGCACG |
| 00187753- | | ID | |
| ENST000 | | NO: | |
| 00613665- | | 1019 | |
| 27 | | | |
| ENSG000 | 1.586904311 | SEQ | GUGCGAAGGAAACCGAGUGGGAUCGGAACCGUAGC |
| 00187790- | | ID | GGUUGAGCUGCUGCUGCUACGGAUAUCUGACAGAA |
| ENST000 | | NO: | GCCUUCGGUGGUUGUCGGCCUA |
| 00556036- | | 1020 | |
| 92 | | | |
| ENSG000 | 1.130407808 | SEQ | ACUAGGCUCUCAGCCAGGG |
| 00188038- | | ID | |
| ENST000 | | NO: | |
| 00339176- | | 1021 | |
| 19 | | | |
| ENSG000 | 1.178508086 | SEQ | UGCAGUCCUGUCUCGUUUCCCUCUUUCUCUCUCUCU |
| 00188269- | | ID | CUCUGAUAGUCACUUCAAUCAA |
| ENST000 | | NO: | |
| 00594432- | | 1022 | |
| 58 | | | |
| ENSG000 | 1.114829554 | SEQ | ACUUCUGCAGCACAGCUCCCUUCCCAGGACGUGAAA |
| 00188373- | | ID | AUCUGCCUUCUCACC |
| ENST000 | | NO: | |
| 00372126- | | 1023 | |
| 51 | | | |
| ENSG000 | 1.136596257 | SEQ | AGAGAACCUGGAGCCUAAGGUUUAGGCUCACCCAU |
| 00188379- | | ID | UUCAACCAGUCUAGCAGCAUCUGCAACAUCUACA |
| ENST000 | | NO: | |
| 00380206- | | 1024 | |
| 69 | | | |
| ENSG000 | 1.171829026 | SEQ | AAUCCUCCUCUUCUCCCUUCCUGAAGCUGCACGCUG |
| 00188467- | | ID | CAGUAAGAGCACAGCAGAA |
| ENST000 | | NO: | |
| 00341459- | | 1025 | |
| 55 | | | |
| ENSG000 | 1.245989307 | SEQ | UGCAGUAAGAGCACAGCAGAA |
| 00188467- | | ID | |
| ENST000 | | NO: | |
| 00449382- | | 1026 | |
| 21 | | | |
| ENSG000 | 1.437532998 | SEQ | UAAUUAUUGAGUGCUCUGCCUGCUUAGGACUCUGC |
| 00188629- | | ID | CCAGCCAGGAAGGAAACCUACAGGAGGAAGA |
| ENST000 | | NO: | |
| 00589262- | | 1027 | |
| 66 | | | |
| ENSG000 | 1.958972325 | SEQ | GUUUUCUUAGUAUAGUUCAUUCUCAAUAAUAUUUC |
| 00188649- | | ID | UUAUUUAUUUAGAAAGUUGAAAUAAAAGGACCAUC |
| ENST000 | | NO: | |
| 00636965- | | 1028 | |
| 70 | | | |
| ENSG000 | 1.385334897 | SEQ | ACUUUGCUGAUUUAGCUUAGUGAAGAGGAACCAGA |
| 00188820- | | ID | AAUUUGUCCUUGAAUA |
| ENST000 | | NO: | |
| 00368606- | | 1029 | |
| 51 | | | |
| ENSG000 | 1.117127511 | SEQ | AGUGAACUUCUGAUAGGGUCUUAUUU |
| 00188992- | | ID | |
| ENST000 | | NO: | |
| 00344577- | | 1030 | |
| 26 | | | |
| ENSG000 | 1.186798237 | SEQ | CCUUUUUGGUGAAUUGAGUGCUGUUUUUGCUUUUC |
| 00188992- | | ID | UCAGAUUCCAA |
| ENST000 | | NO: | |
| 00536861- | | 1031 | |
| 46 | | | |
| ENSG000 | 1.097273628 | SEQ | AAGACAGGCAUCUCAAAUCGGCUGAUUCUGCAUCU |
| 00189058- | | ID | GGAAACUGCCUUCAUCUUGAAAGAAAAGCUCCAGG |
| ENST000 | | NO: | UCCCUUCUCCAGCCACCCAGCCCCAAG |
| 00343267- | | 1032 | |
| 97 | | | |
| ENSG000 | 1.214331453 | SEQ | AUUCUCUGGAGUUUGA |
| 00189144- | | ID | |
| ENST000 | | NO: | |
| 00489148- | | 1033 | |
| 16 | | | |
| ENSG000 | 1.223880777 | SEQ | UCAGAUCACAGAUUUCCCUGCCAA |
| 00189181- | | ID | |
| ENST000 | | NO: | |
| 00342623- | | 1034 | |
| 24 | | | |
| ENSG000 | 1.185393876 | SEQ | CUGCUCAGCACUAGGCCGGCAGAAUCUGAGCG |
| 00189430- | | ID | |
| ENST000 | | NO: | |
| 00598576- | | 1035 | |
| 32 | | | |
| ENSG000 | 1.520503024 | SEQ | AACACUCACAAGAGAGCAGAGGAGCCCCAAGUCUU |
| 00196209- | | ID | GGGGACCACAGAAGAGUCC |
| ENST000 | | NO: | |
| 00608073- | | 1036 | |
| 54 | | | |
| ENSG000 | 1.988764021 | SEQ | AGAAGGGUACAAAUCUUUUUUCUUUCUCCAAGGAG |
| 00196353- | | ID | AAUCUUUUGAGCACAAGUA |
| ENST000 | | NO: | |
| 00505881- | | 1037 | |
| 54 | | | |
| ENSG000 | 1.279951085 | SEQ | CUGAGUCACAUUGAGGUCUCUUGCUUUAGGAACUG |
| 00196391- | | ID | AGUACGCUUGAUA |
| ENST000 | | NO: | |
| 00558586- | | 1038 | |
| 48 | | | |
| ENSG000 | 1.856926054 | SEQ | ACACUCGCUUCUGGAACGUCUGAGGUUAUCAAUAA |
| 00196565- | | ID | GCUCCUAGUCCAGACGCC |
| ENST000 | | NO: | |
| 00336906- | | 1039 | |
| 53 | | | |
| ENSG000 | 1.143503127 | SEQ | GCAUUAUCCACUAGAUACGUCGAAGAAGAGGGAAA |
| 00196628- | | ID | CCAAUUAGGGUCGAAAUAA |
| ENST000 | | NO: | |
| 00568147- | | 1040 | |
| 54 | | | |
| ENSG000 | 1.121501726 | SEQ | GAAAAGUCAACUGAAGGAAUUGUUGUGAAAAAAGG |
| 00196628- | | ID | GACAGCGAGUUUGAAAUAAAAGUUGUUAGAGUGGU |
| ENST000 | | NO: | ACUGAGGAGAAAAAAGAAUCCGAGACC |
| 00628689- | | 1041 | |
| 97 | | | |
| ENSG000 | 1.233556294 | SEQ | AUUGGAAGUACAGUUUUUGGAAGUGUGGAGCAGUU |
| 00196628- | | ID | UGGCUAAGAAUAGGA |
| ENST000 | | NO: | |
| 00630828- | | 1042 | |
| 50 | | | |
| ENSG000 | 1.128029078 | SEQ | ACGGCCGUCGCC |
| 00196683- | | ID | |
| ENST000 | | NO: | |
| 00372879- | | 1043 | |
| 12 | | | |
| ENSG000 | 1.447781298 | SEQ | UGGAUACACUGGAAAAAGUUCUUGCUGGGCAACCA |
| 00196712- | | ID | AAGGACACAAGUAGAUUAG |
| ENST000 | | NO: | |
| 00490416- | | 1044 | |
| 54 | | | |
| ENSG000 | 1.670486941 | SEQ | ACAAGAGAACAACUUGCUCCUUCCACAGGAAGCUGC |
| 00196844- | | ID | ACCUGACAGAAGCUCCAGG |
| ENST000 | | NO: | |
| 00358524- | | 1045 | |
| 55 | | | |
| ENSG000 | 1.178078321 | SEQ | CUGUUUGGAGAGACUCAGCCAUC |
| 00196890- | | ID | |
| ENST000 | | NO: | |
| 00620438- | | 1046 | |
| 23 | | | |
| ENSG000 | 1.113116982 | SEQ | GGGAGACUGGAGUCGAAGGCCGUGAGGUAUUUUUC |
| 00197223- | | ID | UAAGUCAGCCAUA |
| ENST000 | | NO: | |
| 00355848- | | 1047 | |
| 48 | | | |
| ENSG000 | 1.173655751 | SEQ | ACAAAGCGUUGGGUGAGACUCCUCUUGCUCGUC |
| 00197238- | | ID | |
| ENST000 | | NO: | |
| 00355057- | | 1048 | |
| 33 | | | |
| ENSG000 | 1.178970809 | SEQ | CAAGGCAUUCUUCCAGUUCCUCAGCCUCCUCUUUCC |
| 00197309- | | ID | UUCCUAGGACUGGCUUCC |
| ENST000 | | NO: | |
| 00318666- | | 1049 | |
| 54 | | | |
| ENSG000 | 1.116606471 | SEQ | UCCUUGCACAAGAGGAAGUAUAUCCAAUUCUUGCC |
| 00197309- | | ID | AGGCAUUGAGACUGGCUUCC |
| ENST000 | | NO: | |
| 00641546- | | 1050 | |
| 55 | | | |
| ENSG000 | 1.142287306 | SEQ | CCUUUCUCUCCUCUGAGCCACAACUAAAGUAGUUUU |
| 00197428- | | ID | CUACUUUUCCCUUUCUACUUUCCUAGACCCUGGAUU |
| ENST000 | | NO: | UUGU |
| 00357605- | | 1051 | |
| 76 | | | |
| ENSG000 | 1.117052404 | SEQ | GUCUUUGGUAGUUUUUUUGCACUAACUUCAGGAGC |
| 00197465- | | ID | CAGCUCGUGAUCUCAGG |
| ENST000 | | NO: | |
| 00358615- | | 1052 | |
| 52 | | | |
| ENSG000 | 1.225835677 | SEQ | AGUGGCAAGCGCGGGCAGGACCGCGUUGCGUCAUC |
| 00197548- | | ID | GGGGCGCGCGCCUCAGAGAGAGCUGUGGUUGCCGG |
| ENST000 | | NO: | AAGUUGAGCGGCGGCAAGAAAUA |
| 00451830- | | 1053 | |
| 93 | | | |
| ENSG000 | 1.428994846 | SEQ | CUCAAAACUGCCAGUGUGAGAGGAUUUGGAAAUCA |
| 00197580- | | ID | CUGGAUCUGCUCAAUACAAAA |
| ENST000 | | NO: | |
| 00527939- | | 1054 | |
| 56 | | | |
| ENSG000 | 1.237656909 | SEQ | ACUGUUUAGUAGUACUCAAAACUGCCAGUGUGAGA |
| 00197580- | | ID | GGAUUUGGAAAUCACUGGAUCUGCUCAAUACAAAA |
| ENST000 | | NO: | |
| 00531003- | | 1055 | |
| 70 | | | |
| ENSG000 | 2.54485032 | SEQ | GUGAAUAUUAGUCAUCAUUUAUAUUUUAUUACUAU |
| 00197603- | | ID | UAUUGUUUACAGAAUCUUGGGACUGUUUUACUUAA |
| ENST000 | | NO: | UUGGUCAUUGAUAGCUUAACAAAC |
| 00508244- | | 1056 | |
| 94 | | | |
| ENSG000 | 1.326741738 | SEQ | ACAGUGAUAUCCUGAGAGAAG |
| 00197885- | | ID | |
| ENST000 | | NO: | |
| 00614374- | | 1057 | |
| 21 | | | |
| ENSG000 | 1.368717994 | SEQ | AGAGAACCUAGAGCCCAAGGUUCAGAGUCACCCAUC |
| 00197919- | | ID | UCAGCAAGCCCAGAAGUAUCUGCAAUAUCUACG |
| ENST000 | | NO: | |
| 00276927- | | 1058 | |
| 69 | | | |
| ENSG000 | 1.21685797 | SEQ | CCUUCGCGGCGCUUCCUAGUUCGGCUGGUUCUUCUG |
| 00198001- | | ID | UCGCCGGCUUCAGCAGCCCGCGCCCGGGCAGGAAUA |
| ENST000 | | NO: | GAAG |
| 00550615- | | 1059 | |
| 76 | | | |
| ENSG000 | 1.568821266 | SEQ | AAGCAGAUUCUAAAAGCAGUUUCUCUUCAGAACAU |
| 00198015- | | ID | CUUUUUUCAUACCACUUGAUAAGCAUCUUGAAACA |
| ENST000 | | NO: | CC |
| 00548545- | | 1060 | |
| 72 | | | |
| ENSG000 | 1.252271504 | SEQ | GUUGAGGUCAAGUAGUAGCGUUGGGCUGCGGCAGC |
| 00198033- | | ID | GGAGGAGCUCAAC |
| ENST000 | | NO: | |
| 00618094- | | 1061 | |
| 48 | | | |
| ENSG000 | 1.400423915 | SEQ | CUUACCGUGGCGAGUUCCGCGCUCA |
| 00198056- | | ID | |
| ENST000 | | NO: | |
| 00338193- | | 1062 | |
| 25 | | | |
| ENSG000 | 1.159667994 | SEQ | GGUAGACGCGAUCUGCUGGCUACUACCGGCCUUCCC |
| 00198062- | | ID | UGGCUGUUAAAAGCAG |
| ENST000 | | NO: | |
| 00343518- | | 1063 | |
| 52 | | | |
| ENSG000 | 1.519311248 | SEQ | UUAAAGUACUUGGAGUUUUUAUAGGUUGGCACUCA |
| 00198064- | | ID | UCAGUAGCCCCUGUUCUCAUUCUGCAA |
| ENST000 | | NO: | |
| 00564219- | | 1064 | |
| 62 | | | |
| ENSG000 | 1.243385767 | SEQ | AGACUGGUCUUCUGAGGACCUCUGCCCUCUACACAG |
| 00198131- | | ID | CGGCCUCUUCAGGUGCAGGGAGGAA |
| ENST000 | | NO: | |
| 00269829- | | 1065 | |
| 61 | | | |
| ENSG000 | 1.364844039 | SEQ | GAAAACUCAUUUGCUCUUGGAGCAGUCAGGCAGUG |
| 00198157- | | ID | ACUGCCUUCGGCUUUUUUUCUGCUGACUAAGAUCU |
| ENST000 | | NO: | CCUAUAGAGAGCUACAACA |
| 00373250- | | 1066 | |
| 89 | | | |
| ENSG000 | 1.287988673 | SEQ | UUCUCUCUGACCAGCACC |
| 00198223- | | ID | |
| ENST000 | | NO: | |
| 00381500- | | 1067 | |
| 18 | | | |
| ENSG000 | 1.136884293 | SEQ | GUCAUUGGCACC |
| 00198231- | | ID | |
| ENST000 | | NO: | |
| 00578137- | | 1068 | |
| 12 | | | |
| ENSG000 | 1.327651757 | SEQ | AUUGGAGACCCUUUUCACAAG |
| 00198242- | | ID | |
| ENST000 | | NO: | |
| 00422514- | | 1069 | |
| 21 | | | |
| ENSG000 | 1.212784343 | SEQ | CUAAAACCUGCCAGCAGUACUUUUGAGUUUUUUUU |
| 00198363- | | ID | UUUGUUUUGUUUUACUUUAGCAUUUAUUAUUCAGU |
| ENST000 | | NO: | GAUUGAAGAAAUCAAA |
| 00389204- | | 1070 | |
| 86 | | | |
| ENSG000 | 1.295356884 | SEQ | AGUCAAACUCAGAAUCUUCUCAGUGUAACUCAGCU |
| 00198390- | | ID | GAACUCACAUCUCCCAUCAAC |
| ENST000 | | NO: | |
| 00355459- | | 1071 | |
| 56 | | | |
| ENSG000 | 1.262455064 | SEQ | AGCAUAACUUAUAGAGAAGCUAGUAAGUUCUGAGA |
| 00198398- | | ID | AAGAAGGAAAUAAACACAGGCACCAAACCACUAUC |
| ENST000 | | NO: | CUAAGUUGACUGUCCUUUAAAU |
| 00354905- | | 1072 | |
| 92 | | | |
| ENSG000 | 1.447695994 | SEQ | ACAUUCGAAAAACUCACUGUCUUGGAAACCUACCU |
| 00198443- | | ID | AGAACCUCCACCCUCUGACACC |
| ENST000 | | NO: | |
| 00398472- | | 1073 | |
| 57 | | | |
| ENSG000 | 1.285407624 | SEQ | CUUUCAAAGAGACAUAUUAGUCAAGGAAGCAACUC |
| 00198633- | | ID | GGAAGAGGAAAGAAAGGAAGUCAGGA |
| ENST000 | | NO: | |
| 00332323- | | 1074 | |
| 61 | | | |
| ENSG000 | 1.357838037 | SEQ | UCUUUUCUGCCGGGUA |
| 00198839- | | ID | |
| ENST000 | | NO: | |
| 00450657- | | 1075 | |
| 16 | | | |
| ENSG000 | 1.200607499 | SEQ | CCCAGGCAGACUGAGGAGUCCCUGUGUUUUUGGCA |
| 00203757- | | ID | AAGGUAGCUCUACUAGAAAU |
| ENST000 | | NO: | |
| 00368145- | | 1076 | |
| 55 | | | |
| ENSG000 | 1.220477114 | SEQ | ACAUUGCCAGCAUCUUCUCUCCUCACUCACCCUUCC |
| 00203782- | | ID | UGGUGCUUUGGGCUCUCCUUCCUUCUCAGACAAG |
| ENST000 | | NO: | |
| 00368742- | | 1077 | |
| 70 | | | |
| ENSG000 | 1.211073127 | SEQ | UAAACUCCUGGUACUUGAGCACUGAUCUGCUUUGG |
| 00203785- | | ID | AGAACCUGAUUCUGAGACUCCAGCAGG |
| ENST000 | | NO: | |
| 00368750- | | 1078 | |
| 62 | | | |
| ENSG000 | 1.255133038 | SEQ | AUAGGAAUUUAGUGAUUGACA |
| 00203963- | | ID | |
| ENST000 | | NO: | |
| 00371006- | | 1079 | |
| 21 | | | |
| ENSG000 | 1.172272144 | SEQ | ACAGUGCUCAUUGUGCCUGCGCUGCCAGCAGCCAGG |
| 00204161- | | ID | AGCCAGGAGCCAAGAGCAGAGCGCCAGCAGUAACU |
| ENST000 | | NO: | UGGGGGUCAGC |
| 00374151- | | 1080 | |
| 82 | | | |
| ENSG000 | 1.094469431 | SEQ | CUGGAAGGAAGCAGG |
| 00204305- | | ID | |
| ENST000 | | NO: | |
| 00375056- | | 1081 | |
| 15 | | | |
| ENSG000 | 1.241533366 | SEQ | AAUCAAGGAGAGUCUCUGGACACUAUAAUUCCUGU |
| 00204361- | | ID | GAGAACACGAGAAG |
| ENST000 | | NO: | |
| 00389586- | | 1082 | |
| 49 | | | |
| ENSG000 | 1.226415559 | SEQ | AUUCAGUUGGCCACUGCUGAGCAGCUGAGAAGGUG |
| 00204516- | | ID | GCGACGUAGGGGCC |
| ENST000 | | NO: | |
| 00252229- | | 1083 | |
| 49 | | | |
| ENSG000 | 1.803398624 | SEQ | AUAAACAAACAUUGAUUGCUUUCCUAUCUUACAUU |
| 00204703- | | ID | UUUUUAGGAGUGCGAAAUAAGUGAGUCAUC |
| ENST000 | | NO: | |
| 00377173- | | 1084 | |
| 65 | | | |
| ENSG000 | 1.217365165 | SEQ | ACGAGUUCUUCCGGGGCGGAGGUCACC |
| 00204822- | | ID | |
| ENST000 | | NO: | |
| 00409710- | | 1085 | |
| 27 | | | |
| ENSG000 | 1.181049777 | SEQ | AGAUUUUGGGAAACUCACCUCUUAACAGAAGCCCA |
| 00204873- | | ID | CCCUCCAUCCCUGACACC |
| ENST000 | | NO: | |
| 00411528- | | 1086 | |
| 53 | | | |
| ENSG000 | 1.302969659 | SEQ | AGAAGGCACUCCAUUCUGGCUUUCACAAAUACAGC |
| 00204897- | | ID | UCUGCAACGCGUUUGCCCUGAUACC |
| ENST000 | | NO: | |
| 00312150- | | 1087 | |
| 60 | | | |
| ENSG000 | 2.012761787 | SEQ | CACACUCUGCCUUUGGACGUGAGAGAGAGCGCACCU |
| 00205097- | | ID | UUCACUUGAGCUUCAAC |
| ENST000 | | NO: | |
| 00378763- | | 1088 | |
| 53 | | | |
| ENSG000 | 1.327723269 | SEQ | ACCAGAACCCUGAAUUCCGAGAUAGAGGGAGUGUU |
| 00205108- | | ID | GAUCUCAGUCUAAGUAAGUUUAGACC |
| ENST000 | | NO: | |
| 00378788- | | 1089 | |
| 61 | | | |
| ENSG000 | 1.616305036 | SEQ | GUGCAGCAUUGUACUGCAAGUCAAUCGAUACAAUA |
| 00205111- | | ID | AUUUAAGUCACUUCAGCUAUA |
| ENST000 | | NO: | |
| 00419111- | | 1090 | |
| 56 | | | |
| ENSG000 | 1.138413812 | SEQ | AUAACUCAGCCUCUCCAGAGUGCAGCCACCAGUACC |
| 00205279- | | ID | UCCGCAGAUUGAGUAGUGAAGAAAAGAAAACCAGG |
| ENST000 | | NO: | AUAUCCUGUGCUCUGGCUUCCCUGGACC |
| 00620385- | | 1091 | |
| 99 | | | |
| ENSG000 | 1.286185086 | SEQ | UUUCUUAGUUGGAAGUUGUGGUAGUUCUCCAGGCU |
| 00205352- | | ID | GGAGGACACACCUAAAC |
| ENST000 | | NO: | |
| 00549135- | | 1092 | |
| 52 | | | |
| ENSG000 | 1.196031431 | SEQ | AGACUGCGAAGGAGAACGCAGCAAGCCCAGGCGGC |
| 00205352- | | ID | GGUGGAAAGGCUGGAGGACACACCUAAAC |
| ENST000 | | NO: | |
| 00549581- | | 1093 | |
| 64 | | | |
| ENSG000 | 1.234513358 | SEQ | CGAACACCUCCAAC |
| 00205403- | | ID | |
| ENST000 | | NO: | |
| 00512148- | | 1094 | |
| 14 | | | |
| ENSG000 | 1.400709177 | SEQ | AGUCCUGCUUCUCUUCCCUCUCUCCUCCAGCCUCUC |
| 00205420- | | ID | ACACUCUCCUCAGCUCUCUCAUCUCCUGGAACC |
| ENST000 | | NO: | |
| 00330722- | | 1095 | |
| 69 | | | |
| ENSG000 | 1.511096092 | SEQ | GAGAGACUACAACUCCCAGUAGUCACCGCG |
| 00205456- | | ID | |
| ENST000 | | NO: | |
| 00569631- | | 1096 | |
| 30 | | | |
| ENSG000 | 1.511096092 | SEQ | GAGAGACUACAACUCCCAGUAGUCACCGCG |
| 00205457- | | ID | |
| ENST000 | | NO: | |
| 00360260- | | 1097 | |
| 30 | | | |
| ENSG000 | 1.110139908 | SEQ | AGACACACACAGCCUCUCUGCCCACCUCUGCUUCCU |
| 00206073- | | ID | CUAGGAACACAGGAGUUCCAGAUCACAUCGAGUUC |
| ENST000 | | NO: | ACC |
| 00341074- | | 1098 | |
| 74 | | | |
| ENSG000 | 1.533402897 | SEQ | AUUGAACUCCAUCUUCUGACACC |
| 00206106- | | ID | |
| ENST000 | | NO: | |
| 00382830- | | 1099 | |
| 23 | | | |
| ENSG000 | 1.094469431 | SEQ | CUGGAAGGAAGCAGG |
| 00206320- | | ID | |
| ENST000 | | NO: | |
| 00383279- | | 1100 | |
| 15 | | | |
| ENSG000 | 1.185659153 | SEQ | GGGGAACAUAGAGUGCUGAAGACAGAAUCUAGAGC |
| 00206355- | | ID | CUUCAAAUAAGUUGGAG |
| ENST000 | | NO: | |
| 00449364- | | 1101 | |
| 52 | | | |
| ENSG000 | 1.209507245 | SEQ | AAUCACUCCGGAGACUGAGCC |
| 00206378- | | ID | |
| ENST000 | | NO: | |
| 00383379- | | 1102 | |
| 21 | | | |
| ENSG000 | 1.133412347 | SEQ | GCUGACGUGAAG |
| 00206406- | | ID | |
| ENST000 | | NO: | |
| 00620798- | | 1103 | |
| 12 | | | |
| ENSG000 | 1.499177611 | SEQ | GGGGAACAUAGAGUGCUGGAGACAGAAUCUAGAGC |
| 00206457- | | ID | CUUCAAAUAAGUUGGAG |
| ENST000 | | NO: | |
| 00429943- | | 1104 | |
| 52 | | | |
| ENSG000 | 1.38117268 | SEQ | GCCGGAAGCGGAAAUAGCACCGGGCGCCGCCACAGU |
| 00206490- | | ID | AGCUGUAACUGCCACCGCG |
| ENST000 | | NO: | |
| 00424994- | | 1105 | |
| 55 | | | |
| ENSG000 | 1.803398624 | SEQ | AUAAACAAACAUUGAUUGCUUUCCUAUCUUACAUU |
| 00206524- | | ID | UUUUUAGGAGUGCGAAAUAAGUGAGUCAUC |
| ENST000 | | NO: | |
| 00383653- | | 1106 | |
| 65 | | | |
| ENSG000 | 1.099729526 | SEQ | UCAGCUCCCAGAGUGAUCCUGGACAGG |
| 00206526- | | ID | |
| ENST000 | | NO: | |
| 00383656- | | 1107 | |
| 27 | | | |
| ENSG000 | 1.168348872 | SEQ | AUAAAAUCUGUGCUGUCAAACUGAUUAGGAACUGA |
| 00211599- | | ID | CUACCACCUGCAGGUCAGGGCCAAGGUU |
| ENST000 | | NO: | |
| 00390244- | | 1108 | |
| 63 | | | |
| ENSG000 | 1.552173531 | SEQ | AUCUUAAAAGAGGUUCUUUCUCUGGGAGUUGGCAG |
| 00211611- | | ID | UAGCAAAACUGACAAGUCAAGGCAGGAAG |
| ENST000 | | NO: | |
| 00390256- | | 1109 | |
| 64 | | | |
| ENSG000 | 1.110137154 | SEQ | CAAGUCACAGAUACAGAAGACCCCUCCGUCCUGGAG |
| 00211724- | | ID | CACCUGCC |
| ENST000 | | NO: | |
| 00390371- | | 1110 | |
| 44 | | | |
| ENSG000 | 1.580879837 | SEQ | AUUCUUUCUUCAAAGCAGCC |
| 00211753- | | ID | |
| ENST000 | | NO: | |
| 00390400- | | 1111 | |
| 20 | | | |
| ENSG000 | 1.263523387 | SEQ | CACUGUGAUUUCUUCAGUUUAAGGAUCAAGACCAU |
| 00211793- | | ID | UAUUUGGGUAACACACUAAAG |
| ENST000 | | NO: | |
| 00390441- | | 1112 | |
| 56 | | | |
| ENSG000 | 1.837518927 | SEQ | AUUAUUUUUUUUUCGUGUUUAAGGUUUGAAUCCUC |
| 00211794- | | ID | AGUGAACCAGGGCAGAAAAGA |
| ENST000 | | NO: | |
| 00390442- | | 1113 | |
| 56 | | | |
| ENSG000 | 1.140021391 | SEQ | ACAGAAGUGGCGCCUCUGAGAAAAGAAGGUUGGAA |
| 00211800- | | ID | UUAUCGUAAUUUGUUUCUAGGCUGAGAUACCAGC |
| ENST000 | | NO: | |
| 00390448- | | 1114 | |
| 69 | | | |
| ENSG000 | 1.800550963 | SEQ | ACUAUUUCUAUUGAUCUGGAAGAAGCUUGUACCAG |
| 00211806- | | ID | GCAACCCAUUUAGGAGAAGUUGGAGUAAGAGGGAG |
| ENST000 | | NO: | AGGGAG |
| 00390454- | | 1115 | |
| 76 | | | |
| ENSG000 | 1.65030921 | SEQ | AGCAUCAUCCAGAAACCACAUCCCUCCGCUAGAGAA |
| 00211968- | | ID | GCCCCUGACGGCACAGUUCCUCACU |
| ENST000 | | NO: | |
| 00390628- | | 1116 | |
| 61 | | | |
| ENSG000 | 1.364868797 | SEQ | UCACAACCAGUGAUAUUAGGCUUGCAUUUUCUUUC |
| 00212126- | | ID | UUUUUUUCUGCUCAGAU |
| ENST000 | | NO: | |
| 00506868- | | 1117 | |
| 52 | | | |
| ENSG000 | 1.285281798 | SEQ | AUUCACACCUCAGAAGCCUUAUCCUUCUUUUCAAUC |
| 00212899- | | ID | AAACCCAUAAAUACCACAGACUCUAAUAGCC |
| ENST000 | | NO: | |
| 00391586- | | 1118 | |
| 67 | | | |
| ENSG000 | 1.553759704 | SEQ | GUAGCCAUUCCUGAAUUUCUUUCAGCACUGGGAAA |
| 00213139- | | ID | ACCAGUCUAGUCACCAAAA |
| ENST000 | | NO: | |
| 00307944- | | 1119 | |
| 54 | | | |
| ENSG000 | 1.099147505 | SEQ | AUUUACUUCCUGCGGGUGCACAGGCUGUGGUCGUC |
| 00213246- | | ID | UAUCUCCCUGUUGUUCUUCCCAUCGGCGAAG |
| ENST000 | | NO: | |
| 00225504- | | 1120 | |
| 66 | | | |
| ENSG000 | 1.236604787 | SEQ | GUUGCUGGGAAG |
| 00213380- | | ID | |
| ENST000 | | NO: | |
| 00306875- | | 1121 | |
| 12 | | | |
| ENSG000 | 1.144582434 | SEQ | UCCUAUCGAUAAGGUUCAGUUAGCCAAGGUGGAGG |
| 00213424- | | ID | AGUGGAAAAGA |
| ENST000 | | NO: | |
| 00581564- | | 1122 | |
| 46 | | | |
| ENSG000 | 1.337584474 | SEQ | CUAGUCUGCAUCUGAGUAAC |
| 00213619- | | ID | |
| ENST000 | | NO: | |
| 00530295- | | 1123 | |
| 20 | | | |
| ENSG000 | 1.117156616 | SEQ | GUCUCUGGUUUCUGGCCCCUUGUCUGCAGAG |
| 00213886- | | ID | |
| ENST000 | | NO: | |
| 00377050- | | 1124 | |
| 31 | | | |
| ENSG000 | 1.13486989 | SEQ | ACAUAUCUGCUUCCGACACAGCUGCAAUCACUAGCA |
| 00213931- | | ID | AGCUCUCAGGCCUGGCAUC |
| ENST000 | | NO: | |
| 00396895- | | 1125 | |
| 55 | | | |
| ENSG000 | 1.856926054 | SEQ | ACACUCGCUUCUGGAACGUCUGAGGUUAUCAAUAA |
| 00213934- | | ID | GCUCCUAGUCCAGACGCC |
| ENST000 | | NO: | |
| 00330597- | | 1126 | |
| 53 | | | |
| ENSG000 | 1.426053561 | SEQ | CAGCGCUUGAGGCUUGAAGAGGGAGACAAGCUAAA |
| 00214050- | | ID | AGAGGAUAUCCAGUUGUUUC |
| ENST000 | | NO: | |
| 00517673- | | 1127 | |
| 55 | | | |
| ENSG000 | 1.281011584 | SEQ | GUAAGUGUGAGAAAUUUGGCAAUAAAACCACCUAU |
| 00214285- | | ID | CUUUACAGAUUUUGGGAAGUCCAAA |
| ENST000 | | NO: | |
| 00398023- | | 1128 | |
| 60 | | | |
| ENSG000 | 1.192857193 | SEQ | GAGUGGAGAGGAAAAACAAAUUAUUUUUAAGUAUU |
| 00214511- | | ID | CACGGCAACCACAUUUAUAUCCUAUUGUUACUAGA |
| ENST000 | | NO: | GAAAAAA |
| 00398455- | | 1129 | |
| 77 | | | |
| ENSG000 | 1.100097911 | SEQ | CAGUUGUACACC |
| 00214782- | | ID | |
| ENST000 | | NO: | |
| 00529108- | | 1130 | |
| 12 | | | |
| ENSG000 | 1.199888668 | SEQ | UCAUUGCAGAGUCGAAAGCC |
| 00214944- | | ID | |
| ENST000 | | NO: | |
| 00426542- | | 1131 | |
| 20 | | | |
| ENSG000 | 1.101420533 | SEQ | CAGCUCUCGGCUCUGCAGCGGAGCAUAGGCGACCAC |
| 00215045- | | ID | CACCGAAAGAGGCCACC |
| ENST000 | | NO: | |
| 00452113- | | 1132 | |
| 53 | | | |
| ENSG000 | 1.173258273 | SEQ | ACAGUGAACUCUGAUUCCUGGAAUUGUUACCAGGC |
| 00215262- | | ID | GACCACGGCGUCAUCAAAGUACCUGGCAAUUCCGUC |
| ENST000 | | NO: | UACUGAGUUCUCGAAC |
| 00523973- | | 1133 | |
| 87 | | | |
| ENSG000 | 1.094037583 | SEQ | AUUUUCUCAAGCAAUUUUUAAAUUUCAUUCUUUUA |
| 00215343- | | ID | CCCUAUUGAGCUUUUCUUAGUGCCUAAAGUGUCUG |
| ENST000 | | NO: | CAAGUGGAAAUCCAGAGGAAGACAGAGAGAAACUG |
| 00400085- | | 1134 | AGUUCCUGACA |
| 116 | | | |
| ENSG000 | 1.189210838 | SEQ | ACAGGUUAGUAGUGCCAGUGAGCCGCCUUAGAUAG |
| 00215421- | | ID | AAGCAUCGUCAGCACUUUAUUA |
| ENST000 | | NO: | |
| 00309902- | | 1135 | |
| 57 | | | |
| ENSG000 | 1.511500077 | SEQ | UUUGUUGAGAGUGGAGUUGGAAAGUGUUAGCACUU |
| 00215529- | | ID | UGCCAGACUUUGCCAGCAAGAUUAACUGAGGAGAU |
| ENST000 | | NO: | CAAAUUGAGUCAGGGUCAAGGCUA |
| 00400522- | | 1136 | |
| 94 | | | |
| ENSG000 | 1.317594256 | SEQ | AGGCCCUGUUUCCCUGAGCUCUCACCUCUGAUACAA |
| 00221840- | | ID | GCCUUAAAGAAGAGUAA |
| ENST000 | | NO: | |
| 00319760- | | 1137 | |
| 53 | | | |
| ENSG000 | 1.346037672 | SEQ | CCCAUUCUUGGCGCAGAAACCUGAAAGUCAGGAUC |
| 00221937- | | ID | AAAGAGA |
| ENST000 | | NO: | |
| 00408947- | | 1138 | |
| 42 | | | |
| ENSG000 | 1.312935781 | SEQ | CCUUCCUGGAGGUUUUAUCACCCACACUACAUAG |
| 00221954- | | ID | |
| ENST000 | | NO: | |
| 00335238- | | 1139 | |
| 34 | | | |
| ENSG000 | 1.185659153 | SEQ | GGGGAACAUAGAGUGCUGAAGACAGAAUCUAGAGC |
| 00223533- | | ID | CUUCAAAUAAGUUGGAG |
| ENST000 | | NO: | |
| 00456365- | | 1140 | |
| 52 | | | |
| ENSG000 | 1.185659153 | SEQ | GGGGAACAUAGAGUGCUGAAGACAGAAUCUAGAGC |
| 00224180- | | ID | CUUCAAAUAAGUUGGAG |
| ENST000 | | NO: | |
| 00439536- | | 1141 | |
| 52 | | | |
| ENSG000 | 1.133412347 | SEQ | GCUGACGUGAAG |
| 00224398- | | ID | |
| ENST000 | | NO: | |
| 00621203- | | 1142 | |
| 12 | | | |
| ENSG000 | 1.133412347 | SEQ | GCUGACGUGAAG |
| 00224774- | | ID | |
| ENST000 | | NO: | |
| 00622139- | | 1143 | |
| 12 | | | |
| ENSG000 | 1.384930042 | SEQ | GGUAUCUUAAAAGAGGUUCUUUCUCUGGGAGUUGG |
| 00225523- | | ID | CAGUAGCAAAACUGACAAGUCAAGGCAGGAAG |
| ENST000 | | NO: | |
| 00436451- | | 1144 | |
| 67 | | | |
| ENSG000 | 1.803398624 | SEQ | AUAAACAAACAUUGAUUGCUUUCCUAUCUUACAUU |
| 00225736- | | ID | UUUUUAGGAGUGCGAAAUAAGUGAGUCAUC |
| ENST000 | | NO: | |
| 00456140- | | 1145 | |
| 65 | | | |
| ENSG000 | 1.141243899 | SEQ | UCCUCACAAUUACUCUACAGCUCAGAACAGCAACUG |
| 00225890- | | ID | CUGAGGCUGCCUUGGGAAGAAG |
| ENST000 | | NO: | |
| 00416403- | | 1146 | |
| 58 | | | |
| ENSG000 | 2.012761787 | SEQ | CACACUCUGCCUUUGGACGUGAGAGAGAGCGCACCU |
| 00225899- | | ID | UUCACUUGAGCUUCAAC |
| ENST000 | | NO: | |
| 00425520- | | 1147 | |
| 53 | | | |
| ENSG000 | 1.38117268 | SEQ | GCCGGAAGCGGAAAUAGCACCGGGCGCCGCCACAGU |
| 00225989- | | ID | AGCUGUAACUGCCACCGCG |
| ENST000 | | NO: | |
| 00441688- | | 1148 | |
| 55 | | | |
| ENSG000 | 1.803398624 | SEQ | AUAAACAAACAUUGAUUGCUUUCCUAUCUUACAUU |
| 00226832- | | ID | UUUUUAGGAGUGCGAAAUAAGUGAGUCAUC |
| ENST000 | | NO: | |
| 00415976- | | 1149 | |
| 65 | | | |
| ENSG000 | 1.613901653 | SEQ | ACCUUCGCCCUAAGUCCUUUACCAAUAGUGCGGCAC |
| 00227322- | | ID | UCCGGUAGCGACGAAUCCAUCCUGCACUGACACGC |
| ENST000 | | NO: | |
| 00531100- | | 1150 | |
| 71 | | | |
| ENSG000 | 1.12821117 | SEQ | UUCAGUCUCA |
| 00227507- | | ID | |
| ENST000 | | NO: | |
| 00446745- | | 1151 | |
| 10 | | | |
| ENSG000 | 1.302467484 | SEQ | AGAAAACCUACAACCCAAGGUUCAGUGUUACCCCUC |
| 00228083- | | ID | AUCAACCAGCCCAGCAGCAUCUUCGGGAUUCCCA |
| ENST000 | | NO: | |
| 00380222- | | 1152 | |
| 70 | | | |
| ENSG000 | 1.209507245 | SEQ | AAUCACUCCGGAGACUGAGCC |
| 00228263- | | ID | |
| ENST000 | | NO: | |
| 00425238- | | 1153 | |
| 21 | | | |
| ENSG000 | 1.233260608 | SEQ | CUCUGACUUCGGGCUUGUUCGCUGGUGGCGUCGGA |
| 00228474- | | ID | GCCGAGCCGGACUGGUCAGG |
| ENST000 | | NO: | |
| 00456793- | | 1154 | |
| 55 | | | |
| ENSG000 | 1.133412347 | SEQ | GCUGACGUGAAG |
| 00228875- | | ID | |
| ENST000 | | NO: | |
| 00614974- | | 1155 | |
| 12 | | | |
| ENSG000 | 1.094469431 | SEQ | CUGGAAGGAAGCAGG |
| 00229058- | | ID | |
| ENST000 | | NO: | |
| 00412470- | | 1156 | |
| 15 | | | |
| ENSG000 | 1.209507245 | SEQ | AAUCACUCCGGAGACUGAGCC |
| 00229077- | | ID | |
| ENST000 | | NO: | |
| 00415517- | | 1157 | |
| 21 | | | |
| ENSG000 | 1.094469431 | SEQ | CUGGAAGGAAGCAGG |
| 00230514- | | ID | |
| ENST000 | | NO: | |
| 00412794- | | 1158 | |
| 15 | | | |
| ENSG000 | 1.099729526 | SEQ | UCAGCUCCCAGAGUGAUCCUGGACAGG |
| 00231041- | | ID | |
| ENST000 | | NO: | |
| 00421470- | | 1159 | |
| 27 | | | |
| ENSG000 | 1.38117268 | SEQ | GCCGGAAGCGGAAAUAGCACCGGGCGCCGCCACAGU |
| 00231129- | | ID | AGCUGUAACUGCCACCGCG |
| ENST000 | | NO: | |
| 00426591- | | 1160 | |
| 55 | | | |
| ENSG000 | 1.094469431 | SEQ | CUGGAAGGAAGCAGG |
| 00231268- | | ID | |
| ENST000 | | NO: | |
| 00418313- | | 1161 | |
| 15 | | | |
| ENSG000 | 1.803398624 | SEQ | AUAAACAAACAUUGAUUGCUUUCCUAUCUUACAUU |
| 00231319- | | ID | UUUUUAGGAGUGCGAAAUAAGUGAGUCAUC |
| ENST000 | | NO: | |
| 00419873- | | 1162 | |
| 65 | | | |
| ENSG000 | 1.209507245 | SEQ | AAUCACUCCGGAGACUGAGCC |
| 00231479- | | ID | |
| ENST000 | | NO: | |
| 00453831- | | 1163 | |
| 21 | | | |
| ENSG000 | 1.141489866 | SEQ | AAAGUUUAUUCAGCUUUUCGAUCCGCCAUCUGCAG |
| 00231767- | | ID | UGGAGCCGCCACCAAA |
| ENST000 | | NO: | |
| 00647225- | | 1164 | |
| 51 | | | |
| ENSG000 | 1.38117268 | SEQ | GCCGGAAGCGGAAAUAGCACCGGGCGCCGCCACAGU |
| 00232169- | | ID | AGCUGUAACUGCCACCGCG |
| ENST000 | | NO: | |
| 00429395- | | 1165 | |
| 55 | | | |
| ENSG000 | 1.209507245 | SEQ | AAUCACUCCGGAGACUGAGCC |
| 00232180- | | ID | |
| ENST000 | | NO: | |
| 00442152- | | 1166 | |
| 21 | | | |
| ENSG000 | 1.740603439 | SEQ | GAAGGAAAGAGUAACUUGAGUUUCACUUCUUAGUG |
| 00232869- | | ID | CCUUUUCUCAGGGGAGAGGCCAUCACUUGAAG |
| ENST000 | | NO: | |
| 00422143- | | 1167 | |
| 67 | | | |
| ENSG000 | 1.803398624 | SEQ | AUAAACAAACAUUGAUUGCUUUCCUAUCUUACAUU |
| 00233054- | | ID | UUUUUAGGAGUGCGAAAUAAGUGAGUCAUC |
| ENST000 | | NO: | |
| 00420178- | | 1168 | |
| 65 | | | |
| ENSG000 | 1.803398624 | SEQ | AUAAACAAACAUUGAUUGCUUUCCUAUCUUACAUU |
| 00233180- | | ID | UUUUUAGGAGUGCGAAAUAAGUGAGUCAUC |
| ENST000 | | NO: | |
| 00444830- | | 1169 | |
| 65 | | | |
| ENSG000 | 1.099729526 | SEQ | UCAGCUCCCAGAGUGAUCCUGGACAGG |
| 00233566- | | ID | |
| ENST000 | | NO: | |
| 00438403- | | 1170 | |
| 27 | | | |
| ENSG000 | 1.803398624 | SEQ | AUAAACAAACAUUGAUUGCUUUCCUAUCUUACAUU |
| 00233687- | | ID | UUUUUAGGAGUGCGAAAUAAGUGAGUCAUC |
| ENST000 | | NO: | |
| 00450817- | | 1171 | |
| 65 | | | |
| ENSG000 | 1.17875259 | SEQ | GUUGACAGAGCUACCGUCUUCCUGUUUUUUUCCUCC |
| 00233822- | | ID | AAUUUUCCGGCAGUUACUCCCAGUC |
| ENST000 | | NO: | |
| 00606613- | | 1172 | |
| 61 | | | |
| ENSG000 | 1.185659153 | SEQ | GGGGAACAUAGAGUGCUGAAGACAGAAUCUAGAGC |
| 00234114- | | ID | CUUCAAAUAAGUUGGAG |
| ENST000 | | NO: | |
| 00444737- | | 1173 | |
| 52 | | | |
| ENSG000 | 1.094469431 | SEQ | CUGGAAGGAAGCAGG |
| 00234729- | | ID | |
| ENST000 | | NO: | |
| 00434502- | | 1174 | |
| 15 | | | |
| ENSG000 | 1.209507245 | SEQ | AAUCACUCCGGAGACUGAGCC |
| 00235336- | | ID | |
| ENST000 | | NO: | |
| 00417894- | | 1175 | |
| 21 | | | |
| ENSG000 | 1.38117268 | SEQ | GCCGGAAGCGGAAAUAGCACCGGGCGCCGCCACAGU |
| 00236149- | | ID | AGCUGUAACUGCCACCGCG |
| ENST000 | | NO: | |
| 00423030- | | 1176 | |
| 55 | | | |
| ENSG000 | 1.38117268 | SEQ | GCCGGAAGCGGAAAUAGCACCGGGCGCCGCCACAGU |
| 00236342- | | ID | AGCUGUAACUGCCACCGCG |
| ENST000 | | NO: | |
| 00418552- | | 1177 | |
| 55 | | | |
| ENSG000 | 1.141243899 | SEQ | UCCUCACAAUUACUCUACAGCUCAGAACAGCAACUG |
| 00236418- | | ID | CUGAGGCUGCCUUGGGAAGAAG |
| ENST000 | | NO: | |
| 00442315- | | 1178 | |
| 58 | | | |
| ENSG000 | 1.099729526 | SEQ | UCAGCUCCCAGAGUGAUCCUGGACAGG |
| 00237313- | | ID | |
| ENST000 | | NO: | |
| 00434916- | | 1179 | |
| 27 | | | |
| ENSG000 | 1.094469431 | SEQ | CUGGAAGGAAGCAGG |
| 00237405- | | ID | |
| ENST000 | | NO: | |
| 00441753- | | 1180 | |
| 15 | | | |
| ENSG000 | 1.733843671 | SEQ | ACAAUUGCUCUACAGCUCAGAGCAGCAACUGCUGA |
| 00237541- | | ID | GGCUGCCUUGGGAAGAAG |
| ENST000 | | NO: | |
| 00374940- | | 1181 | |
| 53 | | | |
| ENSG000 | 1.099729526 | SEQ | UCAGCUCCCAGAGUGAUCCUGGACAGG |
| 00237634- | | ID | |
| ENST000 | | NO: | |
| 00438996- | | 1182 | |
| 27 | | | |
| ENSG000 | 1.150959433 | SEQ | UUAGUUCUUCCUGAGGUCUGAGCACCCUCCAAACUG |
| 00237700- | | ID | AGUCCAGAUCUGAGUUUUUCCUCGGAGAUUUGUCA |
| ENST000 | | NO: | GA |
| 00437300- | | 1183 | |
| 73 | | | |
| ENSG000 | 1.35564606 | SEQ | UCUUCAUUCUUUCCGCCAUCUUGAUUCUUUGUCACU |
| 00238269- | | ID | GACCGAGACUCAGCCGUGGGAAAU |
| ENST000 | | NO: | |
| 00374974- | | 1184 | |
| 60 | | | |
| ENSG000 | 1.429580126 | SEQ | GACAGUUUGAGGUAAGGCGUUGUAUAUAUUGCAGA |
| 00239389- | | ID | UAGCUCUGAGGUUUUUGGAGUGUACC |
| ENST000 | | NO: | |
| 00409494- | | 1185 | |
| 61 | | | |
| ENSG000 | 1.270018906 | SEQ | AGGCUGGACACACUUCAGUCAGGAGUCAGACCCUG |
| 00240671- | | ID | UCAGGACACAGCAUAGAC |
| ENST000 | | NO: | |
| 00495489- | | 1186 | |
| 53 | | | |
| ENSG000 | 1.29421234 | SEQ | UCUCUCUUAUCUUCUGGCCUGCCUGGAGUUUCAAG |
| 00241186- | | ID | GGAACAAGUACAGAGUGUGAUUUGGAUC |
| ENST000 | | NO: | |
| 00542931- | | 1187 | |
| 63 | | | |
| ENSG000 | 1.365944978 | SEQ | UGGAAAAUCCCU |
| 00241322- | | ID | |
| ENST000 | | NO: | |
| 00630868- | | 1188 | |
| 12 | | | |
| ENSG000 | 1.123661671 | SEQ | ACAUUUCAGGGACACC |
| 00241644- | | ID | |
| ENST000 | | NO: | |
| 00013222- | | 1189 | |
| 16 | | | |
| ENSG000 | 1.498382268 | SEQ | CUGGGAAGCGCC |
| 00242110- | | ID | |
| ENST000 | | NO: | |
| 00382072- | | 1190 | |
| 12 | | | |
| ENSG000 | 1.221715055 | SEQ | UUUUCGUCGACUCUUACCGGUUGGCUGGGCCAGCU |
| 00242247- | | ID | GCGCCGCGGCUCACAGCUGACG |
| ENST000 | | NO: | |
| 00437119- | | 1191 | |
| 57 | | | |
| ENSG000 | 1.223653081 | SEQ | AAACCUUUUUAGGGAGUCCAAGGUACAGUCGCCGC |
| 00242372- | | ID | GUGCGGAGCUUGUUACUGGUUACUUGGCCUC |
| ENST000 | | NO: | |
| 00415116- | | 1192 | |
| 66 | | | |
| ENSG000 | 1.705298272 | SEQ | AUAGGUUGGCACUCAUCAGUAGCCCCUGUUCUCAU |
| 00243716- | | ID | UCUGCAA |
| ENST000 | | NO: | |
| 00517347- | | 1193 | |
| 42 | | | |
| ENSG000 | 1.321173039 | SEQ | UAAACUCCUGGUACCUAAGCACCGAUCUGCCUUGGA |
| 00244094- | | ID | GAACCUGAUUCUGAGACUCCAGCAGG |
| ENST000 | | NO: | |
| 00468739- | | 1194 | |
| 62 | | | |
| ENSG000 | 1.749866577 | SEQ | AAACCUUCCAAACCCACCUUCCUGGAAAUCCACCCA |
| 00244537- | | ID | GAACCUCCACCCUCUGACACC |
| ENST000 | | NO: | |
| 00377726- | | 1195 | |
| 57 | | | |
| ENSG000 | 1.391560559 | SEQ | UGAAGACAGCCACACAGAUAUUGCACAGACUAUUU |
| 00248329- | | ID | ACAGAUCGUUUGGUUUACAUUGAGAGUCAUUGCUC |
| ENST000 | | NO: | UACUUUUGUGCGGUAGGAAA |
| 00510062- | | 1196 | |
| 90 | | | |
| ENSG000 | 2.044843971 | SEQ | UCCACUGGCCAGCAUUCUCCUCUAUUAGACUAGAAC |
| 00249437- | | ID | UGUGGAUAAACCUCAGAAA |
| ENST000 | | NO: | |
| 00508426- | | 1197 | |
| 55 | | | |
| ENSG000 | 1.206064157 | SEQ | ACUCAGAAGACUGGACACAAUUCCGAAGGUCGCCCA |
| 00249861- | | ID | GAAGGAGAGGACA |
| ENST000 | | NO: | |
| 00392051- | | 1198 | |
| 49 | | | |
| ENSG000 | 1.367711867 | SEQ | AAGACUGGACACAAUUCCGAAGGUCGCCCAGAAGG |
| 00249861- | | ID | AGAGGACA |
| ENST000 | | NO: | |
| 00594480- | | 1199 | |
| 43 | | | |
| ENSG000 | 1.269420374 | SEQ | GUCUUUGGUAGUUUUUUUGCACUAACUUCAGGAAC |
| 00250361- | | ID | CAGCUCAGUAUCUCAGG |
| ENST000 | | NO: | |
| 00506679- | | 1200 | |
| 52 | | | |
| ENSG000 | 1.399051287 | SEQ | ACAAGAGCAAGCAGAACCUCUCUGACCGCUCGCGCC |
| 00251184- | | ID | AGGCCUACACUUUCCACCUC |
| ENST000 | | NO: | |
| 00482796- | | 1201 | |
| 56 | | | |
| ENSG000 | 1.570950729 | SEQ | CUGGCCAGAUCUCUCUACACAGGUAAAAGAGUAAG |
| 00254658- | | ID | CCAACAAUACAA |
| ENST000 | | NO: | |
| 00641465- | | 1202 | |
| 47 | | | |
| ENSG000 | 1.094703228 | SEQ | AUUGCAAGUGUAACACACAUUGCAAGUGCCAACGU |
| 00255501- | | ID | GAGGAGCC |
| ENST000 | | NO: | |
| 00530950- | | 1203 | |
| 43 | | | |
| ENSG000 | 1.13946171 | SEQ | GUGGUGGCAGAGGUGCUUGUGUUUUUGUCGGUACA |
| 00255508- | | ID | GGAGAGUCGCU |
| ENST000 | | NO: | |
| 00496634- | | 1204 | |
| 46 | | | |
| ENSG000 | 1.240199969 | SEQ | GUUGCUACCGGA |
| 00256061- | | ID | |
| ENST000 | | NO: | |
| 00524160- | | 1205 | |
| 12 | | | |
| ENSG000 | 1.283101369 | SEQ | AUUGAGUAAAAUUGGGUUACCCUGAGGACUCUUGC |
| 00256394- | | ID | UUAACUGAAAUCACAA |
| ENST000 | | NO: | |
| 00537611- | | 1206 | |
| 51 | | | |
| ENSG000 | 1.316464457 | SEQ | UUUCCGGUGCAGGUGGGGAAA |
| 00256591- | | ID | |
| ENST000 | | NO: | |
| 00544801- | | 1207 | |
| 21 | | | |
| ENSG000 | 1.781446799 | SEQ | ACAGUGCGCAGUUGUGCGGCUGUGCUUUGGCUCUU |
| 00256646- | | ID | CGGGUAAAG |
| ENST000 | | NO: | |
| 00676779- | | 1208 | |
| 44 | | | |
| ENSG000 | 1.146990359 | SEQ | CAUCUCCAGGCUCCAUCUCUUGAACCAGUGAUCCCA |
| 00257008- | | ID | GCGUUGUUAGCA |
| ENST000 | | NO: | |
| 00335666- | | 1209 | |
| 48 | | | |
| ENSG000 | 1.218398343 | SEQ | AUUGCUAAGCCAUCCUUCAGACAGAGAGGGAGCGG |
| 00257335- | | ID | CUGCAAGAGGUAAGUAGAG |
| ENST000 | | NO: | |
| 00475668- | | 1210 | |
| 54 | | | |
| ENSG000 | 1.09796851 | SEQ | CUCACUCCGUCUCAAUAGUUCUCAAG |
| 00257923- | | ID | |
| ENST000 | | NO: | |
| 00292538- | | 1211 | |
| 26 | | | |
| ENSG000 | 1.14068138 | SEQ | CACUCCGUCUCAAUAGUUCUCAAG |
| 00257923- | | ID | |
| ENST000 | | NO: | |
| 00622516- | | 1212 | |
| 24 | | | |
| ENSG000 | 1.295409723 | SEQ | ACAGCGGUCACGUGAC |
| 00258311- | | ID | |
| ENST000 | | NO: | |
| 00550412- | | 1213 | |
| 16 | | | |
| ENSG000 | 1.383552553 | SEQ | CGUAUCGCUUGGAGUGAAGGUCCUACCGCGGCAGU |
| 00258405- | | ID | AUUGAUUUCUAAAGACUC |
| ENST000 | | NO: | |
| 00601120- | | 1214 | |
| 53 | | | |
| ENSG000 | 1.133412347 | SEQ | GCUGACGUGAAG |
| 00258543- | | ID | |
| ENST000 | | NO: | |
| 00620322- | | 1215 | |
| 12 | | | |
| ENSG000 | 1.133412347 | SEQ | GCUGACGUGAAG |
| 00258589- | | ID | |
| ENST000 | | NO: | |
| 00614071- | | 1216 | |
| 12 | | | |
| ENSG000 | 1.31134311 | SEQ | GUCUCCCAGACAGUGGCUCAGCCUUCAGGACUCUCU |
| 00258873- | | ID | UCCAGC |
| ENST000 | | NO: | |
| 00554048- | | 1217 | |
| 42 | | | |
| ENSG000 | 1.229055946 | SEQ | GAGAACGAAAG |
| 00259494- | | ID | |
| ENST000 | | NO: | |
| 00312475- | | 1218 | |
| 11 | | | |
| ENSG000 | 1.179253942 | SEQ | CCGGGCCAGUUAGAUUUGGAGGUUCAACUUCAAC |
| 00260238- | | ID | |
| ENST000 | | NO: | |
| 00490491- | | 1219 | |
| 34 | | | |
| ENSG000 | 1.511096092 | SEQ | GAGAGACUACAACUCCCAGUAGUCACCGCG |
| 00261509- | | ID | |
| ENST000 | | NO: | |
| 00569741- | | 1220 | |
| 30 | | | |
| ENSG000 | 1.340249819 | SEQ | ACUCUCUUGCUCACAGUCCACG |
| 00261857- | | ID | |
| ENST000 | | NO: | |
| 00263369- | | 1221 | |
| 22 | | | |
| ENSG000 | 1.321392507 | SEQ | CUAGGCCUCUGUGAUUGUUGAGGAGGGGGCUGUCA |
| 00261857- | | ID | CUGGGAAAGUUUCCACG |
| ENST000 | | NO: | |
| 00597784- | | 1222 | |
| 52 | | | |
| ENSG000 | 1.13138728 | SEQ | CCUCCCACUCUGCCUAGAGUC |
| 00262152- | | ID | |
| ENST000 | | NO: | |
| 00573315- | | 1223 | |
| 21 | | | |
| ENSG000 | 1.35823703 | SEQ | ACAGUCCCUCUGAGCUGCACGGAGACCUCGCAGGCC |
| 00262243- | | ID | CCCGGAACUGUCGCCCUUCCAGG |
| ENST000 | | NO: | |
| 00571922- | | 1224 | |
| 59 | | | |
| ENSG000 | 1.378656618 | SEQ | AGAAAGGAACACAGUAAACUGAAUUGAUCCGUUUA |
| 00262406- | | ID | GAAGUUUACA |
| ENST000 | | NO: | |
| 00571244- | | 1225 | |
| 45 | | | |
| ENSG000 | 1.185966521 | SEQ | AGAGGAGGAAAUUGUUCCUCGUCUGAUAAGACAAC |
| 00262418- | | ID | AGUGGAGAAAGGACGCAGUCUGUUUCUUAGGGACA |
| ENST000 | | NO: | CGGCUGACUUCCAGAUAGUACC |
| 00573477- | | 1226 | |
| 92 | | | |
| ENSG000 | 1.13357079 | SEQ | GCUGUGAGGACGUGUUCCGAGGAAGCCAGAGCCGG |
| 00262633- | | ID | AGCCGUGGCCUGCGGGGCCGGCGAC |
| ENST000 | | NO: | |
| 00571841- | | 1227 | |
| 60 | | | |
| ENSG000 | 1.116135805 | SEQ | AAGGUCAAGUAUAGCAUCUGCCUAGAGUCAAACCU |
| 00262635- | | ID | CCGUGCUUCUCAGACAGUGCCUUUUCACC |
| ENST000 | | NO: | |
| 00573403- | | 1228 | |
| 64 | | | |
| ENSG000 | 1.71541121 | SEQ | UAGAUCGUCAGGAAAAGCCUAAAGAUUAGACUGUA |
| 00262771- | | ID | AGAAAAGAAAAUAGAAGCC |
| ENST000 | | NO: | |
| 00575059- | | 1229 | |
| 54 | | | |
| ENSG000 | 1.133412347 | SEQ | GCUGACGUGAAG |
| 00263020- | | ID | |
| ENST000 | | NO: | |
| 00617558- | | 1230 | |
| 12 | | | |
| ENSG000 | 1.281767423 | SEQ | CAACCAUCCUGAAGCUACAGGUGCUCCCUCCUGGAA |
| 00263057- | | ID | UCUCCA |
| ENST000 | | NO: | |
| 00576098- | | 1231 | |
| 42 | | | |
| ENSG000 | 1.124660387 | SEQ | ACUAUAAAGACAGUAAAAAGAUCAGUGGUUAUCUU |
| 00263353- | | ID | UGCAGACGCCACCAUCACUGUGAGCCCUGUACUAUC |
| ENST000 | | NO: | AGCC |
| 00577856- | | 1232 | |
| 75 | | | |
| ENSG000 | 1.146281715 | SEQ | CCUUUUGCGGCCAUCACCGAAGCGGGAGCGGGUAA |
| 00263809- | | ID | GGAUUCGGCGGCCAAA |
| ENST000 | | NO: | |
| 00582471- | | 1233 | |
| 51 | | | |
| ENSG000 | 1.452842383 | SEQ | AUCGAUAAGGUUCAGUUAGCCAAGGUGGAGGAGUG |
| 00264058- | | ID | GAAAAGA |
| ENST000 | | NO: | |
| 00476049- | | 1234 | |
| 42 | | | |
| ENSG000 | 1.160205569 | SEQ | GAGAUCUCGAUCGAAGGCGAG |
| 00265241- | | ID | |
| ENST000 | | NO: | |
| 00632555- | | 1235 | |
| 21 | | | |
| ENSG000 | 1.196291602 | SEQ | GUGAGUGGAAGCUCUGGUGCAGCAGU |
| 00266200- | | ID | |
| ENST000 | | NO: | |
| 00579578- | | 1236 | |
| 26 | | | |
| ENSG000 | 1.168317499 | SEQ | GAGAACCCUUCCGGUGGGCUAGGUACUGAGCGCGC |
| 00266472- | | ID | GAGGCUCUACAGAGUGAAGGUUUAAAUCCAAGGUC |
| ENST000 | | NO: | |
| 00614145- | | 1237 | |
| 70 | | | |
| ENSG000 | 1.400626424 | SEQ | ACGCUGGGUUCUGCUUUUCCAGAAUCCUGCCCUUUC |
| 00267508- | | ID | CAAAAGGAAGACGGU |
| ENST000 | | NO: | |
| 00591679- | | 1238 | |
| 51 | | | |
| ENSG000 | 1.231330075 | SEQ | UGUGGGGAGGGUACCUCUCACAGAAGAGUCUUAGU |
| 00267561- | | ID | ACUGGCAUCAGGGGAAGGUCACCAAGUCUUUCCUG |
| ENST000 | | NO: | UAC |
| 00370548- | | 1239 | |
| 73 | | | |
| ENSG000 | 1.193216716 | SEQ | ACUAUAAAGACAGUAAAAAGAUCAGUGGUUAUCUU |
| 00270339- | | ID | UGCAGACGCCACCAUCGCUGUGAGCCCUGUACUAUC |
| ENST000 | | NO: | AGCC |
| 00584068- | | 1240 | |
| 75 | | | |
| ENSG000 | 1.452536745 | SEQ | ACCCUCUCACAUCCCAGUCUCUGAUCAGGGAAAGCA |
| 00270806- | | ID | GGGCACAGCCUUGGGAAGA |
| ENST000 | | NO: | |
| 00605587- | | 1241 | |
| 55 | | | |
| ENSG000 | 1.199203721 | SEQ | AAAGUGCUGUGGGGAGUGAUCACAUUUUUCACAAC |
| 00271254- | | ID | AUCCCUGGCUCCACCUCUUCUGCCACAAACGUCAGC |
| ENST000 | | NO: | |
| 00612640- | | 1242 | |
| 71 | | | |
| ENSG000 | 1.265660288 | SEQ | CGACUUUCCCGAUCGCCAGGCAGGAGUUUCUCUCGG |
| 00272196- | | ID | UGACUACUAUCGCUGUC |
| ENST000 | | NO: | |
| 00607355- | | 1243 | |
| 53 | | | |
| ENSG000 | 1.995713916 | SEQ | AAGCUAGGGCCACC |
| 00272414- | | ID | |
| ENST000 | | NO: | |
| 00509377- | | 1244 | |
| 14 | | | |
| ENSG000 | 2.074341985 | SEQ | CCGCCUGUCUGCACCGGCAGCACC |
| 00273518- | | ID | |
| ENST000 | | NO: | |
| 00619271- | | 1245 | |
| 24 | | | |
| ENSG000 | 1.173655751 | SEQ | ACAAAGCGUUGGGUGAGACUCCUCUUGCUCGUC |
| 00273542- | | ID | |
| ENST000 | | NO: | |
| 00611927- | | 1246 | |
| 33 | | | |
| ENSG000 | 2.074341985 | SEQ | CCGCCUGUCUGCACCGGCAGCACC |
| 00273775- | | ID | |
| ENST000 | | NO: | |
| 00615036- | | 1247 | |
| 24 | | | |
| ENSG000 | 1.112044721 | SEQ | CUCUUUCUCAGUGACCGGGUGGUUUGCUUAGGCGC |
| 00274005- | | ID | AGACGGGGAAGCGGAGCCAAC |
| ENST000 | | NO: | |
| 00617291- | | 1248 | |
| 56 | | | |
| ENSG000 | 1.535124669 | SEQ | CCUGUCUGCACAGACAGCACC |
| 00274108- | | ID | |
| ENST000 | | NO: | |
| 00617593- | | 1249 | |
| 21 | | | |
| ENSG000 | 2.074341985 | SEQ | CCGCCUGUCUGCACCGGCAGCACC |
| 00274146- | | ID | |
| ENST000 | | NO: | |
| 00614221- | | 1250 | |
| 24 | | | |
| ENSG000 | 1.511096092 | SEQ | GAGAGACUACAACUCCCAGUAGUCACCGCG |
| 00274175- | | ID | |
| ENST000 | | NO: | |
| 00616638- | | 1251 | |
| 30 | | | |
| ENSG000 | 2.039968479 | SEQ | AGCUGGGGCGCAGCCGCCUGUCUGCACCGGCAGCAC |
| 00274283- | | ID | C |
| ENST000 | | NO: | |
| 00610591- | | 1252 | |
| 37 | | | |
| ENSG000 | 2.039968479 | SEQ | AGCUGGGGCGCAGCCGCCUGUCUGCACCGGCAGCAC |
| 00274465- | | ID | C |
| ENST000 | | NO: | |
| 00620498- | | 1253 | |
| 37 | | | |
| ENSG000 | 1.107255084 | SEQ | UUCUUCAGCCUUAACCUAAGGUCUCAUACUCGGAGC |
| 00274571- | | ID | ACU |
| ENST000 | | NO: | |
| 00610853- | | 1254 | |
| 39 | | | |
| ENSG000 | 1.283579852 | SEQ | GGGUCUUCUCAAGGAAAUCCCAAUCUCUAUUCCGA |
| 00274729- | | ID | AGAGUCUUCCCUAAAAG |
| ENST000 | | NO: | |
| 00611610- | | 1255 | |
| 52 | | | |
| ENSG000 | 1.238040775 | SEQ | AUUAACACUUGGGUAACUUACCCUUCACAAUCCAUC |
| 00274749- | | ID | UAAAUCCUUCUCAAUUGCUGCCACC |
| ENST000 | | NO: | |
| 00621162- | | 1256 | |
| 61 | | | |
| ENSG000 | 1.174777913 | SEQ | AUUUACUUUGCAGAGUAACU |
| 00274750- | | ID | |
| ENST000 | | NO: | |
| 00614911- | | 1257 | |
| 20 | | | |
| ENSG000 | 2.074341985 | SEQ | CCGCCUGUCUGCACCGGCAGCACC |
| 00274920- | | ID | |
| ENST000 | | NO: | |
| 00621759- | | 1258 | |
| 24 | | | |
| ENSG000 | 2.012761787 | SEQ | CACACUCUGCCUUUGGACGUGAGAGAGAGCGCACCU |
| 00274972- | | ID | UUCACUUGAGCUUCAAC |
| ENST000 | | NO: | |
| 00611713- | | 1259 | |
| 53 | | | |
| ENSG000 | 1.511096092 | SEQ | GAGAGACUACAACUCCCAGUAGUCACCGCG |
| 00275034- | | ID | |
| ENST000 | | NO: | |
| 00611321- | | 1260 | |
| 30 | | | |
| ENSG000 | 2.039968479 | SEQ | AGCUGGGGCGCAGCCGCCUGUCUGCACCGGCAGCAC |
| 00275037- | | ID | C |
| ENST000 | | NO: | |
| 00615875- | | 1261 | |
| 37 | | | |
| ENSG000 | 1.185393876 | SEQ | CUGCUCAGCACUAGGCCGGCAGAAUCUGAGCG |
| 00275156- | | ID | |
| ENST000 | | NO: | |
| 00611318- | | 1262 | |
| 32 | | | |
| ENSG000 | 1.585246933 | SEQ | AAAAGAAUUAUCACAUUGCACAAGG |
| 00275190- | | ID | |
| ENST000 | | NO: | |
| 00627047- | | 1263 | |
| 25 | | | |
| ENSG000 | 1.185393876 | SEQ | CUGCUCAGCACUAGGCCGGCAGAAUCUGAGCG |
| 0027552- | | ID | |
| ENST000 | | NO: | |
| 00614280- | | 1264 | |
| 32 | | | |
| ENSG000 | 1.296134122 | SEQ | AGCUCGGUCGCGGCUGCCUGUCUGCUCCGGCAGCAC |
| 00275522- | | ID | C |
| ENST000 | | NO: | |
| 00613465- | | 1265 | |
| 37 | | | |
| ENSG000 | 2.074341985 | SEQ | CCGCCUGUCUGCACCGGCAGCACC |
| 00275545- | | ID | |
| ENST000 | | NO: | |
| 00622691- | | 1266 | |
| 24 | | | |
| ENSG000 | 1.120167319 | SEQ | ACCGGCAGCACC |
| 00275566- | | ID | |
| ENST000 | | NO: | |
| 00613612- | | 1267 | |
| 12 | | | |
| ENSG000 | 1.294164141 | SEQ | ACGUAGACCGUCUGCUGGGGGCGCCGGCACCAUUUG |
| 00275600- | | ID | GAGUACGUGAGGAAGAAAA |
| ENST000 | | NO: | |
| 00616581- | | 1268 | |
| 55 | | | |
| ENSG000 | 2.039968479 | SEQ | AGCUGGGGCGCAGCCGCCUGUCUGCACCGGCAGCAC |
| 00275608- | | ID | C |
| ENST000 | | NO: | |
| 00622416- | | 1269 | |
| 37 | | | |
| ENSG000 | 1.185296402 | SEQ | GCAGCAGUUUUCUACGUCUGAAAUUUUUUAGUUCU |
| 00275618- | | ID | CUGGAACCCAGAAUUUGCUAAGAG |
| ENST000 | | NO: | |
| 00631651- | | 1270 | |
| 59 | | | |
| ENSG000 | 1.185393876 | SEQ | CUGCUCAGCACUAGGCCGGCAGAAUCUGAGCG |
| 00275637- | | ID | |
| ENST000 | | NO: | |
| 00616589- | | 1271 | |
| 32 | | | |
| ENSG000 | 2.074341985 | SEQ | CCGCCUGUCUGCACCGGCAGCACC |
| 00275717- | | ID | |
| ENST000 | | NO: | |
| 00619277- | | 1272 | |
| 24 | | | |
| ENSG000 | 1.106047389 | SEQ | UUAGCUGAACUGAGCUGAAAUCCUAAAGGCCGCGG |
| 00275748- | | ID | AGUCGGCGGUGUUGUAGGUAGCGGUACCUUGAGUG |
| ENST000 | | NO: | GCAACAGAAUUCGAUUAAAUUACA |
| 00626328- | | 1273 | |
| 94 | | | |
| ENSG000 | 2.074341985 | SEQ | CCGCCUGUCUGCACCGGCAGCACC |
| 00275786- | | ID | |
| ENST000 | | NO: | |
| 00619931- | | 1274 | |
| 24 | | | |
| ENSG000 | 1.184025816 | SEQ | AGUAGAUCCUGGCCUGGACCUGAA |
| 00275791- | | ID | |
| ENST000 | | NO: | |
| 00611462- | | 1275 | |
| 24 | | | |
| ENSG000 | 1.351209686 | SEQ | GAAGGCUAGACCCGGUUUACUGGAAUUGCUCUGGC |
| 00275903- | | ID | GAUCGAGGGAUCCUAGUACACCGCAAUC |
| ENST000 | | NO: | |
| 00631787- | | 1276 | |
| 63 | | | |
| ENSG000 | 1.525878034 | SEQ | ACCCGGUUUACUGGAAUUGCUCUGGCGAUCGAGGG |
| 00275903- | | ID | AUCCUAGUACACCGCAAUC |
| ENST000 | | NO: | |
| 00631987- | | 1277 | |
| 54 | | | |
| ENSG000 | 2.044843971 | SEQ | UCCACUGGCCAGCAUUCUCCUCUAUUAGACUAGAAC |
| 00276068- | | ID | UGUGGAUAAACCUCAGAAA |
| ENST000 | | NO: | |
| 00615009- | | 1278 | |
| 55 | | | |
| ENSG000 | 1.105796845 | SEQ | AGAAGGACGCAGGCAGCAAAGAGUAGUCAGUCCCU |
| 00276085- | | ID | UCUUGGCUCUGCUGACACUCGAGCCCACAUUCCAUC |
| ENST000 | | NO: | ACCUGCUCCCAAUC |
| 00619989- | | 1279 | |
| 85 | | | |
| ENSG000 | 1.364868797 | SEQ | UCACAACCAGUGAUAUUAGGCUUGCAUUUUCUUUC |
| 00276167- | | ID | UUUUUUUCUGCUCAGAU |
| ENST000 | | NO: | |
| 00613831- | | 1280 | |
| 52 | | | |
| ENSG000 | 1.460575417 | SEQ | AGAAAGUUUAAAGUGCUGAGUGAUUUGCACUAGCU |
| 00276262- | | ID | GUAUAGAUCUUUGGUGGAGGAGGAAAGUGGAAAAU |
| ENST000 | | NO: | UAUUAAUUUCAGAGC |
| 00632852- | | 1281 | |
| 85 | | | |
| ENSG000 | 1.296134122 | SEQ | AGCUCGGUCGCGGCUGCCUGUCUGCUCCGGCAGCAC |
| 00276310- | | ID | C |
| ENST000 | | NO: | |
| 00610627- | | 1282 | |
| 37 | | | |
| ENSG000 | 2.039968479 | SEQ | AGCUGGGGCGCAGCCGCCUGUCUGCACCGGCAGCAC |
| 00276498- | | ID | C |
| ENST000 | | NO: | |
| 00621225- | | 1283 | |
| 37 | | | |
| ENSG000 | 2.039968479 | SEQ | AGCUGGGGCGCAGCCGCCUGUCUGCACCGGCAGCAC |
| 00276534- | | ID | C |
| ENST000 | | NO: | |
| 00618890- | | 1284 | |
| 37 | | | |
| ENSG000 | 1.157998465 | SEQ | UUCUUUUUUGAGGUUUUGAGGUUUUGCUGAAGAUA |
| 00276559- | | ID | UUCCUGAAGAAUCAUCCCAGGUGCCACACUAAAAA |
| ENST000 | | NO: | A |
| 00621712- | | 1285 | |
| 71 | | | |
| ENSG000 | 2.319422253 | SEQ | GUGAAUUAUAGUUUAAGUUACUGCAAGUCCUAAAC |
| 00276618- | | ID | UACGGAGUGGAACUAUUACAGUUUAUA |
| ENST000 | | NO: | |
| 00621806- | | 1286 | |
| 62 | | | |
| ENSG000 | 1.422729345 | SEQ | AGCGCAGCGGCGUCCGAGGCAACAAG |
| 00276657- | | ID | |
| ENST000 | | NO: | |
| 00632107- | | 1287 | |
| 26 | | | |
| ENSG000 | 1.440297711 | SEQ | AAAUUCAGGGUCCAGCUCACAGUGGAAAUACUUUC |
| 00276775- | | ID | UGAGAGUCAGUGACCUCCUGCACAAGAAC |
| ENST000 | | NO: | |
| 00455737- | | 1288 | |
| 64 | | | |
| ENSG000 | 1.474102416 | SEQ | AUUAAUAUUAAGAUUGGAAGUUUGUGUCUUUUGCU |
| 00276776- | | ID | GGAUAUUGGAAAUUGAAGUUA |
| ENST000 | | NO: | |
| 00610758- | | 1289 | |
| 56 | | | |
| ENSG000 | 1.19630846 | SEQ | UUUGCUCAGUUUCACAGAGGGCCUGGUCUGGAAUA |
| 00276953- | | ID | UUCCACAUCUGCUCUCACUCUGCC |
| ENST000 | | NO: | |
| 00617347- | | 1290 | |
| 59 | | | |
| ENSG000 | 2.359433846 | SEQ | AGUCUUCAUUUAGGUCCGAAUUCCCGGCAUAUAAG |
| 00276966- | | ID | AAUACUACCGUCGCUUGUUUUUCAGAUUUUUGCGG |
| ENST000 | | NO: | CUAUUUUCGUUGGUGUGUUGGUC |
| 00615164- | | 1291 | |
| 93 | | | |
| ENSG000 | 1.186722356 | SEQ | GGUGGAAGAAGUAAAGAAAAGAAAGAGUAAAGGAG |
| 00277140- | | ID | UUUGAAUCACUUUGCAGUGCUGU |
| ENST000 | | NO: | |
| 00631398- | | 1292 | |
| 58 | | | |
| ENSG000 | 1.294164141 | SEQ | ACGUAGACCGUCUGCUGGGGGCGCCGGCACCAUUUG |
| 00277161- | | ID | GAGUACGUGAGGAAGAAAA |
| ENST000 | | NO: | |
| 00620233- | | 1293 | |
| 55 | | | |
| ENSG000 | 2.074341985 | SEQ | CCGCCUGUCUGCACCGGCAGCACC |
| 00277175- | | ID | |
| ENST000 | | NO: | |
| 00616424- | | 1294 | |
| 24 | | | |
| ENSG000 | 1.198507693 | SEQ | GCAUUUCUCUUUAGGUUGUGGACGAAGUGUUUAUU |
| 00277224- | | ID | UAUC |
| ENST000 | | NO: | |
| 00356530- | | 1295 | |
| 39 | | | |
| ENSG000 | 1.185393876 | SEQ | CUGCUCAGCACUAGGCCGGCAGAAUCUGAGCG |
| 00277334- | | ID | |
| ENST000 | | NO: | |
| 00616389- | | 1296 | |
| 32 | | | |
| ENSG000 | 1.105796845 | SEQ | AGAAGGACGCAGGCAGCAAAGAGUAGUCAGUCCCU |
| 00277336- | | ID | UCUUGGCUCUGCUGACACUCGAGCCCACAUUCCAUC |
| ENST000 | | NO: | ACCUGCUCCCAAUC |
| 00621940- | | 1297 | |
| 85 | | | |
| ENSG000 | 1.185393876 | SEQ | CUGCUCAGCACUAGGCCGGCAGAAUCUGAGCG |
| 00277442- | | ID | |
| ENST000 | | NO: | |
| 00616747- | | 1298 | |
| 32 | | | |
| ENSG000 | 1.103341541 | SEQ | UCAGGGCAGUAGAACAUAUUUUAAUCAAGUCCCCU |
| 00277530- | | ID | UGAUAAAAGAGCUUGAUUCCAGCUACUGCACUUCC |
| ENST000 | | NO: | UUCAUCAGCUCUCCACUG |
| 00615431- | | 1299 | |
| 88 | | | |
| ENSG000 | 1.296134122 | SEQ | AGCUCGGUCGCGGCUGCCUGUCUGCUCCGGCAGCAC |
| 00277616- | | ID | C |
| ENST000 | | NO: | |
| 00612069- | | 1300 | |
| 37 | | | |
| ENSG000 | 1.105796845 | SEQ | AGAAGGACGCAGGCAGCAAAGAGUAGUCAGUCCCU |
| 00277768- | | ID | UCUUGGCUCUGCUGACACUCGAGCCCACAUUCCAUC |
| ENST000 | | NO: | ACCUGCUCCCAAUC |
| 00615140- | | 1301 | |
| 85 | | | |
| ENSG000 | 1.351209686 | SEQ | GAAGGCUAGACCCGGUUUACUGGAAUUGCUCUGGC |
| 00277791- | | ID | GAUCGAGGGAUCCUAGUACACCGCAAUC |
| ENST000 | | NO: | |
| 00613870- | | 1302 | |
| 63 | | | |
| ENSG000 | 1.525878034 | SEQ | ACCCGGUUUACUGGAAUUGCUCUGGCGAUCGAGGG |
| 00277791- | | ID | AUCCUAGUACACCGCAAUC |
| ENST000 | | NO: | |
| 00620309- | | 1303 | |
| 54 | | | |
| ENSG000 | 1.105796845 | SEQ | AGAAGGACGCAGGCAGCAAAGAGUAGUCAGUCCCU |
| 00277796- | | ID | UCUUGGCUCUGCUGACACUCGAGCCCACAUUCCAUC |
| ENST000 | | NO: | ACCUGCUCCCAAUC |
| 00612067- | | 1304 | |
| 85 | | | |
| ENSG000 | 1.185393876 | SEQ | CUGCUCAGCACUAGGCCGGCAGAAUCUGAGCG |
| 00278025- | | ID | |
| ENST000 | | NO: | |
| 00619741- | | 1305 | |
| 32 | | | |
| ENSG000 | 1.460575417 | SEQ | AGAAAGUUUAAAGUGCUGAGUGAUUUGCACUAGCU |
| 00278174- | | ID | GUAUAGAUCUUUGGUGGAGGAGGAAAGUGGAAAAU |
| ENST000 | | NO: | UAUUAAUUUCAGAGC |
| 00626883- | | 1306 | |
| 85 | | | |
| ENSG000 | 2.074341985 | SEQ | CCGCCUGUCUGCACCGGCAGCACC |
| 00278368- | | ID | |
| ENST000 | | NO: | |
| 00614464- | | 1307 | |
| 24 | | | |
| ENSG000 | 1.284513079 | SEQ | UCGACAGUCCACAAAGGCACAGUAUAAAAACGGUG |
| 00278373- | | ID | GGAAUCAGAGCACUUCAGCUCCAAUUGCUCUAGUU |
| ENST000 | | NO: | UUAGAAUUGCCUCUUUUUCAAG |
| 00622133- | | 1308 | |
| 92 | | | |
| ENSG000 | 1.242970876 | SEQ | GUAGUUUCAUUACAUUUUCUUGUGGCGAUUUUCCC |
| 00278463- | | ID | UUAUCAGAAGUAGUU |
| ENST000 | | NO: | |
| 00615868- | | 1309 | |
| 50 | | | |
| ENSG000 | 1.611121882 | SEQ | ACAGAAAGUAGCUCAUUCCUCCUAACUGGGUAAUA |
| 00278494- | | ID | GACAGUGGUGGGGCCUGGAAAAGAUUUCAAAU |
| ENST000 | | NO: | |
| 00632541- | | 1310 | |
| 67 | | | |
| ENSG000 | 1.296134122 | SEQ | AGCUCGGUCGCGGCUGCCUGUCUGCUCCGGCAGCAC |
| 00278503- | | ID | C |
| ENST000 | | NO: | |
| 00618507- | | 1311 | |
| 37 | | | |
| ENSG000 | 2.044843971 | SEQ | UCCACUGGCCAGCAUUCUCCUCUAUUAGACUAGAAC |
| 00278613- | | ID | UGUGGAUAAACCUCAGAAA |
| ENST000 | | NO: | |
| 00610590- | | 1312 | |
| 55 | | | |
| ENSG000 | 1.331947963 | SEQ | CCACCACCAGCAAACAAGGGGCAGCGUGGAGGAGU |
| 00278685- | | ID | GGACACA |
| ENST000 | | NO: | |
| 00615129- | | 1313 | |
| 42 | | | |
| ENSG000 | 1.120167319 | SEQ | ACCGGCAGCACC |
| 00278707- | | ID | |
| ENST000 | | NO: | |
| 00610296- | | 1314 | |
| 12 | | | |
| ENSG000 | 1.296134122 | SEQ | AGCUCGGUCGCGGCUGCCUGUCUGCUCCGGCAGCAC |
| 00278755- | | ID | C |
| ENST000 | | NO: | |
| 00619424- | | 1315 | |
| 37 | | | |
| ENSG000 | 1.511096092 | SEQ | GAGAGACUACAACUCCCAGUAGUCACCGCG |
| 00278848- | | ID | |
| ENST000 | | NO: | |
| 00617705- | | 1316 | |
| 30 | | | |
| ENSG000 | 2.074341985 | SEQ | CCGCCUGUCUGCACCGGCAGCACC |
| 00278856- | | ID | |
| ENST000 | | NO: | |
| 00611567- | | 1317 | |
| 24 | | | |
| ENSG000 | 1.371076129 | SEQ | AGAGAUAGAGUCUUCCCUGGCAUUGCAGGAGAGAA |
| 00281886- | | ID | UCUGAAGGG |
| ENST000 | | NO: | |
| 00627547- | | 1318 | |
| 44 | | | |
| ENSG000 | 1.89043467 | SEQ | GCCUUAUCUGACUCUUGUUUUCAACAGAGUUUGUC |
| 00281938- | | ID | CUCUCACUUCUGGAGAAG |
| ENST000 | | NO: | |
| 00413683- | | 1319 | |
| 53 | | | |
| ENSG000 | 1.323598894 | SEQ | ACUCAGUGUACCUUCUAGUCCCGCC |
| 00282019- | | ID | |
| ENST000 | | NO: | |
| 00633130- | | 1320 | |
| 25 | | | |
| ENSG000 | 1.150959433 | SEQ | UUAGUUCUUCCUGAGGUCUGAGCACCCUCCAAACUG |
| 00282125- | | ID | AGUCCAGAUCUGAGUUUUUCCUCGGAGAUUUGUCA |
| ENST000 | | NO: | GA |
| 00632108- | | 1321 | |
| 73 | | | |
| ENSG000 | 1.440297711 | SEQ | AAAUUCAGGGUCCAGCUCACAGUGGAAAUACUUUC |
| 00282223- | | ID | UGAGAGUCAGUGACCUCCUGCACAAGAAC |
| ENST000 | | NO: | |
| 00631379- | | 1322 | |
| 64 | | | |
| ENSG000 | 1.65030921 | SEQ | AGCAUCAUCCAGAAACCACAUCCCUCCGCUAGAGAA |
| 00282337- | | ID | GCCCCUGACGGCACAGUUCCUCACU |
| ENST000 | | NO: | |
| 00632614- | | 1323 | |
| 61 | | | |
| ENSG000 | 1.19630846 | SEQ | UUUGCUCAGUUUCACAGAGGGCCUGGUCUGGAAUA |
| 00282354- | | ID | UUCCACAUCUGCUCUCACUCUGCC |
| ENST000 | | NO: | |
| 00631824- | | 1324 | |
| 59 | | | |
| ENSG000 | 1.740603439 | SEQ | GAAGGAAAGAGUAACUUGAGUUUCACUUCUUAGUG |
| 00282628- | | ID | CCUUUUCUCAGGGGAGAGGCCAUCACUUGAAG |
| ENST000 | | NO: | |
| 00634198- | | 1325 | |
| 67 | | | |
| ENSG000 | 1.303791986 | SEQ | AGUCUCCUUCACCACCCAGCUGGGAUCUCAGGGCUU |
| 00282651- | | ID | CCUUUUCUGUCCUCCUCCAGG |
| ENST000 | | NO: | |
| 00632950- | | 1326 | |
| 57 | | | |
| ENSG000 | 1.280063899 | SEQ | UCACCAAGUCACAGACCCAGAGGACCCCUCCAUCCU |
| 00282719- | | ID | GCAGUUCCUGCC |
| ENST000 | | NO: | |
| 00632016- | | 1327 | |
| 48 | | | |
| ENSG000 | 1.580879837 | SEQ | AUUCUUUCUUCAAAGCAGCC |
| 00282812- | | ID | |
| ENST000 | | NO: | |
| 00619125- | | 1328 | |
| 20 | | | |
| ENSG000 | 1.4862564 | SEQ | CUUUCAAAGCCAAUUUGAAUAAACAACUCUUCCCUC |
| 00283154- | | ID | UCAAAGUGGCAAGAGUAACUCUUCAGCACC |
| ENST000 | | NO: | |
| 00412423- | | 1329 | |
| 66 | | | |
| ENSG000 | 1.150959433 | SEQ | UUAGUUCUUCCUGAGGUCUGAGCACCCUCCAAACUG |
| 00283488- | | ID | AGUCCAGAUCUGAGUUUUUCCUCGGAGAUUUGUCA |
| ENST000 | | NO: | GA |
| 00636272- | | 1330 | |
| 73 | | | |
| ENSG000 | 1.339719472 | SEQ | AAGUUGCUUUGGUUUCUAUUACUUUGUUAAGUCAU |
| 00284021- | | ID | UCCUUCUGCAAAGGACUGCCUGGCAGGUGUCAAAG |
| ENST000 | | NO: | GCAGUGGUGGCCACAGAGGCGGUGGAGAG |
| 00640356- | | 1331 | |
| 99 | | | |
| ENSG000 | 1.276933056 | SEQ | UGGUUUCUAUUACUUUGUUAAGUCAUUCCUUCUGC |
| 00284021- | | ID | AAAGGACUGCCUGGCAGGUGUCAAAGGCAGUGGUG |
| ENST000 | | NO: | GCCACAGAGGCGGUGGAGAG |
| 00640466- | | 1332 | |
| 90 | | | |
| ENSG000 | 1.282810866 | SEQ | CUGGAGUCUCCGCGGGCAGAUCUCAUAUUUUGGAU |
| 00284770- | | ID | UCUGGAUAUAUUAUA |
| ENST000 | | NO: | |
| 00645662- | | 1333 | |
| 50 | | | |
| ENSG000 | 1.102748702 | SEQ | ACACCUUCUGAGGCAGCGGC |
| 00284810- | | ID | |
| ENST000 | | NO: | |
| 00643904- | | 1334 | |
| 20 | | | |
| ENSG000 | 1.950052408 | SEQ | ACACACUCGCUUCUGGAACGUCUGAGGUUAUCAAU |
| 00284931- | | ID | AAGCUCCUAGUCCAGACGCC |
| ENST000 | | NO: | |
| 00642908- | | 1335 | |
| 55 | | | |
| ENSG000 | 2.012761787 | SEQ | CACACUCUGCCUUUGGACGUGAGAGAGAGCGCACCU |
| 00285063- | | ID | UUCACUUGAGCUUCAAC |
| ENST000 | | NO: | |
| 00644173- | | 1336 | |
| 53 | | | |
| ENSG000 | 1.176135501 | SEQ | ACAUAUCCACUCCUGCUCUCCCUCCUGCAGGUGACC |
| 00285251- | | ID | CCAGCC |
| ENST000 | | NO: | |
| 00647159- | | 1337 | |
| 42 | | | |
| ENSG000 | 1.103341541 | SEQ | UCAGGGCAGUAGAACAUAUUUUAAUCAAGUCCCCU |
| 00285432- | | ID | UGAUAAAAGAGCUUGAUUCCAGCUACUGCACUUCC |
| ENST000 | | NO: | UUCAUCAGCUCUCCACUG |
| 00642785- | | 1338 | |
| 88 | | | |
| ENSG000 | 1.211761788 | SEQ | UUCGCAGCGGACCGAAGAGAAGAAAAGUAGGCCAG |
| 00286070- | | ID | AGCCGAACUCUCUUCCUGCCAAG |
| ENST000 | | NO: | |
| 00439775- | | 1339 | |
| 58 | | | |
| ENSG000 | 1.344062718 | SEQ | ACACUUUUGUGUGUGCUCUCAUUGCAA |
| 00287080- | | ID | |
| ENST000 | | NO: | |
| 00612966- | | 1340 | |
| 27 | | | |
| ENSG000 | 1.427199975 | SEQ | ACCUCACCAUCAAGAAAGGCCCUGGUCCAGGAGUUG |
| 00287363- | | ID | CAAGGUGUGAGUAAAUCACAGGGCUGGGGAAGGUC |
| ENST000 | | NO: | AGCUCGGGGUCACAAGAAGCCUG |
| 00372963- | | 1341 | |
| 94 | | | |
| ENSG000 | 1.237967637 | SEQ | CCGCUGAAGUGGAGCAGGUACAGUCACAGCUGUGG |
| 00288362- | | ID | GGACAGC |
| ENST000 | | NO: | |
| 00671845- | | 1342 | |
| 42 | | | |

### Enumerated Embodiments

In some aspects, the present invention is directed to the following non-limiting embodiments:

Embodiment 1: A method of modulating a translational efficiency of a messenger RNA (mRNA) molecule, the method comprising: modifying a 5′-UTR of the mRNA molecule to include a translational enhancer or a translational silencer.

Embodiment 2: The method of Embodiment 1, wherein modifying the 5′-UTR of the mRNA molecule comprises modifying a sequence of a DNA molecule encoding the mRNA molecule.

Embodiment 3: The method of any one of Embodiments 1-2, wherein the translational enhancer or the translational silence is a translational enhancer or a translational silencer for the translational machinery of a yeast species, optionally a yeast species selected from the group consisting of Saccharomyces cerevisiae, Pichia pastoris, Hansenda polymorpha, Yarrowia lipolytica, Arxula adeninivorans, Kluyveromyces lactis, and Schizosaccharomyces pombe.

Embodiment 4: The method of Embodiment 3, wherein the 5′-UTR of the mRNA molecule is modified to include the translational enhancer, and wherein the translational enhancer includes at least one sequence selected from the group consisting of SEQ ID NOs: 1-3.

Embodiment 5: The method of Embodiment 3, wherein the 5′-UTR of the mRNA molecule is modified to include the translational silencer, and wherein the translational silencer includes at least one sequence selected from the group consisting of SEQ ID NOs: 4-6.

Embodiment 6: The method of any one of Embodiment 1-2, wherein the translational enhancer or the translational silence is a translational enhancer or a translational silencer for the translational machinery of a mammal, optionally a human.

Embodiment 7: The method of Embodiment 6, wherein the 5′-UTR of the mRNA molecule is modified to include the translational enhancer, and wherein the translational enhancer includes at least one sequence selected from the group consisting of SEQ ID NOs: 7-18, 29-51 and 56-1342.

Embodiment 8: The method of Embodiment 6, wherein the 5′-UTR of the mRNA molecule is modified to include the translational silencer, and wherein the translational silencer includes at least one sequence selected from the group consisting of SEQ ID NOs: 19-28.

Embodiment 9: The method of any one of Embodiment 1-8, wherein the 5′-UTR of the mRNA molecule is further modified to comprise a modified nucleobase.

Embodiment 10: The method of Embodiment 9, wherein the modified nucleobase comprises N6-methyladenosine, inosine, N1-propylpseudouridine, N1-methoxymethylpseudouridine, N1-ethylpseudouridine, 5-methoxycytidine, 5-hydroxyuridine, 5-carboxyuridine, 5-formyluridine, 5-hydroxycytidine, 5-hyrdoxymethylcytidine, 5-hydroxymethyluridine, 5-formylcytidine, 5-carboxycytidine, N4-methylcytidine, pseudoisocytidine, 2-thiocytidine, 4-thiocytidine, N1-methylpseudouridine, pseudouridine, 5-methyluridine, 5-methoxyuridine, dihydrouridine, 5-methylcytidine, 4-thiouridine, 2-thiouridine, uridine-5′-O-(1-thiophosphate), 5-aminoallyluridine, or 4-acetylcytidine.

Embodiment 11: The method of any one of Embodiment 1-10, wherein the 5′-UTR of the mRNA molecule is modified to include the translational enhancer, and wherein a ribosome retention score (RRS) of the modified mRNA molecule is 3× or higher than an RRS of the unmodified mRNA molecule.

Embodiment 12: The method of any one of Embodiments 1-10, wherein the 5′-UTR of the mRNA molecule is modified to include the translational silencer, and wherein a ribosome retention score (RRS) of the modified mRNA molecule is 0.3× or lower than an RRS of the unmodified mRNA molecule.

Embodiment 13: An mRNA molecule comprising a 5′-UTR, wherein the 5′-UTR comprises: a translational enhancer comprising at least one sequence selected from the group consisting of SEQ ID NOs: 1-3, 7-18, 29-51 and 56-1342, or a translational silencer comprising at least one selected from the group consisting of SEQ ID NOs: 4-6 and 19-28; and a modified nucleobase.

Embodiment 14: The mRNA molecule of Embodiment 13, wherein the modified nucleobase comprises N6-methyladenosine, inosine, N1-propylpseudouridine, N1-methoxymethylpseudouridine, N1-ethylpseudouridine, 5-methoxycytidine, 5-hydroxyuridine, 5-carboxyuridine, 5-formyluridine, 5-hydroxycytidine, 5-hyrdoxymethylcytidine, 5-hydroxymethyluridine, 5-formylcytidine, 5-carboxycytidine, N4-methylcytidine, pseudoisocytidine, 2-thiocytidine, 4-thiocytidine, N1-methylpseudouridine, pseudouridine, 5-methyluridine, 5-methoxyuridine, dihydrouridine, 5-methylcytidine, 4-thiouridine, 2-thiouridine, uridine-5′-O-(1-thiophosphate), 5-aminoallyluridine, or 4-acetylcytidine.

Embodiment 15: The mRNA molecule of any of Embodiments 13-14, wherein the 5′-UTR comprises the translational enhancer, and wherein a ribosome retention score (RRS) of the mRNA molecule is 3× or higher than an RRS of an mRNA molecule that does not comprise the translational enhancer or the modified nucleobase but otherwise has the same sequence.

Embodiment 16: The mRNA molecule of any of Embodiments 13-15, wherein the 5′-UTR comprises at least one sequences selected from the group consisting of SEQ ID NOs: 29-50.

Embodiment 17: The mRNA molecule of any of Embodiment 13-14, wherein the 5′-UTR comprises the translational silencer, and wherein an RRS of the mRNA molecule is 0.3× or lower than an RRS of an mRNA molecule that does not comprise the translational silencer or the modified nucleobase but otherwise has the same sequence.

Embodiment 18: The mRNA molecule of any one of Embodiment 13-17, wherein the mRNA molecule encodes a therapeutic peptide or a therapeutic protein.

Embodiment 19: The mRNA molecule of Embodiment 18, wherein the therapeutic peptide or the therapeutic protein comprises a vaccine.

Embodiment 20: A method of constructing an mRNA molecule for producing a therapeutic peptide or a therapeutic protein, the method comprising including a translational enhancer at a 5′ UTR to the 5′ side of a coding region encoding the therapeutic peptide or a therapeutic protein.

Embodiment 21: The method of Embodiment 20, wherein the therapeutic peptide or a therapeutic protein comprises a vaccine.

Embodiment 22: A yeast cell for expressing a protein, comprising an mRNA molecule or a nucleotide encoding the mRNA molecule, wherein the mRNA molecule comprises: a 5′-UTR comprising at least one yeast translational enhancer; and a coding region encoding the protein, or a coding region for introducing a nucleotide sequence for encoding the protein.

Embodiment 23: The yeast cell of Embodiment 22, wherein the 5′-UTR comprises at least one sequence selected from the group consisting of SEQ ID NOs: 1-3.

Embodiment 24: The yeast of any one of Embodiment 22-23, wherein the yeast cell is a Saccharomyces cerevisiae cell, a Pichia pastoris cell, a Hansenula polymorpha cell, a Yarrowia lipolytica cell, an Arxula adeninivorans cell, a Kluyveromyces lactis cell, or a Schizosaccharomyces pombe cell.

The foregoing outlines features of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions, and alterations herein without departing from the spirit and scope of the present disclosure.

1. A method of modulating a translational efficiency of a messenger RNA (mRNA) molecule, the method comprising:
modifying a 5′-UTR of the mRNA molecule to include a translational enhancer or a translational silencer.

2. The method of claim 1, wherein modifying the 5′-UTR of the mRNA molecule comprises modifying a sequence of a DNA molecule encoding the mRNA molecule.

3. The method of claim 1, wherein the translational enhancer or the translational silencer is a translational enhancer or a translational silencer for the translational machinery of a yeast species, optionally a yeast species selected from the group consisting of Saccharomyces cerevisiae, Pichia pastoris, Hansenula polymorpha, Yarrowia lipolytica, Arxula adeninivorans, Kluyveromyces lactis, and Schizosaccharomyces pombe.

4. The method of claim 3, wherein the 5′-UTR of the mRNA molecule is modified to include the translational enhancer, and wherein the translational enhancer includes at least one sequence selected from the group consisting of SEQ ID NOs: 1-3.

5. The method of claim 3, wherein the 5′-UTR of the mRNA molecule is modified to include the translational silencer, and wherein the translational silencer includes at least one sequence selected from the group consisting of SEQ ID NOs: 4-6.

6. The method of claim 1, wherein the translational enhancer or the translational silence is a translational enhancer or a translational silencer for the translational machinery of a mammal, optionally a human.

7. The method of claim 6, wherein the 5′-UTR of the mRNA molecule is modified to include the translational enhancer, and wherein the translational enhancer includes at least one sequence selected from the group consisting of SEQ ID NOs: 7-18, 29-51 and 56-1342.

8. The method of claim 6, wherein the 5′-UTR of the mRNA molecule is modified to include the translational silencer, and wherein the translational silencer includes at least one sequence selected from the group consisting of SEQ ID NOs: 19-28.

9. The method of claim 1, wherein the 5′-UTR of the mRNA molecule is further modified to comprise a modified nucleobase.

10. The method of claim 9, wherein the modified nucleobase comprises N6-methyladenosine, inosine, N1-propylpseudouridine, N1-methoxymethylpseudouridine, N1-ethylpseudouridine, 5-methoxycytidine, 5-hydroxyuridine, 5-carboxyuridine, 5-formyluridine, 5-hydroxycytidine, 5-hyrdoxymethylcytidine, 5-hydroxymethyluridine, 5-formylcytidine, 5-carboxycytidine, N4-methylcytidine, pseudoisocytidine, 2-thiocytidine, 4-thiocytidine, N1-methylpseudouridine, pseudouridine, 5-methyluridine, 5-methoxyuridine, dihydrouridine, 5-methylcytidine, 4-thiouridine, 2-thiouridine, uridine-5′-O-(1-thiophosphate), 5-aminoallyluridine, or 4-acetylcytidine.

11. The method of claim 1, wherein the 5′-UTR of the mRNA molecule is modified to include the translational enhancer, and wherein a ribosome retention score (RRS) of the modified mRNA molecule is 3× or higher than an RRS of the unmodified mRNA molecule.

12. The method of claim 1, wherein the 5′-UTR of the mRNA molecule is modified to include the translational silencer, and wherein a ribosome retention score (RRS) of the modified mRNA molecule is 0.3× or lower than an RRS of the unmodified mRNA molecule.

13. An mRNA molecule comprising a 5′-UTR, wherein the 5′-UTR comprises:
a translational enhancer comprising at least one sequence selected from the group consisting of SEQ ID NOs: 1-3, 7-18, 29-51 and 56-1342, or a translational silencer comprising at least one selected from the group consisting of SEQ ID NOs: 4-6 and 19-28; and
a modified nucleobase.

14. The mRNA molecule of claim 13, wherein the modified nucleobase comprises N6-methyladenosine, inosine, N1-propylpseudouridine, N1-methoxymethylpseudouridine, N1-ethylpseudouridine, 5-methoxycytidine, 5-hydroxyuridine, 5-carboxyuridine, 5-formyluridine, 5-hydroxycytidine, 5-hyrdoxymethylcytidine, 5-hydroxymethyluridine, 5-formylcytidine, 5-carboxycytidine, N4-methylcytidine, pseudoisocytidine, 2-thiocytidine, 4-thiocytidine, N1-methylpseudouridine, pseudouridine, 5-methyluridine, 5-methoxyuridine, dihydrouridine, 5-methylcytidine, 4-thiouridine, 2-thiouridine, uridine-5′-O-(1-thiophosphate), 5-aminoallyluridine, or 4-acetylcytidine.

15. The mRNA molecule of claim 13, wherein the 5′-UTR comprises the translational enhancer, and wherein a ribosome retention score (RRS) of the mRNA molecule is 3× or higher than an RRS of an mRNA molecule that does not comprise the translational enhancer or the modified nucleobase but otherwise has the same sequence.

16. The mRNA molecule of claim 13, wherein the 5′-UTR comprises at least one sequences selected from the group consisting of SEQ ID NOs: 29-50.

17. The mRNA molecule of claim 13, wherein the 5′-UTR comprises the translational silencer, and wherein an RRS of the mRNA molecule is 0.3× or lower than an RRS of an mRNA molecule that does not comprise the translational silencer or the modified nucleobase but otherwise has the same sequence.

18. The mRNA molecule of claim 13, wherein the mRNA molecule encodes a therapeutic peptide or a therapeutic protein.

19. The mRNA molecule of claim 18, wherein the therapeutic peptide or the therapeutic protein comprises a vaccine.

20. A method of constructing an mRNA molecule for producing a therapeutic peptide or a therapeutic protein, the method comprising including a translational enhancer at a 5′ UTR to the 5′ side of a coding region encoding the therapeutic peptide or a therapeutic protein.

21. The method of claim 20, wherein the therapeutic peptide or a therapeutic protein comprises a vaccine.

22. A yeast cell for expressing a protein, comprising an mRNA molecule or a nucleotide encoding the mRNA molecule, wherein the mRNA molecule comprises:
a 5′-UTR comprising at least one yeast translational enhancer; and
a coding region encoding the protein, or a coding region for introducing a nucleotide sequence for encoding the protein.

23. The yeast cell of claim 22, wherein the 5′-UTR comprises at least one sequence selected from the group consisting of SEQ ID NOs: 1-3.

24. The yeast of claim 22, wherein the yeast cell is a Saccharomyces cerevisiae cell, a Pichia pastoris cell, a Hansenula polymorpha cell, a Yarrowia lipolytica cell, an Arxula adeninivorans cell, a Kluyveromyces lactis cell, or a Schizosaccharomyces pombe cell.


SEQUENCE LISTING (1342 sequences)

SEQ ID NO: 1-28 - skipped (28 entries)

SEQ ID NO: 29 - RNA, 182 nt, synthetic construct
  source 1..182: mol_type = other RNA
cccccactggctgctctgaaaagccatctttgcattgttcctcatccgcctccttgctcgccgcagccgcctccgccgcgcgcctcctccgccgccgcggactccggcagctttatcgccagagtccctgaactctcgctttctttttaatcccctgcatcggatcaccggcgtgccccacc

SEQ ID NO: 30 - RNA, 140 nt, synthetic construct
  source 1..140: mol_type = other RNA
attgttctacaagctaaattattccttcaaaggagaggagaattgtataaggtcacagacgtggatccaaagccggagcctgagcagaggtggccacgctggagggtcttgacctaaagtgtggtcagcgatactgccta

SEQ ID NO: 31 - RNA, 223 nt, synthetic construct
  source 1..223: mol_type = other RNA
agttgcagtctggtagttgtcgctggccgtgtgacggctcgctgttgccctgaaggcaggcgagccagctgcccaggaaaggtggaaagtggtagaagctgacccctgagccctggcaggtctttaagtgcgtttgtgcagccgatttcaaggctaagagagaaagactgcctctgatccctgaaggaagaaaaaaaaaaaaaaaacaggaaaaaaactcaac

SEQ ID NO: 32 - RNA, 173 nt, synthetic construct
  source 1..173: mol_type = other RNA
gacgtgttttataggcggcataaagcgaaagtgcaggcagctgtggaaggcgaagttcaatcccagagtccgccccctgaattggggcctttccggaggaggaagctctgaaaaacagggggcccagtgccattccgcagggaattgtcgcttgcgttcagctgttctacaca

SEQ ID NO: 33 - RNA, 123 nt, synthetic construct
  source 1..123: mol_type = other RNA
agttcagtttgtctctcttgagcttggagaaaatcatccgtagtgcctccccgggggacacgtagaggagagaaaagcgaccaagataaaagtggacagaagaataagcgagactttttatcc

SEQ ID NO: 34 - RNA, 171 nt, synthetic construct
  source 1..171: mol_type = other RNA
aggcatcagcaatctatcagggaacggcggtggccggtgcggcgtgttcggtggcggctctggccgctcaggcgcctgcggctgggtgagcgcacgcgaggcggcgaggcggcagcgtgtttctaggtcgtggcgtcgggcttccggagctttggcggcagctaggggagg

SEQ ID NO: 35 - RNA, 100 nt, synthetic construct
  source 1..100: mol_type = other RNA
aaaacagaaaccataaggcctacctcccagggttgctgtgagaattgagggggaggatatttggatttgacctgcattttggaatttatctacacttaaa

SEQ ID NO: 36 - RNA, 128 nt, synthetic construct
  source 1..128: mol_type = other RNA
attagtacagcacccagaggtcttcggagcaaagctgttaaagaagtggggtccatttgagccccaactttgccttccttaggtccccttgggcttgtctagccctaggagttccaacaagctaagaa

SEQ ID NO: 37 - RNA, 187 nt, synthetic construct
  source 1..187: mol_type = other RNA
agtgtgcagtaagagataacctggcaagtggtagtggcgcttctcgggtgctgtgcttcacgttttggtactaaaggccgagactgttgtggcgacggcgacctctacggcaacggcttaagctctcggaggagtggcagagtacgatctgaaggaggggcttctggttagcccaggttccatcata

SEQ ID NO: 38 - RNA, 205 nt, synthetic construct
  source 1..205: mol_type = other RNA
ggaaacgcggagtgtcctggagcttgacgcgctggagcttgagaggtctttcaggactgattttggtggaagaagacagcaggcggtgggcattcactgtgctggtgggaagtctggttacattaagaaaggaattctagcaaagttattctgtaaatacaacagaatcagttgcttttgttaaatatattggtgcaagttcaac

SEQ ID NO: 39 - RNA, 155 nt, synthetic construct
  source 1..155: mol_type = other RNA
aattcatatttgagtgggcggtggcgattggtgttggcggtctggctcagctgggcagggggtaactttactgatttgggggtggtttttagtttaatttttcttttctagcttcccatcgacggtcagtgcgcacgttgtaatcagctgaggcc

SEQ ID NO: 40 - RNA, 100 nt, synthetic construct
  source 1..100: mol_type = other RNA
agtctgcattgagcttggctcaggaaagaagggaaatccaggcggggcctgttgggcccaggtcttgagctcttttggctccagagttcccagcacagtc

SEQ ID NO: 41 - RNA, 169 nt, synthetic construct
  source 1..169: mol_type = other RNA
agacagcctgggaggggagaaggagttggagctcaagttggagacagcgaggagaaacctgccatagccagggtgtgtctttgatcctcttcaggaggtgaggagaagccagaggtccttggtgtgccctcagaaatctgcctgcagttctcaccaagccgctgtgaaa

SEQ ID NO: 42 - RNA, 111 nt, synthetic construct
  source 1..111: mol_type = other RNA
ttttttttttttttttttttttttttttcctttcccctccccctccctctccaagccggaggggtcctgaggtgacagcgcctgcaactgaaatttcagcagcgggagaag

SEQ ID NO: 43 - RNA, 214 nt, synthetic construct
  source 1..214: mol_type = other RNA
acactgactaaagatattggaagagtgaagatcagagaattttaagtctgaaatttggcatcactgccctgaacaatataaccttagttggcataaactactcatacagacaaaggcattatccatcacaataagtaactttttgtctttatttcaaccggacaatcgtgattagaaaagctcctgtgacaaaattcaagaaaacctgacataa

SEQ ID NO: 44 - RNA, 203 nt, synthetic construct
  source 1..203: mol_type = other RNA
aaacgcggagtgtcctggagcttgacgcgctggagcttgagaggtctttcaggactgattttggtggaagaagacagcaggcggtgggcattcactgtgctggtgggaagtctggttacattaagaaaggaattctagcaaagttattctgtaaatacaacagaatcagttgcttttgttaaatatattggtgcaagttcaac

SEQ ID NO: 45 - RNA, 100 nt, synthetic construct
  source 1..100: mol_type = other RNA
gaggctgcagcagcacatacaggagctagaggcccaccttgaggctgaggagggtgcgcggcagaagctgcagctggagaaggtgacgacagaggcaaaa

SEQ ID NO: 46 - RNA, 100 nt, synthetic construct
  source 1..100: mol_type = other RNA
attctgcgcctgcgcgcggctacagcacggttcgtttttcctttagtcaggaaggacgttggtgttgaggttagcatacgtatcaaggacagtaactacc

SEQ ID NO: 47 - RNA, 100 nt, synthetic construct
  source 1..100: mol_type = other RNA
gagagagagagagaaaaaaaaggtcaggtaggaagaagaaaaatagactttctgtgactgtatactcttgaaatctcccgcaggttctaagcagggcaaa

SEQ ID NO: 48 - RNA, 100 nt, synthetic construct
  source 1..100: mol_type = other RNA
cagaattttctcacataaatactgaggaagaccctgccctctcctcacttctctggacttggccctgagctggacctggtccactggggtaggcagggcg

SEQ ID NO: 49 - RNA, 103 nt, synthetic construct
  source 1..103: mol_type = other RNA
agtctgatttacggctctgctgaaaaccgcttcgctcccgcagcatcagcaccggaggcggcggctgcagcagcagcagcaacaagtcgggacttttagagta

SEQ ID NO: 50 - RNA, 210 nt, synthetic construct
  source 1..210: mol_type = other RNA
attagtcactgcgcccaggagccagtttacacctagcgaaggcggaggcattggacaagcctgggtctgagcagacagaaccccttcctgcgaccctggagtcccaatcgtgctgccattttccttctgcccaggactctccagtcctcagtcaccttggacaaagaagtgtggatcctcagattccatcttttccaactccaaggtgcc

SEQ ID NO: 51 - RNA, 100 nt, synthetic construct
  source 1..100: mol_type = other RNA
ctggaaggtctgggggactgagggaagcgaagagaaaagcaagagcaattaaatatctcacattcttgaaggtggcattgaagagcactaagatcggaag

SEQ ID NO: 52 - RNA, 22 nt, synthetic construct
  source 1..22: mol_type = other RNA
aaggcgtgtttgctgacgtttc

SEQ ID NO: 53 - RNA, 20 nt, synthetic construct
  source 1..20: mol_type = other RNA
cacccgttcctaccaagacc

SEQ ID NO: 54 - RNA, 25 nt, synthetic construct
  source 1..25: mol_type = other RNA
tgggcgcgttatttatcggagttgc

SEQ ID NO: 55 - RNA, 24 nt, synthetic construct
  source 1..24: mol_type = other RNA
gagcccatatccttgcctgatacc

SEQ ID NO: 56 - RNA, 48 nt, synthetic construct
  source 1..48: mol_type = other RNA
ctgcttggctttgaggaagagtggcagtactgcctcactgcataaggg

SEQ ID NO: 57 - RNA, 24 nt, synthetic construct
  source 1..24: mol_type = other RNA
gaagctctcctgtttgacgaaagt

SEQ ID NO: 58 - RNA, 62 nt, synthetic construct
  source 1..62: mol_type = other RNA
atagcctagatcgagctccctgtgtgcaccgcgcgctgcccgagaggtcaaccagaatcaag

SEQ ID NO: 59 - RNA, 88 nt, synthetic construct
  source 1..88: mol_type = other RNA
tagaagagtgatatttgtgtgagagcctgggtcaggcttattttgagacgtgggccccactgaggtcattatccaacttttagaggtg

SEQ ID NO: 60 - RNA, 52 nt, synthetic construct
  source 1..52: mol_type = other RNA
aggactgagttctgaattaactaaagtaacttgtctggtttgcagagtgctg

SEQ ID NO: 61 - RNA, 79 nt, synthetic construct
  source 1..79: mol_type = other RNA
tcagaaactgttagttggcaaaggagttaccatctcctattaaacgggcaaggacaagttcccttttctgagagtgctg

SEQ ID NO: 62 - RNA, 12 nt, synthetic construct
  source 1..12: mol_type = other RNA
acttcggcggac

SEQ ID NO: 63 - RNA, 92 nt, synthetic construct
  source 1..92: mol_type = other RNA
actagtgcttcctagtgtcagggccagctgtgtagtggctcggtgtgatttgttagctctttgaggcagggtaccctcctcaggatttcgat

SEQ ID NO: 64 - RNA, 29 nt, synthetic construct
  source 1..29: mol_type = other RNA
acaacaaagcttccagagggagaggaagg

SEQ ID NO: 65 - RNA, 76 nt, synthetic construct
  source 1..76: mol_type = other RNA
tgggaaataaagtcatttgtagttttttctctcttgcacagcctaaaagtaccaagttgtgatttcagtggaataa

SEQ ID NO: 66 - RNA, 48 nt, synthetic construct
  source 1..48: mol_type = other RNA
acagaagactggacacaattccgaagagctgcccagaaggagagaaca

SEQ ID NO: 67 - RNA, 67 nt, synthetic construct
  source 1..67: mol_type = other RNA
tgctgctctcggtttagtccaagatcagcgatatcacgcgtcccccggagcatcgcgtgcaggagcc

SEQ ID NO: 68 - RNA, 66 nt, synthetic construct
  source 1..66: mol_type = other RNA
gctgctctcggtttagtccaagatcagcgatatcacgcgtcccccggagcatcgcgtgcaggagcc

SEQ ID NO: 69 - RNA, 28 nt, synthetic construct
  source 1..28: mol_type = other RNA
agctcctggcgcctgcgatagcggcaag

SEQ ID NO: 70 - RNA, 22 nt, synthetic construct
  source 1..22: mol_type = other RNA
ctgccttgctctgccaggaacc

SEQ ID NO: 71 - RNA, 16 nt, synthetic construct
  source 1..16: mol_type = other RNA
gtgaaaggttgcgaag

SEQ ID NO: 72 - RNA, 44 nt, synthetic construct
  source 1..44: mol_type = other RNA
aagaagcggagatttaacggggacgtgcggccagagctggggaa

SEQ ID NO: 73 - RNA, 63 nt, synthetic construct
  source 1..63: mol_type = other RNA
tctcttacttcaaagactgaacattcttcacgatctgaaaggaaaagaagagattctttcggg

SEQ ID NO: 74 - RNA, 53 nt, synthetic construct
  source 1..53: mol_type = other RNA
tcgggcattaaaaaatatttaatcattcagttgttgagactcattcttgagtt

SEQ ID NO: 75 - RNA, 71 nt, synthetic construct
  source 1..71: mol_type = other RNA
attctacagtgcaagtagctttgattgtcagggttcaggttccctggttgtgaaaatacagtagataaatc

SEQ ID NO: 76 - RNA, 55 nt, synthetic construct
  source 1..55: mol_type = other RNA
gttgcttgggcgcttctccgctgcgtgtaggtgaagggggcttcctgaccgagac

SEQ ID NO: 77 - RNA, 60 nt, synthetic construct
  source 1..60: mol_type = other RNA
tacagtttgaagacagtaatacctggaattgagcatcagttctgtcattcttaaatttgc

SEQ ID NO: 78 - RNA, 88 nt, synthetic construct
  source 1..88: mol_type = other RNA
aatcccttaattaaatagcttcccctctacaggcttttgaagtggtagcagttcctcctaactcctgccagaaacagctctcctcaac

SEQ ID NO: 79 - RNA, 51 nt, synthetic construct
  source 1..51: mol_type = other RNA
agaccacagagccctgggttgtggaagaggtggctgttccctgtcatcagt

SEQ ID NO: 80 - RNA, 74 nt, synthetic construct
  source 1..74: mol_type = other RNA
gtttaaaattatccaactgccatagagctaaattcttttttggaaaattgaaccgaacttctactgaatacaag

SEQ ID NO: 81 - RNA, 73 nt, synthetic construct
  source 1..73: mol_type = other RNA
gaaagttagttgctttcttaacctcatcaaattcagtaagatttagccgaggcccagtccacaaatcatcaaa

SEQ ID NO: 82 - RNA, 46 nt, synthetic construct
  source 1..46: mol_type = other RNA
gttaagctagtccagagccgagtccaaattttattccggtacaggg

SEQ ID NO: 83 - RNA, 53 nt, synthetic construct
  source 1..53: mol_type = other RNA
agaattcagaactttgcttctaaaaaggtccctccagctacaaggtgggcacc

SEQ ID NO: 84 - RNA, 108 nt, synthetic construct
  source 1..108: mol_type = other RNA
agagttagccgaggccgccatattgaataagcgacccggcctcctagggggtcgtcgtggtccagacagtttagcagaacagcctccgcggctccggggagaagcaat

SEQ ID NO: 85 - RNA, 89 nt, synthetic construct
  source 1..89: mol_type = other RNA
atattgaataagcgacccggcctcctagggggtcgtcgtggtccagacagtttagcagaacagcctccgcggctccggggagaagcaat

SEQ ID NO: 86 - RNA, 42 nt, synthetic construct
  source 1..42: mol_type = other RNA
tattaatagaagagtcttcgttccagacgcagtccaggaatc

SEQ ID NO: 87 - RNA, 44 nt, synthetic construct
  source 1..44: mol_type = other RNA
cttcatttcccatcgtgctgaggcgggtggcagtgcggagaagg

SEQ ID NO: 88 - RNA, 54 nt, synthetic construct
  source 1..54: mol_type = other RNA
gcagatcagcggaagtgctaaagtaaggtgctatactttacttaaaaggtgaaa

SEQ ID NO: 89 - RNA, 82 nt, synthetic construct
  source 1..82: mol_type = other RNA
aacagttgccttttcctctcacagaaggaagatttcattttgtttgagacgagaaaccaaaccacacaccaaagagaggggt

SEQ ID NO: 90 - RNA, 18 nt, synthetic construct
  source 1..18: mol_type = other RNA
acgtggacgtcaacagca

SEQ ID NO: 91 - RNA, 58 nt, synthetic construct
  source 1..58: mol_type = other RNA
aattgtaaagagagagaagtggataaatcagtgctgctttctttaggacgaaagaagt

SEQ ID NO: 92 - RNA, 66 nt, synthetic construct
  source 1..66: mol_type = other RNA
taggtttcaattgtaaagagagagaagtggataaatcagtgctgctttctttaggacgaaagaagt

SEQ ID NO: 93 - RNA, 25 nt, synthetic construct
  source 1..25: mol_type = other RNA
agatttgctctcactgctcccaaaa

SEQ ID NO: 94 - RNA, 53 nt, synthetic construct
  source 1..53: mol_type = other RNA
agttttgagcatacttctgaactggcttttgttgagactatcagtatagaagc

SEQ ID NO: 95 - RNA, 62 nt, synthetic construct
  source 1..62: mol_type = other RNA
gagacagttgaaagttctctacaacagaagccaagaaggaagccgtctatcttgtggcgatc

SEQ ID NO: 96 - RNA, 12 nt, synthetic construct
  source 1..12: mol_type = other RNA
tttctccccgag

SEQ ID NO: 97 - RNA, 60 nt, synthetic construct
  source 1..60: mol_type = other RNA
gagaataaaaacagtgcttttgagtaaaaagtcatttaacagattccagttggagtgctc

SEQ ID NO: 98 - RNA, 20 nt, synthetic construct
  source 1..20: mol_type = other RNA
atattgcaggacgctgcatc

SEQ ID NO: 99 - RNA, 65 nt, synthetic construct
  source 1..65: mol_type = other RNA
agtcaattagttaaagactctgacatctgctaggcgggaggcaagagacctaacaagcaggggcc

SEQ ID NO: 100 - RNA, 51 nt, synthetic construct
  source 1..51: mol_type = other RNA
ttcacttcttcaactgagagtgacagtatataatctccattgagtcaaaga

SEQ ID NO: 101 - RNA, 74 nt, synthetic construct
  source 1..74: mol_type = other RNA
agacacacatagcctctctgcccacctctgcttcctctaggaacacaggagttccagatcacatcgagttcacc

SEQ ID NO: 102 - RNA, 54 nt, synthetic construct
  source 1..54: mol_type = other RNA
gtcagtcccagtgggaagttcaacaggcaggggcagcactgcagagatttcatc

SEQ ID NO: 103 - RNA, 51 nt, synthetic construct
  source 1..51: mol_type = other RNA
acgctttgcggctgtcgtcggagaggcatctgggttcggactggggccgcc

SEQ ID NO: 104 - RNA, 12 nt, synthetic construct
  source 1..12: mol_type = other RNA
gtcctcgcagcc

SEQ ID NO: 105 - RNA, 25 nt, synthetic construct
  source 1..25: mol_type = other RNA
acctggccgagcaggaggcgccatc

SEQ ID NO: 106 - RNA, 22 nt, synthetic construct
  source 1..22: mol_type = other RNA
caaagctggctctgcaggggac

SEQ ID NO: 107 - RNA, 20 nt, synthetic construct
  source 1..20: mol_type = other RNA
ctgctctgttaaaagcgaaa

SEQ ID NO: 108 - RNA, 12 nt, synthetic construct
  source 1..12: mol_type = other RNA
ccctccgggaag

SEQ ID NO: 109 - RNA, 67 nt, synthetic construct
  source 1..67: mol_type = other RNA
agagagagggggatttattcaaagttgtttccaattccttcaaaacctcaaaccaggtggctagtgt

SEQ ID NO: 110 - RNA, 20 nt, synthetic construct
  source 1..20: mol_type = other RNA
ttggctccagacaaataaac

SEQ ID NO: 111 - RNA, 64 nt, synthetic construct
  source 1..64: mol_type = other RNA
cgatagacgaggacgccaacagcagcggagaaacgtttctctttcctctcagtttgcgcacacc

SEQ ID NO: 112 - RNA, 49 nt, synthetic construct
  source 1..49: mol_type = other RNA
gagaaagctgtctaggtagagttcagtcagcccagtgacctcagcagcc

SEQ ID NO: 113 - RNA, 27 nt, synthetic construct
  source 1..27: mol_type = other RNA
tgacaagaccacaatctgatcccaaag

SEQ ID NO: 114 - RNA, 62 nt, synthetic construct
  source 1..62: mol_type = other RNA
gattgtctgtgctctgagcaggctgaagtgactggacgagacgcactgttggagaaataaaa

SEQ ID NO: 115 - RNA, 59 nt, synthetic construct
  source 1..59: mol_type = other RNA
attttcttcaatcacatctgaataaatcacttgaagaaagcttatagcttcattgcacc

SEQ ID NO: 116 - RNA, 72 nt, synthetic construct
  source 1..72: mol_type = other RNA
agttgaaaacttacagagtcctgaggctttcagactgaaaaaggctttcttctgtcactgacaatcgccacc

SEQ ID NO: 117 - RNA, 93 nt, synthetic construct
  source 1..93: mol_type = other RNA
cgctgagagcagcaaggccagagttgaaaacttacagagtcctgaggctttcagactgaaaaaggctttcttctgtcactgacaatcgccacc

SEQ ID NO: 118 - RNA, 69 nt, synthetic construct
  source 1..69: mol_type = other RNA
tcatcgttttgaagagttctgcgttttgccagtcacctctcaactgtgtgccaaaggactccagtaaag

SEQ ID NO: 119 - RNA, 55 nt, synthetic construct
  source 1..55: mol_type = other RNA
cacagagcgacagagacatttattgttatttgttttttggtggcaaaaagggaaa

SEQ ID NO: 120 - RNA, 70 nt, synthetic construct
  source 1..70: mol_type = other RNA
agccgcactcgccagcgactcatctctccagcgggtttttttttgtttgtcgtgtgcgatcctcacactc

SEQ ID NO: 121 - RNA, 85 nt, synthetic construct
  source 1..85: mol_type = other RNA
agtgtttgataaagacagctcctcttaggaagaactgtcatccccaaacacatagagagacactctctgtctctcgattacaatc

SEQ ID NO: 122 - RNA, 61 nt, synthetic construct
  source 1..61: mol_type = other RNA
aatccgcattcagttgtcgaggagtgccagtcaccttcagtttctggagctggccgtcaac

SEQ ID NO: 123 - RNA, 56 nt, synthetic construct
  source 1..56: mol_type = other RNA
cttttcgcgcgaaacctggttgttgctgtagtggcggagaggatcgtggtactgct

SEQ ID NO: 124 - RNA, 57 nt, synthetic construct
  source 1..57: mol_type = other RNA
acttttcgcgcgaaacctggttgttgctgtagtggcggagaggatcgtggtactgct

SEQ ID NO: 125 - RNA, 90 nt, synthetic construct
  source 1..90: mol_type = other RNA
aaagggaagcgaagccggagctgcgggcgctttttctgcccgcggtgtctcagattcattcttaaggaactgagaacttaatcttccaaa

SEQ ID NO: 126 - RNA, 85 nt, synthetic construct
  source 1..85: mol_type = other RNA
agaaagtttaaagtgctgagtgatttgcactagctgtatagatctttggtggaggaggaaagtggaaaattattaatttcagagc

SEQ ID NO: 127 - RNA, 15 nt, synthetic construct
  source 1..15: mol_type = other RNA
accgagctgcctgcc

SEQ ID NO: 128 - RNA, 23 nt, synthetic construct
  source 1..23: mol_type = other RNA
ccgatactcagccctctgcgacc

SEQ ID NO: 129 - RNA, 51 nt, synthetic construct
  source 1..51: mol_type = other RNA
tagagttgaagaacagactcaaaccaggtttttatttaattagttaaaaat

SEQ ID NO: 130 - RNA, 54 nt, synthetic construct
  source 1..54: mol_type = other RNA
agcagacactggaagttgaagtcattgggaggtgagcccttaaggagagcccaa

SEQ ID NO: 131 - RNA, 81 nt, synthetic construct
  source 1..81: mol_type = other RNA
tatttttatttacagtttttgttcaactaggcaacttaaaatagttttgctttagttataaaatattgtaagtgtagagta

SEQ ID NO: 132 - RNA, 34 nt, synthetic construct
  source 1..34: mol_type = other RNA
tctctctcgctcgttccctaacattaaagagaaa

SEQ ID NO: 133 - RNA, 32 nt, synthetic construct
  source 1..32: mol_type = other RNA
ctagacgtttccttggctcttctggcgccaaa

SEQ ID NO: 134 - RNA, 33 nt, synthetic construct
  source 1..33: mol_type = other RNA
tctagacgtttccttggctcttctggcgccaaa

SEQ ID NO: 135 - RNA, 79 nt, synthetic construct
  source 1..79: mol_type = other RNA
agagacacgaatagttttcagccgcaacaggctgcgtttcagccggaagagtgaaagggcaccttgaaaacgcaagttt

SEQ ID NO: 136 - RNA, 94 nt, synthetic construct
  source 1..94: mol_type = other RNA
agatcgtaaacaaaacccaggacgccaggcagatttctgtgttaacaagaaaaaagagtttgagttggatactctcagtaaatccgagctgcgg

SEQ ID NO: 137 - RNA, 52 nt, synthetic construct
  source 1..52: mol_type = other RNA
cgctttttccgcgggcgcttgataacgcgggtgaggcgtggagggcggcgcc

SEQ ID NO: 138 - RNA, 62 nt, synthetic construct
  source 1..62: mol_type = other RNA
ttatatttaagcagtgagagtatttacgtcaagatcaacttattcttcaaatcatttcactc

SEQ ID NO: 139 - RNA, 48 nt, synthetic construct
  source 1..48: mol_type = other RNA
ctggtttcttgccttaaggagcccattgcctttcccgctgaagtctag

SEQ ID NO: 140 - RNA, 59 nt, synthetic construct
  source 1..59: mol_type = other RNA
agcagtttctgagctgcaaaaactagtttctaaacagagagttaattgttaaatccagt

SEQ ID NO: 141 - RNA, 31 nt, synthetic construct
  source 1..31: mol_type = other RNA
actttcttttgtaaagttggtttctacaaag

SEQ ID NO: 142 - RNA, 48 nt, synthetic construct
  source 1..48: mol_type = other RNA
aaaaacagcaactctaaactggctctggatagttcataagctgtggcc

SEQ ID NO: 143 - RNA, 97 nt, synthetic construct
  source 1..97: mol_type = other RNA
agccttcgggccttgtagtaggtagcagtaaacagcagatttgagtatccacaatcctttcagtattaaattttcagtaaaataaaattacttgtat

SEQ ID NO: 144 - RNA, 16 nt, synthetic construct
  source 1..16: mol_type = other RNA
aaaataactagcaacc

SEQ ID NO: 145 - RNA, 42 nt, synthetic construct
  source 1..42: mol_type = other RNA
attgtgcttccaccactgccaataacaaaataactagcaacc

SEQ ID NO: 146 - RNA, 72 nt, synthetic construct
  source 1..72: mol_type = other RNA
aatttgaagggtttcttgtagagagaaagtcacattttgacgtacatttgattcaagtgcagggtgtctttt

SEQ ID NO: 147 - RNA, 44 nt, synthetic construct
  source 1..44: mol_type = other RNA
cagaggtgctgttatcagaatagtcttctgggtataaattaaaa

SEQ ID NO: 148 - RNA, 23 nt, synthetic construct
  source 1..23: mol_type = other RNA
gagaatcccggtggtgagcgggg

SEQ ID NO: 149 - RNA, 85 nt, synthetic construct
  source 1..85: mol_type = other RNA
tttgtttctgtcaggctgtccctggccggggttctgtaacgcttgtgtgggccgcaggtggaggtgttgggaaagcgcggaggag

SEQ ID NO: 150 - RNA, 21 nt, synthetic construct
  source 1..21: mol_type = other RNA
cccctttggagctacttcctc

SEQ ID NO: 151 - RNA, 45 nt, synthetic construct
  source 1..45: mol_type = other RNA
gtttttctttttttaagtaacaagtaaaccatacaaattgtcaac

SEQ ID NO: 152 - RNA, 20 nt, synthetic construct
  source 1..20: mol_type = other RNA
gctgtcctcttcagctcaag

SEQ ID NO: 153 - RNA, 45 nt, synthetic construct
  source 1..45: mol_type = other RNA
ctggaagaggatcttgggcgccgccaggttctgtggacaatcaca

SEQ ID NO: 154 - RNA, 54 nt, synthetic construct
  source 1..54: mol_type = other RNA
attatacttcttacagtgaggagccagagcttccaggttctgtggacaatcaca

SEQ ID NO: 155 - RNA, 55 nt, synthetic construct
  source 1..55: mol_type = other RNA
gtgagaggacattgtgataagtgaagtgaaagagctttgtcagctgcacaggtga

SEQ ID NO: 156 - RNA, 67 nt, synthetic construct
  source 1..67: mol_type = other RNA
acagaaagtagctcattcctcctaactgggtaatagacagtggtggggcctggaaaagatttcaaat

SEQ ID NO: 157 - RNA, 29 nt, synthetic construct
  source 1..29: mol_type = other RNA
agctacagctacctccggacgctctcaag

SEQ ID NO: 158 - RNA, 99 nt, synthetic construct
  source 1..99: mol_type = other RNA
cagatactttggtgttgaacatttgggatagaagagagaaatttaaaattaaaaattttccatatttctatcacaaatattggtgtgcaactgaggaac

SEQ ID NO: 159 - RNA, 92 nt, synthetic construct
  source 1..92: mol_type = other RNA
agcttcaagtgaagttaatctgaggtgaagcaaacagaaaattgtggaggttttgttggtggagataagtaattaacatttcgaaacaggaa

SEQ ID NO: 160 - RNA, 97 nt, synthetic construct
  source 1..97: mol_type = other RNA
gaagagagaaatttaaaattaaaaattttccatatttctatcacaaatattggtaatttgacaacagttaatttgattgtcgtcttaataaaattta

SEQ ID NO: 161 - RNA, 94 nt, synthetic construct
  source 1..94: mol_type = other RNA
acagcttcaagtgaagttaatctgaggtgaagcaaacagaaaattgtggaggttttgttggtggagataagtaattaacatttcgaaacaggaa

SEQ ID NO: 162 - RNA, 48 nt, synthetic construct
  source 1..48: mol_type = other RNA
cagatttgttttcctctgagtggttttcagcttgctggttaattgaaa

SEQ ID NO: 163 - RNA, 15 nt, synthetic construct
  source 1..15: mol_type = other RNA
aagcggcgagtctcc

SEQ ID NO: 164 - RNA, 57 nt, synthetic construct
  source 1..57: mol_type = other RNA
atatacgggctccaggctgaacggctcgggccacttacacaccactgcctgataacc

SEQ ID NO: 165 - RNA, 46 nt, synthetic construct
  source 1..46: mol_type = other RNA
ttcagcaacccgtctgaacagatctggcgtcctggagaattctaga

SEQ ID NO: 166 - RNA, 20 nt, synthetic construct
  source 1..20: mol_type = other RNA
cgcgccttcctgcggctaag

SEQ ID NO: 167 - RNA, 66 nt, synthetic construct
  source 1..66: mol_type = other RNA
acaatctgcctctgaagctggagatactagctgcagagctcaggggagctgctccacatcaccgac

SEQ ID NO: 168 - RNA, 48 nt, synthetic construct
  source 1..48: mol_type = other RNA
ccttttataagtaacctactactcctgcaataacagcataatctattc

SEQ ID NO: 169 - RNA, 60 nt, synthetic construct
  source 1..60: mol_type = other RNA
ttctggaggctgggaagtccaagaccagttggctcgcatctgacttcagtgaagttcctt

SEQ ID NO: 170 - RNA, 12 nt, synthetic construct
  source 1..12: mol_type = other RNA
cccgcttccaaa

SEQ ID NO: 171 - RNA, 74 nt, synthetic construct
  source 1..74: mol_type = other RNA
caggaagctagcagagtttgcttctgagacgaattctgttacagtacagtgcatttagtaggatcaagttaatt

SEQ ID NO: 172 - RNA, 34 nt, synthetic construct
  source 1..34: mol_type = other RNA
attgcagcaagtaagccaacaaggtcttttcagg

SEQ ID NO: 173 - RNA, 42 nt, synthetic construct
  source 1..42: mol_type = other RNA
agaacagacgtttgaacagagcaggcttctgaggtctccaaa

SEQ ID NO: 174 - RNA, 22 nt, synthetic construct
  source 1..22: mol_type = other RNA
gttgccactctctctcctgtca

SEQ ID NO: 175 - RNA, 40 nt, synthetic construct
  source 1..40: mol_type = other RNA
actatcgcttacctacctccctctgcaggaacccggcgat

SEQ ID NO: 176 - RNA, 23 nt, synthetic construct
  source 1..23: mol_type = other RNA
gctaagtttttggccgcttcaag

SEQ ID NO: 177 - RNA, 23 nt, synthetic construct
  source 1..23: mol_type = other RNA
agcactctgacctagcagtcaac

SEQ ID NO: 178 - RNA, 25 nt, synthetic construct
  source 1..25: mol_type = other RNA
ctagcactctgacctagcagtcaac

SEQ ID NO: 179 - RNA, 63 nt, synthetic construct
  source 1..63: mol_type = other RNA
ttattttggcagccttatccaagtctggtacaacatagcaaagagaacaggctagtaaataag

SEQ ID NO: 180 - RNA, 62 nt, synthetic construct
  source 1..62: mol_type = other RNA
acaagccagccagtccctgcagctccacaaactgacccatcctgggccttgttctccacaga

SEQ ID NO: 181 - RNA, 25 nt, synthetic construct
  source 1..25: mol_type = other RNA
ccaactgggtcctgaagtagctgaa

SEQ ID NO: 182 - RNA, 54 nt, synthetic construct
  source 1..54: mol_type = other RNA
attgcatcactgcactccagcttgtagtacagtgagacagtgtcttaaggaaag

SEQ ID NO: 183 - RNA, 90 nt, synthetic construct
  source 1..90: mol_type = other RNA
aaggagttcaagttgcttgccagactaacttacttttaaaagtaagcagccttccaagttaagagagtattttaaatttcagattttatc

SEQ ID NO: 184 - RNA, 21 nt, synthetic construct
  source 1..21: mol_type = other RNA
cagcccatccacaacctgctc

SEQ ID NO: 185 - RNA, 64 nt, synthetic construct
  source 1..64: mol_type = other RNA
tttttccagtgaaacaaaacgtaagaatctgagtttgtttttcaaagatcactaaattttagtt

SEQ ID NO: 186 - RNA, 53 nt, synthetic construct
  source 1..53: mol_type = other RNA
ttcttggccttggcaaactttggagattggggacagccagtagaggtgacttc

SEQ ID NO: 187 - RNA, 60 nt, synthetic construct
  source 1..60: mol_type = other RNA
gcgtctgagcattgttagtcgttattttcaagctttgctaacggtttttaaacctttgag

SEQ ID NO: 188 - RNA, 42 nt, synthetic construct
  source 1..42: mol_type = other RNA
tgcagtttttaagaagaaaaagaaggccctgagtcaaagaag

SEQ ID NO: 189 - RNA, 70 nt, synthetic construct
  source 1..70: mol_type = other RNA
aaaacaggaaataggtgtttcatatatacggctctaaccttctctctctgcaccttccttctgtcaatag

SEQ ID NO: 190 - RNA, 19 nt, synthetic construct
  source 1..19: mol_type = other RNA
accttccttctgtcaatag

SEQ ID NO: 191 - RNA, 56 nt, synthetic construct
  source 1..56: mol_type = other RNA
atcttgcttgtagtcctctttccttttcacctttccattgttcctgacgagtagaa

SEQ ID NO: 192 - RNA, 53 nt, synthetic construct
  source 1..53: mol_type = other RNA
acagcagacgtaccctccctcgctgcctgcctgcagcctgccctgcagtcagg

SEQ ID NO: 193 - RNA, 56 nt, synthetic construct
  source 1..56: mol_type = other RNA
accagcccagcctttcagtgcaggctccagccctccacccccacccgagttgcagg

SEQ ID NO: 194 - RNA, 52 nt, synthetic construct
  source 1..52: mol_type = other RNA
ttattacctcccttttaaagagtaggaagctctgacacagaaaagctaaata

SEQ ID NO: 195 - RNA, 23 nt, synthetic construct
  source 1..23: mol_type = other RNA
aagaaatagaacagaattcagcc

SEQ ID NO: 196 - RNA, 90 nt, synthetic construct
  source 1..90: mol_type = other RNA
atatagtagtcagcaaccggaggagtcacgttgggggaacggcagaaagcagctatccgtttacactactttgctctaacagctatctta

SEQ ID NO: 197 - RNA, 62 nt, synthetic construct
  source 1..62: mol_type = other RNA
gcggagggagtggaacctcgatattggtggtgtccatcgtgggcagcggactaataaaggcc

SEQ ID NO: 198 - RNA, 19 nt, synthetic construct
  source 1..19: mol_type = other RNA
ccagcgaagacaggctgag

SEQ ID NO: 199 - RNA, 37 nt, synthetic construct
  source 1..37: mol_type = other RNA
agaaccgttcgcggaggaaaggcgaactagtgttggg

SEQ ID NO: 200 - RNA, 94 nt, synthetic construct
  source 1..94: mol_type = other RNA
acagttgtgttgggctcacaccagtgaaccgaagctctggattctgagagtctgaggattccgtgaagatctcagacttgggctcagagcaagg

SEQ ID NO: 201 - RNA, 24 nt, synthetic construct
  source 1..24: mol_type = other RNA
agcctcgtgaccttttaccccaac

SEQ ID NO: 202 - RNA, 90 nt, synthetic construct
  source 1..90: mol_type = other RNA
actagggcaatctgccggaagagtcaggttctgtgtagttctccgcgtcttccgcggagcgggtgtgcagggcctgcagcattgaactag

SEQ ID NO: 203 - RNA, 12 nt, synthetic construct
  source 1..12: mol_type = other RNA
cctggcaatccc

SEQ ID NO: 204 - RNA, 44 nt, synthetic construct
  source 1..44: mol_type = other RNA
agtgttggttcagtaagttttaccatcaattcaagtaatcataa

SEQ ID NO: 205 - RNA, 28 nt, synthetic construct
  source 1..28: mol_type = other RNA
aagtagcgctgcggttggcagcggcggg

SEQ ID NO: 206 - RNA, 39 nt, synthetic construct
  source 1..39: mol_type = other RNA
actctctgttcttgctatctaagtgccgctgcttccgcc

SEQ ID NO: 207 - RNA, 66 nt, synthetic construct
  source 1..66: mol_type = other RNA
aatttgagactcagcctagtccaggcaagctactggcacctgctgctctcaactaacctccacaca

SEQ ID NO: 208 - RNA, 21 nt, synthetic construct
  source 1..21: mol_type = other RNA
ggaatcgggagattcgggacc

SEQ ID NO: 209 - RNA, 13 nt, synthetic construct
  source 1..13: mol_type = other RNA
agactgttccaag

SEQ ID NO: 210 - RNA, 99 nt, synthetic construct
  source 1..99: mol_type = other RNA
ttacaaggatcacaggggttggtacaagttgcttgataatctgcagcattctctctgagatttagctgccattgtgttgccaaatttctgcagttcagt

SEQ ID NO: 211 - RNA, 45 nt, synthetic construct
  source 1..45: mol_type = other RNA
acacgcgccctacctcggagtgtgtggcgccagtagtcagggaag

SEQ ID NO: 212 - RNA, 92 nt, synthetic construct
  source 1..92: mol_type = other RNA
gttagttctacagaagaggcagaaaaacaagagataacaaaggctccgtttcctttctgtgagagaaggcttttgtctttcctcctgctacg

SEQ ID NO: 213 - RNA, 12 nt, synthetic construct
  source 1..12: mol_type = other RNA
acgaggtgggcc

SEQ ID NO: 214 - RNA, 42 nt, synthetic construct
  source 1..42: mol_type = other RNA
cagactgtggttttgtgcttgctcaccaaagctaacctcagc

SEQ ID NO: 215 - RNA, 54 nt, synthetic construct
  source 1..54: mol_type = other RNA
ggcagcgggacgcaccatttcagttgtgttcttggttcatttcgtgtctcggcg

SEQ ID NO: 216 - RNA, 92 nt, synthetic construct
  source 1..92: mol_type = other RNA
aaaggggccttcaagaagagagttctcagagcagactcagctttaggtgctgaaagtaatttctcaagatcatcactccattcagaaggcta

SEQ ID NO: 217 - RNA, 56 nt, synthetic construct
  source 1..56: mol_type = other RNA
actcagcctcccttccccagccgtgacagcactggagcctttcggacacctggacc

SEQ ID NO: 218 - RNA, 57 nt, synthetic construct
  source 1..57: mol_type = other RNA
ttccttacctgctttgagtctgtggaaggagacaggaattctggatctgcatctcca

SEQ ID NO: 219 - RNA, 27 nt, synthetic construct
  source 1..27: mol_type = other RNA
cttggaccttggccctcgctttccagg

SEQ ID NO: 220 - RNA, 78 nt, synthetic construct
  source 1..78: mol_type = other RNA
gagaatcagattgtgcccacaaggcggaaagttacgacagcgggttccggtgaccacgaaggcggcaaaggcgacgga

SEQ ID NO: 221 - RNA, 12 nt, synthetic construct
  source 1..12: mol_type = other RNA
attccatcccac

SEQ ID NO: 222 - RNA, 21 nt, synthetic construct
  source 1..21: mol_type = other RNA
acgtgtgctattccatcccac

SEQ ID NO: 223 - RNA, 59 nt, synthetic construct
  source 1..59: mol_type = other RNA
gcagcagttttctacgtctgaaattttttagttctctggaacccagaatttgctaagag

SEQ ID NO: 224 - RNA, 46 nt, synthetic construct
  source 1..46: mol_type = other RNA
acattaaaaataacatttttctctcttgtagtgtgactgtgggaag

SEQ ID NO: 225 - RNA, 69 nt, synthetic construct
  source 1..69: mol_type = other RNA
gctgtcctaggattgctggcttttgtttttgctgctgagcctggagctcagctcagactgagaggagag

SEQ ID NO: 226 - RNA, 27 nt, synthetic construct
  source 1..27: mol_type = other RNA
gtagtacgaatccgtcaggccggaacc

SEQ ID NO: 227 - RNA, 98 nt, synthetic construct
  source 1..98: mol_type = other RNA
ttaaaaaaacagtttcctgctagtgccctgaagattcctgccaagagaatatttggtgataattttgatccagtccagagttcagagcattccttcaa

SEQ ID NO: 228 - RNA, 55 nt, synthetic construct
  source 1..55: mol_type = other RNA
tgttgcagacccaggagtctctgaaggcttccgcggctttggggccggttgtgag

SEQ ID NO: 229 - RNA, 60 nt, synthetic construct
  source 1..60: mol_type = other RNA
gaagacgcttcctgggtttgaggagttcagtgactgctattgaaccaccaaaagtccatt

SEQ ID NO: 230 - RNA, 54 nt, synthetic construct
  source 1..54: mol_type = other RNA
gtgcaaacatctcaagccagttcagagtttgctgtttcctcaagttgcaggtct

SEQ ID NO: 231 - RNA, 15 nt, synthetic construct
  source 1..15: mol_type = other RNA
cttttctttggcaag

SEQ ID NO: 232 - RNA, 24 nt, synthetic construct
  source 1..24: mol_type = other RNA
acagactcccttttctttggcaag

SEQ ID NO: 233 - RNA, 22 nt, synthetic construct
  source 1..22: mol_type = other RNA
agactcccttttctttggcaag

SEQ ID NO: 234 - RNA, 11 nt, synthetic construct
  source 1..11: mol_type = other RNA
cttccgcgtgc

SEQ ID NO: 235 - RNA, 15 nt, synthetic construct
  source 1..15: mol_type = other RNA
ttggctgctagagcg

SEQ ID NO: 236 - RNA, 22 nt, synthetic construct
  source 1..22: mol_type = other RNA
agtggcgttggctgctagagcg

SEQ ID NO: 237 - RNA, 20 nt, synthetic construct
  source 1..20: mol_type = other RNA
tggcgttggctgctagagcg

SEQ ID NO: 238 - RNA, 92 nt, synthetic construct
  source 1..92: mol_type = other RNA
acccgcaaaccagatcgccgctccggagcccccagcccactgggagggctcgcggttactgcaaggattctggcgaagtaggtctaaataga

SEQ ID NO: 239 - RNA, 15 nt, synthetic construct
  source 1..15: mol_type = other RNA
acccggagcgggaag

SEQ ID NO: 240 - RNA, 92 nt, synthetic construct
  source 1..92: mol_type = other RNA
ggtcctaattcgggaaacgtgcaccttgactaattcggttttctgtggctacgagagcaggcttggcgggcggaggcgccagcggagttctc

SEQ ID NO: 241 - RNA, 15 nt, synthetic construct
  source 1..15: mol_type = other RNA
acacgattctccaac

SEQ ID NO: 242 - RNA, 20 nt, synthetic construct
  source 1..20: mol_type = other RNA
tctagtccacgctcggagcc

SEQ ID NO: 243 - RNA, 16 nt, synthetic construct
  source 1..16: mol_type = other RNA
aacaccctcctggagg

SEQ ID NO: 244 - RNA, 67 nt, synthetic construct
  source 1..67: mol_type = other RNA
ggaaaagctcgataattacccagcctaaccatttctcaggtgcttgcgaggtgatcagaaggcaaag

SEQ ID NO: 245 - RNA, 11 nt, synthetic construct
  source 1..11: mol_type = other RNA
aagacgccaag

SEQ ID NO: 246 - RNA, 37 nt, synthetic construct
  source 1..37: mol_type = other RNA
taagaccggccccttccggttacgaaaccttagcaag

SEQ ID NO: 247 - RNA, 14 nt, synthetic construct
  source 1..14: mol_type = other RNA
cttcctttgacaag

SEQ ID NO: 248 - RNA, 55 nt, synthetic construct
  source 1..55: mol_type = other RNA
actcccttcctcttttctgctcacacaggaagccctggaagctgcttcctcagac

SEQ ID NO: 249 - RNA, 58 nt, synthetic construct
  source 1..58: mol_type = other RNA
agcctccagcggtggctacagttgaaaccctcacaccacgcaggaagaggtcatcatc

SEQ ID NO: 250 - RNA, 29 nt, synthetic construct
  source 1..29: mol_type = other RNA
acagcagctctacccctcacgacgcagac

SEQ ID NO: 251 - RNA, 92 nt, synthetic construct
  source 1..92: mol_type = other RNA
gggtttggtggcagaaggagagtgtgggggaggcaggggtcttctccctgatcgtcttctcctccctgctgaccgacggctaccagaacaag

SEQ ID NO: 252 - RNA, 12 nt, synthetic construct
  source 1..12: mol_type = other RNA
cttcgactcgct

SEQ ID NO: 253 - RNA, 15 nt, synthetic construct
  source 1..15: mol_type = other RNA
cccctgcctctgagg

SEQ ID NO: 254 - RNA, 47 nt, synthetic construct
  source 1..47: mol_type = other RNA
agaggacacttccttttgcgggtggcggcgaacgcggagagcacgcc

SEQ ID NO: 255 - RNA, 27 nt, synthetic construct
  source 1..27: mol_type = other RNA
aagaggagctggtgagaagacagcgaa

SEQ ID NO: 256 - RNA, 13 nt, synthetic construct
  source 1..13: mol_type = other RNA
attgtgggattgg

SEQ ID NO: 257 - RNA, 12 nt, synthetic construct
  source 1..12: mol_type = other RNA
cctgttgcagcc

SEQ ID NO: 258 - RNA, 21 nt, synthetic construct
  source 1..21: mol_type = other RNA
gtgggaaccttgacaagcagc

SEQ ID NO: 259 - RNA, 22 nt, synthetic construct
  source 1..22: mol_type = other RNA
actgtgctctgcgagccgaatc

SEQ ID NO: 260 - RNA, 47 nt, synthetic construct
  source 1..47: mol_type = other RNA
ataaacattgggctgcacatagagacttaattttagatttagacaaa

SEQ ID NO: 261 - RNA, 74 nt, synthetic construct
  source 1..74: mol_type = other RNA
ataaattgtgcaagggtgctatagacgcacaaacgaccgcgagccacaaatcaagcacacatatcaaaaaacaa

SEQ ID NO: 262 - RNA, 54 nt, synthetic construct
  source 1..54: mol_type = other RNA
tagatcgtcaggaaaagcctaaagattagactgtaagaaaagaaaatagaagcc

SEQ ID NO: 263 - RNA, 29 nt, synthetic construct
  source 1..29: mol_type = other RNA
atctttgcagattatcctgtggaaggaaa

SEQ ID NO: 264 - RNA, 62 nt, synthetic construct
  source 1..62: mol_type = other RNA
tacagctttgattttataacatcctgctaatttttgttcttacagaaatacactttcgggaa

SEQ ID NO: 265 - RNA, 23 nt, synthetic construct
  source 1..23: mol_type = other RNA
aaaatcaattctcacacgctggg

SEQ ID NO: 266 - RNA, 89 nt, synthetic construct
  source 1..89: mol_type = other RNA
tcattgcggctggcgttcccaatacagacgcatcgtttcttttttaatactccctaagaaagggaataaccttcaagctggcgggagca

SEQ ID NO: 267 - RNA, 28 nt, synthetic construct
  source 1..28: mol_type = other RNA
ctggttcctctcggagcggagacggcaa

SEQ ID NO: 268 - RNA, 89 nt, synthetic construct
  source 1..89: mol_type = other RNA
caattcctgtctggattatattgctttttcatttagcatccctggctggctcagtgagaattccagtattcctggttattgttttcctg

SEQ ID NO: 269 - RNA, 22 nt, synthetic construct
  source 1..22: mol_type = other RNA
gagccggaggaggtggagagta

SEQ ID NO: 270 - RNA, 42 nt, synthetic construct
  source 1..42: mol_type = other RNA
acagcctgcggggaacgtagctagctgcaagcagaggccggc

SEQ ID NO: 271 - RNA, 49 nt, synthetic construct
  source 1..49: mol_type = other RNA
cttcttggtgaatttttggagtaagccattaaattaattgcttgccatc

SEQ ID NO: 272 - RNA, 53 nt, synthetic construct
  source 1..53: mol_type = other RNA
agtggtgtggtaccgggtacccggagacgtgtatcggacggtgggccgcagcc

SEQ ID NO: 273 - RNA, 59 nt, synthetic construct
  source 1..59: mol_type = other RNA
aagctcttactgcaggcagattgaagcgtcttactttccgaaaattggagtgtgacaat

SEQ ID NO: 274 - RNA, 12 nt, synthetic construct
  source 1..12: mol_type = other RNA
gtgagcggcaac

SEQ ID NO: 275 - RNA, 22 nt, synthetic construct
  source 1..22: mol_type = other RNA
atttcaacactacacttgcaca

SEQ ID NO: 276 - RNA, 62 nt, synthetic construct
  source 1..62: mol_type = other RNA
agtttgcagttgagcccaaactgaagtctgtctgtagaaggaaacaacaaactttgtacttt

SEQ ID NO: 277 - RNA, 12 nt, synthetic construct
  source 1..12: mol_type = other RNA
gttctccgtaag

SEQ ID NO: 278 - RNA, 46 nt, synthetic construct
  source 1..46: mol_type = other RNA
attgtgccctaccagagaggacagagtggcaccgcctccagtggca

SEQ ID NO: 279 - RNA, 64 nt, synthetic construct
  source 1..64: mol_type = other RNA
tgcatttggtattagctagtggaagcagtagttagtgttgaagtgcattgctgcagctggtagc

SEQ ID NO: 280 - RNA, 31 nt, synthetic construct
  source 1..31: mol_type = other RNA
agccggagccgtggcctgcggggccggcgac

SEQ ID NO: 281 - RNA, 57 nt, synthetic construct
  source 1..57: mol_type = other RNA
ctttattagagagatacttgttgaccgacgaagtcagtggggcagccgctaagaata

SEQ ID NO: 282 - RNA, 49 nt, synthetic construct
  source 1..49: mol_type = other RNA
attgggagcctttgggggtttattctcttcccttctaacttgattaaga

SEQ ID NO: 283 - RNA, 85 nt, synthetic construct
  source 1..85: mol_type = other RNA
cacaatctacacagcagccttgaaggaaaagacgccagacttcagacgtctctctcctcgggcgccctgcaaggccgcaggcagg

SEQ ID NO: 284 - RNA, 53 nt, synthetic construct
  source 1..53: mol_type = other RNA
aaaatatcagagtcaccagagacacaaggcttgaagctctgtcctgggaaaat

SEQ ID NO: 285 - RNA, 25 nt, synthetic construct
  source 1..25: mol_type = other RNA
aaaagaattatcacattgcacaagg

SEQ ID NO: 286 - RNA, 48 nt, synthetic construct
  source 1..48: mol_type = other RNA
ttataaagctaaagatcacaacttattttctagatatatcatacgaaa

SEQ ID NO: 287 - RNA, 84 nt, synthetic construct
  source 1..84: mol_type = other RNA
agagctgagagaggaaaagaacacagatctcgcagtgttcagatttttctttttaggtccaggagtaagatatatcatacgaaa

SEQ ID NO: 288 - RNA, 66 nt, synthetic construct
  source 1..66: mol_type = other RNA
ccgaggagctgaggaagcctagagctctcagaagcagtcctttgagctggtgtaggggcactcaga

SEQ ID NO: 289 - RNA, 30 nt, synthetic construct
  source 1..30: mol_type = other RNA
tcctattgggttaacactggaggctctaag

SEQ ID NO: 290 - RNA, 46 nt, synthetic construct
  source 1..46: mol_type = other RNA
acagggcagacctgtagtgttcgtttattcctggggttgtcatatc

SEQ ID NO: 291 - RNA, 12 nt, synthetic construct
  source 1..12: mol_type = other RNA
accggctgagcc

SEQ ID NO: 292 - RNA, 52 nt, synthetic construct
  source 1..52: mol_type = other RNA
ggctcagagaggttaagtgactcagccaaggtcaaacagctatctaggagcc

SEQ ID NO: 293 - RNA, 42 nt, synthetic construct
  source 1..42: mol_type = other RNA
tagtaagtggtggagccaggactcaaagccagtctaggagcc

SEQ ID NO: 294 - RNA, 39 nt, synthetic construct
  source 1..39: mol_type = other RNA
attattcttttgaagattcttcgttgtcaagccgccaaa

SEQ ID NO: 295 - RNA, 59 nt, synthetic construct
  source 1..59: mol_type = other RNA
acagcggcttccttgatccttgccacccgcgactgaacaccgacagcagcagcctcacc

SEQ ID NO: 296 - RNA, 78 nt, synthetic construct
  source 1..78: mol_type = other RNA
gtcactttcagtaattcaaaggcaatttaccagtgatttctgggtgctggggctgatattttttgtgcatatttaaga

SEQ ID NO: 297 - RNA, 25 nt, synthetic construct
  source 1..25: mol_type = other RNA
ctttcgttgcctgatcgccgccatc

SEQ ID NO: 298 - RNA, 50 nt, synthetic construct
  source 1..50: mol_type = other RNA
tcccgattgactggcctgcttggcaaggcaagtagcggcggcgcttcaag

SEQ ID NO: 299 - RNA, 20 nt, synthetic construct
  source 1..20: mol_type = other RNA
gttgtcactcgactaccaag

SEQ ID NO: 300 - RNA, 21 nt, synthetic construct
  source 1..21: mol_type = other RNA
actaggcggtttggtaacacg

SEQ ID NO: 301 - RNA, 92 nt, synthetic construct
  source 1..92: mol_type = other RNA
ccggaatagtgagccctgggcgcgggggcggcgtgtcaggtggttgcggagctgaatcatattctaagaactcagccactcaggtatccacc

SEQ ID NO: 302 - RNA, 62 nt, synthetic construct
  source 1..62: mol_type = other RNA
gcgtgtcaggtggttgcggagctgaatcatattctaagaactcagccactcaggtatccacc

SEQ ID NO: 303 - RNA, 23 nt, synthetic construct
  source 1..23: mol_type = other RNA
actgtggttctgcgcttgtcatc

SEQ ID NO: 304 - RNA, 53 nt, synthetic construct
  source 1..53: mol_type = other RNA
acagggcagtgtagttccagaaaataggactgaccaagaagcagaaaagcaag

SEQ ID NO: 305 - RNA, 54 nt, synthetic construct
  source 1..54: mol_type = other RNA
attagtatctcttgggctgctgtgagtgacacacgctgagtggggtgaagggaa

SEQ ID NO: 306 - RNA, 67 nt, synthetic construct
  source 1..67: mol_type = other RNA
aggtgatcatttcaaaccaagcatcagcaacaattaaaaatattcacttggtatctgtagtttaata

SEQ ID NO: 307 - RNA, 23 nt, synthetic construct
  source 1..23: mol_type = other RNA
cttggggaccctggaccactatc

SEQ ID NO: 308 - RNA, 57 nt, synthetic construct
  source 1..57: mol_type = other RNA
gtccatctcatagcaggcacaaactcatccatccccagttgattggaagaaacaacg

SEQ ID NO: 309 - RNA, 64 nt, synthetic construct
  source 1..64: mol_type = other RNA
gaagaagaagaagcttttgattggacagtcgctagtccgacctcagagaactcagccaatcata

SEQ ID NO: 310 - RNA, 53 nt, synthetic construct
  source 1..53: mol_type = other RNA
aacgtcatctaggagcaccgagcagcttggctaaaagtaagggtgtcgtgctg

SEQ ID NO: 311 - RNA, 97 nt, synthetic construct
  source 1..97: mol_type = other RNA
attatttcatcatttgggcttgccctttcagttggagcactgtgattggacccaagttggcaagagtggaagaccaggggacagaacagaaatcccc

SEQ ID NO: 312 - RNA, 67 nt, synthetic construct
  source 1..67: mol_type = other RNA
caaacgtgtttgcggaaaggagtttgggttccatcttttcatttccccagcgcagctttctgtagaa

SEQ ID NO: 313 - RNA, 42 nt, synthetic construct
  source 1..42: mol_type = other RNA
agttaaagtaggagttagtactgttcagtttttttcttagaa

SEQ ID NO: 314 - RNA, 59 nt, synthetic construct
  source 1..59: mol_type = other RNA
agaagtttggctcaattaagaaacatcagggagataaattcaacccagtgtgtctaaaa

SEQ ID NO: 315 - RNA, 93 nt, synthetic construct
  source 1..93: mol_type = other RNA
aagactgcatccggctccaggaaaagcgagtgggatatcccaatctttggactgcatcctggttgcctctactgtggtcacctttgggaagaa

SEQ ID NO: 316 - RNA, 48 nt, synthetic construct
  source 1..48: mol_type = other RNA
caagtatattagctagtacagcacgtagtagcctggggtcctacaggt

SEQ ID NO: 317 - RNA, 14 nt, synthetic construct
  source 1..14: mol_type = other RNA
acttcggggccaag

SEQ ID NO: 318 - RNA, 19 nt, synthetic construct
  source 1..19: mol_type = other RNA
ccttttggtacgctccaag

SEQ ID NO: 319 - RNA, 53 nt, synthetic construct
  source 1..53: mol_type = other RNA
gtgaggccgacagctccaaactccgtccccagtcctcactcctactccaagtg

SEQ ID NO: 320 - RNA, 56 nt, synthetic construct
  source 1..56: mol_type = other RNA
ggaagacggcagtttggcgacatttctcggccgaagggccatttgcttttgcggag

SEQ ID NO: 321 - RNA, 21 nt, synthetic construct
  source 1..21: mol_type = other RNA
cctttcctttccgcgtgtaga

SEQ ID NO: 322 - RNA, 70 nt, synthetic construct
  source 1..70: mol_type = other RNA
acttccggttctctgtcagtcgcgagcgaacgaccaagagggtgttcgactgctagagccgagcgaagcg

SEQ ID NO: 323 - RNA, 65 nt, synthetic construct
  source 1..65: mol_type = other RNA
tgcatcgttagcttctcctgataaactaattgcctcacattgtcactgcaaatcgacacctatta

SEQ ID NO: 324 - RNA, 44 nt, synthetic construct
  source 1..44: mol_type = other RNA
agtcactttctttgccaaaggcaaacgcagaacgtttcagagcc

SEQ ID NO: 325 - RNA, 44 nt, synthetic construct
  source 1..44: mol_type = other RNA
attggaactgtcacccggaggacaggctgtcagagctggacacc

SEQ ID NO: 326 - RNA, 89 nt, synthetic construct
  source 1..89: mol_type = other RNA
gttaaatcaatttctgtttgctttcttctcttctctgtaggttttgagcaaaattttggactgtgaagcaaggcattggtgaagacaaa

SEQ ID NO: 327 - RNA, 36 nt, synthetic construct
  source 1..36: mol_type = other RNA
aaagtgctgagttaaagtacatttctggatttggga

SEQ ID NO: 328 - RNA, 64 nt, synthetic construct
  source 1..64: mol_type = other RNA
gtatttgagttctaccgtcagtcctggcattatttctctctctacaaggagccttaggagcact

SEQ ID NO: 329 - RNA, 42 nt, synthetic construct
  source 1..42: mol_type = other RNA
ctttcttttcgccatcttttgtctttccgtggagctgtcgcc

SEQ ID NO: 330 - RNA, 19 nt, synthetic construct
  source 1..19: mol_type = other RNA
tttccgtggagctgtcgcc

SEQ ID NO: 331 - RNA, 83 nt, synthetic construct
  source 1..83: mol_type = other RNA
gtgtagtgttttgagtccggcttccatttagcgtagaagcaatttaattttaattagtttttttccaatttagaagataatac

SEQ ID NO: 332 - RNA, 53 nt, synthetic construct
  source 1..53: mol_type = other RNA
aatttgcgcgctctctttctgctgctccccagctctcggatacagccgacacc

SEQ ID NO: 333 - RNA, 23 nt, synthetic construct
  source 1..23: mol_type = other RNA
agtaagggtcgttggtgggaaag

SEQ ID NO: 334 - RNA, 70 nt, synthetic construct
  source 1..70: mol_type = other RNA
agaatataacagcactcccaaagaactgggtactcaacactgagcagatctgttctttgagctaaaaacc

SEQ ID NO: 335 - RNA, 65 nt, synthetic construct
  source 1..65: mol_type = other RNA
taaaatagcccttctgttactgtccggggctgcggggtgagaggccagggcttgcttccggcgtc

SEQ ID NO: 336 - RNA, 73 nt, synthetic construct
  source 1..73: mol_type = other RNA
agaagccaactcagactcagccaacagagattgttgatttgcctcttaagcaagagattcattgcagctcagc

SEQ ID NO: 337 - RNA, 58 nt, synthetic construct
  source 1..58: mol_type = other RNA
gcgagtctctttgcggcgctacactagagcagagtacgagtctgaggcggagggagta

SEQ ID NO: 338 - RNA, 53 nt, synthetic construct
  source 1..53: mol_type = other RNA
attttgtttttgtgtttttgcagggaggagctcttcctgcaggcgtggaaacc

SEQ ID NO: 339 - RNA, 70 nt, synthetic construct
  source 1..70: mol_type = other RNA
gacagctccgcgagccccctacaactcgtttccttccgttcaccttcgcagggcggcgactggcggcgcg

SEQ ID NO: 340 - RNA, 15 nt, synthetic construct
  source 1..15: mol_type = other RNA
aacagttcctagaaa

SEQ ID NO: 341 - RNA, 47 nt, synthetic construct
  source 1..47: mol_type = other RNA
attgcagggtctttctgagttgatttcttatagcacctctcaagaaa

SEQ ID NO: 342 - RNA, 74 nt, synthetic construct
  source 1..74: mol_type = other RNA
aagtagaagaagcccttagcctaagtaagcgataagatttatcagcaagactgttgaacgcataactgcccaag

SEQ ID NO: 343 - RNA, 55 nt, synthetic construct
  source 1..55: mol_type = other RNA
attgcaattccaaaagttgtaagacgggatattctcttctgtgctgtcaagggac

SEQ ID NO: 344 - RNA, 35 nt, synthetic construct
  source 1..35: mol_type = other RNA
agaagcggcaggagcagcgttggcaccggcgaacc

SEQ ID NO: 345 - RNA, 21 nt, synthetic construct
  source 1..21: mol_type = other RNA
aagagaaccctcggcggcaag

SEQ ID NO: 346 - RNA, 53 nt, synthetic construct
  source 1..53: mol_type = other RNA
ctctgcagcataatttaggcattggaggagctgtggttgtaacactctacaag

SEQ ID NO: 347 - RNA, 99 nt, synthetic construct
  source 1..99: mol_type = other RNA
agaaagagtctggtttcgagttttggagatccttgttttttagtgaacacagttctgtaaaattttcataagattccttggcaataacatacgcttgtg

SEQ ID NO: 348 - RNA, 39 nt, synthetic construct
  source 1..39: mol_type = other RNA
attttgcctttttgttaggtttcctaaagacaaaaaaaa

SEQ ID NO: 349 - RNA, 63 nt, synthetic construct
  source 1..63: mol_type = other RNA
agtctcacttcagttccttttgcagtaagagctcagaatcaaaagaggaaaccaacccctaag

SEQ ID NO: 350 - RNA, 59 nt, synthetic construct
  source 1..59: mol_type = other RNA
ctttttctagccaggctctcaactgtctcctgcgttgctgggaagttctggaaggaagc

SEQ ID NO: 351 - RNA, 49 nt, synthetic construct
  source 1..49: mol_type = other RNA
agagctccctgtactttcccaacagtatcgctctttatattcatcgtga

SEQ ID NO: 352 - RNA, 108 nt, synthetic construct
  source 1..108: mol_type = other RNA
agaaggtttccggttccggtgtaacgttcgggctccgtctcaggggctgaagtttgtgaggtgtagtattgagtcctgtttgagctattgttctctttttcctgaaaa

SEQ ID NO: 353 - RNA, 20 nt, synthetic construct
  source 1..20: mol_type = other RNA
accccgtggagcacgccgat

SEQ ID NO: 354 - RNA, 55 nt, synthetic construct
  source 1..55: mol_type = other RNA
attacctggctggtgtttgcttgttctggagtgatcttctgactggaaaagaact

SEQ ID NO: 355 - RNA, 24 nt, synthetic construct
  source 1..24: mol_type = other RNA
cttgccttgcgctgcgcgctcacc

SEQ ID NO: 356 - RNA, 12 nt, synthetic construct
  source 1..12: mol_type = other RNA
agagcagagacc

SEQ ID NO: 357 - RNA, 36 nt, synthetic construct
  source 1..36: mol_type = other RNA
tctgtgcttgaaattctgtgactcttccctgtatct

SEQ ID NO: 358 - RNA, 53 nt, synthetic construct
  source 1..53: mol_type = other RNA
agcctgtgcaagtgatttgggagaggaaggggagagtagttaactggttcggc

SEQ ID NO: 359 - RNA, 20 nt, synthetic construct
  source 1..20: mol_type = other RNA
gagacgcagaggcggacaag

SEQ ID NO: 360 - RNA, 62 nt, synthetic construct
  source 1..62: mol_type = other RNA
acagtggtggggacaacctgagtgctcagtcgtaaagaggaaaggcagaatttttccttgct

SEQ ID NO: 361 - RNA, 74 nt, synthetic construct
  source 1..74: mol_type = other RNA
tagcagaagctctacaagtagtattcattcagtttcttactctgttgtccaggctagagtacagtggcacgatc

SEQ ID NO: 362 - RNA, 63 nt, synthetic construct
  source 1..63: mol_type = other RNA
cttcctcgctttctctgtctctctgtctctctctctctctctccctcgtccactcccccaaac

SEQ ID NO: 363 - RNA, 54 nt, synthetic construct
  source 1..54: mol_type = other RNA
gaggaagccgactaggcgaattcacttactgaccggcctgggctgctctgagac

SEQ ID NO: 364 - RNA, 66 nt, synthetic construct
  source 1..66: mol_type = other RNA
caagttttaagcaagaagaaggaaaaactgacagaagagcttcagaaactacagaaagacatagag

SEQ ID NO: 365 - RNA, 67 nt, synthetic construct
  source 1..67: mol_type = other RNA
accagtaagtcgtgtatttagcattcattcatcaaagcctccggagtcctcctacgtgccctgcact

SEQ ID NO: 366 - RNA, 69 nt, synthetic construct
  source 1..69: mol_type = other RNA
gtaggtgagctcgtgaaacaatagtaagaggagaaaatagccttttaaggaaattggcccacagaaagg

SEQ ID NO: 367 - RNA, 44 nt, synthetic construct
  source 1..44: mol_type = other RNA
caggccctcatcaccttttttcaagtcaagatttcatcccatac

SEQ ID NO: 368 - RNA, 33 nt, synthetic construct
  source 1..33: mol_type = other RNA
ttctttcccgagcttggaacttcgttatccgcg

SEQ ID NO: 369 - RNA, 21 nt, synthetic construct
  source 1..21: mol_type = other RNA
tacgcctcttcattcccgtcc

SEQ ID NO: 370 - RNA, 95 nt, synthetic construct
  source 1..95: mol_type = other RNA
ataagttcctagttagacacaggacctgctgggccacagaaaggaggctctgggtagacgcactagattactggataaatcacttcaatttccca

SEQ ID NO: 371 - RNA, 44 nt, synthetic construct
  source 1..44: mol_type = other RNA
tagctcagtgttgacagctcagagagagaaagatctgagggaag

SEQ ID NO: 372 - RNA, 42 nt, synthetic construct
  source 1..42: mol_type = other RNA
cgctgcactgtctctccgacgcaagactgtcccggcccggat

SEQ ID NO: 373 - RNA, 62 nt, synthetic construct
  source 1..62: mol_type = other RNA
acagaagttagccacctttcttaacggtccgggaaaccagaggtctctccagctactcaggg

SEQ ID NO: 374 - RNA, 64 nt, synthetic construct
  source 1..64: mol_type = other RNA
gtgtatttgaataaaccaggttggcaaatcatactatagctgaaagaattggcaggaactgaaa

SEQ ID NO: 375 - RNA, 93 nt, synthetic construct
  source 1..93: mol_type = other RNA
gtctaagaggcagacagatacattgttcttcactggtcagctgaaagtcaattcatcatcagtgatattcaaaaatcacaagtctttgctggc

SEQ ID NO: 376 - RNA, 55 nt, synthetic construct
  source 1..55: mol_type = other RNA
attgctgcagacgctcaccccagacactcactgcaccggagtgagcgcgaccatc

SEQ ID NO: 377 - RNA, 64 nt, synthetic construct
  source 1..64: mol_type = other RNA
gttgttgtagttccgagagacacgtgaggttctgctacgtcattaccaggcacgcgcaggaaac

SEQ ID NO: 378 - RNA, 97 nt, synthetic construct
  source 1..97: mol_type = other RNA
acaaatcaatccacgggtacagaagtctgacttcccgacactcaatttcacagggtgtctcttgaagagctgccagttttgtaatcaaccacgaacg

SEQ ID NO: 379 - RNA, 12 nt, synthetic construct
  source 1..12: mol_type = other RNA
taattcgccaaa

SEQ ID NO: 380 - RNA, 55 nt, synthetic construct
  source 1..55: mol_type = other RNA
agccttgttcagacgcccagtgacctgccgaggtcggcagcacagagctctggag

SEQ ID NO: 381 - RNA, 53 nt, synthetic construct
  source 1..53: mol_type = other RNA
tatattgagagtgtccacgtctcctcgctgaaccttaggaatcccttggcacc

SEQ ID NO: 382 - RNA, 20 nt, synthetic construct
  source 1..20: mol_type = other RNA
aggcggtggggcccacagcc

SEQ ID NO: 383 - RNA, 59 nt, synthetic construct
  source 1..59: mol_type = other RNA
gtctggctaagtttgacaatagtggaaagtgacaagataaaataattgtgggtagcttt

SEQ ID NO: 384 - RNA, 48 nt, synthetic construct
  source 1..48: mol_type = other RNA
ctggtgtagctcgtccggttaccttcaggaagattggataaccaagaa

SEQ ID NO: 385 - RNA, 72 nt, synthetic construct
  source 1..72: mol_type = other RNA
agttttcttggaggcttttttggtccaatttgtgagatcgatattgttcttaagtagtgggaaaccaggaaa

SEQ ID NO: 386 - RNA, 58 nt, synthetic construct
  source 1..58: mol_type = other RNA
taaatattttttgctttagtttttcttataaggctaaaaattcacaaagcatatatca

SEQ ID NO: 387 - RNA, 47 nt, synthetic construct
  source 1..47: mol_type = other RNA
gagagtcaggtggattcttgggcatttgctattaacttccttccaaa

SEQ ID NO: 388 - RNA, 73 nt, synthetic construct
  source 1..73: mol_type = other RNA
acacgcctttcttattggataccggcttttcagtgggccgagcgccgctgggtaggcggaagtagccgcaggc

SEQ ID NO: 389 - RNA, 14 nt, synthetic construct
  source 1..14: mol_type = other RNA
aagtagccgcaggc

SEQ ID NO: 390 - RNA, 27 nt, synthetic construct
  source 1..27: mol_type = other RNA
ctttcctgcctctgattccgggctgtc

SEQ ID NO: 391 - RNA, 25 nt, synthetic construct
  source 1..25: mol_type = other RNA
acgacaacttccggaagaggtcgcc

SEQ ID NO: 392 - RNA, 87 nt, synthetic construct
  source 1..87: mol_type = other RNA
tgagtttgggtcaatcagtttctcaagttatttaaccagttgtttttaaattttttaatttagttatcaaattgagaatctgagtgt

SEQ ID NO: 393 - RNA, 68 nt, synthetic construct
  source 1..68: mol_type = other RNA
aactgagtcagttgaattacatatctccctgataggtgaaaaccattcactaggagaggagaaacaca

SEQ ID NO: 394 - RNA, 54 nt, synthetic construct
  source 1..54: mol_type = other RNA
agtctgttctgacagtttgagattacttattgtcttttctgggaagacaaaaac

SEQ ID NO: 395 - RNA, 55 nt, synthetic construct
  source 1..55: mol_type = other RNA
gcatcctgcttcgtcaggtttataccactttatttggtgtgctgtgttagtcacc

SEQ ID NO: 396 - RNA, 21 nt, synthetic construct
  source 1..21: mol_type = other RNA
ccggaccctgcagccgcagag

SEQ ID NO: 397 - RNA, 45 nt, synthetic construct
  source 1..45: mol_type = other RNA
acgctgtttttccttttcgttggcgctttatagctacacagtgct

SEQ ID NO: 398 - RNA, 101 nt, synthetic construct
  source 1..101: mol_type = other RNA
gagacagaaggttgtgttggggaactgaaggagtttctgtgggcggacagggaaccctgcgtttctactgtgtgattctgccaccttcctggcccgacgcc

SEQ ID NO: 399 - RNA, 70 nt, synthetic construct
  source 1..70: mol_type = other RNA
aacagtttgagctctggcatagaagaggctggtggctattttgtccttgggctgcctgttttcaggcgcc

SEQ ID NO: 400 - RNA, 54 nt, synthetic construct
  source 1..54: mol_type = other RNA
cggagccgccgagagccttagccgacggaaactggacactggaccggcagcgcc

SEQ ID NO: 401 - RNA, 85 nt, synthetic construct
  source 1..85: mol_type = other RNA
tagtaaagtgttcaagctggcttattcaggtctgctgagatctctactggtttgcttttcagatccctcgtggttcgagaacaga

SEQ ID NO: 402 - RNA, 59 nt, synthetic construct
  source 1..59: mol_type = other RNA
acagcaacttccttgatccctgccacgcacgactgaacacagacagcagccgcctcgcc

SEQ ID NO: 403 - RNA, 68 nt, synthetic construct
  source 1..68: mol_type = other RNA
aaaggatcaagcatccctgagtttcaaacagaaacttgcactgaatacattcaaagaaccatcaagaa

SEQ ID NO: 404 - RNA, 45 nt, synthetic construct
  source 1..45: mol_type = other RNA
ttactttcttactcttgggtcagttcctgctacagctatcccacc

SEQ ID NO: 405 - RNA, 58 nt, synthetic construct
  source 1..58: mol_type = other RNA
ctcaacagcagcacttccgggttgggagaaaggtggcggcgctttcggagggaataaa

SEQ ID NO: 406 - RNA, 54 nt, synthetic construct
  source 1..54: mol_type = other RNA
aacaacatcctgggattgggacacactttctgggcactgctggccagtcccaaa

SEQ ID NO: 407 - RNA, 42 nt, synthetic construct
  source 1..42: mol_type = other RNA
cttcattccattttctgttgagtaataaactcaacgttgaaa

SEQ ID NO: 408 - RNA, 34 nt, synthetic construct
  source 1..34: mol_type = other RNA
acacatccgggcttctgctactagtgagaggaag

SEQ ID NO: 409 - RNA, 12 nt, synthetic construct
  source 1..12: mol_type = other RNA
ccggcgttcaag

SEQ ID NO: 410 - RNA, 27 nt, synthetic construct
  source 1..27: mol_type = other RNA
ccttttttcttttttccggcgttcaag

SEQ ID NO: 411 - RNA, 59 nt, synthetic construct
  source 1..59: mol_type = other RNA
ctcaacactgggtcagaagtcacggccagacaagaaacggggtgttctgagaatcagcc

SEQ ID NO: 412 - RNA, 46 nt, synthetic construct
  source 1..46: mol_type = other RNA
ctgaggagtacagagaacactcttcctgttcctctgcagctgaagg

SEQ ID NO: 413 - RNA, 31 nt, synthetic construct
  source 1..31: mol_type = other RNA
agattcaaggcttcctctctggcacccaacc

SEQ ID NO: 414 - RNA, 39 nt, synthetic construct
  source 1..39: mol_type = other RNA
tccgctgtggtcatttaggaaatcgtaaatcagttgaag

SEQ ID NO: 415 - RNA, 26 nt, synthetic construct
  source 1..26: mol_type = other RNA
tttaggaaatcgtaaatcagttgaag

SEQ ID NO: 416 - RNA, 59 nt, synthetic construct
  source 1..59: mol_type = other RNA
acagtaggtaggaagtctcagaggaaggagcccctccctgggagccagacccccacacc

SEQ ID NO: 417 - RNA, 73 nt, synthetic construct
  source 1..73: mol_type = other RNA
acattgtgtctattgtatcccttggctggtgtatttgtacatctctcgggacgtgaaattgacagtgaaaagt

SEQ ID NO: 418 - RNA, 62 nt, synthetic construct
  source 1..62: mol_type = other RNA
aagtgaataaccaagtttaacctgttttaattgaattgcattttgacgaattatcaataaaa

SEQ ID NO: 419 - RNA, 62 nt, synthetic construct
  source 1..62: mol_type = other RNA
tcagtgaggagattctagttatcaacagaaagaacaaagatcagggctgcctaattgctgac

SEQ ID NO: 420 - RNA, 104 nt, synthetic construct
  source 1..104: mol_type = other RNA
agtggttgattcttcaccacactgaaaccattaggaaaaatccttgtggttaacagcagaggcttcagagtgtaacctgtactcgggcctagaaattatttaaa

SEQ ID NO: 421 - RNA, 46 nt, synthetic construct
  source 1..46: mol_type = other RNA
tatagccttgcagaacttctacttgcctgcctccctgcctctggcc

SEQ ID NO: 422 - RNA, 42 nt, synthetic construct
  source 1..42: mol_type = other RNA
attgcagcctccctgattccagaaggtaccttggtgtctgag

SEQ ID NO: 423 - RNA, 16 nt, synthetic construct
  source 1..16: mol_type = other RNA
atactgccttcctgta

SEQ ID NO: 424 - RNA, 47 nt, synthetic construct
  source 1..47: mol_type = other RNA
ataaatacagcttgactcagccactgtagtactgactccccggggac

SEQ ID NO: 425 - RNA, 66 nt, synthetic construct
  source 1..66: mol_type = other RNA
tctctacctggaaattggtacagagattcagagtctttgtccaggaagacactttggagtagaaga

SEQ ID NO: 426 - RNA, 23 nt, synthetic construct
  source 1..23: mol_type = other RNA
acgcacagttgtgaccatcaagt

SEQ ID NO: 427 - RNA, 48 nt, synthetic construct
  source 1..48: mol_type = other RNA
ttgaaaccgggaagtttgctctaacatttccagagaaggtattaagtc

SEQ ID NO: 428 - RNA, 89 nt, synthetic construct
  source 1..89: mol_type = other RNA
gtgagtctgggattgaattttcctaggactccgtttcaaggttaattcgaaagtcttgtggaagatctggaagaaacaggtattaagtc

SEQ ID NO: 429 - RNA, 56 nt, synthetic construct
  source 1..56: mol_type = other RNA
ctagagagttcagcctacaaaggacacaatctctcttcttcaaattcttccccaaa

SEQ ID NO: 430 - RNA, 51 nt, synthetic construct
  source 1..51: mol_type = other RNA
ctgaacggagtccacacaggcagacttgaccttgaccagatagtcttcaag

SEQ ID NO: 431 - RNA, 16 nt, synthetic construct
  source 1..16: mol_type = other RNA
cctttctcctgcagcc

SEQ ID NO: 432 - RNA, 54 nt, synthetic construct
  source 1..54: mol_type = other RNA
acagcgttgtgcaaagtaagtccttccactgaaccgaggcactgttatagaaga

SEQ ID NO: 433 - RNA, 11 nt, synthetic construct
  source 1..11: mol_type = other RNA
acagaaccgcc

SEQ ID NO: 434 - RNA, 69 nt, synthetic construct
  source 1..69: mol_type = other RNA
aaggaactctatcacaatttgaaaagataagcaaaagtttgatttccagacactacagaagaagtaaaa

SEQ ID NO: 435 - RNA, 94 nt, synthetic construct
  source 1..94: mol_type = other RNA
atagagttgaagtattgataacaccaaggaactctatcacaatttgaaaagataagcaaaagtttgatttccagacactacagaagaagtaaaa

SEQ ID NO: 436 - RNA, 23 nt, synthetic construct
  source 1..23: mol_type = other RNA
ctcttccgtcgcagagtttcgcc

SEQ ID NO: 437 - RNA, 19 nt, synthetic construct
  source 1..19: mol_type = other RNA
ataccttagaccctcagtc

SEQ ID NO: 438 - RNA, 55 nt, synthetic construct
  source 1..55: mol_type = other RNA
acactcgcggcacaggctgacagtattgtcgggggctattctctctcccaggaac

SEQ ID NO: 439 - RNA, 49 nt, synthetic construct
  source 1..49: mol_type = other RNA
agtctagggaaagtcattcagtggagttgatcttggctcacaggggacg

SEQ ID NO: 440 - RNA, 62 nt, synthetic construct
  source 1..62: mol_type = other RNA
acacagcttcttggtgctccaaagacgggcggtaggaggaagatcaaagaagcgagaaagtg

SEQ ID NO: 441 - RNA, 57 nt, synthetic construct
  source 1..57: mol_type = other RNA
attttaggcagctattgcttcagaagtcacagtctgttccaccagcctttgccaagg

SEQ ID NO: 442 - RNA, 12 nt, synthetic construct
  source 1..12: mol_type = other RNA
gttgctgccgcg

SEQ ID NO: 443 - RNA, 21 nt, synthetic construct
  source 1..21: mol_type = other RNA
agcttctcctgggccggcgcc

SEQ ID NO: 444 - RNA, 59 nt, synthetic construct
  source 1..59: mol_type = other RNA
attcatcttccaggctctccttccatcaagtctctcatccctagcgctctgggtcctta

SEQ ID NO: 445 - RNA, 61 nt, synthetic construct
  source 1..61: mol_type = other RNA
gaagagtaacgctactacttctgtttccttttgagtctgctctgacaccccggtgggggaa

SEQ ID NO: 446 - RNA, 52 nt, synthetic construct
  source 1..52: mol_type = other RNA
ccgaacacgagcaccacaggcgcccgaaggccggaacaggcgtttagagaaa

SEQ ID NO: 447 - RNA, 17 nt, synthetic construct
  source 1..17: mol_type = other RNA
agcagagccagagcagg

SEQ ID NO: 448 - RNA, 54 nt, synthetic construct
  source 1..54: mol_type = other RNA
agtctctgaaaactcttcagacactgaggggcaccagaggagcagactacaaga

SEQ ID NO: 449 - RNA, 27 nt, synthetic construct
  source 1..27: mol_type = other RNA
agcttccgggtttcctgggctactacg

SEQ ID NO: 450 - RNA, 24 nt, synthetic construct
  source 1..24: mol_type = other RNA
ctagcggctctccccgcgtccaag

SEQ ID NO: 451 - RNA, 20 nt, synthetic construct
  source 1..20: mol_type = other RNA
gcggggcctctaccggcccg

SEQ ID NO: 452 - RNA, 53 nt, synthetic construct
  source 1..53: mol_type = other RNA
aaaccaaatactcagctgcgttaggaattccttctgattcttgctgcaggaaa

SEQ ID NO: 453 - RNA, 67 nt, synthetic construct
  source 1..67: mol_type = other RNA
gcattgccgtttttgctcagaatcgcgactaaggctttttttttttctccctctgaacggttaggct

SEQ ID NO: 454 - RNA, 65 nt, synthetic construct
  source 1..65: mol_type = other RNA
attgccgtttttgctcagaatcgcgactaaggctttttttttttctccctctgaacggttaggct

SEQ ID NO: 455 - RNA, 93 nt, synthetic construct
  source 1..93: mol_type = other RNA
acttcaagtctttttcttttaacggattgatcttttgctagatagagacaaaatatcagtgtgaattacagcaaacccctattccagtctgtt

SEQ ID NO: 456 - RNA, 56 nt, synthetic construct
  source 1..56: mol_type = other RNA
gtttcagtttgaagaaaatcagtgttggggttgcaggagacctaaacacagtcacc

SEQ ID NO: 457 - RNA, 24 nt, synthetic construct
  source 1..24: mol_type = other RNA
acagtctctgcagagaaatcaaag

SEQ ID NO: 458 - RNA, 55 nt, synthetic construct
  source 1..55: mol_type = other RNA
atctttccagcctcagtcggacgggcgcggagacgcttctggaaggaacgccgcg

SEQ ID NO: 459 - RNA, 93 nt, synthetic construct
  source 1..93: mol_type = other RNA
aaaaaagctctgtgctggctggagccccctcagtgtgcaggcttagagggactaggctgggtgtggagctgcagcgtatccacaggccccagg

SEQ ID NO: 460 - RNA, 60 nt, synthetic construct
  source 1..60: mol_type = other RNA
atcagcaagctccagtgctacgtgtccctggcattttaggtgtcggttgggtaggcagtc

SEQ ID NO: 461 - RNA, 60 nt, synthetic construct
  source 1..60: mol_type = other RNA
gagttctgatttgaagactaatagtaatatatctagtttttcagaagaagtgaagtcaag

SEQ ID NO: 462 - RNA, 11 nt, synthetic construct
  source 1..11: mol_type = other RNA
ttggagagact

SEQ ID NO: 463 - RNA, 24 nt, synthetic construct
  source 1..24: mol_type = other RNA
ccgaaagcctgcagagagcagaac

SEQ ID NO: 464 - RNA, 51 nt, synthetic construct
  source 1..51: mol_type = other RNA
aaaagagcttaaaaagtaaaactgtgctttgtcctagactgttcctttgat

SEQ ID NO: 465 - RNA, 92 nt, synthetic construct
  source 1..92: mol_type = other RNA
aaagcgtggcagcgcaggcgcaagcgcagagagcggaggcggtggtggtggcggccgctggccagtgctggctacagcaaacatcattcaag

SEQ ID NO: 466 - RNA, 46 nt, synthetic construct
  source 1..46: mol_type = other RNA
cagcgcaccaggtgtctttaagagtgattgaagagaataattcaaa

SEQ ID NO: 467 - RNA, 67 nt, synthetic construct
  source 1..67: mol_type = other RNA
ggcgaggagaggcacaccactattgttttaatttgcttttacctgattcctggcagacttctgcgtt

SEQ ID NO: 468 - RNA, 94 nt, synthetic construct
  source 1..94: mol_type = other RNA
tcctattcattctcttgattttttttaaagaaaacttcttttgaaaatttccatccaattccaaacaagctaggacgaacaaagggacctcagc

SEQ ID NO: 469 - RNA, 25 nt, synthetic construct
  source 1..25: mol_type = other RNA
agcgatctccgagcgaggcggcaag

SEQ ID NO: 470 - RNA, 55 nt, synthetic construct
  source 1..55: mol_type = other RNA
aatttaaggctggacacagagtcagcaacacacaagacatcatcttgaaggaagg

SEQ ID NO: 471 - RNA, 97 nt, synthetic construct
  source 1..97: mol_type = other RNA
agtaaaggcaaagtacttttcaaacattctgcagctgggagtacttctttttttaattttttaaaatagttcaaggtgatttgtaatccacagcaaa

SEQ ID NO: 472 - RNA, 55 nt, synthetic construct
  source 1..55: mol_type = other RNA
acagagctagccggacgtcctccgactcaagatatttgtcaaatattttcagaag

SEQ ID NO: 473 - RNA, 76 nt, synthetic construct
  source 1..76: mol_type = other RNA
atattcacccttcagtaggagtagcacagagcaagaagctagccacgctgggctcctcattgcctagctcagttcc

SEQ ID NO: 474 - RNA, 94 nt, synthetic construct
  source 1..94: mol_type = other RNA
tttccaggttaactaaggtctcagctgtaaaccaaaagtgagaggagacattaagattttcattcttaccgggttgtcttcttcctgaagagca

SEQ ID NO: 475 - RNA, 73 nt, synthetic construct
  source 1..73: mol_type = other RNA
aaagcagtggtgttttaccactctgcagagaaagctaaggttgctaaccaaacaaaataactttacactgaca

SEQ ID NO: 476 - RNA, 58 nt, synthetic construct
  source 1..58: mol_type = other RNA
actcaacctctgaagtttagtgacactattgggacgaaaaaggactgtgctggcccaa

SEQ ID NO: 477 - RNA, 92 nt, synthetic construct
  source 1..92: mol_type = other RNA
ggaaaaagcagtacatttagaagtagaagacttagcttcaaatccctactccttcacttactaattttgtgatttggaaatatccgcgcaag

SEQ ID NO: 478 - RNA, 48 nt, synthetic construct
  source 1..48: mol_type = other RNA
cagtgcaactgaaacttttgcattactatactactgagaatatctaac

SEQ ID NO: 479 - RNA, 24 nt, synthetic construct
  source 1..24: mol_type = other RNA
ttttgtcttccttgaacagccaca

SEQ ID NO: 480 - RNA, 53 nt, synthetic construct
  source 1..53: mol_type = other RNA
gcaaatactagtagaaaaggttattggaattctgttcggtgtttaagccagca

SEQ ID NO: 481 - RNA, 92 nt, synthetic construct
  source 1..92: mol_type = other RNA
acacattttaggtagttgattagaaaacatacttgtcagaattgtctggctggattaatttgctaatttgaccttcttcatcatttgagttg

SEQ ID NO: 482 - RNA, 52 nt, synthetic construct
  source 1..52: mol_type = other RNA
cttcaagattatcaataatcggagatacgtatattttatttgtaaagaaaac

SEQ ID NO: 483 - RNA, 42 nt, synthetic construct
  source 1..42: mol_type = other RNA
cagggcccgtgttgtgcttttggcccaggtaggtggacagac

SEQ ID NO: 484 - RNA, 57 nt, synthetic construct
  source 1..57: mol_type = other RNA
ttcaaagaatatagagtgttttgaaaagttcagtctgtcccttcattgaattttaga

SEQ ID NO: 485 - RNA, 21 nt, synthetic construct
  source 1..21: mol_type = other RNA
attctcagcgctgattttgag

SEQ ID NO: 486 - RNA, 46 nt, synthetic construct
  source 1..46: mol_type = other RNA
acatatcccaggaggcaagtcgcaggggagcacggaatcgtgaaaa

SEQ ID NO: 487 - RNA, 53 nt, synthetic construct
  source 1..53: mol_type = other RNA
ggagacagagatcccaggacaaagttgcagcaggcccctgagcagttgtagaa

SEQ ID NO: 488 - RNA, 21 nt, synthetic construct
  source 1..21: mol_type = other RNA
cagatagatttttctcccagc

SEQ ID NO: 489 - RNA, 44 nt, synthetic construct
  source 1..44: mol_type = other RNA
gttgcttggacttggccctattctgtatcaggtcggagcagagc

SEQ ID NO: 490 - RNA, 61 nt, synthetic construct
  source 1..61: mol_type = other RNA
ttctcaccctgcctttgttactgggaaaccgaacgcggcgctgtggctttcagcttggatc

SEQ ID NO: 491 - RNA, 55 nt, synthetic construct
  source 1..55: mol_type = other RNA
ccctgcctttgttactgggaaaccgaacgcggcgctgtggctttcagcttggatc

SEQ ID NO: 492 - RNA, 93 nt, synthetic construct
  source 1..93: mol_type = other RNA
gatatttggtctttgcctggctgagggacacaaagcaagaagagggagacagtcagatagtttgcaggatattcaagaaagaaggattttagc

SEQ ID NO: 493 - RNA, 45 nt, synthetic construct
  source 1..45: mol_type = other RNA
aatactgggatcttcagtggcaggaggagtaatcagaagacggag

SEQ ID NO: 494 - RNA, 114 nt, synthetic construct
  source 1..114: mol_type = other RNA
atttccctttttaccaagaagaaagatttgttcagcctggtgacgcttcagaagagaagagtttaacgacattgatattttgaaatattttgtgactcaaattatagcaatacg

SEQ ID NO: 495 - RNA, 59 nt, synthetic construct
  source 1..59: mol_type = other RNA
actgttttgcaaacatagcatcaacatagaagttattttgtaggctccaggagtcagcg

SEQ ID NO: 496 - RNA, 56 nt, synthetic construct
  source 1..56: mol_type = other RNA
aacgcttccttcgcggggctttgtgggtagccgactggggtctcctggcgacgacc

SEQ ID NO: 497 - RNA, 12 nt, synthetic construct
  source 1..12: mol_type = other RNA
tagagagaggaa

SEQ ID NO: 498 - RNA, 44 nt, synthetic construct
  source 1..44: mol_type = other RNA
cagagagttcggcttatttataggaattgcttgaagccagagtc

SEQ ID NO: 499 - RNA, 37 nt, synthetic construct
  source 1..37: mol_type = other RNA
cagtcattcccttgtagggacaactagaagaaatttt

SEQ ID NO: 500 - RNA, 90 nt, synthetic construct
  source 1..90: mol_type = other RNA
agaaattctgcaaaataagttggtctctaggatacataagttgcattttagtaattccttattttaacccctgctttaggaatcagtaac

SEQ ID NO: 501 - RNA, 33 nt, synthetic construct
  source 1..33: mol_type = other RNA
agtcggtgaagggcgagcggcgcggcggctgcg

SEQ ID NO: 502 - RNA, 57 nt, synthetic construct
  source 1..57: mol_type = other RNA
ctcttcctgagaaacgagcaaacctgaaagctactctctcagcttcagagggaaaaa

SEQ ID NO: 503 - RNA, 42 nt, synthetic construct
  source 1..42: mol_type = other RNA
atttactccatcctttatagtgagtctacaggacgaagagga

SEQ ID NO: 504 - RNA, 92 nt, synthetic construct
  source 1..92: mol_type = other RNA
atcactgaccttggaagctgctggagccacgattcagtcccctggactgtagataaagaccctttcttgccaggtgctgagacaaccacact

SEQ ID NO: 505 - RNA, 27 nt, synthetic construct
  source 1..27: mol_type = other RNA
accaacgctgtcgccacctctgtagcc

SEQ ID NO: 506 - RNA, 17 nt, synthetic construct
  source 1..17: mol_type = other RNA
actgctttcttgggaag

SEQ ID NO: 507 - RNA, 61 nt, synthetic construct
  source 1..61: mol_type = other RNA
agacagttcctgcagcgcttaccgcctggcctctcggttccgcggcgcaccggagggcagc

SEQ ID NO: 508 - RNA, 34 nt, synthetic construct
  source 1..34: mol_type = other RNA
acacgctgcaaaaggctttccaagaatcctcgac

SEQ ID NO: 509 - RNA, 15 nt, synthetic construct
  source 1..15: mol_type = other RNA
acagaatactgaaaa

SEQ ID NO: 510 - RNA, 57 nt, synthetic construct
  source 1..57: mol_type = other RNA
acagagctgcagctcttcagggaagaaatcaaaacaagatcacaagaatactgaaaa

SEQ ID NO: 511 - RNA, 53 nt, synthetic construct
  source 1..53: mol_type = other RNA
cgcctcttctctttttgggaacgtggcgagcaaaaaagtcttgagccagagtg

SEQ ID NO: 512 - RNA, 48 nt, synthetic construct
  source 1..48: mol_type = other RNA
ttcaaggttacccatctcaagtagcctagcaatattggcaacatccca

SEQ ID NO: 513 - RNA, 23 nt, synthetic construct
  source 1..23: mol_type = other RNA
agtgtcgatccctcagccagggc

SEQ ID NO: 514 - RNA, 19 nt, synthetic construct
  source 1..19: mol_type = other RNA
ccgggcgagcgcagccaac

SEQ ID NO: 515 - RNA, 24 nt, synthetic construct
  source 1..24: mol_type = other RNA
attgagatcacatagccaactctg

SEQ ID NO: 516 - RNA, 126 nt, synthetic construct
  source 1..126: mol_type = other RNA
attacacattcaaagtatccatttgttctcaagaggtagcaagaaacagtatccacagtggactccggggctcctacagacttggcacagcttcctacagtcttgaaacagccctgttgttctgtc

SEQ ID NO: 517 - RNA, 10 nt, synthetic construct
  source 1..10: mol_type = other RNA
atttctcgcc

SEQ ID NO: 518 - RNA, 18 nt, synthetic construct
  source 1..18: mol_type = other RNA
acactacgccagaacaag

SEQ ID NO: 519 - RNA, 93 nt, synthetic construct
  source 1..93: mol_type = other RNA
aacgaagaggcacacaggaggtcggagaacacaatatcaacagggccagcgggtgaggtaccccaggtaccagacaaggaaaccaaagccaca

SEQ ID NO: 520 - RNA, 58 nt, synthetic construct
  source 1..58: mol_type = other RNA
agaagaggacgttgtcacagataaagagccaggctcaccagctcctgacgcagtcatc

SEQ ID NO: 521 - RNA, 11 nt, synthetic construct
  source 1..11: mol_type = other RNA
gtgatcagctc

SEQ ID NO: 522 - RNA, 55 nt, synthetic construct
  source 1..55: mol_type = other RNA
attattttctaaattctgtgctaacagaaccggcttcagctgaagaaagagagga

SEQ ID NO: 523 - RNA, 44 nt, synthetic construct
  source 1..44: mol_type = other RNA
atactttcagtttcagtcacacaagaagggaggagagaaaagcc

SEQ ID NO: 524 - RNA, 47 nt, synthetic construct
  source 1..47: mol_type = other RNA
cagtaaataaaaacacaactattgattcagagaaaggaaaattgatc

SEQ ID NO: 525 - RNA, 16 nt, synthetic construct
  source 1..16: mol_type = other RNA
cctttccctgttagac

SEQ ID NO: 526 - RNA, 96 nt, synthetic construct
  source 1..96: mol_type = other RNA
aacagttaatcctcaatttaagcctgatctaacccctagaaacagtataggatatattctgttaaagagtgaaaaaagtgaaaatctctgccattt

SEQ ID NO: 527 - RNA, 53 nt, synthetic construct
  source 1..53: mol_type = other RNA
actcagaggccgtccaagacactggcaagccgcagaagcccagttcgccggcc

SEQ ID NO: 528 - RNA, 30 nt, synthetic construct
  source 1..30: mol_type = other RNA
acagagcaggttcttttacagggagccacc

SEQ ID NO: 529 - RNA, 46 nt, synthetic construct
  source 1..46: mol_type = other RNA
gtcacctctgacaaaattctggggacgctgggaacactgaatcaac

SEQ ID NO: 530 - RNA, 72 nt, synthetic construct
  source 1..72: mol_type = other RNA
attgtttgaatttgaaaacggtaacatcgcagtgctgctcgcgggtctggctagtcaggcgaagtttgcaga

SEQ ID NO: 531 - RNA, 126 nt, synthetic construct
  source 1..126: mol_type = other RNA
gcaggagtctacacccaggcggtggctatacttattaaagttggcgggagctgagcgatcagtcggtcctgggctggctgtggtgtagggcgttgcaggcagcgctcacctcctcaggccggcaga

SEQ ID NO: 532 - RNA, 92 nt, synthetic construct
  source 1..92: mol_type = other RNA
gcatttatatagcagagtaagaatcaggaaagaagcacttgtaaggaaatatagcatccattgtgaaagtggaaaagtaaagataattcatc

SEQ ID NO: 533 - RNA, 55 nt, synthetic construct
  source 1..55: mol_type = other RNA
ctcagtcaagctgaagtgaaaacgagaccaaggtctagctctactgttggtactt

SEQ ID NO: 534 - RNA, 45 nt, synthetic construct
  source 1..45: mol_type = other RNA
gaaatcttttaggcctttagagtttgttctggaaaaactttagta

SEQ ID NO: 535 - RNA, 23 nt, synthetic construct
  source 1..23: mol_type = other RNA
acattgactgctctgctgctgca

SEQ ID NO: 536 - RNA, 75 nt, synthetic construct
  source 1..75: mol_type = other RNA
gggccctgacagttgaaaggaaggaagttgccctaatattctacagttgttttatcgttgctactgattaggtcc

SEQ ID NO: 537 - RNA, 60 nt, synthetic construct
  source 1..60: mol_type = other RNA
aaagaatttctcaggctcaaaatccaatacaggagtgacttggaactccattctatcact

SEQ ID NO: 538 - RNA, 13 nt, synthetic construct
  source 1..13: mol_type = other RNA
attgtgggaaaag

SEQ ID NO: 539 - RNA, 93 nt, synthetic construct
  source 1..93: mol_type = other RNA
aagtctgttcagctgcctgtttgaagaaagtttattttttaaaaactagttttgcagttggctgaagagagacagtgaaatattgaagaacgt

SEQ ID NO: 540 - RNA, 92 nt, synthetic construct
  source 1..92: mol_type = other RNA
acttccgggctcggcagctagactagcgggggcagcctcagagcggctacctgtgcaccctgcctccggctctcctgagcagagagatcctg

SEQ ID NO: 541 - RNA, 14 nt, synthetic construct
  source 1..14: mol_type = other RNA
tagggccgctgtgg

SEQ ID NO: 542 - RNA, 56 nt, synthetic construct
  source 1..56: mol_type = other RNA
gtcttggagagaggctgtgtgcattagctacaaatagtaacattttcaaaatcaac

SEQ ID NO: 543 - RNA, 92 nt, synthetic construct
  source 1..92: mol_type = other RNA
actgaagacttactccctttgcctgtgtggatatagttcttggagagaggctgtgtgcattagctacaaatagtaacattttcaaaatcaac

SEQ ID NO: 544 - RNA, 59 nt, synthetic construct
  source 1..59: mol_type = other RNA
gagtcatactccagtggtttagttgataaagtatcttggcatagaagagaatttaaaga

SEQ ID NO: 545 - RNA, 62 nt, synthetic construct
  source 1..62: mol_type = other RNA
agtctcctttaaggagagcttgtaggcttctaatttcacatttcagcaagctggctaaagac

SEQ ID NO: 546 - RNA, 42 nt, synthetic construct
  source 1..42: mol_type = other RNA
ccgctgaagtggagcaggtacagtcacagctgtggggacagc

SEQ ID NO: 547 - RNA, 53 nt, synthetic construct
  source 1..53: mol_type = other RNA
gtgaggccgaggtgactgcagagcggctcgcgagttctgtgatagtagcaaca

SEQ ID NO: 548 - RNA, 124 nt, synthetic construct
  source 1..124: mol_type = other RNA
tccacgtttctttagcgggcagtggtaaaaagagtatttaagatcggtcgagtttgagtggctgaggcagttttggtttcaaggcaatcgatacagaaagtgcactgactggtggtgtcaaccc

SEQ ID NO: 549 - RNA, 70 nt, synthetic construct
  source 1..70: mol_type = other RNA
gtggtttcctagaaacagtattgtttattggcattgatctcacagtctggtgaggacttctttactgata

SEQ ID NO: 550 - RNA, 44 nt, synthetic construct
  source 1..44: mol_type = other RNA
gcctaaagtaaaattcagctcgaagtacagcagtataaagacag

SEQ ID NO: 551 - RNA, 34 nt, synthetic construct
  source 1..34: mol_type = other RNA
caataaagttttatatttattttgtgaaggtgcc

SEQ ID NO: 552 - RNA, 28 nt, synthetic construct
  source 1..28: mol_type = other RNA
acttgccgatagtggtgacggcggcaac

SEQ ID NO: 553 - RNA, 85 nt, synthetic construct
  source 1..85: mol_type = other RNA
agcgatatccggacacagatcacagcgttctttctgtttgtttgcaggggctggtgagtctgggaaaagcactatcgtcaaacag

SEQ ID NO: 554 - RNA, 25 nt, synthetic construct
  source 1..25: mol_type = other RNA
tcagaaccatcaagtagagtcaaac

SEQ ID NO: 555 - RNA, 46 nt, synthetic construct
  source 1..46: mol_type = other RNA
caaccctgaagttcatagttagctactttcaactggaagctacaac

SEQ ID NO: 556 - RNA, 60 nt, synthetic construct
  source 1..60: mol_type = other RNA
actcgcctcattttctctaagcagagcaacttcgcggacagcggagctcgcccagcagtg

SEQ ID NO: 557 - RNA, 23 nt, synthetic construct
  source 1..23: mol_type = other RNA
actgggcggaccgcggcggcagc

SEQ ID NO: 558 - RNA, 37 nt, synthetic construct
  source 1..37: mol_type = other RNA
actttcactcaccgcctgtccttcctgacacctcacc

SEQ ID NO: 559 - RNA, 51 nt, synthetic construct
  source 1..51: mol_type = other RNA
gtgacagagcgctttacgacagttgctttgaggcagtaccggaggagaaag

SEQ ID NO: 560 - RNA, 19 nt, synthetic construct
  source 1..19: mol_type = other RNA
ctgggagtcgttagtgagg

SEQ ID NO: 561 - RNA, 62 nt, synthetic construct
  source 1..62: mol_type = other RNA
acatttgcgggaacgcagagcggagcgtggagagcggagcgaagctggataacaggggaccg

SEQ ID NO: 562 - RNA, 57 nt, synthetic construct
  source 1..57: mol_type = other RNA
agtgtgtggctgcttctggactcaaggaggaggagagagattccgcgagccgacacc

SEQ ID NO: 563 - RNA, 18 nt, synthetic construct
  source 1..18: mol_type = other RNA
ctcttcctgctctccatc

SEQ ID NO: 564 - RNA, 92 nt, synthetic construct
  source 1..92: mol_type = other RNA
agtccttggcgtttcctttggcggattttctgttttcggaagttgctgggttcgttttattcagcggcagtggtgctttcccgaatctcaga

SEQ ID NO: 565 - RNA, 61 nt, synthetic construct
  source 1..61: mol_type = other RNA
gttttcggaagttgctgggttcgttttattcagcggcagtggtgctttcccgaatctcaga

SEQ ID NO: 566 - RNA, 91 nt, synthetic construct
  source 1..91: mol_type = other RNA
acagtcatagctcttagcttctgtgtaagaagttgtgagctccttctggaaacatttgcagttacattaagtaaagtgtaaagtcacagta

SEQ ID NO: 567 - RNA, 41 nt, synthetic construct
  source 1..41: mol_type = other RNA
gttattttgagcaatagtttttggaaaggttggttttcatc

SEQ ID NO: 568 - RNA, 18 nt, synthetic construct
  source 1..18: mol_type = other RNA
acgagcctttccggtgct

SEQ ID NO: 569 - RNA, 20 nt, synthetic construct
  source 1..20: mol_type = other RNA
ctcttgcgccctggtccaag

SEQ ID NO: 570 - RNA, 11 nt, synthetic construct
  source 1..11: mol_type = other RNA
acagtgctggg

SEQ ID NO: 571 - RNA, 42 nt, synthetic construct
  source 1..42: mol_type = other RNA
agaagtgtctgacaaaacaaagaaacaaaacctcagtgaaaa

SEQ ID NO: 572 - RNA, 63 nt, synthetic construct
  source 1..63: mol_type = other RNA
caaagactggctctgctgttaagaagttgtacttaaagcggaggagctaagccacctgccaaa

SEQ ID NO: 573 - RNA, 37 nt, synthetic construct
  source 1..37: mol_type = other RNA
agaattgttaaaatctttgtgaagcacaccactgaag

SEQ ID NO: 574 - RNA, 85 nt, synthetic construct
  source 1..85: mol_type = other RNA
agaactgagctcatctttgcggggttagctagccgttgagtccaagtctccttggcaagacagtagcttagagaaacgagatcga

SEQ ID NO: 575 - RNA, 45 nt, synthetic construct
  source 1..45: mol_type = other RNA
ctctttacacagaacagtttaacttttgtgcttctgggcagaggt

SEQ ID NO: 576 - RNA, 12 nt, synthetic construct
  source 1..12: mol_type = other RNA
ctgggcagaggt

SEQ ID NO: 577 - RNA, 42 nt, synthetic construct
  source 1..42: mol_type = other RNA
aaaaagagcccttgaagctggaagacgggagaggacaaaagc

SEQ ID NO: 578 - RNA, 93 nt, synthetic construct
  source 1..93: mol_type = other RNA
aagagagaatctcttatagttgctgatacaggtttcaactttctgtgctgtggggtcagtggtaagagagcagcatctggaatcaagagaccc

SEQ ID NO: 579 - RNA, 17 nt, synthetic construct
  source 1..17: mol_type = other RNA
attctgtgcctgtgaac

SEQ ID NO: 580 - RNA, 85 nt, synthetic construct
  source 1..85: mol_type = other RNA
attttcgagttggtgagacaccgcggcagtcgacttggaagagtctctggtggtagaggtggacttagtagttttaagcagaacc

SEQ ID NO: 581 - RNA, 70 nt, synthetic construct
  source 1..70: mol_type = other RNA
gtgagaggaggctgcaacgccgagcggaggaggcaggaaccggagcgcgagcagtagctgggtgggcacc

SEQ ID NO: 582 - RNA, 61 nt, synthetic construct
  source 1..61: mol_type = other RNA
acacatctcactcatccttctactcgtgacgcttcccagctctggctttttgaaagcaaag

SEQ ID NO: 583 - RNA, 61 nt, synthetic construct
  source 1..61: mol_type = other RNA
gtcagtgtggaggagactgagtattctaccttgtaaatactgttatttgtatatactgtaa

SEQ ID NO: 584 - RNA, 64 nt, synthetic construct
  source 1..64: mol_type = other RNA
ggaaggaagtgtttttaaaactacgtgaggcatcagaatccgaaagccactttagtcttagcaa

SEQ ID NO: 585 - RNA, 12 nt, synthetic construct
  source 1..12: mol_type = other RNA
gcctccaggatc

SEQ ID NO: 586 - RNA, 51 nt, synthetic construct
  source 1..51: mol_type = other RNA
cctcacaccaacaactgcacctactttcttacagggttggagtcctataac

SEQ ID NO: 587 - RNA, 58 nt, synthetic construct
  source 1..58: mol_type = other RNA
gtgtaagacactgaagacactgcagagcaaggtgcttattccagaggcgttacaaaac

SEQ ID NO: 588 - RNA, 86 nt, synthetic construct
  source 1..86: mol_type = other RNA
tagactataagcttttggaaaacagggactagttcttcttcaacaagttgtacacaacaagtagcaggttgtttggcacagtgcag

SEQ ID NO: 589 - RNA, 82 nt, synthetic construct
  source 1..82: mol_type = other RNA
acagtcttgagtcattcattctcaaggacacttgatccactgccagagaggcccagaattttctaacttactgtgtggcaga

SEQ ID NO: 590 - RNA, 53 nt, synthetic construct
  source 1..53: mol_type = other RNA
agtagcgggcggcagagctggagtgaagggagctagtggtagcagtcaccatt

SEQ ID NO: 591 - RNA, 15 nt, synthetic construct
  source 1..15: mol_type = other RNA
tctttccgggacaac

SEQ ID NO: 592 - RNA, 10 nt, synthetic construct
  source 1..10: mol_type = other RNA
agagaagaag

SEQ ID NO: 593 - RNA, 61 nt, synthetic construct
  source 1..61: mol_type = other RNA
accacttttacagtgtcacctacaggagagaggaggtgctgcaagactctctggtagaaaa

SEQ ID NO: 594 - RNA, 95 nt, synthetic construct
  source 1..95: mol_type = other RNA
actgttttttcaaagagattgcttactccaccaggagaagtatatctacctattctaccactggtgagtatctggactagtccaactggtaggaa

SEQ ID NO: 595 - RNA, 53 nt, synthetic construct
  source 1..53: mol_type = other RNA
gtacaggcggcacggtttgagtgcagagatattttctttccaaactgttcaaa

SEQ ID NO: 596 - RNA, 53 nt, synthetic construct
  source 1..53: mol_type = other RNA
ggtttaattcttcagatcacattgagagtaaaggtattaaaataaaggagact

SEQ ID NO: 597 - RNA, 47 nt, synthetic construct
  source 1..47: mol_type = other RNA
tattttcagaatttcagaaattgaaaagggataagaggaaggagaaa

SEQ ID NO: 598 - RNA, 30 nt, synthetic construct
  source 1..30: mol_type = other RNA
ctatattctctgttctgcattataagcaac

SEQ ID NO: 599 - RNA, 93 nt, synthetic construct
  source 1..93: mol_type = other RNA
acaagcgccttacgaaaggcggcaaaaagggagccaagaagaaaggttgatccattttctaagaaagattggtagtagttgaaagcacctgct

SEQ ID NO: 600 - RNA, 37 nt, synthetic construct
  source 1..37: mol_type = other RNA
ctcttccggtctttctggtctcggccgcagaagcgag

SEQ ID NO: 601 - RNA, 92 nt, synthetic construct
  source 1..92: mol_type = other RNA
tataggttttaaaagtaattccaagacaccattacaaagaaagccggactcttttcttataactgagctcagccaaggaaactcttgcacaa

SEQ ID NO: 602 - RNA, 69 nt, synthetic construct
  source 1..69: mol_type = other RNA
agcagcttgtattcttagaaacgcagcagacagcaactttagcaggtgatttcaatcaagtcagctgaa

SEQ ID NO: 603 - RNA, 52 nt, synthetic construct
  source 1..52: mol_type = other RNA
acgagaggaagcttaaatctgtcgtttgaatttaggaccacctcggttcaca

SEQ ID NO: 604 - RNA, 61 nt, synthetic construct
  source 1..61: mol_type = other RNA
agtgtcgagtcagctgtagtgacaatctcagagcagcttctacaccacagccatttccagc

SEQ ID NO: 605 - RNA, 39 nt, synthetic construct
  source 1..39: mol_type = other RNA
cttttctgaaaacatcagcattctgccatatctggaata

SEQ ID NO: 606 - RNA, 14 nt, synthetic construct
  source 1..14: mol_type = other RNA
acttcgctgtgaag

SEQ ID NO: 607 - RNA, 62 nt, synthetic construct
  source 1..62: mol_type = other RNA
tagtagtaggctcccttcccacctcagtcattggtttgctcgctcccttagttgagtaatca

SEQ ID NO: 608 - RNA, 29 nt, synthetic construct
  source 1..29: mol_type = other RNA
tctttctctctttctcattctctttcaat

SEQ ID NO: 609 - RNA, 54 nt, synthetic construct
  source 1..54: mol_type = other RNA
ggaaacttatttaaggagtgctgacttaccgtatcaggtgataagaagtacatc

SEQ ID NO: 610 - RNA, 92 nt, synthetic construct
  source 1..92: mol_type = other RNA
ttttagtggtggagtcaatttatttctgaaacgatctcatttacctgaagtaggagctcatatttattttcaggatttagtaggaatccaag

SEQ ID NO: 611 - RNA, 24 nt, synthetic construct
  source 1..24: mol_type = other RNA
actcaagttcggaacgttccgaag

SEQ ID NO: 612 - RNA, 95 nt, synthetic construct
  source 1..95: mol_type = other RNA
gatcgcttcaggttccaaaagtacaacagttttacagtcgtcgtagcatcagattaagttggcagcctgagtctctctagagaagacacggttca

SEQ ID NO: 613 - RNA, 42 nt, synthetic construct
  source 1..42: mol_type = other RNA
gtagattagctcaacactcgagaatcattgcagcttcaagga

SEQ ID NO: 614 - RNA, 67 nt, synthetic construct
  source 1..67: mol_type = other RNA
gagcacggaacttattcctgcccagagttcagacacagccagaaattaccatttcatacttcacaga

SEQ ID NO: 615 - RNA, 53 nt, synthetic construct
  source 1..53: mol_type = other RNA
aaaattagaaaatttaaacttcttcagtaggacgtgttggaacacctcatttt

SEQ ID NO: 616 - RNA, 92 nt, synthetic construct
  source 1..92: mol_type = other RNA
gcggccgatcagaggagcaggaactggaaccctggccgagtccgaaaaaagccagatctggaaggtggctgcggaacggttttaagcggaag

SEQ ID NO: 617 - RNA, 57 nt, synthetic construct
  source 1..57: mol_type = other RNA
ccgagtccgaaaaaagccagatctggaaggtggctgcggaacggttttaagcggaag

SEQ ID NO: 618 - RNA, 46 nt, synthetic construct
  source 1..46: mol_type = other RNA
gcagctgagcgttggaagccattttggctgcaacctacctgaagcg

SEQ ID NO: 619 - RNA, 57 nt, synthetic construct
  source 1..57: mol_type = other RNA
attcggcagccagactccttgaaataccctttcagtaatcattcaaccaacgcttcc

SEQ ID NO: 620 - RNA, 62 nt, synthetic construct
  source 1..62: mol_type = other RNA
acagtactcaacctacttcaagggaagattgggatctttttggctctctgcctctaaacaga

SEQ ID NO: 621 - RNA, 18 nt, synthetic construct
  source 1..18: mol_type = other RNA
aagaggactccaagcgcc

SEQ ID NO: 622 - RNA, 12 nt, synthetic construct
  source 1..12: mol_type = other RNA
actccaagcgcc

SEQ ID NO: 623 - RNA, 21 nt, synthetic construct
  source 1..21: mol_type = other RNA
cctctttttccggctggaacc

SEQ ID NO: 624 - RNA, 25 nt, synthetic construct
  source 1..25: mol_type = other RNA
tctttcttcctgtctgcttggaaag

SEQ ID NO: 625 - RNA, 21 nt, synthetic construct
  source 1..21: mol_type = other RNA
aagagacgcttccttctgatc

SEQ ID NO: 626 - RNA, 61 nt, synthetic construct
  source 1..61: mol_type = other RNA
acctctggtgccaaagggcggcgcagcggctgccgagctcggccctggaggcggcgagaac

SEQ ID NO: 627 - RNA, 53 nt, synthetic construct
  source 1..53: mol_type = other RNA
cggcgtgttggtcccagcggttcagctgaggtagggacgtgccgtaggccgga

SEQ ID NO: 628 - RNA, 44 nt, synthetic construct
  source 1..44: mol_type = other RNA
gtcttattctttttagtggagttttccaaggattgtcttcagtc

SEQ ID NO: 629 - RNA, 44 nt, synthetic construct
  source 1..44: mol_type = other RNA
cagtgctgcaatagaacttttcagcagactacaagaagcagtaa

SEQ ID NO: 630 - RNA, 12 nt, synthetic construct
  source 1..12: mol_type = other RNA
gtctgccgggcc

SEQ ID NO: 631 - RNA, 97 nt, synthetic construct
  source 1..97: mol_type = other RNA
cattataagttcagtagtacaacattttgtgagagaagtctgtataaaaagcaattgctcttttgtttccaaaacaggtattacaagtggataaata

SEQ ID NO: 632 - RNA, 50 nt, synthetic construct
  source 1..50: mol_type = other RNA
attcactcgctcttttcattcacgaaggtagtgaggcctagtggaaagcc

SEQ ID NO: 633 - RNA, 33 nt, synthetic construct
  source 1..33: mol_type = other RNA
attcacgaaggtagtgaggcctagtggaaagcc

SEQ ID NO: 634 - RNA, 11 nt, synthetic construct
  source 1..11: mol_type = other RNA
attccgtgttc

SEQ ID NO: 635 - RNA, 25 nt, synthetic construct
  source 1..25: mol_type = other RNA
aagctgagagggcccgcgggtaggc

SEQ ID NO: 636 - RNA, 19 nt, synthetic construct
  source 1..19: mol_type = other RNA
ttagggattccggttcaca

SEQ ID NO: 637 - RNA, 60 nt, synthetic construct
  source 1..60: mol_type = other RNA
ggaaagtcacaagcctcttgagtcataaaaacagctgggctcccttggagacagagcgcc

SEQ ID NO: 638 - RNA, 25 nt, synthetic construct
  source 1..25: mol_type = other RNA
gcattcccttgggcagagagtggag

SEQ ID NO: 639 - RNA, 55 nt, synthetic construct
  source 1..55: mol_type = other RNA
tggcatcttccttactttgtccatcctccggactcgcgatcttccttccggagcc

SEQ ID NO: 640 - RNA, 63 nt, synthetic construct
  source 1..63: mol_type = other RNA
acaaggaggcaggcaagacagcaaggcatagagacaacatagagctaagtaaagccagtggaa

SEQ ID NO: 641 - RNA, 61 nt, synthetic construct
  source 1..61: mol_type = other RNA
aaggaggcaggcaagacagcaaggcatagagacaacatagagctaagtaaagccagtggaa

SEQ ID NO: 642 - RNA, 64 nt, synthetic construct
  source 1..64: mol_type = other RNA
atttcagtttatacttaataaaacaaaacatacctgtatacacacacattcactcacattgaag

SEQ ID NO: 643 - RNA, 94 nt, synthetic construct
  source 1..94: mol_type = other RNA
aagtaaaagtggtcaaacaacttaacagtgtgccagttttcctaagcgtgcttgccactgattgtaagtcttttattgtttggcttcttgataa

SEQ ID NO: 644 - RNA, 19 nt, synthetic construct
  source 1..19: mol_type = other RNA
ctttctcagaaaaccaaat

SEQ ID NO: 645 - RNA, 53 nt, synthetic construct
  source 1..53: mol_type = other RNA
tgtctttaaaaaataactgtatactaagcaaaactcaggtcttaaaaataaat

SEQ ID NO: 646 - RNA, 89 nt, synthetic construct
  source 1..89: mol_type = other RNA
ggagtgtgtctgaggaaacacatcccggacaccacttagggttagtctttctgagctcttcacatctctgactaattatcatcattacc

SEQ ID NO: 647 - RNA, 55 nt, synthetic construct
  source 1..55: mol_type = other RNA
ttcctacgacaaaagatacatcatcaagactattaccagtgaagacgtggccgaa

SEQ ID NO: 648 - RNA, 51 nt, synthetic construct
  source 1..51: mol_type = other RNA
cttccctggccttgccctctctctttctgccctgtagccgcgggcgtccaa

SEQ ID NO: 649 - RNA, 62 nt, synthetic construct
  source 1..62: mol_type = other RNA
gtctgacggcacttaaggtctaaaaaagaaggaagttgtttctgatcatctgaatcttaatc

SEQ ID NO: 650 - RNA, 44 nt, synthetic construct
  source 1..44: mol_type = other RNA
ctgtggtttaagggactgtgctcggggaagacgtgctgtcagcg

SEQ ID NO: 651 - RNA, 62 nt, synthetic construct
  source 1..62: mol_type = other RNA
agctggggagtttgcagatctgatctgtcagcggggactgagagcaaatcgggacattcatc

SEQ ID NO: 652 - RNA, 60 nt, synthetic construct
  source 1..60: mol_type = other RNA
tcacaatcacattgagccaaaacgcatccagtgttttctccagttacaaataaaacgaat

SEQ ID NO: 653 - RNA, 64 nt, synthetic construct
  source 1..64: mol_type = other RNA
gataatcttttccttcttattcattgcagaagcttcaaaaaggacacactagatttaattagaa

SEQ ID NO: 654 - RNA, 48 nt, synthetic construct
  source 1..48: mol_type = other RNA
agtatcccgatattttaaaagctgagatctccactacaagattaaggt

SEQ ID NO: 655 - RNA, 63 nt, synthetic construct
  source 1..63: mol_type = other RNA
acgcgctctactcctgtaacggaaaggtcgcggcttgtgtgcctgcgggcagccgtgccgaga

SEQ ID NO: 656 - RNA, 47 nt, synthetic construct
  source 1..47: mol_type = other RNA
ttatacttcagttgttcagtcagattggaccctgtaaaagctgtaaa

SEQ ID NO: 657 - RNA, 95 nt, synthetic construct
  source 1..95: mol_type = other RNA
gcacatttggctatttttcaaagagagcagttagttcaagaaagtgcttattttgttttttattcttctttttgtggtctatttaggaaaacaaa

SEQ ID NO: 658 - RNA, 55 nt, synthetic construct
  source 1..55: mol_type = other RNA
agtctggtctggagctgggacagcagctgctgctgtggaaaggccagctggcaag

SEQ ID NO: 659 - RNA, 21 nt, synthetic construct
  source 1..21: mol_type = other RNA
acctttccgcgggccgcgggg

SEQ ID NO: 660 - RNA, 12 nt, synthetic construct
  source 1..12: mol_type = other RNA
aaaaatctcagt

SEQ ID NO: 661 - RNA, 10 nt, synthetic construct
  source 1..10: mol_type = other RNA
agccgcagct

SEQ ID NO: 662 - RNA, 92 nt, synthetic construct
  source 1..92: mol_type = other RNA
gcagtccaattcatcagttgtctttagtttactcagcatcagctactaacatacctgaacgaagatcttgttctaagacattgtagttgaag

SEQ ID NO: 663 - RNA, 39 nt, synthetic construct
  source 1..39: mol_type = other RNA
agacttggtatagtgggagagcctgactgagagtggatc

SEQ ID NO: 664 - RNA, 55 nt, synthetic construct
  source 1..55: mol_type = other RNA
atcttgattcctgctgtgtcttaactgaccattgtcttaaattctagagtggatc

SEQ ID NO: 665 - RNA, 19 nt, synthetic construct
  source 1..19: mol_type = other RNA
agaataaacatttctggga

SEQ ID NO: 666 - RNA, 73 nt, synthetic construct
  source 1..73: mol_type = other RNA
gtgaagtagagtgagcattagcctggcagacagctagggtaatagagtacttccagtggaagtcaaagcacaa

SEQ ID NO: 667 - RNA, 62 nt, synthetic construct
  source 1..62: mol_type = other RNA
gtgaattatagtttaagttactgcaagtcctaaactacggagtggaactattacagtttata

SEQ ID NO: 668 - RNA, 55 nt, synthetic construct
  source 1..55: mol_type = other RNA
caggagttcctccttggccgcggctgtagctgagagaagggacaggaggtcagcc

SEQ ID NO: 669 - RNA, 45 nt, synthetic construct
  source 1..45: mol_type = other RNA
acttccggttaagaagtcaacactcaggtctgaaaattgaacaag

SEQ ID NO: 670 - RNA, 42 nt, synthetic construct
  source 1..42: mol_type = other RNA
acctttaatttcctcgaaaacgcctgtaactcggctgaagcc

SEQ ID NO: 671 - RNA, 67 nt, synthetic construct
  source 1..67: mol_type = other RNA
ttacgacactgtccacctttaatttcctcgaaaacgcctgtaactcggctgaagcttcagtaccttt

SEQ ID NO: 672 - RNA, 65 nt, synthetic construct
  source 1..65: mol_type = other RNA
atttgccttctcttcacttggggagtatcaatttgccacagcagtctctggtcccagcccacccc

SEQ ID NO: 673 - RNA, 54 nt, synthetic construct
  source 1..54: mol_type = other RNA
taaggtaattcgtagtcaagaagctacacgtaattaaagttgcaggagtaaaag

SEQ ID NO: 674 - RNA, 52 nt, synthetic construct
  source 1..52: mol_type = other RNA
aattgactttgtgatcattacagaaagtctggtgtaaggtgttcagaagaca

SEQ ID NO: 675 - RNA, 56 nt, synthetic construct
  source 1..56: mol_type = other RNA
catcaattgactttgtgatcattacagaaagtctggtgtaaggtgttcagaagaca

SEQ ID NO: 676 - RNA, 37 nt, synthetic construct
  source 1..37: mol_type = other RNA
acactcagttttgaacacacggtgacacctgagaagc

SEQ ID NO: 677 - RNA, 69 nt, synthetic construct
  source 1..69: mol_type = other RNA
attcatttgagtgggaatctcaaagcagttgagtaggcagaaaaaagaacctcttcattaaggattaaa

SEQ ID NO: 678 - RNA, 51 nt, synthetic construct
  source 1..51: mol_type = other RNA
taatcagctgtaagtgccgtcactggatagaaggtccgtccttgggtctgt

SEQ ID NO: 679 - RNA, 56 nt, synthetic construct
  source 1..56: mol_type = other RNA
atacaaaaacatcatagttaagtaaactcaccagatttggcatctgctctttggtg

SEQ ID NO: 680 - RNA, 54 nt, synthetic construct
  source 1..54: mol_type = other RNA
agtaactgtgtttctagatttttcctccagctttcctgagagaaacaggtcaaa

SEQ ID NO: 681 - RNA, 83 nt, synthetic construct
  source 1..83: mol_type = other RNA
atacagtttctctttgccagactagactaaagaaggagcactaatttattctgataaaacaggtctagtcagctaccaggaca

SEQ ID NO: 682 - RNA, 81 nt, synthetic construct
  source 1..81: mol_type = other RNA
acagtttctctttgccagactagactaaagaaggagcactaatttattctgataaaacaggtctagtcagctaccaggaca

SEQ ID NO: 683 - RNA, 49 nt, synthetic construct
  source 1..49: mol_type = other RNA
agctgtttttcgtcttccctaggctatttctgccgggcgctccgcgaag

SEQ ID NO: 684 - RNA, 76 nt, synthetic construct
  source 1..76: mol_type = other RNA
tattgctagaccaccttgcttggaattaaaagttacacagttatttttcccagaacagttattttcagcttaagga

SEQ ID NO: 685 - RNA, 93 nt, synthetic construct
  source 1..93: mol_type = other RNA
acttttccagatctaggagcatcagtgacacctcgtcagagagtctggtacggtttggacggcgctgtggacgggcgaaggaaagtacagggg

SEQ ID NO: 686 - RNA, 51 nt, synthetic construct
  source 1..51: mol_type = other RNA
ttttcaggccaaaaggtgtgttttgctgacttcaagcatccctgctacaaa

SEQ ID NO: 687 - RNA, 60 nt, synthetic construct
  source 1..60: mol_type = other RNA
gcgtggcagttgaaagagtggcgggagaagttgcagggaattatttgatagcacatactg

SEQ ID NO: 688 - RNA, 59 nt, synthetic construct
  source 1..59: mol_type = other RNA
tgccaatcattgtgacatttttctgatttcttttttttaggtaactgaatattggatac

SEQ ID NO: 689 - RNA, 54 nt, synthetic construct
  source 1..54: mol_type = other RNA
agttgtttctgtaacagcagaaagtgcactcactaggagtagtcagaattcaaa

SEQ ID NO: 690 - RNA, 12 nt, synthetic construct
  source 1..12: mol_type = other RNA
cttccgggcaaa

SEQ ID NO: 691 - RNA, 14 nt, synthetic construct
  source 1..14: mol_type = other RNA
ctcttccgggcaaa

SEQ ID NO: 692 - RNA, 21 nt, synthetic construct
  source 1..21: mol_type = other RNA
gttcgccctaaaggtaccaat

SEQ ID NO: 693 - RNA, 23 nt, synthetic construct
  source 1..23: mol_type = other RNA
tcgttcgccctaaaggtaccaat

SEQ ID NO: 694 - RNA, 20 nt, synthetic construct
  source 1..20: mol_type = other RNA
ttcgccctaaaggtaccaat

SEQ ID NO: 695 - RNA, 94 nt, synthetic construct
  source 1..94: mol_type = other RNA
tgacagaagcgtcaattcctgggagtagttcgttggttttctttcccctcatccttttgcctgctcccggcgaggggtggctttgatttcggcg

SEQ ID NO: 696 - RNA, 62 nt, synthetic construct
  source 1..62: mol_type = other RNA
gaagtttgagcctctttggtagcaggaggctggaagaaaggacagaagtagctctggctgtg

SEQ ID NO: 697 - RNA, 53 nt, synthetic construct
  source 1..53: mol_type = other RNA
agagctggaatctctgcacgggccttggaaaacgactgtcttcttctgccaaa

SEQ ID NO: 698 - RNA, 48 nt, synthetic construct
  source 1..48: mol_type = other RNA
agtttgaacttacaatacttaagtcctggcaagtcataagtggggaaa

SEQ ID NO: 699 - RNA, 37 nt, synthetic construct
  source 1..37: mol_type = other RNA
acaatacttaagtcctggcaagtcataagtggggaaa

SEQ ID NO: 700 - RNA, 70 nt, synthetic construct
  source 1..70: mol_type = other RNA
gagttcacctttcaacatcaccagtttgaacttacaatacttaagtcctggcaagtcataagtggggaaa

SEQ ID NO: 701 - RNA, 63 nt, synthetic construct
  source 1..63: mol_type = other RNA
cggctgcagaggggaaggcggctaccagtgtaaagccagagctgaggttcttgatagtccaca

SEQ ID NO: 702 - RNA, 53 nt, synthetic construct
  source 1..53: mol_type = other RNA
gacagtttcccgatttgaggtgaaaccagtaagagaaaatagaatacttaata

SEQ ID NO: 703 - RNA, 12 nt, synthetic construct
  source 1..12: mol_type = other RNA
gctgttgccatc

SEQ ID NO: 704 - RNA, 30 nt, synthetic construct
  source 1..30: mol_type = other RNA
acactttgccacagaaagcagtgagcaaga

SEQ ID NO: 705 - RNA, 70 nt, synthetic construct
  source 1..70: mol_type = other RNA
actttattacagaccttggaagcaagaggattgcattcagcctagttcctggttgctggccaaagggatc

SEQ ID NO: 706 - RNA, 55 nt, synthetic construct
  source 1..55: mol_type = other RNA
tttgtacttcctgagagaaggcagagaagaagaagtttttattacatagagtaac

SEQ ID NO: 707 - RNA, 44 nt, synthetic construct
  source 1..44: mol_type = other RNA
agaaaggagcagcaactggaaaacaagcattgcattgcatcagg

SEQ ID NO: 708 - RNA, 46 nt, synthetic construct
  source 1..46: mol_type = other RNA
acagcctggactcagagctcaagtctgaactctacctccagacaga

SEQ ID NO: 709 - RNA, 91 nt, synthetic construct
  source 1..91: mol_type = other RNA
atacacaagaagtttcaggagtcagaggagggttgagtataattagaagcagtctttccactgaacttcccgacaacatttgttagtcaga

SEQ ID NO: 710 - RNA, 16 nt, synthetic construct
  source 1..16: mol_type = other RNA
tctctttctggtcaaa

SEQ ID NO: 711 - RNA, 56 nt, synthetic construct
  source 1..56: mol_type = other RNA
ttaaataagtagggattgtttaaaaaaaaaagctgtaatagtttatctgggggtgg

SEQ ID NO: 712 - RNA, 66 nt, synthetic construct
  source 1..66: mol_type = other RNA
tctagcgaaatagtgctacagagagccctgctacgcgtcgggtccaggttactgaagctaaaggag

SEQ ID NO: 713 - RNA, 44 nt, synthetic construct
  source 1..44: mol_type = other RNA
attgacttgagtcaaaacatcacaaaggctccatattatacagt

SEQ ID NO: 714 - RNA, 42 nt, synthetic construct
  source 1..42: mol_type = other RNA
actcccctggcagaccccgccaagcgcctcggagcgcgcagg

SEQ ID NO: 715 - RNA, 62 nt, synthetic construct
  source 1..62: mol_type = other RNA
ggtcagagagaagcaataattattattaacatttattaacgatcaataaacttgatcgcatt

SEQ ID NO: 716 - RNA, 29 nt, synthetic construct
  source 1..29: mol_type = other RNA
tttacttttcttttattaggttgtttgct

SEQ ID NO: 717 - RNA, 21 nt, synthetic construct
  source 1..21: mol_type = other RNA
cttactttgcaaagaaggaag

SEQ ID NO: 718 - RNA, 73 nt, synthetic construct
  source 1..73: mol_type = other RNA
caggacgtcttccagagtgtgttttttggctacagtgttttcagtatcctctttggcctcctggctgacagat

SEQ ID NO: 719 - RNA, 65 nt, synthetic construct
  source 1..65: mol_type = other RNA
gttttatctccctttctgttttgtttgtgaccaggcataaagcagtcaccttcaacagttacaga

SEQ ID NO: 720 - RNA, 46 nt, synthetic construct
  source 1..46: mol_type = other RNA
aggagagttgccaaactgttaagagtggttatttctgagcagaaga

SEQ ID NO: 721 - RNA, 76 nt, synthetic construct
  source 1..76: mol_type = other RNA
acagagcagtgataactacctgccagtgtctcttaggagtgaggtacctggagttcggggaccccaacctgtgaca

SEQ ID NO: 722 - RNA, 24 nt, synthetic construct
  source 1..24: mol_type = other RNA
agtgttctaactattaacgctacg

SEQ ID NO: 723 - RNA, 50 nt, synthetic construct
  source 1..50: mol_type = other RNA
cctgtcattcgttcgtcctcagtgcagggcaacaggactttaggttcaag

SEQ ID NO: 724 - RNA, 55 nt, synthetic construct
  source 1..55: mol_type = other RNA
ttctttttggattgaagccttttccccacgactctgaaagaggacagcgttccca

SEQ ID NO: 725 - RNA, 93 nt, synthetic construct
  source 1..93: mol_type = other RNA
attagatatttcatcttactgcagagcatagaatcaataggagcttctccttagggagctgctttgaaacagcttccagctgatcttaccaag

SEQ ID NO: 726 - RNA, 19 nt, synthetic construct
  source 1..19: mol_type = other RNA
gtccattcttccggtggag

SEQ ID NO: 727 - RNA, 12 nt, synthetic construct
  source 1..12: mol_type = other RNA
cttccggtggag

SEQ ID NO: 728 - RNA, 55 nt, synthetic construct
  source 1..55: mol_type = other RNA
agtctgggagcctggaggactagcgaggaggagttgagagaacggagcggacgcc

SEQ ID NO: 729 - RNA, 15 nt, synthetic construct
  source 1..15: mol_type = other RNA
gctaccgtgactaag

SEQ ID NO: 730 - RNA, 60 nt, synthetic construct
  source 1..60: mol_type = other RNA
atcttcttgccattcagttgtagatctttgaagactctgattctctgagactgaggagag

SEQ ID NO: 731 - RNA, 64 nt, synthetic construct
  source 1..64: mol_type = other RNA
tttttcctctgcgaatagtgttttctgattaactactagttagttctagtccaaaaaaagaaac

SEQ ID NO: 732 - RNA, 21 nt, synthetic construct
  source 1..21: mol_type = other RNA
gcaggtcccaccttgtggagg

SEQ ID NO: 733 - RNA, 71 nt, synthetic construct
  source 1..71: mol_type = other RNA
acttccggtgttacctgtgtcgttaccgggagctgtaaacaaggtgtgcaagcatctgaagagctgccggg

SEQ ID NO: 734 - RNA, 53 nt, synthetic construct
  source 1..53: mol_type = other RNA
gtcgttaccgggagctgtaaacaaggtgtgcaagcatctgaagagctgccggg

SEQ ID NO: 735 - RNA, 86 nt, synthetic construct
  source 1..86: mol_type = other RNA
ctcaacttccctagtcagtgacgcggcgccggccagaaatccgaccggaccgggctcgggggagcgtgagttgcagttaaaagaag

SEQ ID NO: 736 - RNA, 12 nt, synthetic construct
  source 1..12: mol_type = other RNA
ctggccttctcc

SEQ ID NO: 737 - RNA, 25 nt, synthetic construct
  source 1..25: mol_type = other RNA
ccagacctggagggcttgggtaaaa

SEQ ID NO: 738 - RNA, 62 nt, synthetic construct
  source 1..62: mol_type = other RNA
acttccggaacaagcgtcgcgtttctgaggagaaactcttggtgagaattcccagagtgata

SEQ ID NO: 739 - RNA, 16 nt, synthetic construct
  source 1..16: mol_type = other RNA
ctagtttctaaggatc

SEQ ID NO: 740 - RNA, 56 nt, synthetic construct
  source 1..56: mol_type = other RNA
acactggcccttgctttggagtcaggaggcctctcttcttcccacagagccctggg

SEQ ID NO: 741 - RNA, 97 nt, synthetic construct
  source 1..97: mol_type = other RNA
cttttattgcttttgtacaagaccagacaggatctcatttgttaaacgtggtaccaattgggtgtcttaacacaggagcagaacttcctagagcaga

SEQ ID NO: 742 - RNA, 23 nt, synthetic construct
  source 1..23: mol_type = other RNA
cagccagtcctgagcacctaacc

SEQ ID NO: 743 - RNA, 40 nt, synthetic construct
  source 1..40: mol_type = other RNA
gtcaccgctttcgcttctgcttgagtagtcaagtgaaaaa

SEQ ID NO: 744 - RNA, 69 nt, synthetic construct
  source 1..69: mol_type = other RNA
tggagaaaagccaaacaattgcagttttccatattttagagaaaataaattaatcagtctcagttttct

SEQ ID NO: 745 - RNA, 53 nt, synthetic construct
  source 1..53: mol_type = other RNA
tgacagcagccagctcaggaggcagaggacgagagcgatcgggctgcacacac

SEQ ID NO: 746 - RNA, 91 nt, synthetic construct
  source 1..91: mol_type = other RNA
ttatctgcagagatcaggccaaagggagctctagaaggttgttggggagcactaaactggagaataagagaagatagtttaagtccgtacc

SEQ ID NO: 747 - RNA, 44 nt, synthetic construct
  source 1..44: mol_type = other RNA
cagacaggagccgaacgggggcttccgctcagcagagaggcaag

SEQ ID NO: 748 - RNA, 25 nt, synthetic construct
  source 1..25: mol_type = other RNA
tcttccctatccctagttccaaaag

SEQ ID NO: 749 - RNA, 55 nt, synthetic construct
  source 1..55: mol_type = other RNA
agtgtccagaagtctggagcgtaggtgaaaggacaaaagccagacacatttcaac

SEQ ID NO: 750 - RNA, 44 nt, synthetic construct
  source 1..44: mol_type = other RNA
agcatatttcattttctgtcattggactttgagccattagaacc

SEQ ID NO: 751 - RNA, 93 nt, synthetic construct
  source 1..93: mol_type = other RNA
tgaactgaaatctccttgttgctttgaaatcttagaagagagcccactaattcaaggactcttactgtgggagcaactgctggttctatcaca

SEQ ID NO: 752 - RNA, 63 nt, synthetic construct
  source 1..63: mol_type = other RNA
cagcactccaccaaagcctctgcctcagccttactgtgagtctggttgacagtagcttctaag

SEQ ID NO: 753 - RNA, 62 nt, synthetic construct
  source 1..62: mol_type = other RNA
taaactcctggtactctagcaccgatctgctttggagaacctgatcctgagactccagcagg

SEQ ID NO: 754 - RNA, 45 nt, synthetic construct
  source 1..45: mol_type = other RNA
tataatttcgtacagcaacttgcggagagatttgtgagccctgaa

SEQ ID NO: 755 - RNA, 56 nt, synthetic construct
  source 1..56: mol_type = other RNA
acagctggcattcagcctccagagcaccagcactggcactggcactggcacacgct

SEQ ID NO: 756 - RNA, 22 nt, synthetic construct
  source 1..22: mol_type = other RNA
cttcctcggaactgttgcccac

SEQ ID NO: 757 - RNA, 80 nt, synthetic construct
  source 1..80: mol_type = other RNA
agaatacggtgagaaattccaggagcacagaagtcagcgggcatctcctggagacaggcagacatttggtctcagcagcc

SEQ ID NO: 758 - RNA, 44 nt, synthetic construct
  source 1..44: mol_type = other RNA
ctttcttggccagaggcgccggttggactcacgggcggggcagt

SEQ ID NO: 759 - RNA, 27 nt, synthetic construct
  source 1..27: mol_type = other RNA
gagaattgcttcctgagaaggtaagta

SEQ ID NO: 760 - RNA, 46 nt, synthetic construct
  source 1..46: mol_type = other RNA
ctcttcctgtctttgtggctccggaaaggcgtttgggagtccaacg

SEQ ID NO: 761 - RNA, 52 nt, synthetic construct
  source 1..52: mol_type = other RNA
actgtcagctttgaacactgaacgcgaggactgttaactgtttctggcaaac

SEQ ID NO: 762 - RNA, 73 nt, synthetic construct
  source 1..73: mol_type = other RNA
agtatattagtgctaatttccctccgtttgtcctagcttttctcttctgtcaaccccacacgcctttggcaca

SEQ ID NO: 763 - RNA, 19 nt, synthetic construct
  source 1..19: mol_type = other RNA
acagaagccgtttctcatc

SEQ ID NO: 764 - RNA, 63 nt, synthetic construct
  source 1..63: mol_type = other RNA
agaatccagcactccaagcactgctgtcttctcacagagtcttgaagccagagcagcgccagg

SEQ ID NO: 765 - RNA, 51 nt, synthetic construct
  source 1..51: mol_type = other RNA
accttcaggccagtcagccagtttccgggagccctaattgcacagaagccc

SEQ ID NO: 766 - RNA, 11 nt, synthetic construct
  source 1..11: mol_type = other RNA
aggtggaggag

SEQ ID NO: 767 - RNA, 69 nt, synthetic construct
  source 1..69: mol_type = other RNA
gtatctgcccatcttagattcttcctagcaataattctgagagactctggattgtgggccgaagtgaga

SEQ ID NO: 768 - RNA, 12 nt, synthetic construct
  source 1..12: mol_type = other RNA
caaagaagaacc

SEQ ID NO: 769 - RNA, 44 nt, synthetic construct
  source 1..44: mol_type = other RNA
agagatagagtcttccctggcattgcaggagagaatctgaaggg

SEQ ID NO: 770 - RNA, 92 nt, synthetic construct
  source 1..92: mol_type = other RNA
tcattcaagtgagtaagttaagaagtgccaagggtgaggatctcctttagaagaggagaagccttgcatcaacctcttgggcctcagccaag

SEQ ID NO: 771 - RNA, 52 nt, synthetic construct
  source 1..52: mol_type = other RNA
aggtgaaaaattagacgatttcaagaattggagctgaatctgagtttaagta

SEQ ID NO: 772 - RNA, 55 nt, synthetic construct
  source 1..55: mol_type = other RNA
gtaagttttttttggttgctgacaaagttctttttattccaagcaggactataat

SEQ ID NO: 773 - RNA, 64 nt, synthetic construct
  source 1..64: mol_type = other RNA
ttacactaagaactctttgttacatcaggtattgcactctcagtctcgccagtttgaagtcaga

SEQ ID NO: 774 - RNA, 68 nt, synthetic construct
  source 1..68: mol_type = other RNA
attataaactcatttctgttttattttactgaagaacccattaaaagagccagtaaatcctgtggaaa

SEQ ID NO: 775 - RNA, 48 nt, synthetic construct
  source 1..48: mol_type = other RNA
agaaacattcagattctccagagaaagttgtatttacaggagacagca

SEQ ID NO: 776 - RNA, 60 nt, synthetic construct
  source 1..60: mol_type = other RNA
acagcacgctgccacgccgacgcagacccctctctgcacgccagcccgcccgcacccacc

SEQ ID NO: 777 - RNA, 42 nt, synthetic construct
  source 1..42: mol_type = other RNA
acatatccactcctgctctccctcctgcaggtgaccccagcc

SEQ ID NO: 778 - RNA, 49 nt, synthetic construct
  source 1..49: mol_type = other RNA
actctgcatttctttcccgccctctgcagtagtgtttgaagtggtgaac

SEQ ID NO: 779 - RNA, 72 nt, synthetic construct
  source 1..72: mol_type = other RNA
agcacggttcgtttttcctttagtcaggaaggacgttggtgttgagcatacgtatcaaggacagtaactacc

SEQ ID NO: 780 - RNA, 17 nt, synthetic construct
  source 1..17: mol_type = other RNA
tttccggctccggtgtc

SEQ ID NO: 781 - RNA, 31 nt, synthetic construct
  source 1..31: mol_type = other RNA
agggcggagcgggcggcagacccggagcagc

SEQ ID NO: 782 - RNA, 51 nt, synthetic construct
  source 1..51: mol_type = other RNA
agtcatctttatccctttaagcagtgtttattttctgagcaaggagtcatc

SEQ ID NO: 783 - RNA, 19 nt, synthetic construct
  source 1..19: mol_type = other RNA
acattcaacctctgccacc

SEQ ID NO: 784 - RNA, 69 nt, synthetic construct
  source 1..69: mol_type = other RNA
aggaagagagtcaggaggcgatttttacctcagagtttgaaatttacactgctttgaaagaaagaagat

SEQ ID NO: 785 - RNA, 75 nt, synthetic construct
  source 1..75: mol_type = other RNA
agacctctcgggcgagcgcggcgcagcgcagattaaaaatcaagaaatataaaccagagttagcagtttcttgac

SEQ ID NO: 786 - RNA, 44 nt, synthetic construct
  source 1..44: mol_type = other RNA
attaaaaatcaagaaatataaaccagagttagcagtttcttgac

SEQ ID NO: 787 - RNA, 52 nt, synthetic construct
  source 1..52: mol_type = other RNA
gagaagtaggcgatttcggaggagtgagaaatagccccgagtcccgtggaaa

SEQ ID NO: 788 - RNA, 12 nt, synthetic construct
  source 1..12: mol_type = other RNA
cttccttgtaag

SEQ ID NO: 789 - RNA, 64 nt, synthetic construct
  source 1..64: mol_type = other RNA
ttccaggatcagaagtactgaggagcagagccaatcttctctgtttggaaatagtggcttggaa

SEQ ID NO: 790 - RNA, 122 nt, synthetic construct
  source 1..122: mol_type = other RNA
agttgttctcaggtgtttgggcttgttgttccgtatactcagtgggttcgcggccgccggcccagtgaggctgggttcgaggagctggagcgggaaactggagcttaaattctggcggcgag

SEQ ID NO: 791 - RNA, 59 nt, synthetic construct
  source 1..59: mol_type = other RNA
gcgagtttgacggggtcctaagagtgctgctgggcgaccacttcctgacagaagaaaag

SEQ ID NO: 792 - RNA, 59 nt, synthetic construct
  source 1..59: mol_type = other RNA
actcttgttgcatcatcagcgtgcacctccacgagtaaacaggtctgggctacaaaagt

SEQ ID NO: 793 - RNA, 94 nt, synthetic construct
  source 1..94: mol_type = other RNA
gtcagcttcacattctcaggaactctccttctttgggtctggctgaagttgaggatctcttactctctaggccacggaattaacccgagcaggc

SEQ ID NO: 794 - RNA, 65 nt, synthetic construct
  source 1..65: mol_type = other RNA
caaacccagtcttgtcaatagcgggtttcaccctccaccagttcagtctgttgcctgtgtcagac

SEQ ID NO: 795 - RNA, 50 nt, synthetic construct
  source 1..50: mol_type = other RNA
agaaaaaagaaatcctctttttagaacatctcttggtggtaccatcagaa

SEQ ID NO: 796 - RNA, 21 nt, synthetic construct
  source 1..21: mol_type = other RNA
gtttctctccgcaagcgcgcg

SEQ ID NO: 797 - RNA, 22 nt, synthetic construct
  source 1..22: mol_type = other RNA
gtcagagggccggagcgagaag

SEQ ID NO: 798 - RNA, 19 nt, synthetic construct
  source 1..19: mol_type = other RNA
aaacgtgaagaaggtgaag

SEQ ID NO: 799 - RNA, 76 nt, synthetic construct
  source 1..76: mol_type = other RNA
aaacagataccagtgagtagtttgaaacaggaacaaaatcttctgaaagctcggagcagaagcctttttggtcaac

SEQ ID NO: 800 - RNA, 27 nt, synthetic construct
  source 1..27: mol_type = other RNA
ctctctttgtccaacgtagaagagaca

SEQ ID NO: 801 - RNA, 92 nt, synthetic construct
  source 1..92: mol_type = other RNA
gaaatctgtctttgttgctctgggagaggggactcctggaagttgtctgtgaataaagactagccgaagatactgtggcctcagtaatagga

SEQ ID NO: 802 - RNA, 34 nt, synthetic construct
  source 1..34: mol_type = other RNA
tcttcccttctttgtagttcaggattcgcccaaa

SEQ ID NO: 803 - RNA, 22 nt, synthetic construct
  source 1..22: mol_type = other RNA
ctttttcgtctgggctgccaac

SEQ ID NO: 804 - RNA, 46 nt, synthetic construct
  source 1..46: mol_type = other RNA
cagaagatacttgtgttttcctgtagttgacaaaaaacggtagaag

SEQ ID NO: 805 - RNA, 54 nt, synthetic construct
  source 1..54: mol_type = other RNA
agtgtctttatccctcttttgcacagtcagcttctgcagctctcccgggctagc

SEQ ID NO: 806 - RNA, 80 nt, synthetic construct
  source 1..80: mol_type = other RNA
aacactcctgaggagaaagaaagagagggagggagagaaaaagagagagagagaaacaaaaaaccaaagagagagaaaaa

SEQ ID NO: 807 - RNA, 82 nt, synthetic construct
  source 1..82: mol_type = other RNA
ggaggttttctcttgggtttgtttggattttttttttcagcttttgtctgaattttggttttatttttctggggcagagaaa

SEQ ID NO: 808 - RNA, 22 nt, synthetic construct
  source 1..22: mol_type = other RNA
cagcccttgccagcatccagcc

SEQ ID NO: 809 - RNA, 53 nt, synthetic construct
  source 1..53: mol_type = other RNA
gcggagcggcaggagtcaggaggagctggcgtgggagaccgacggcctttggg

SEQ ID NO: 810 - RNA, 44 nt, synthetic construct
  source 1..44: mol_type = other RNA
cgggccaagcctaccgggggtcacaatttgggtctgcagcagaa

SEQ ID NO: 811 - RNA, 55 nt, synthetic construct
  source 1..55: mol_type = other RNA
acagtgaaaaagcagtctggctcccgaggtccaccccttataccccaaggtccag

SEQ ID NO: 812 - RNA, 39 nt, synthetic construct
  source 1..39: mol_type = other RNA
ttcttcagccttaacctaaggtctcatactcggagcact

SEQ ID NO: 813 - RNA, 44 nt, synthetic construct
  source 1..44: mol_type = other RNA
acagcccaccagtgaccacgaaggctgtgctgcttgccctgttg

SEQ ID NO: 814 - RNA, 61 nt, synthetic construct
  source 1..61: mol_type = other RNA
aaacgtgccccttcaccggaaccgagcaacggtaactgtgccccttcaccggaaccgagca

SEQ ID NO: 815 - RNA, 49 nt, synthetic construct
  source 1..49: mol_type = other RNA
tagtgcaagtaagaaagaaactgaatttgtcattttcacctgaagaaaa

SEQ ID NO: 816 - RNA, 65 nt, synthetic construct
  source 1..65: mol_type = other RNA
agtggcatccttccctttctaatcagagattttcttcctcagagattttggcctagatttgcaaa

SEQ ID NO: 817 - RNA, 59 nt, synthetic construct
  source 1..59: mol_type = other RNA
acactcttcgtcatcacctctcctattcgcctggacaagctcagttttgcaggagcacc

SEQ ID NO: 818 - RNA, 34 nt, synthetic construct
  source 1..34: mol_type = other RNA
gtaaatccgccatcttcctgcggcgcgttgcgac

SEQ ID NO: 819 - RNA, 66 nt, synthetic construct
  source 1..66: mol_type = other RNA
agagacgaccttaagtggacagagattctagaacaggagaaataaaacagctatcgtttgggagca

SEQ ID NO: 820 - RNA, 79 nt, synthetic construct
  source 1..79: mol_type = other RNA
tgcgtatacatacatactagttagttgcgtttagttgtgtctgtgtgttttttttttaacagacgaagcttcacaaaag

SEQ ID NO: 821 - RNA, 22 nt, synthetic construct
  source 1..22: mol_type = other RNA
agagcatagcacctgcagcaag

SEQ ID NO: 822 - RNA, 94 nt, synthetic construct
  source 1..94: mol_type = other RNA
gctttaatagggagatcaagagtaatttgagttataatcagaatcctagagtgaggaggagcacctctagtccttgaggtggaaagaactggaa

SEQ ID NO: 823 - RNA, 20 nt, synthetic construct
  source 1..20: mol_type = other RNA
acaccttctgaggcagcggc

SEQ ID NO: 824 - RNA, 73 nt, synthetic construct
  source 1..73: mol_type = other RNA
ttaagtcgttccctctacaaaggacttcctagtgggtgtgaaaggcagcggtggccacagaggcggcggagag

SEQ ID NO: 825 - RNA, 99 nt, synthetic construct
  source 1..99: mol_type = other RNA
aagttgctttggtttctatttctttgttaagtcgttccctctacaaaggacttcctagtgggtgtgaaaggcagcggtggccacagaggcggcggagag

SEQ ID NO: 826 - RNA, 54 nt, synthetic construct
  source 1..54: mol_type = other RNA
gattattaagttcctgcaacttaactgggaactgatcaagatttcaagctaaag

SEQ ID NO: 827 - RNA, 20 nt, synthetic construct
  source 1..20: mol_type = other RNA
acagagtctctttccagtcc

SEQ ID NO: 828 - RNA, 12 nt, synthetic construct
  source 1..12: mol_type = other RNA
acaagctcagcg

SEQ ID NO: 829 - RNA, 55 nt, synthetic construct
  source 1..55: mol_type = other RNA
agaacaaccagctggatcagttctcacaggagctacagcgcggagactgggaaac

SEQ ID NO: 830 - RNA, 70 nt, synthetic construct
  source 1..70: mol_type = other RNA
gtaatagtgttttaagaatatttcagcgtagtagaggatttaagcagatcgctttgcctactttaattat

SEQ ID NO: 831 - RNA, 62 nt, synthetic construct
  source 1..62: mol_type = other RNA
gttttaagaatatttcagcgtagtagaggatttaagcagatcgctttgcctactttaattat

SEQ ID NO: 832 - RNA, 68 nt, synthetic construct
  source 1..68: mol_type = other RNA
aatagtgttttaagaatatttcagcgtagtagaggatttaagcagatcgctttgcctactttaattat

SEQ ID NO: 833 - RNA, 63 nt, synthetic construct
  source 1..63: mol_type = other RNA
ataacacctagtttgagtcaacctggttaagtacaaatagtagaaggcttctcattcaggtcc

SEQ ID NO: 834 - RNA, 11 nt, synthetic construct
  source 1..11: mol_type = other RNA
actgaagagtc

SEQ ID NO: 835 - RNA, 22 nt, synthetic construct
  source 1..22: mol_type = other RNA
tcagagctctcaagcacccacg

SEQ ID NO: 836 - RNA, 48 nt, synthetic construct
  source 1..48: mol_type = other RNA
gttagtagtaccgtcgctttcctggatagtggctatcttagcaaagca

SEQ ID NO: 837 - RNA, 57 nt, synthetic construct
  source 1..57: mol_type = other RNA
tccagacaggactgtccagagcaacggtgtgccagagagactgagaagtacccagaa

SEQ ID NO: 838 - RNA, 54 nt, synthetic construct
  source 1..54: mol_type = other RNA
aaactcctctttgattcttctagctgtttcactattgggcaaccagacaccaga

SEQ ID NO: 839 - RNA, 76 nt, synthetic construct
  source 1..76: mol_type = other RNA
actctcctgtaaaacgctagagcggcgagttgttacctgcgtcctctgacctgagagcgaaggggaaagcggcgag

SEQ ID NO: 840 - RNA, 21 nt, synthetic construct
  source 1..21: mol_type = other RNA
gcagttccggtgagtccgtgg

SEQ ID NO: 841 - RNA, 93 nt, synthetic construct
  source 1..93: mol_type = other RNA
agctacaaccaagtaagcaagtgtcagggctcaccaaccagtcaaggacagggcaggagaagaggaacctgcaaagacatattttgttccaaa

SEQ ID NO: 842 - RNA, 69 nt, synthetic construct
  source 1..69: mol_type = other RNA
aagtcattccttctgcaaaggactgcctggcaggtgtgaaaggcagcggtggccacagaggcggtggag

SEQ ID NO: 843 - RNA, 55 nt, synthetic construct
  source 1..55: mol_type = other RNA
agcccttgcgccctgtttgtgccagcaggcaccagcgaaccaaaagcaggtgtcc

SEQ ID NO: 844 - RNA, 39 nt, synthetic construct
  source 1..39: mol_type = other RNA
ccggaacgtctttttgcggacgccctcggagcagccgcg

SEQ ID NO: 845 - RNA, 57 nt, synthetic construct
  source 1..57: mol_type = other RNA
acacacacatctgcacctcaaccacagactacacttgctgaactggctcctggggcc

SEQ ID NO: 846 - RNA, 39 nt, synthetic construct
  source 1..39: mol_type = other RNA
ctgcttttctccagctctgtcaactcaacctttcccacc

SEQ ID NO: 847 - RNA, 63 nt, synthetic construct
  source 1..63: mol_type = other RNA
acaggcccttggttccatcctctgccatctactccactgttcagacacctcctaacctccgtc

SEQ ID NO: 848 - RNA, 42 nt, synthetic construct
  source 1..42: mol_type = other RNA
acagatctgcctcttctccaagaaactcaaccactagtgaca

SEQ ID NO: 849 - RNA, 61 nt, synthetic construct
  source 1..61: mol_type = other RNA
gctgtttagaactattgtaccgctgccagtgactgtctgtattagggttgggagcaggaac

SEQ ID NO: 850 - RNA, 67 nt, synthetic construct
  source 1..67: mol_type = other RNA
tagggacgggaagtcgcgaccagagccattggagggcgcggggactgcaaccctaatcagagcccaa

SEQ ID NO: 851 - RNA, 96 nt, synthetic construct
  source 1..96: mol_type = other RNA
agaagtcctgtagggagagtggcagttaccttctctccgtctctctctcggggactctatttgtggctggtggagctgtcttaaacgaagagaacc

SEQ ID NO: 852 - RNA, 33 nt, synthetic construct
  source 1..33: mol_type = other RNA
cgagcacttggattcagcttcttcatttccaac

SEQ ID NO: 853 - RNA, 23 nt, synthetic construct
  source 1..23: mol_type = other RNA
ccagtgaagtcctgggcagaaga

SEQ ID NO: 854 - RNA, 68 nt, synthetic construct
  source 1..68: mol_type = other RNA
cacttgaccttccatcagtaagacggacttttctaatctgttcctggagatattaagtgaatacagtc

SEQ ID NO: 855 - RNA, 56 nt, synthetic construct
  source 1..56: mol_type = other RNA
ctctttctcagtgaccgggtggtttgcttaggcgcagacggggaagcggagccaac

SEQ ID NO: 856 - RNA, 60 nt, synthetic construct
  source 1..60: mol_type = other RNA
taattcagccacatctcctccttctcactcaactcagacctctcaggataacttggtgaa

SEQ ID NO: 857 - RNA, 56 nt, synthetic construct
  source 1..56: mol_type = other RNA
cctgaccgtagtgcggccagcagttgcaggcagacggagcagagcggtcagggatc

SEQ ID NO: 858 - RNA, 61 nt, synthetic construct
  source 1..61: mol_type = other RNA
acagcattcctggagcccaggctcttttccacagaggaggaaagagcaggcagcagagacc

SEQ ID NO: 859 - RNA, 91 nt, synthetic construct
  source 1..91: mol_type = other RNA
ttgagtaagttcactggtgtgtcatcagtttgcagttctatcatccactttagttggtgcttttctaactttatctccctctagtagcata

SEQ ID NO: 860 - RNA, 42 nt, synthetic construct
  source 1..42: mol_type = other RNA
gcagaggtcaccgtccaggttggacagcagcacctttgagcg

SEQ ID NO: 861 - RNA, 20 nt, synthetic construct
  source 1..20: mol_type = other RNA
gccgtccgcggaaatttgaa

SEQ ID NO: 862 - RNA, 57 nt, synthetic construct
  source 1..57: mol_type = other RNA
aagccttccaaaccctccttcctggaaacccacccagaacctccaccctctgacacc

SEQ ID NO: 863 - RNA, 66 nt, synthetic construct
  source 1..66: mol_type = other RNA
tggaagtgctcaagtgtggtatacactcaggtgcatttttccaatcacaatattctggactgaaat

SEQ ID NO: 864 - RNA, 65 nt, synthetic construct
  source 1..65: mol_type = other RNA
aagattatagagacttgctttagaaccacaagaagaaagaggaggccggcttttcagctagcatc

SEQ ID NO: 865 - RNA, 55 nt, synthetic construct
  source 1..55: mol_type = other RNA
aatcctttctttcagctggagtgctcctcaggagccagccccacccttagaaaag

SEQ ID NO: 866 - RNA, 94 nt, synthetic construct
  source 1..94: mol_type = other RNA
ttcttctttttttctcaagagaatatttgacctgagttaattcttggatctctgaggtcttctacacagaagtatatttagagagcaattcaga

SEQ ID NO: 867 - RNA, 43 nt, synthetic construct
  source 1..43: mol_type = other RNA
aaactaagagtagtgctgcagcaagctccctcttggattcaga

SEQ ID NO: 868 - RNA, 99 nt, synthetic construct
  source 1..99: mol_type = other RNA
aagttgctttggtttctattactttgttaagtcattccttctgcaaaggactgcctggcaggtgtcaaaggcagtggtggccacagaggcggtggagag

SEQ ID NO: 869 - RNA, 90 nt, synthetic construct
  source 1..90: mol_type = other RNA
tggtttctattactttgttaagtcattccttctgcaaaggactgcctggcaggtgtcaaaggcagtggtggccacagaggcggtggagag

SEQ ID NO: 870 - RNA, 62 nt, synthetic construct
  source 1..62: mol_type = other RNA
catcgctctgtgtttcctgctccaggtttcgcgtctcgcatcttcccagaccctcgggcgcg

SEQ ID NO: 871 - RNA, 52 nt, synthetic construct
  source 1..52: mol_type = other RNA
ctgtttggagttggaagccgagacctaaagttggggggtgatctctgaggag

SEQ ID NO: 872 - RNA, 54 nt, synthetic construct
  source 1..54: mol_type = other RNA
gtgagacttccagatcttctctggtgaagtgtgtttcctgcaacgatcacgaac

SEQ ID NO: 873 - RNA, 98 nt, synthetic construct
  source 1..98: mol_type = other RNA
acatcacagaagtagaggcagaccttgcttcagtagcaagctcatctctggaacaaactggcaaagcatctctgctggtgttcatcagaacagacacc

SEQ ID NO: 874 - RNA, 71 nt, synthetic construct
  source 1..71: mol_type = other RNA
agacagtcatctcaggagcagaaagaaaagagctcccaaagtctatatctattcaggggctctcaagaaca

SEQ ID NO: 875 - RNA, 71 nt, synthetic construct
  source 1..71: mol_type = other RNA
atttcctgctcttgaatatctggttgaactacttaagcttaatttgttaaactccggggctctcaagaaca

SEQ ID NO: 876 - RNA, 12 nt, synthetic construct
  source 1..12: mol_type = other RNA
ggaataggaatc

SEQ ID NO: 877 - RNA, 16 nt, synthetic construct
  source 1..16: mol_type = other RNA
gcgaggaataggaatc

SEQ ID NO: 878 - RNA, 25 nt, synthetic construct
  source 1..25: mol_type = other RNA
gtaggaccggcgaggaataggaatc

SEQ ID NO: 879 - RNA, 52 nt, synthetic construct
  source 1..52: mol_type = other RNA
ttgctttatttctcattgctacaggtcatcttaagtttctcagataatactg

SEQ ID NO: 880 - RNA, 20 nt, synthetic construct
  source 1..20: mol_type = other RNA
actgggttgctgatccagct

SEQ ID NO: 881 - RNA, 55 nt, synthetic construct
  source 1..55: mol_type = other RNA
agaatttgatttgactatatttaccatatcagattcacattcagtcctcagcaaa

SEQ ID NO: 882 - RNA, 51 nt, synthetic construct
  source 1..51: mol_type = other RNA
attggattcctgatctgattgaagcccagaagtgacagagtcactggccac

SEQ ID NO: 883 - RNA, 23 nt, synthetic construct
  source 1..23: mol_type = other RNA
ttgggattttggagttgagcacc

SEQ ID NO: 884 - RNA, 97 nt, synthetic construct
  source 1..97: mol_type = other RNA
taagtgtacagtcttaggccttctgaagcagcatttgaagctgcagtcctgaaaaccagtcaggccggaagagtagataaagaaatatttatttgag

SEQ ID NO: 885 - RNA, 27 nt, synthetic construct
  source 1..27: mol_type = other RNA
tttgttttgtagattggttgtttcatt

SEQ ID NO: 886 - RNA, 12 nt, synthetic construct
  source 1..12: mol_type = other RNA
ccctccattgct

SEQ ID NO: 887 - RNA, 55 nt, synthetic construct
  source 1..55: mol_type = other RNA
agtttgttgaagttcgtgactgcttcactctctcattcttagcttgaatttggaa

SEQ ID NO: 888 - RNA, 61 nt, synthetic construct
  source 1..61: mol_type = other RNA
atttttagtttgttgaagttcgtgactgcttcactctctcattcttagcttgaatttggaa

SEQ ID NO: 889 - RNA, 89 nt, synthetic construct
  source 1..89: mol_type = other RNA
agcccagaaaggagccacggggagaagcggagtttcttctttgtggcatttgtggaatattgtgtgccaaaaaaaaatcaggacttgtc

SEQ ID NO: 890 - RNA, 53 nt, synthetic construct
  source 1..53: mol_type = other RNA
tcttccttgcggaaatttgcatcgtgggcttgtagggactgcttcactgagcc

SEQ ID NO: 891 - RNA, 94 nt, synthetic construct
  source 1..94: mol_type = other RNA
gtcagtggaggcagcagcggtagaggcggcggcgaggactggcgacggcgaggagatagtgtctgccttctggagagctgaccaaacactaagg

SEQ ID NO: 892 - RNA, 66 nt, synthetic construct
  source 1..66: mol_type = other RNA
agtatattctgttctcttgggacattacagttttgcatagacaaggtgaacatttctcaaaacacc

SEQ ID NO: 893 - RNA, 42 nt, synthetic construct
  source 1..42: mol_type = other RNA
gcaagtagtacctcagggtaaactgccagaaggctaagggaa

SEQ ID NO: 894 - RNA, 12 nt, synthetic construct
  source 1..12: mol_type = other RNA
gtcacagaggag

SEQ ID NO: 895 - RNA, 95 nt, synthetic construct
  source 1..95: mol_type = other RNA
acttagttaaatttcagtaactattatatccgttttcctcggagtgagagaaaactctttttagatatcatctgagaggtagttaatttggcacc

SEQ ID NO: 896 - RNA, 54 nt, synthetic construct
  source 1..54: mol_type = other RNA
acactcattttagccagtgtccaggctatcagcagagaaagacaggtgggcagc

SEQ ID NO: 897 - RNA, 24 nt, synthetic construct
  source 1..24: mol_type = other RNA
gttgccaggacgacgcctgcgaag

SEQ ID NO: 898 - RNA, 54 nt, synthetic construct
  source 1..54: mol_type = other RNA
actattgggcgttacgcgagggcggggccggttgccaggacgacgcctgcgaag

SEQ ID NO: 899 - RNA, 27 nt, synthetic construct
  source 1..27: mol_type = other RNA
aacgcggttaccgtggaaaccgcggcc

SEQ ID NO: 900 - RNA, 18 nt, synthetic construct
  source 1..18: mol_type = other RNA
cccctcgtctcctccaag

SEQ ID NO: 901 - RNA, 43 nt, synthetic construct
  source 1..43: mol_type = other RNA
agtagaaaagtttcttttctttgaagtacagaactacagcata

SEQ ID NO: 902 - RNA, 51 nt, synthetic construct
  source 1..51: mol_type = other RNA
tctgcagagtgagagcaaactaaaagagaaaaatcaaagttgactacaata

SEQ ID NO: 903 - RNA, 89 nt, synthetic construct
  source 1..89: mol_type = other RNA
ctttcctctcctgactaagtttctctggcttccctgaggctgcaggtgttaatctggggggccctgggccctgagccggcagcagaaat

SEQ ID NO: 904 - RNA, 55 nt, synthetic construct
  source 1..55: mol_type = other RNA
aggttcgatacaagaggctgttttcctagcgtggcttgctgcctttggtaagaac

SEQ ID NO: 905 - RNA, 55 nt, synthetic construct
  source 1..55: mol_type = other RNA
atttgcctctgcaacttgactctcctctaggaagagtactccagagagcagggat

SEQ ID NO: 906 - RNA, 50 nt, synthetic construct
  source 1..50: mol_type = other RNA
ctatctgaccacattgttaaacttggttagttttgcaggttaaagaggaa

SEQ ID NO: 907 - RNA, 24 nt, synthetic construct
  source 1..24: mol_type = other RNA
atagtaaaggtcggcccgcgcaag

SEQ ID NO: 908 - RNA, 12 nt, synthetic construct
  source 1..12: mol_type = other RNA
cctctttagtgc

SEQ ID NO: 909 - RNA, 13 nt, synthetic construct
  source 1..13: mol_type = other RNA
ccgagcccgagcc

SEQ ID NO: 910 - RNA, 64 nt, synthetic construct
  source 1..64: mol_type = other RNA
actcttggaggaaggcatcagttaaggacactggaaaagacaggctcttttgttctggttccta

SEQ ID NO: 911 - RNA, 59 nt, synthetic construct
  source 1..59: mol_type = other RNA
gaaagttctatagtctctatattctgttatcaagtctttcaacaacttctttctacaga

SEQ ID NO: 912 - RNA, 74 nt, synthetic construct
  source 1..74: mol_type = other RNA
agaaggtcacaaagcttgcagggtaagtgacacaacttgaaactgcttggccctctttaaaaagaaataataaa

SEQ ID NO: 913 - RNA, 73 nt, synthetic construct
  source 1..73: mol_type = other RNA
aagtgatttgcttaaaatttctcacacaaagctacatttactggaggatctttctataaaaaataaacagagg

SEQ ID NO: 914 - RNA, 20 nt, synthetic construct
  source 1..20: mol_type = other RNA
tggccaggcccaggaaatcc

SEQ ID NO: 915 - RNA, 55 nt, synthetic construct
  source 1..55: mol_type = other RNA
acagagaacctcagctggacaaagcagccttgatctgagtgagctaactgacaca

SEQ ID NO: 916 - RNA, 53 nt, synthetic construct
  source 1..53: mol_type = other RNA
tcatatttgtgctccttcacgggagggcagggaggttcaacggaccttaaaac

SEQ ID NO: 917 - RNA, 93 nt, synthetic construct
  source 1..93: mol_type = other RNA
tctaggttttcacatcctttctagtagtttcgccatctatagcttttatcaacaattgtggtttaatcaattagaaatattaaccaggagaaa

SEQ ID NO: 918 - RNA, 64 nt, synthetic construct
  source 1..64: mol_type = other RNA
aattttaatcttcttttcttagaactatcttggttggcatcatcaggccctgagagcacagtgc

SEQ ID NO: 919 - RNA, 56 nt, synthetic construct
  source 1..56: mol_type = other RNA
ccgcgagcttacaccggcttctctctgtcctcagcccgcgcgccgccatcgccgtc

SEQ ID NO: 920 - RNA, 55 nt, synthetic construct
  source 1..55: mol_type = other RNA
gtgagttgcagtttgtgtgaggatcccggggccgccgcgtcgctcgggccccgcc

SEQ ID NO: 921 - RNA, 28 nt, synthetic construct
  source 1..28: mol_type = other RNA
ttgcctttaggttttccgaaggtcaaca

SEQ ID NO: 922 - RNA, 26 nt, synthetic construct
  source 1..26: mol_type = other RNA
atttgtgacttcttcagaccactttc

SEQ ID NO: 923 - RNA, 100 nt, synthetic construct
  source 1..100: mol_type = other RNA
attctctgcctctgaaggctccagaggctgtgggaagcagcacatcagcgacagctcctggctgctggactccgcagggagggaaggaagattggtcgca

SEQ ID NO: 924 - RNA, 68 nt, synthetic construct
  source 1..68: mol_type = other RNA
aaaagtaaaaccagttgttgtggagaaataagaggacctgctcctccaatttaaagtcagtttggcca

SEQ ID NO: 925 - RNA, 94 nt, synthetic construct
  source 1..94: mol_type = other RNA
atagttctgcagtcacaagggctttgtagtcagtcttagctgctggaacattcaaggagacaaaaaggcacagaagatctccacacatactttg

SEQ ID NO: 926 - RNA, 64 nt, synthetic construct
  source 1..64: mol_type = other RNA
agagagggagtcggtgtggacgcgaggagccgggcgcttagaacagaggcttgcacaggtggag

SEQ ID NO: 927 - RNA, 108 nt, synthetic construct
  source 1..108: mol_type = other RNA
actgaagcagcagctcagtttctcactccgaagtggcagcagccagagagggagtcggtgtggacgcgaggagccgggcgcttagaacagaggcttgcacaggtggag

SEQ ID NO: 928 - RNA, 88 nt, synthetic construct
  source 1..88: mol_type = other RNA
aaagcggccagtttttacatatttcttgattttgtttgttttctcgtgagcttaggccgctggttttggtgatttttgtctgattgca

SEQ ID NO: 929 - RNA, 61 nt, synthetic construct
  source 1..61: mol_type = other RNA
gattttgtttgttttctcgtgagcttaggccgctggttttggtgatttttgtctgattgca

SEQ ID NO: 930 - RNA, 48 nt, synthetic construct
  source 1..48: mol_type = other RNA
cttggaccaatcagccttgtgtttataggaattatttctgttcactgt

SEQ ID NO: 931 - RNA, 60 nt, synthetic construct
  source 1..60: mol_type = other RNA
aacaacagctgagtcagacagagtatctgtacactgttttataccaagaaatcatttaca

SEQ ID NO: 932 - RNA, 95 nt, synthetic construct
  source 1..95: mol_type = other RNA
acaggcagtaaccacgagtctcagccccttagctgaacttttaaacagagatttcttctatttcagacagacactgagactgaggcacccattcc

SEQ ID NO: 933 - RNA, 33 nt, synthetic construct
  source 1..33: mol_type = other RNA
ttctcttcaggagtaaacttctggaaaaataga

SEQ ID NO: 934 - RNA, 69 nt, synthetic construct
  source 1..69: mol_type = other RNA
ttctgtggtggttccaacctgtgataactgagaacaatacaaatagagatttgaaattcagtttgaatc

SEQ ID NO: 935 - RNA, 39 nt, synthetic construct
  source 1..39: mol_type = other RNA
cttgtacctgtcgttcgcagtggctcactttcccaagcc

SEQ ID NO: 936 - RNA, 44 nt, synthetic construct
  source 1..44: mol_type = other RNA
ccctctgtctttaggggctgagatttagtccatctgcctctgcc

SEQ ID NO: 937 - RNA, 59 nt, synthetic construct
  source 1..59: mol_type = other RNA
agtcacttgccatttctcataacagcgtcagagagaaagaactgactgaaacgtttgag

SEQ ID NO: 938 - RNA, 28 nt, synthetic construct
  source 1..28: mol_type = other RNA
tctcagcagtttaaagcaattgaacatc

SEQ ID NO: 939 - RNA, 87 nt, synthetic construct
  source 1..87: mol_type = other RNA
tgttgatagcccattgtgcaaaataagtgataaaattcaaagttcttgagtgtgctgaggtccagtgggaaaaacaagaaaactaag

SEQ ID NO: 940 - RNA, 61 nt, synthetic construct
  source 1..61: mol_type = other RNA
aagaaagtgggtcagcaggagttccagcatctcagaattgtggtgaaaaccaatacaaaaa

SEQ ID NO: 941 - RNA, 77 nt, synthetic construct
  source 1..77: mol_type = other RNA
ttctttttttaacgtttccacttttagtattatttcagagatcgcggattcaggtgtggctctagtagcaagtgaat

SEQ ID NO: 942 - RNA, 61 nt, synthetic construct
  source 1..61: mol_type = other RNA
ctttattccggaagttgctctcagaggcagcgtgcgggtgtgctctttgtgaaattccacc

SEQ ID NO: 943 - RNA, 53 nt, synthetic construct
  source 1..53: mol_type = other RNA
gcactcgacggctgactggagcagcggatataagtcaaagagtacagcagaaa

SEQ ID NO: 944 - RNA, 69 nt, synthetic construct
  source 1..69: mol_type = other RNA
gtacaggttatttcatttctggctgcagttttgcataaaaagggaactcgtgaggatattgaaaataac

SEQ ID NO: 945 - RNA, 24 nt, synthetic construct
  source 1..24: mol_type = other RNA
cttttctttgctggatctggaggc

SEQ ID NO: 946 - RNA, 57 nt, synthetic construct
  source 1..57: mol_type = other RNA
gagtagttctaccattctcttttgagtcatatagagagtgcgcaagttatagcacag

SEQ ID NO: 947 - RNA, 62 nt, synthetic construct
  source 1..62: mol_type = other RNA
aaaatactaaccagccagagaaacagaatcctaactatttctgaggaaactgcaggtccaaa

SEQ ID NO: 948 - RNA, 19 nt, synthetic construct
  source 1..19: mol_type = other RNA
ttattagcaagcagcaaat

SEQ ID NO: 949 - RNA, 58 nt, synthetic construct
  source 1..58: mol_type = other RNA
tattaacaaaatcagttaagcagtagaaaggtgggttcttctgaagataaagtgaacc

SEQ ID NO: 950 - RNA, 23 nt, synthetic construct
  source 1..23: mol_type = other RNA
ctgcggaagttcaactattcaac

SEQ ID NO: 951 - RNA, 65 nt, synthetic construct
  source 1..65: mol_type = other RNA
aaaacactgactagtaaggaggaagacattgaagaaggaaagccatctatctctgtaagaggaca

SEQ ID NO: 952 - RNA, 46 nt, synthetic construct
  source 1..46: mol_type = other RNA
actgatataaatctgcttaggaggcctgattcacagacgctacagg

SEQ ID NO: 953 - RNA, 69 nt, synthetic construct
  source 1..69: mol_type = other RNA
actcgctttcctcggattcccggagttgggggatttttgacatcttgtgaagcagaactacaggagctc

SEQ ID NO: 954 - RNA, 11 nt, synthetic construct
  source 1..11: mol_type = other RNA
aaggagagaaa

SEQ ID NO: 955 - RNA, 55 nt, synthetic construct
  source 1..55: mol_type = other RNA
agagggaaagcgagagggagacggacgttgagagaacgaggaggaaggagagaaa

SEQ ID NO: 956 - RNA, 57 nt, synthetic construct
  source 1..57: mol_type = other RNA
aacattaccagtttcaacttactagtctcaagtccaggcaacgtacaggcaggggaa

SEQ ID NO: 957 - RNA, 21 nt, synthetic construct
  source 1..21: mol_type = other RNA
agcaaggttccttggaagaag

SEQ ID NO: 958 - RNA, 51 nt, synthetic construct
  source 1..51: mol_type = other RNA
aattggtttagtatctgtatagtggcaatactctgtcctgcagtaggcagc

SEQ ID NO: 959 - RNA, 22 nt, synthetic construct
  source 1..22: mol_type = other RNA
acaaaccggacccgcaaccacc

SEQ ID NO: 960 - RNA, 46 nt, synthetic construct
  source 1..46: mol_type = other RNA
cattgtggctggcatagtatacatacttggttaaatataagggagg

SEQ ID NO: 961 - RNA, 65 nt, synthetic construct
  source 1..65: mol_type = other RNA
aatagcagcagtacagccttctgaatttggtacactgggtctccagattgatcaggaaactttac

SEQ ID NO: 962 - RNA, 63 nt, synthetic construct
  source 1..63: mol_type = other RNA
aggctgacagagaaatttttcattttctttcttgtttgactgtagtcaaacatcaaactaaga

SEQ ID NO: 963 - RNA, 44 nt, synthetic construct
  source 1..44: mol_type = other RNA
tcatttccatcaagtgtgcagcagtggtctctctgtagcaggcc

SEQ ID NO: 964 - RNA, 90 nt, synthetic construct
  source 1..90: mol_type = other RNA
accactggtcttcttgtgcttatccgggcaagaacttatcgaaatacaatagaagtttttacttagaagagattttcagggagaagtgaa

SEQ ID NO: 965 - RNA, 30 nt, synthetic construct
  source 1..30: mol_type = other RNA
gagagactacaactcccagtagtcaccgcg

SEQ ID NO: 966 - RNA, 31 nt, synthetic construct
  source 1..31: mol_type = other RNA
tataacaagaggattgcctgatccagccaag

SEQ ID NO: 967 - RNA, 12 nt, synthetic construct
  source 1..12: mol_type = other RNA
caaacagggaca

SEQ ID NO: 968 - RNA, 12 nt, synthetic construct
  source 1..12: mol_type = other RNA
acttgccgaagc

SEQ ID NO: 969 - RNA, 33 nt, synthetic construct
  source 1..33: mol_type = other RNA
ctggcagataagtgctctggttgtacagtgagg

SEQ ID NO: 970 - RNA, 56 nt, synthetic construct
  source 1..56: mol_type = other RNA
agatcgtcgcctatcctgcttgcgcagccgctatcgcgtctgcccaccgacgaagc

SEQ ID NO: 971 - RNA, 70 nt, synthetic construct
  source 1..70: mol_type = other RNA
ctgcattttgtgatcctctatttcaacactggtccagttagcagtagagttgtcacagccgacagaggca

SEQ ID NO: 972 - RNA, 92 nt, synthetic construct
  source 1..92: mol_type = other RNA
tgctgggattacaggcgtgagccaccgcgtctggcctcagccaaggtttttaagtaacatatttcagcattggctctacagcgttgcagaac

SEQ ID NO: 973 - RNA, 61 nt, synthetic construct
  source 1..61: mol_type = other RNA
acacatctcactcatccttctactcgtgacacttcccagttctggctttttgaaagcaaag

SEQ ID NO: 974 - RNA, 59 nt, synthetic construct
  source 1..59: mol_type = other RNA
aacgcaaaacctgctctttagatttcgagcttattctcttctagcagtttcttgccacc

SEQ ID NO: 975 - RNA, 19 nt, synthetic construct
  source 1..19: mol_type = other RNA
ctttcccggaggctggcag

SEQ ID NO: 976 - RNA, 90 nt, synthetic construct
  source 1..90: mol_type = other RNA
atacgagttactttttttaagtccgttgaaacagagttaatttcctttagcacacaagtcttagagacaaaagaaaaaaaggtctgcaac

SEQ ID NO: 977 - RNA, 20 nt, synthetic construct
  source 1..20: mol_type = other RNA
agaaacctgtgtagccagag

SEQ ID NO: 978 - RNA, 43 nt, synthetic construct
  source 1..43: mol_type = other RNA
ctgagcgcagttggagttcggaggaaaagggctcagattgaag

SEQ ID NO: 979 - RNA, 94 nt, synthetic construct
  source 1..94: mol_type = other RNA
aggacacatttagtcagagtagcttataagagaccgttccctccgccttcttcctcagaggaagtttcttgaagaatcttatcaaaaattagca

SEQ ID NO: 980 - RNA, 92 nt, synthetic construct
  source 1..92: mol_type = other RNA
agaaggatccatctaacatttactgaaataaggaagagtctttattcaatctaaaagtgaaaaaagaaccttttagacattattattataaa

SEQ ID NO: 981 - RNA, 12 nt, synthetic construct
  source 1..12: mol_type = other RNA
ctgtgtcttacc

SEQ ID NO: 982 - RNA, 56 nt, synthetic construct
  source 1..56: mol_type = other RNA
actcggcctaggaatttcccttatctccttcgcagtgcagctccttcaacctcgcc

SEQ ID NO: 983 - RNA, 55 nt, synthetic construct
  source 1..55: mol_type = other RNA
acctgcccagtgccacttccttctctctgtccctgtgggcccagcagctgccagg

SEQ ID NO: 984 - RNA, 56 nt, synthetic construct
  source 1..56: mol_type = other RNA
ttttgctctctgggattggtttcttaaagaatctggatcctttttaatagttcaaa

SEQ ID NO: 985 - RNA, 56 nt, synthetic construct
  source 1..56: mol_type = other RNA
gagaaagttgtctgcgtctcgaggcgagttggcggagctgtgcgcgcggcggggcg

SEQ ID NO: 986 - RNA, 54 nt, synthetic construct
  source 1..54: mol_type = other RNA
atccctctcagctcttgctaagtgatcactagcttgcctccaagctctctcact

SEQ ID NO: 987 - RNA, 25 nt, synthetic construct
  source 1..25: mol_type = other RNA
ttggttttgccactattgtttcatt

SEQ ID NO: 988 - RNA, 24 nt, synthetic construct
  source 1..24: mol_type = other RNA
atctgtttttttcccttctgagca

SEQ ID NO: 989 - RNA, 56 nt, synthetic construct
  source 1..56: mol_type = other RNA
tgcaattaaaggagtcgggtctctaactgttgatctgtttttttcccttctgagca

SEQ ID NO: 990 - RNA, 78 nt, synthetic construct
  source 1..78: mol_type = other RNA
acatttaagaaatacattggttcagtccagaaaggcaggacaacccgacgcgaggcagggagcttccaggttatagca

SEQ ID NO: 991 - RNA, 21 nt, synthetic construct
  source 1..21: mol_type = other RNA
cccccggcagcccggaggagc

SEQ ID NO: 992 - RNA, 22 nt, synthetic construct
  source 1..22: mol_type = other RNA
ccccgcctgatccgtgtccgac

SEQ ID NO: 993 - RNA, 12 nt, synthetic construct
  source 1..12: mol_type = other RNA
cttcctgctgtc

SEQ ID NO: 994 - RNA, 64 nt, synthetic construct
  source 1..64: mol_type = other RNA
aaaagtagtctaccctgaagaccccagatttttgatctgctcttttgagtcagttgatagaata

SEQ ID NO: 995 - RNA, 28 nt, synthetic construct
  source 1..28: mol_type = other RNA
acacctcagttcacaatcctggggcgat

SEQ ID NO: 996 - RNA, 62 nt, synthetic construct
  source 1..62: mol_type = other RNA
ttaaagtacttggagtttttataggttggcactcatcagtagcccctgttctcattctgcaa

SEQ ID NO: 997 - RNA, 46 nt, synthetic construct
  source 1..46: mol_type = other RNA
atttcaaggattctaccatcagaaaagaggccaaacttctatcatc

SEQ ID NO: 998 - RNA, 60 nt, synthetic construct
  source 1..60: mol_type = other RNA
cccagatctcttcagtttttagtcctcattctgtgaaaattgctgtagtctcttccagtt

SEQ ID NO: 999 - RNA, 99 nt, synthetic construct
  source 1..99: mol_type = other RNA
actgtgctggggtgtacatctacactagacaccttcctgcttccctccttccagagcagacctctttgtcaccccgagctccttgtttcttaagcagtc

SEQ ID NO: 1000 - RNA, 95 nt, synthetic construct
  source 1..95: mol_type = other RNA
gtttttctctctgctttctcaacagccagatctgccagtgagcctcaggctttaggaactgaagagtgtttctgaaagatctatccagcactccg

SEQ ID NO: 1001 - RNA, 88 nt, synthetic construct
  source 1..88: mol_type = other RNA
tcagggcagtagaacatattttaatcaagtccccttgataaaagagcttgattccagctactgcacttccttcatcagctctccactg

SEQ ID NO: 1002 - RNA, 49 nt, synthetic construct
  source 1..49: mol_type = other RNA
ccttgccagtccttcagagccattgtagcaggaggcagagtacagagtg

SEQ ID NO: 1003 - RNA, 54 nt, synthetic construct
  source 1..54: mol_type = other RNA
ggctttgggaagatctaaagacccaggaaggtctctgggtgggataaagccaag

SEQ ID NO: 1004 - RNA, 46 nt, synthetic construct
  source 1..46: mol_type = other RNA
caaggttatccatctcaagtagcctagcaatatttgcaacatccca

SEQ ID NO: 1005 - RNA, 34 nt, synthetic construct
  source 1..34: mol_type = other RNA
ctgctatacaactgagcctacctttcttgaaacc

SEQ ID NO: 1006 - RNA, 46 nt, synthetic construct
  source 1..46: mol_type = other RNA
agaatcttcttaggtacactcagctgaactcccatctctcatcagc

SEQ ID NO: 1007 - RNA, 12 nt, synthetic construct
  source 1..12: mol_type = other RNA
aagtggggcagc

SEQ ID NO: 1008 - RNA, 57 nt, synthetic construct
  source 1..57: mol_type = other RNA
agtcacacttcagaaatatcctcctacacctcaactgaaatctaacctcctgaaatc

SEQ ID NO: 1009 - RNA, 94 nt, synthetic construct
  source 1..94: mol_type = other RNA
ctttgccagtccatcttcaaattggaattatagaaagtagagggagggatagtctaccgtctctcactggattggtgccacctaaaacattgtt

SEQ ID NO: 1010 - RNA, 95 nt, synthetic construct
  source 1..95: mol_type = other RNA
tctttgccagtccatcttcaaattggaattatagaaagtagagggagggatagtctaccgtctctcactggattggtgccacctaaaacattgtt

SEQ ID NO: 1011 - RNA, 93 nt, synthetic construct
  source 1..93: mol_type = other RNA
agaaccagtaagcaaattaagtgtatcgattgcaattttgttggactaaccctgctttttttttcttaaggaataaacttgattccttttagt

SEQ ID NO: 1012 - RNA, 42 nt, synthetic construct
  source 1..42: mol_type = other RNA
agtcttttcgctggttattacatcttgcgtttctctgttgtt

SEQ ID NO: 1013 - RNA, 23 nt, synthetic construct
  source 1..23: mol_type = other RNA
gctgtctgtgtcttcaggtcatc

SEQ ID NO: 1014 - RNA, 16 nt, synthetic construct
  source 1..16: mol_type = other RNA
atttgtgacttgtctc

SEQ ID NO: 1015 - RNA, 69 nt, synthetic construct
  source 1..69: mol_type = other RNA
accagttcgcgctctcctccggcagaagtagcagctgcgctcttgctctctgggggagaacctggcgtt

SEQ ID NO: 1016 - RNA, 89 nt, synthetic construct
  source 1..89: mol_type = other RNA
tgaacaattgcagttactaccttagttctgtaaggtttctctctaaatattcagatcatcgtgtttggggaagatactgtgtaagagct

SEQ ID NO: 1017 - RNA, 64 nt, synthetic construct
  source 1..64: mol_type = other RNA
aaaagttgcttcttacaggaatataaatagtttctggaaaggacactgacaacttcaaagcaaa

SEQ ID NO: 1018 - RNA, 31 nt, synthetic construct
  source 1..31: mol_type = other RNA
agccgccgccggggctgtaggcgccaaggcc

SEQ ID NO: 1019 - RNA, 27 nt, synthetic construct
  source 1..27: mol_type = other RNA
ggaattagagaataaaatcccagcacg

SEQ ID NO: 1020 - RNA, 92 nt, synthetic construct
  source 1..92: mol_type = other RNA
gtgcgaaggaaaccgagtgggatcggaaccgtagcggttgagctgctgctgctacggatatctgacagaagccttcggtggttgtcggccta

SEQ ID NO: 1021 - RNA, 19 nt, synthetic construct
  source 1..19: mol_type = other RNA
actaggctctcagccaggg

SEQ ID NO: 1022 - RNA, 58 nt, synthetic construct
  source 1..58: mol_type = other RNA
tgcagtcctgtctcgtttccctctttctctctctctctctgatagtcacttcaatcaa

SEQ ID NO: 1023 - RNA, 51 nt, synthetic construct
  source 1..51: mol_type = other RNA
acttctgcagcacagctcccttcccaggacgtgaaaatctgccttctcacc

SEQ ID NO: 1024 - RNA, 69 nt, synthetic construct
  source 1..69: mol_type = other RNA
agagaacctggagcctaaggtttaggctcacccatttcaaccagtctagcagcatctgcaacatctaca

SEQ ID NO: 1025 - RNA, 55 nt, synthetic construct
  source 1..55: mol_type = other RNA
aatcctcctcttctcccttcctgaagctgcacgctgcagtaagagcacagcagaa

SEQ ID NO: 1026 - RNA, 21 nt, synthetic construct
  source 1..21: mol_type = other RNA
tgcagtaagagcacagcagaa

SEQ ID NO: 1027 - RNA, 66 nt, synthetic construct
  source 1..66: mol_type = other RNA
taattattgagtgctctgcctgcttaggactctgcccagccaggaaggaaacctacaggaggaaga

SEQ ID NO: 1028 - RNA, 70 nt, synthetic construct
  source 1..70: mol_type = other RNA
gttttcttagtatagttcattctcaataatatttcttatttatttagaaagttgaaataaaaggaccatc

SEQ ID NO: 1029 - RNA, 51 nt, synthetic construct
  source 1..51: mol_type = other RNA
actttgctgatttagcttagtgaagaggaaccagaaatttgtccttgaata

SEQ ID NO: 1030 - RNA, 26 nt, synthetic construct
  source 1..26: mol_type = other RNA
agtgaacttctgatagggtcttattt

SEQ ID NO: 1031 - RNA, 46 nt, synthetic construct
  source 1..46: mol_type = other RNA
cctttttggtgaattgagtgctgtttttgcttttctcagattccaa

SEQ ID NO: 1032 - RNA, 97 nt, synthetic construct
  source 1..97: mol_type = other RNA
aagacaggcatctcaaatcggctgattctgcatctggaaactgccttcatcttgaaagaaaagctccaggtcccttctccagccacccagccccaag

SEQ ID NO: 1033 - RNA, 16 nt, synthetic construct
  source 1..16: mol_type = other RNA
attctctggagtttga

SEQ ID NO: 1034 - RNA, 24 nt, synthetic construct
  source 1..24: mol_type = other RNA
tcagatcacagatttccctgccaa

SEQ ID NO: 1035 - RNA, 32 nt, synthetic construct
  source 1..32: mol_type = other RNA
ctgctcagcactaggccggcagaatctgagcg

SEQ ID NO: 1036 - RNA, 54 nt, synthetic construct
  source 1..54: mol_type = other RNA
aacactcacaagagagcagaggagccccaagtcttggggaccacagaagagtcc

SEQ ID NO: 1037 - RNA, 54 nt, synthetic construct
  source 1..54: mol_type = other RNA
agaagggtacaaatcttttttctttctccaaggagaatcttttgagcacaagta

SEQ ID NO: 1038 - RNA, 48 nt, synthetic construct
  source 1..48: mol_type = other RNA
ctgagtcacattgaggtctcttgctttaggaactgagtacgcttgata

SEQ ID NO: 1039 - RNA, 53 nt, synthetic construct
  source 1..53: mol_type = other RNA
acactcgcttctggaacgtctgaggttatcaataagctcctagtccagacgcc

SEQ ID NO: 1040 - RNA, 54 nt, synthetic construct
  source 1..54: mol_type = other RNA
gcattatccactagatacgtcgaagaagagggaaaccaattagggtcgaaataa

SEQ ID NO: 1041 - RNA, 97 nt, synthetic construct
  source 1..97: mol_type = other RNA
gaaaagtcaactgaaggaattgttgtgaaaaaagggacagcgagtttgaaataaaagttgttagagtggtactgaggagaaaaaagaatccgagacc

SEQ ID NO: 1042 - RNA, 50 nt, synthetic construct
  source 1..50: mol_type = other RNA
attggaagtacagtttttggaagtgtggagcagtttggctaagaatagga

SEQ ID NO: 1043 - RNA, 12 nt, synthetic construct
  source 1..12: mol_type = other RNA
acggccgtcgcc

SEQ ID NO: 1044 - RNA, 54 nt, synthetic construct
  source 1..54: mol_type = other RNA
tggatacactggaaaaagttcttgctgggcaaccaaaggacacaagtagattag

SEQ ID NO: 1045 - RNA, 55 nt, synthetic construct
  source 1..55: mol_type = other RNA
acaagagaacaacttgctccttccacaggaagctgcacctgacagaagctccagg

SEQ ID NO: 1046 - RNA, 23 nt, synthetic construct
  source 1..23: mol_type = other RNA
ctgtttggagagactcagccatc

SEQ ID NO: 1047 - RNA, 48 nt, synthetic construct
  source 1..48: mol_type = other RNA
gggagactggagtcgaaggccgtgaggtatttttctaagtcagccata

SEQ ID NO: 1048 - RNA, 33 nt, synthetic construct
  source 1..33: mol_type = other RNA
acaaagcgttgggtgagactcctcttgctcgtc

SEQ ID NO: 1049 - RNA, 54 nt, synthetic construct
  source 1..54: mol_type = other RNA
caaggcattcttccagttcctcagcctcctctttccttcctaggactggcttcc

SEQ ID NO: 1050 - RNA, 55 nt, synthetic construct
  source 1..55: mol_type = other RNA
tccttgcacaagaggaagtatatccaattcttgccaggcattgagactggcttcc

SEQ ID NO: 1051 - RNA, 76 nt, synthetic construct
  source 1..76: mol_type = other RNA
cctttctctcctctgagccacaactaaagtagttttctacttttccctttctactttcctagaccctggattttgt

SEQ ID NO: 1052 - RNA, 52 nt, synthetic construct
  source 1..52: mol_type = other RNA
gtctttggtagtttttttgcactaacttcaggagccagctcgtgatctcagg

SEQ ID NO: 1053 - RNA, 93 nt, synthetic construct
  source 1..93: mol_type = other RNA
agtggcaagcgcgggcaggaccgcgttgcgtcatcggggcgcgcgcctcagagagagctgtggttgccggaagttgagcggcggcaagaaata

SEQ ID NO: 1054 - RNA, 56 nt, synthetic construct
  source 1..56: mol_type = other RNA
ctcaaaactgccagtgtgagaggatttggaaatcactggatctgctcaatacaaaa

SEQ ID NO: 1055 - RNA, 70 nt, synthetic construct
  source 1..70: mol_type = other RNA
actgtttagtagtactcaaaactgccagtgtgagaggatttggaaatcactggatctgctcaatacaaaa

SEQ ID NO: 1056 - RNA, 94 nt, synthetic construct
  source 1..94: mol_type = other RNA
gtgaatattagtcatcatttatattttattactattattgtttacagaatcttgggactgttttacttaattggtcattgatagcttaacaaac

SEQ ID NO: 1057 - RNA, 21 nt, synthetic construct
  source 1..21: mol_type = other RNA
acagtgatatcctgagagaag

SEQ ID NO: 1058 - RNA, 69 nt, synthetic construct
  source 1..69: mol_type = other RNA
agagaacctagagcccaaggttcagagtcacccatctcagcaagcccagaagtatctgcaatatctacg

SEQ ID NO: 1059 - RNA, 76 nt, synthetic construct
  source 1..76: mol_type = other RNA
ccttcgcggcgcttcctagttcggctggttcttctgtcgccggcttcagcagcccgcgcccgggcaggaatagaag

SEQ ID NO: 1060 - RNA, 72 nt, synthetic construct
  source 1..72: mol_type = other RNA
aagcagattctaaaagcagtttctcttcagaacatcttttttcataccacttgataagcatcttgaaacacc

SEQ ID NO: 1061 - RNA, 48 nt, synthetic construct
  source 1..48: mol_type = other RNA
gttgaggtcaagtagtagcgttgggctgcggcagcggaggagctcaac

SEQ ID NO: 1062 - RNA, 25 nt, synthetic construct
  source 1..25: mol_type = other RNA
cttaccgtggcgagttccgcgctca

SEQ ID NO: 1063 - RNA, 52 nt, synthetic construct
  source 1..52: mol_type = other RNA
ggtagacgcgatctgctggctactaccggccttccctggctgttaaaagcag

SEQ ID NO: 1064 - RNA, 62 nt, synthetic construct; identical to SEQ ID NO: 996
  source 1..62: mol_type = other RNA

SEQ ID NO: 1065 - RNA, 61 nt, synthetic construct
  source 1..61: mol_type = other RNA
agactggtcttctgaggacctctgccctctacacagcggcctcttcaggtgcagggaggaa

SEQ ID NO: 1066 - RNA, 89 nt, synthetic construct
  source 1..89: mol_type = other RNA
gaaaactcatttgctcttggagcagtcaggcagtgactgccttcggctttttttctgctgactaagatctcctatagagagctacaaca

SEQ ID NO: 1067 - RNA, 18 nt, synthetic construct
  source 1..18: mol_type = other RNA
ttctctctgaccagcacc

SEQ ID NO: 1068 - RNA, 12 nt, synthetic construct
  source 1..12: mol_type = other RNA
gtcattggcacc

SEQ ID NO: 1069 - RNA, 21 nt, synthetic construct
  source 1..21: mol_type = other RNA
attggagacccttttcacaag

SEQ ID NO: 1070 - RNA, 86 nt, synthetic construct
  source 1..86: mol_type = other RNA
ctaaaacctgccagcagtacttttgagtttttttttttgttttgttttactttagcatttattattcagtgattgaagaaatcaaa

SEQ ID NO: 1071 - RNA, 56 nt, synthetic construct
  source 1..56: mol_type = other RNA
agtcaaactcagaatcttctcagtgtaactcagctgaactcacatctcccatcaac

SEQ ID NO: 1072 - RNA, 92 nt, synthetic construct
  source 1..92: mol_type = other RNA
agcataacttatagagaagctagtaagttctgagaaagaaggaaataaacacaggcaccaaaccactatcctaagttgactgtcctttaaat

SEQ ID NO: 1073 - RNA, 57 nt, synthetic construct
  source 1..57: mol_type = other RNA
acattcgaaaaactcactgtcttggaaacctacctagaacctccaccctctgacacc

SEQ ID NO: 1074 - RNA, 61 nt, synthetic construct
  source 1..61: mol_type = other RNA
ctttcaaagagacatattagtcaaggaagcaactcggaagaggaaagaaaggaagtcagga

SEQ ID NO: 1075 - RNA, 16 nt, synthetic construct
  source 1..16: mol_type = other RNA
tcttttctgccgggta

SEQ ID NO: 1076 - RNA, 55 nt, synthetic construct
  source 1..55: mol_type = other RNA
cccaggcagactgaggagtccctgtgtttttggcaaaggtagctctactagaaat

SEQ ID NO: 1077 - RNA, 70 nt, synthetic construct
  source 1..70: mol_type = other RNA
acattgccagcatcttctctcctcactcacccttcctggtgctttgggctctccttccttctcagacaag

SEQ ID NO: 1078 - RNA, 62 nt, synthetic construct
  source 1..62: mol_type = other RNA
taaactcctggtacttgagcactgatctgctttggagaacctgattctgagactccagcagg

SEQ ID NO: 1079 - RNA, 21 nt, synthetic construct
  source 1..21: mol_type = other RNA
ataggaatttagtgattgaca

SEQ ID NO: 1080 - RNA, 82 nt, synthetic construct
  source 1..82: mol_type = other RNA
acagtgctcattgtgcctgcgctgccagcagccaggagccaggagccaagagcagagcgccagcagtaacttgggggtcagc

SEQ ID NO: 1081 - RNA, 15 nt, synthetic construct
  source 1..15: mol_type = other RNA
ctggaaggaagcagg

SEQ ID NO: 1082 - RNA, 49 nt, synthetic construct
  source 1..49: mol_type = other RNA
aatcaaggagagtctctggacactataattcctgtgagaacacgagaag

SEQ ID NO: 1083 - RNA, 49 nt, synthetic construct
  source 1..49: mol_type = other RNA
attcagttggccactgctgagcagctgagaaggtggcgacgtaggggcc

SEQ ID NO: 1084 - RNA, 65 nt, synthetic construct
  source 1..65: mol_type = other RNA
ataaacaaacattgattgctttcctatcttacatttttttaggagtgcgaaataagtgagtcatc

SEQ ID NO: 1085 - RNA, 27 nt, synthetic construct
  source 1..27: mol_type = other RNA
acgagttcttccggggcggaggtcacc

SEQ ID NO: 1086 - RNA, 53 nt, synthetic construct
  source 1..53: mol_type = other RNA
agattttgggaaactcacctcttaacagaagcccaccctccatccctgacacc

SEQ ID NO: 1087 - RNA, 60 nt, synthetic construct
  source 1..60: mol_type = other RNA
agaaggcactccattctggctttcacaaatacagctctgcaacgcgtttgccctgatacc

SEQ ID NO: 1088 - RNA, 53 nt, synthetic construct
  source 1..53: mol_type = other RNA
cacactctgcctttggacgtgagagagagcgcacctttcacttgagcttcaac

SEQ ID NO: 1089 - RNA, 61 nt, synthetic construct
  source 1..61: mol_type = other RNA
accagaaccctgaattccgagatagagggagtgttgatctcagtctaagtaagtttagacc

SEQ ID NO: 1090 - RNA, 56 nt, synthetic construct
  source 1..56: mol_type = other RNA
gtgcagcattgtactgcaagtcaatcgatacaataatttaagtcacttcagctata

SEQ ID NO: 1091 - RNA, 99 nt, synthetic construct
  source 1..99: mol_type = other RNA
ataactcagcctctccagagtgcagccaccagtacctccgcagattgagtagtgaagaaaagaaaaccaggatatcctgtgctctggcttccctggacc

SEQ ID NO: 1092 - RNA, 52 nt, synthetic construct
  source 1..52: mol_type = other RNA
tttcttagttggaagttgtggtagttctccaggctggaggacacacctaaac

SEQ ID NO: 1093 - RNA, 64 nt, synthetic construct
  source 1..64: mol_type = other RNA
agactgcgaaggagaacgcagcaagcccaggcggcggtggaaaggctggaggacacacctaaac

SEQ ID NO: 1094 - RNA, 14 nt, synthetic construct
  source 1..14: mol_type = other RNA
cgaacacctccaac

SEQ ID NO: 1095 - RNA, 69 nt, synthetic construct
  source 1..69: mol_type = other RNA
agtcctgcttctcttccctctctcctccagcctctcacactctcctcagctctctcatctcctggaacc

SEQ ID NO: 1096 - RNA, 30 nt, synthetic construct; identical to SEQ ID NO: 965
  source 1..30: mol_type = other RNA

SEQ ID NO: 1097 - RNA, 30 nt, synthetic construct; identical to SEQ ID NO: 965
  source 1..30: mol_type = other RNA

SEQ ID NO: 1098 - RNA, 74 nt, synthetic construct
  source 1..74: mol_type = other RNA
agacacacacagcctctctgcccacctctgcttcctctaggaacacaggagttccagatcacatcgagttcacc

SEQ ID NO: 1099 - RNA, 23 nt, synthetic construct
  source 1..23: mol_type = other RNA
attgaactccatcttctgacacc

SEQ ID NO: 1100 - RNA, 15 nt, synthetic construct
  source 1..15: mol_type = other RNA
ctggaaggaagcagg

SEQ ID NO: 1101 - RNA, 52 nt, synthetic construct
  source 1..52: mol_type = other RNA
ggggaacatagagtgctgaagacagaatctagagccttcaaataagttggag

SEQ ID NO: 1102 - RNA, 21 nt, synthetic construct
  source 1..21: mol_type = other RNA
aatcactccggagactgagcc

SEQ ID NO: 1103 - RNA, 12 nt, synthetic construct
  source 1..12: mol_type = other RNA
gctgacgtgaag

SEQ ID NO: 1104 - RNA, 52 nt, synthetic construct
  source 1..52: mol_type = other RNA
ggggaacatagagtgctggagacagaatctagagccttcaaataagttggag

SEQ ID NO: 1105 - RNA, 55 nt, synthetic construct
  source 1..55: mol_type = other RNA
gccggaagcggaaatagcaccgggcgccgccacagtagctgtaactgccaccgcg

SEQ ID NO: 1106 - RNA, 65 nt, synthetic construct; identical to SEQ ID NO: 1084
  source 1..65: mol_type = other RNA

SEQ ID NO: 1107 - RNA, 27 nt, synthetic construct
  source 1..27: mol_type = other RNA
tcagctcccagagtgatcctggacagg

SEQ ID NO: 1108 - RNA, 63 nt, synthetic construct
  source 1..63: mol_type = other RNA
ataaaatctgtgctgtcaaactgattaggaactgactaccacctgcaggtcagggccaaggtt

SEQ ID NO: 1109 - RNA, 64 nt, synthetic construct
  source 1..64: mol_type = other RNA
atcttaaaagaggttctttctctgggagttggcagtagcaaaactgacaagtcaaggcaggaag

SEQ ID NO: 1110 - RNA, 44 nt, synthetic construct
  source 1..44: mol_type = other RNA
caagtcacagatacagaagacccctccgtcctggagcacctgcc

SEQ ID NO: 1111 - RNA, 20 nt, synthetic construct
  source 1..20: mol_type = other RNA
attctttcttcaaagcagcc

SEQ ID NO: 1112 - RNA, 56 nt, synthetic construct
  source 1..56: mol_type = other RNA
cactgtgatttcttcagtttaaggatcaagaccattatttgggtaacacactaaag

SEQ ID NO: 1113 - RNA, 56 nt, synthetic construct
  source 1..56: mol_type = other RNA
attatttttttttcgtgtttaaggtttgaatcctcagtgaaccagggcagaaaaga

SEQ ID NO: 1114 - RNA, 69 nt, synthetic construct
  source 1..69: mol_type = other RNA
acagaagtggcgcctctgagaaaagaaggttggaattatcgtaatttgtttctaggctgagataccagc

SEQ ID NO: 1115 - RNA, 76 nt, synthetic construct
  source 1..76: mol_type = other RNA
actatttctattgatctggaagaagcttgtaccaggcaacccatttaggagaagttggagtaagagggagagggag

SEQ ID NO: 1116 - RNA, 61 nt, synthetic construct
  source 1..61: mol_type = other RNA
agcatcatccagaaaccacatccctccgctagagaagcccctgacggcacagttcctcact

SEQ ID NO: 1117 - RNA, 52 nt, synthetic construct
  source 1..52: mol_type = other RNA
tcacaaccagtgatattaggcttgcattttctttctttttttctgctcagat

SEQ ID NO: 1118 - RNA, 67 nt, synthetic construct
  source 1..67: mol_type = other RNA
attcacacctcagaagccttatccttcttttcaatcaaacccataaataccacagactctaatagcc

SEQ ID NO: 1119 - RNA, 54 nt, synthetic construct
  source 1..54: mol_type = other RNA
gtagccattcctgaatttctttcagcactgggaaaaccagtctagtcaccaaaa

SEQ ID NO: 1120 - RNA, 66 nt, synthetic construct
  source 1..66: mol_type = other RNA
atttacttcctgcgggtgcacaggctgtggtcgtctatctccctgttgttcttcccatcggcgaag

SEQ ID NO: 1121 - RNA, 12 nt, synthetic construct
  source 1..12: mol_type = other RNA
gttgctgggaag

SEQ ID NO: 1122 - RNA, 46 nt, synthetic construct
  source 1..46: mol_type = other RNA
tcctatcgataaggttcagttagccaaggtggaggagtggaaaaga

SEQ ID NO: 1123 - RNA, 20 nt, synthetic construct
  source 1..20: mol_type = other RNA
ctagtctgcatctgagtaac

SEQ ID NO: 1124 - RNA, 31 nt, synthetic construct
  source 1..31: mol_type = other RNA
gtctctggtttctggccccttgtctgcagag

SEQ ID NO: 1125 - RNA, 55 nt, synthetic construct
  source 1..55: mol_type = other RNA
acatatctgcttccgacacagctgcaatcactagcaagctctcaggcctggcatc

SEQ ID NO: 1126 - RNA, 53 nt, synthetic construct; identical to SEQ ID NO: 1039
  source 1..53: mol_type = other RNA

SEQ ID NO: 1127 - RNA, 55 nt, synthetic construct
  source 1..55: mol_type = other RNA
cagcgcttgaggcttgaagagggagacaagctaaaagaggatatccagttgtttc

SEQ ID NO: 1128 - RNA, 60 nt, synthetic construct
  source 1..60: mol_type = other RNA
gtaagtgtgagaaatttggcaataaaaccacctatctttacagattttgggaagtccaaa

SEQ ID NO: 1129 - RNA, 77 nt, synthetic construct
  source 1..77: mol_type = other RNA
gagtggagaggaaaaacaaattatttttaagtattcacggcaaccacatttatatcctattgttactagagaaaaaa

SEQ ID NO: 1130 - RNA, 12 nt, synthetic construct
  source 1..12: mol_type = other RNA
cagttgtacacc

SEQ ID NO: 1131 - RNA, 20 nt, synthetic construct
  source 1..20: mol_type = other RNA
tcattgcagagtcgaaagcc

SEQ ID NO: 1132 - RNA, 53 nt, synthetic construct
  source 1..53: mol_type = other RNA
cagctctcggctctgcagcggagcataggcgaccaccaccgaaagaggccacc

SEQ ID NO: 1133 - RNA, 87 nt, synthetic construct
  source 1..87: mol_type = other RNA
acagtgaactctgattcctggaattgttaccaggcgaccacggcgtcatcaaagtacctggcaattccgtctactgagttctcgaac

SEQ ID NO: 1134 - RNA, 116 nt, synthetic construct
  source 1..116: mol_type = other RNA
attttctcaagcaatttttaaatttcattcttttaccctattgagcttttcttagtgcctaaagtgtctgcaagtggaaatccagaggaagacagagagaaactgagttcctgaca

SEQ ID NO: 1135 - RNA, 57 nt, synthetic construct
  source 1..57: mol_type = other RNA
acaggttagtagtgccagtgagccgccttagatagaagcatcgtcagcactttatta

SEQ ID NO: 1136 - RNA, 94 nt, synthetic construct
  source 1..94: mol_type = other RNA
tttgttgagagtggagttggaaagtgttagcactttgccagactttgccagcaagattaactgaggagatcaaattgagtcagggtcaaggcta

SEQ ID NO: 1137 - RNA, 53 nt, synthetic construct
  source 1..53: mol_type = other RNA
aggccctgtttccctgagctctcacctctgatacaagccttaaagaagagtaa

SEQ ID NO: 1138 - RNA, 42 nt, synthetic construct
  source 1..42: mol_type = other RNA
cccattcttggcgcagaaacctgaaagtcaggatcaaagaga

SEQ ID NO: 1139 - RNA, 34 nt, synthetic construct
  source 1..34: mol_type = other RNA
ccttcctggaggttttatcacccacactacatag

SEQ ID NO: 1140 - RNA, 52 nt, synthetic construct; identical to SEQ ID NO: 1101
  source 1..52: mol_type = other RNA

SEQ ID NO: 1141 - RNA, 52 nt, synthetic construct; identical to SEQ ID NO: 1101
  source 1..52: mol_type = other RNA

SEQ ID NO: 1142 - RNA, 12 nt, synthetic construct
  source 1..12: mol_type = other RNA
gctgacgtgaag

SEQ ID NO: 1143 - RNA, 12 nt, synthetic construct
  source 1..12: mol_type = other RNA
gctgacgtgaag

SEQ ID NO: 1144 - RNA, 67 nt, synthetic construct
  source 1..67: mol_type = other RNA
ggtatcttaaaagaggttctttctctgggagttggcagtagcaaaactgacaagtcaaggcaggaag

SEQ ID NO: 1145 - RNA, 65 nt, synthetic construct; identical to SEQ ID NO: 1084
  source 1..65: mol_type = other RNA

SEQ ID NO: 1146 - RNA, 58 nt, synthetic construct
  source 1..58: mol_type = other RNA
tcctcacaattactctacagctcagaacagcaactgctgaggctgccttgggaagaag

SEQ ID NO: 1147 - RNA, 53 nt, synthetic construct; identical to SEQ ID NO: 1088
  source 1..53: mol_type = other RNA

SEQ ID NO: 1148 - RNA, 55 nt, synthetic construct; identical to SEQ ID NO: 1105
  source 1..55: mol_type = other RNA

SEQ ID NO: 1149 - RNA, 65 nt, synthetic construct; identical to SEQ ID NO: 1084
  source 1..65: mol_type = other RNA

SEQ ID NO: 1150 - RNA, 71 nt, synthetic construct
  source 1..71: mol_type = other RNA
accttcgccctaagtcctttaccaatagtgcggcactccggtagcgacgaatccatcctgcactgacacgc

SEQ ID NO: 1151 - RNA, 10 nt, synthetic construct
  source 1..10: mol_type = other RNA
ttcagtctca

SEQ ID NO: 1152 - RNA, 70 nt, synthetic construct
  source 1..70: mol_type = other RNA
agaaaacctacaacccaaggttcagtgttacccctcatcaaccagcccagcagcatcttcgggattccca

SEQ ID NO: 1153 - RNA, 21 nt, synthetic construct
  source 1..21: mol_type = other RNA
aatcactccggagactgagcc

SEQ ID NO: 1154 - RNA, 55 nt, synthetic construct
  source 1..55: mol_type = other RNA
ctctgacttcgggcttgttcgctggtggcgtcggagccgagccggactggtcagg

SEQ ID NO: 1155 - RNA, 12 nt, synthetic construct
  source 1..12: mol_type = other RNA
gctgacgtgaag

SEQ ID NO: 1156 - RNA, 15 nt, synthetic construct
  source 1..15: mol_type = other RNA
ctggaaggaagcagg

SEQ ID NO: 1157 - RNA, 21 nt, synthetic construct
  source 1..21: mol_type = other RNA
aatcactccggagactgagcc

SEQ ID NO: 1158 - RNA, 15 nt, synthetic construct
  source 1..15: mol_type = other RNA
ctggaaggaagcagg

SEQ ID NO: 1159 - RNA, 27 nt, synthetic construct
  source 1..27: mol_type = other RNA
tcagctcccagagtgatcctggacagg

SEQ ID NO: 1160 - RNA, 55 nt, synthetic construct; identical to SEQ ID NO: 1105
  source 1..55: mol_type = other RNA

SEQ ID NO: 1161 - RNA, 15 nt, synthetic construct
  source 1..15: mol_type = other RNA
ctggaaggaagcagg

SEQ ID NO: 1162 - RNA, 65 nt, synthetic construct; identical to SEQ ID NO: 1084
  source 1..65: mol_type = other RNA

SEQ ID NO: 1163 - RNA, 21 nt, synthetic construct
  source 1..21: mol_type = other RNA
aatcactccggagactgagcc

SEQ ID NO: 1164 - RNA, 51 nt, synthetic construct
  source 1..51: mol_type = other RNA
aaagtttattcagcttttcgatccgccatctgcagtggagccgccaccaaa

SEQ ID NO: 1165 - RNA, 55 nt, synthetic construct; identical to SEQ ID NO: 1105
  source 1..55: mol_type = other RNA

SEQ ID NO: 1166 - RNA, 21 nt, synthetic construct
  source 1..21: mol_type = other RNA
aatcactccggagactgagcc

SEQ ID NO: 1167 - RNA, 67 nt, synthetic construct
  source 1..67: mol_type = other RNA
gaaggaaagagtaacttgagtttcacttcttagtgccttttctcaggggagaggccatcacttgaag

SEQ ID NO: 1168 - RNA, 65 nt, synthetic construct; identical to SEQ ID NO: 1084
  source 1..65: mol_type = other RNA

SEQ ID NO: 1169 - RNA, 65 nt, synthetic construct; identical to SEQ ID NO: 1084
  source 1..65: mol_type = other RNA

SEQ ID NO: 1170 - RNA, 27 nt, synthetic construct
  source 1..27: mol_type = other RNA
tcagctcccagagtgatcctggacagg

SEQ ID NO: 1171 - RNA, 65 nt, synthetic construct; identical to SEQ ID NO: 1084
  source 1..65: mol_type = other RNA

SEQ ID NO: 1172 - RNA, 61 nt, synthetic construct
  source 1..61: mol_type = other RNA
gttgacagagctaccgtcttcctgtttttttcctccaattttccggcagttactcccagtc

SEQ ID NO: 1173 - RNA, 52 nt, synthetic construct; identical to SEQ ID NO: 1101
  source 1..52: mol_type = other RNA

SEQ ID NO: 1174 - RNA, 15 nt, synthetic construct
  source 1..15: mol_type = other RNA
ctggaaggaagcagg

SEQ ID NO: 1175 - RNA, 21 nt, synthetic construct
  source 1..21: mol_type = other RNA
aatcactccggagactgagcc

SEQ ID NO: 1176 - RNA, 55 nt, synthetic construct; identical to SEQ ID NO: 1105
  source 1..55: mol_type = other RNA

SEQ ID NO: 1177 - RNA, 55 nt, synthetic construct; identical to SEQ ID NO: 1105
  source 1..55: mol_type = other RNA

SEQ ID NO: 1178 - RNA, 58 nt, synthetic construct; identical to SEQ ID NO: 1146
  source 1..58: mol_type = other RNA

SEQ ID NO: 1179 - RNA, 27 nt, synthetic construct
  source 1..27: mol_type = other RNA
tcagctcccagagtgatcctggacagg

SEQ ID NO: 1180 - RNA, 15 nt, synthetic construct
  source 1..15: mol_type = other RNA
ctggaaggaagcagg

SEQ ID NO: 1181 - RNA, 53 nt, synthetic construct
  source 1..53: mol_type = other RNA
acaattgctctacagctcagagcagcaactgctgaggctgccttgggaagaag

SEQ ID NO: 1182 - RNA, 27 nt, synthetic construct
  source 1..27: mol_type = other RNA
tcagctcccagagtgatcctggacagg

SEQ ID NO: 1183 - RNA, 73 nt, synthetic construct
  source 1..73: mol_type = other RNA
ttagttcttcctgaggtctgagcaccctccaaactgagtccagatctgagtttttcctcggagatttgtcaga

SEQ ID NO: 1184 - RNA, 60 nt, synthetic construct
  source 1..60: mol_type = other RNA
tcttcattctttccgccatcttgattctttgtcactgaccgagactcagccgtgggaaat

SEQ ID NO: 1185 - RNA, 61 nt, synthetic construct
  source 1..61: mol_type = other RNA
gacagtttgaggtaaggcgttgtatatattgcagatagctctgaggtttttggagtgtacc

SEQ ID NO: 1186 - RNA, 53 nt, synthetic construct
  source 1..53: mol_type = other RNA
aggctggacacacttcagtcaggagtcagaccctgtcaggacacagcatagac

SEQ ID NO: 1187 - RNA, 63 nt, synthetic construct
  source 1..63: mol_type = other RNA
tctctcttatcttctggcctgcctggagtttcaagggaacaagtacagagtgtgatttggatc

SEQ ID NO: 1188 - RNA, 12 nt, synthetic construct
  source 1..12: mol_type = other RNA
tggaaaatccct

SEQ ID NO: 1189 - RNA, 16 nt, synthetic construct
  source 1..16: mol_type = other RNA
acatttcagggacacc

SEQ ID NO: 1190 - RNA, 12 nt, synthetic construct
  source 1..12: mol_type = other RNA
ctgggaagcgcc

SEQ ID NO: 1191 - RNA, 57 nt, synthetic construct
  source 1..57: mol_type = other RNA
ttttcgtcgactcttaccggttggctgggccagctgcgccgcggctcacagctgacg

SEQ ID NO: 1192 - RNA, 66 nt, synthetic construct
  source 1..66: mol_type = other RNA
aaacctttttagggagtccaaggtacagtcgccgcgtgcggagcttgttactggttacttggcctc

SEQ ID NO: 1193 - RNA, 42 nt, synthetic construct
  source 1..42: mol_type = other RNA
ataggttggcactcatcagtagcccctgttctcattctgcaa

SEQ ID NO: 1194 - RNA, 62 nt, synthetic construct
  source 1..62: mol_type = other RNA
taaactcctggtacctaagcaccgatctgccttggagaacctgattctgagactccagcagg

SEQ ID NO: 1195 - RNA, 57 nt, synthetic construct
  source 1..57: mol_type = other RNA
aaaccttccaaacccaccttcctggaaatccacccagaacctccaccctctgacacc

SEQ ID NO: 1196 - RNA, 90 nt, synthetic construct
  source 1..90: mol_type = other RNA
tgaagacagccacacagatattgcacagactatttacagatcgtttggtttacattgagagtcattgctctacttttgtgcggtaggaaa

SEQ ID NO: 1197 - RNA, 55 nt, synthetic construct
  source 1..55: mol_type = other RNA
tccactggccagcattctcctctattagactagaactgtggataaacctcagaaa

SEQ ID NO: 1198 - RNA, 49 nt, synthetic construct
  source 1..49: mol_type = other RNA
actcagaagactggacacaattccgaaggtcgcccagaaggagaggaca

SEQ ID NO: 1199 - RNA, 43 nt, synthetic construct
  source 1..43: mol_type = other RNA
aagactggacacaattccgaaggtcgcccagaaggagaggaca

SEQ ID NO: 1200 - RNA, 52 nt, synthetic construct
  source 1..52: mol_type = other RNA
gtctttggtagtttttttgcactaacttcaggaaccagctcagtatctcagg

SEQ ID NO: 1201 - RNA, 56 nt, synthetic construct
  source 1..56: mol_type = other RNA
acaagagcaagcagaacctctctgaccgctcgcgccaggcctacactttccacctc

SEQ ID NO: 1202 - RNA, 47 nt, synthetic construct
  source 1..47: mol_type = other RNA
ctggccagatctctctacacaggtaaaagagtaagccaacaatacaa

SEQ ID NO: 1203 - RNA, 43 nt, synthetic construct
  source 1..43: mol_type = other RNA
attgcaagtgtaacacacattgcaagtgccaacgtgaggagcc

SEQ ID NO: 1204 - RNA, 46 nt, synthetic construct
  source 1..46: mol_type = other RNA
gtggtggcagaggtgcttgtgtttttgtcggtacaggagagtcgct

SEQ ID NO: 1205 - RNA, 12 nt, synthetic construct
  source 1..12: mol_type = other RNA
gttgctaccgga

SEQ ID NO: 1206 - RNA, 51 nt, synthetic construct
  source 1..51: mol_type = other RNA
attgagtaaaattgggttaccctgaggactcttgcttaactgaaatcacaa

SEQ ID NO: 1207 - RNA, 21 nt, synthetic construct
  source 1..21: mol_type = other RNA
tttccggtgcaggtggggaaa

SEQ ID NO: 1208 - RNA, 44 nt, synthetic construct
  source 1..44: mol_type = other RNA
acagtgcgcagttgtgcggctgtgctttggctcttcgggtaaag

SEQ ID NO: 1209 - RNA, 48 nt, synthetic construct
  source 1..48: mol_type = other RNA
catctccaggctccatctcttgaaccagtgatcccagcgttgttagca

SEQ ID NO: 1210 - RNA, 54 nt, synthetic construct
  source 1..54: mol_type = other RNA
attgctaagccatccttcagacagagagggagcggctgcaagaggtaagtagag

SEQ ID NO: 1211 - RNA, 26 nt, synthetic construct
  source 1..26: mol_type = other RNA
ctcactccgtctcaatagttctcaag

SEQ ID NO: 1212 - RNA, 24 nt, synthetic construct
  source 1..24: mol_type = other RNA
cactccgtctcaatagttctcaag

SEQ ID NO: 1213 - RNA, 16 nt, synthetic construct
  source 1..16: mol_type = other RNA
acagcggtcacgtgac

SEQ ID NO: 1214 - RNA, 53 nt, synthetic construct
  source 1..53: mol_type = other RNA
cgtatcgcttggagtgaaggtcctaccgcggcagtattgatttctaaagactc

SEQ ID NO: 1215 - RNA, 12 nt, synthetic construct
  source 1..12: mol_type = other RNA
gctgacgtgaag

SEQ ID NO: 1216 - RNA, 12 nt, synthetic construct
  source 1..12: mol_type = other RNA
gctgacgtgaag

SEQ ID NO: 1217 - RNA, 42 nt, synthetic construct
  source 1..42: mol_type = other RNA
gtctcccagacagtggctcagccttcaggactctcttccagc

SEQ ID NO: 1218 - RNA, 11 nt, synthetic construct
  source 1..11: mol_type = other RNA
gagaacgaaag

SEQ ID NO: 1219 - RNA, 34 nt, synthetic construct
  source 1..34: mol_type = other RNA
ccgggccagttagatttggaggttcaacttcaac

SEQ ID NO: 1220 - RNA, 30 nt, synthetic construct; identical to SEQ ID NO: 965
  source 1..30: mol_type = other RNA

SEQ ID NO: 1221 - RNA, 22 nt, synthetic construct
  source 1..22: mol_type = other RNA
actctcttgctcacagtccacg

SEQ ID NO: 1222 - RNA, 52 nt, synthetic construct
  source 1..52: mol_type = other RNA
ctaggcctctgtgattgttgaggagggggctgtcactgggaaagtttccacg

SEQ ID NO: 1223 - RNA, 21 nt, synthetic construct
  source 1..21: mol_type = other RNA
cctcccactctgcctagagtc

SEQ ID NO: 1224 - RNA, 59 nt, synthetic construct
  source 1..59: mol_type = other RNA
acagtccctctgagctgcacggagacctcgcaggcccccggaactgtcgcccttccagg

SEQ ID NO: 1225 - RNA, 45 nt, synthetic construct
  source 1..45: mol_type = other RNA
agaaaggaacacagtaaactgaattgatccgtttagaagtttaca

SEQ ID NO: 1226 - RNA, 92 nt, synthetic construct
  source 1..92: mol_type = other RNA
agaggaggaaattgttcctcgtctgataagacaacagtggagaaaggacgcagtctgtttcttagggacacggctgacttccagatagtacc

SEQ ID NO: 1227 - RNA, 60 nt, synthetic construct
  source 1..60: mol_type = other RNA
gctgtgaggacgtgttccgaggaagccagagccggagccgtggcctgcggggccggcgac

SEQ ID NO: 1228 - RNA, 64 nt, synthetic construct
  source 1..64: mol_type = other RNA
aaggtcaagtatagcatctgcctagagtcaaacctccgtgcttctcagacagtgccttttcacc

SEQ ID NO: 1229 - RNA, 54 nt, synthetic construct; identical to SEQ ID NO: 262
  source 1..54: mol_type = other RNA

SEQ ID NO: 1230 - RNA, 12 nt, synthetic construct
  source 1..12: mol_type = other RNA
gctgacgtgaag

SEQ ID NO: 1231 - RNA, 42 nt, synthetic construct
  source 1..42: mol_type = other RNA
caaccatcctgaagctacaggtgctccctcctggaatctcca

SEQ ID NO: 1232 - RNA, 75 nt, synthetic construct
  source 1..75: mol_type = other RNA
actataaagacagtaaaaagatcagtggttatctttgcagacgccaccatcactgtgagccctgtactatcagcc

SEQ ID NO: 1233 - RNA, 51 nt, synthetic construct
  source 1..51: mol_type = other RNA
ccttttgcggccatcaccgaagcgggagcgggtaaggattcggcggccaaa

SEQ ID NO: 1234 - RNA, 42 nt, synthetic construct
  source 1..42: mol_type = other RNA
atcgataaggttcagttagccaaggtggaggagtggaaaaga

SEQ ID NO: 1235 - RNA, 21 nt, synthetic construct
  source 1..21: mol_type = other RNA
gagatctcgatcgaaggcgag

SEQ ID NO: 1236 - RNA, 26 nt, synthetic construct
  source 1..26: mol_type = other RNA
gtgagtggaagctctggtgcagcagt

SEQ ID NO: 1237 - RNA, 70 nt, synthetic construct
  source 1..70: mol_type = other RNA
gagaacccttccggtgggctaggtactgagcgcgcgaggctctacagagtgaaggtttaaatccaaggtc

SEQ ID NO: 1238 - RNA, 51 nt, synthetic construct
  source 1..51: mol_type = other RNA
acgctgggttctgcttttccagaatcctgccctttccaaaaggaagacggt

SEQ ID NO: 1239 - RNA, 73 nt, synthetic construct
  source 1..73: mol_type = other RNA
tgtggggagggtacctctcacagaagagtcttagtactggcatcaggggaaggtcaccaagtctttcctgtac

SEQ ID NO: 1240 - RNA, 75 nt, synthetic construct
  source 1..75: mol_type = other RNA
actataaagacagtaaaaagatcagtggttatctttgcagacgccaccatcgctgtgagccctgtactatcagcc

SEQ ID NO: 1241 - RNA, 55 nt, synthetic construct
  source 1..55: mol_type = other RNA
accctctcacatcccagtctctgatcagggaaagcagggcacagccttgggaaga

SEQ ID NO: 1242 - RNA, 71 nt, synthetic construct
  source 1..71: mol_type = other RNA
aaagtgctgtggggagtgatcacatttttcacaacatccctggctccacctcttctgccacaaacgtcagc

SEQ ID NO: 1243 - RNA, 53 nt, synthetic construct
  source 1..53: mol_type = other RNA
cgactttcccgatcgccaggcaggagtttctctcggtgactactatcgctgtc

SEQ ID NO: 1244 - RNA, 14 nt, synthetic construct
  source 1..14: mol_type = other RNA
aagctagggccacc

SEQ ID NO: 1245 - RNA, 24 nt, synthetic construct
  source 1..24: mol_type = other RNA
ccgcctgtctgcaccggcagcacc

SEQ ID NO: 1246 - RNA, 33 nt, synthetic construct; identical to SEQ ID NO: 1048
  source 1..33: mol_type = other RNA

SEQ ID NO: 1247 - RNA, 24 nt, synthetic construct
  source 1..24: mol_type = other RNA
ccgcctgtctgcaccggcagcacc

SEQ ID NO: 1248 - RNA, 56 nt, synthetic construct; identical to SEQ ID NO: 855
  source 1..56: mol_type = other RNA

SEQ ID NO: 1249 - RNA, 21 nt, synthetic construct
  source 1..21: mol_type = other RNA
cctgtctgcacagacagcacc

SEQ ID NO: 1250 - RNA, 24 nt, synthetic construct
  source 1..24: mol_type = other RNA
ccgcctgtctgcaccggcagcacc

SEQ ID NO: 1251 - RNA, 30 nt, synthetic construct; identical to SEQ ID NO: 965
  source 1..30: mol_type = other RNA

SEQ ID NO: 1252 - RNA, 37 nt, synthetic construct
  source 1..37: mol_type = other RNA
agctggggcgcagccgcctgtctgcaccggcagcacc

SEQ ID NO: 1253 - RNA, 37 nt, synthetic construct; identical to SEQ ID NO: 1252
  source 1..37: mol_type = other RNA

SEQ ID NO: 1254 - RNA, 39 nt, synthetic construct; identical to SEQ ID NO: 812
  source 1..39: mol_type = other RNA

SEQ ID NO: 1255 - RNA, 52 nt, synthetic construct
  source 1..52: mol_type = other RNA
gggtcttctcaaggaaatcccaatctctattccgaagagtcttccctaaaag

SEQ ID NO: 1256 - RNA, 61 nt, synthetic construct
  source 1..61: mol_type = other RNA
attaacacttgggtaacttacccttcacaatccatctaaatccttctcaattgctgccacc

SEQ ID NO: 1257 - RNA, 20 nt, synthetic construct
  source 1..20: mol_type = other RNA
atttactttgcagagtaact

SEQ ID NO: 1258 - RNA, 24 nt, synthetic construct
  source 1..24: mol_type = other RNA
ccgcctgtctgcaccggcagcacc

SEQ ID NO: 1259 - RNA, 53 nt, synthetic construct; identical to SEQ ID NO: 1088
  source 1..53: mol_type = other RNA

SEQ ID NO: 1260 - RNA, 30 nt, synthetic construct; identical to SEQ ID NO: 965
  source 1..30: mol_type = other RNA

SEQ ID NO: 1261 - RNA, 37 nt, synthetic construct; identical to SEQ ID NO: 1252
  source 1..37: mol_type = other RNA

SEQ ID NO: 1262 - RNA, 32 nt, synthetic construct; identical to SEQ ID NO: 1035
  source 1..32: mol_type = other RNA

SEQ ID NO: 1263 - RNA, 25 nt, synthetic construct
  source 1..25: mol_type = other RNA
aaaagaattatcacattgcacaagg

SEQ ID NO: 1264 - RNA, 32 nt, synthetic construct; identical to SEQ ID NO: 1035
  source 1..32: mol_type = other RNA

SEQ ID NO: 1265 - RNA, 37 nt, synthetic construct
  source 1..37: mol_type = other RNA
agctcggtcgcggctgcctgtctgctccggcagcacc

SEQ ID NO: 1266 - RNA, 24 nt, synthetic construct
  source 1..24: mol_type = other RNA
ccgcctgtctgcaccggcagcacc

SEQ ID NO: 1267 - RNA, 12 nt, synthetic construct
  source 1..12: mol_type = other RNA
accggcagcacc

SEQ ID NO: 1268 - RNA, 55 nt, synthetic construct
  source 1..55: mol_type = other RNA
acgtagaccgtctgctgggggcgccggcaccatttggagtacgtgaggaagaaaa

SEQ ID NO: 1269 - RNA, 37 nt, synthetic construct; identical to SEQ ID NO: 1252
  source 1..37: mol_type = other RNA

SEQ ID NO: 1270 - RNA, 59 nt, synthetic construct; identical to SEQ ID NO: 223
  source 1..59: mol_type = other RNA

SEQ ID NO: 1271 - RNA, 32 nt, synthetic construct; identical to SEQ ID NO: 1035
  source 1..32: mol_type = other RNA

SEQ ID NO: 1272 - RNA, 24 nt, synthetic construct
  source 1..24: mol_type = other RNA
ccgcctgtctgcaccggcagcacc

SEQ ID NO: 1273 - RNA, 94 nt, synthetic construct
  source 1..94: mol_type = other RNA
ttagctgaactgagctgaaatcctaaaggccgcggagtcggcggtgttgtaggtagcggtaccttgagtggcaacagaattcgattaaattaca

SEQ ID NO: 1274 - RNA, 24 nt, synthetic construct
  source 1..24: mol_type = other RNA
ccgcctgtctgcaccggcagcacc

SEQ ID NO: 1275 - RNA, 24 nt, synthetic construct
  source 1..24: mol_type = other RNA
agtagatcctggcctggacctgaa

SEQ ID NO: 1276 - RNA, 63 nt, synthetic construct
  source 1..63: mol_type = other RNA
gaaggctagacccggtttactggaattgctctggcgatcgagggatcctagtacaccgcaatc

SEQ ID NO: 1277 - RNA, 54 nt, synthetic construct
  source 1..54: mol_type = other RNA
acccggtttactggaattgctctggcgatcgagggatcctagtacaccgcaatc

SEQ ID NO: 1278 - RNA, 55 nt, synthetic construct; identical to SEQ ID NO: 1197
  source 1..55: mol_type = other RNA

SEQ ID NO: 1279 - RNA, 85 nt, synthetic construct
  source 1..85: mol_type = other RNA
agaaggacgcaggcagcaaagagtagtcagtcccttcttggctctgctgacactcgagcccacattccatcacctgctcccaatc

SEQ ID NO: 1280 - RNA, 52 nt, synthetic construct; identical to SEQ ID NO: 1117
  source 1..52: mol_type = other RNA

SEQ ID NO: 1281 - RNA, 85 nt, synthetic construct; identical to SEQ ID NO: 126
  source 1..85: mol_type = other RNA

SEQ ID NO: 1282 - RNA, 37 nt, synthetic construct; identical to SEQ ID NO: 1265
  source 1..37: mol_type = other RNA

SEQ ID NO: 1283 - RNA, 37 nt, synthetic construct; identical to SEQ ID NO: 1252
  source 1..37: mol_type = other RNA

SEQ ID NO: 1284 - RNA, 37 nt, synthetic construct; identical to SEQ ID NO: 1252
  source 1..37: mol_type = other RNA

SEQ ID NO: 1285 - RNA, 71 nt, synthetic construct
  source 1..71: mol_type = other RNA
ttcttttttgaggttttgaggttttgctgaagatattcctgaagaatcatcccaggtgccacactaaaaaa

SEQ ID NO: 1286 - RNA, 62 nt, synthetic construct; identical to SEQ ID NO: 667
  source 1..62: mol_type = other RNA

SEQ ID NO: 1287 - RNA, 26 nt, synthetic construct
  source 1..26: mol_type = other RNA
agcgcagcggcgtccgaggcaacaag

SEQ ID NO: 1288 - RNA, 64 nt, synthetic construct
  source 1..64: mol_type = other RNA
aaattcagggtccagctcacagtggaaatactttctgagagtcagtgacctcctgcacaagaac

SEQ ID NO: 1289 - RNA, 56 nt, synthetic construct
  source 1..56: mol_type = other RNA
attaatattaagattggaagtttgtgtcttttgctggatattggaaattgaagtta

SEQ ID NO: 1290 - RNA, 59 nt, synthetic construct
  source 1..59: mol_type = other RNA
tttgctcagtttcacagagggcctggtctggaatattccacatctgctctcactctgcc

SEQ ID NO: 1291 - RNA, 93 nt, synthetic construct
  source 1..93: mol_type = other RNA
agtcttcatttaggtccgaattcccggcatataagaatactaccgtcgcttgtttttcagatttttgcggctattttcgttggtgtgttggtc

SEQ ID NO: 1292 - RNA, 58 nt, synthetic construct
  source 1..58: mol_type = other RNA
ggtggaagaagtaaagaaaagaaagagtaaaggagtttgaatcactttgcagtgctgt

SEQ ID NO: 1293 - RNA, 55 nt, synthetic construct; identical to SEQ ID NO: 1268
  source 1..55: mol_type = other RNA

SEQ ID NO: 1294 - RNA, 24 nt, synthetic construct
  source 1..24: mol_type = other RNA
ccgcctgtctgcaccggcagcacc

SEQ ID NO: 1295 - RNA, 39 nt, synthetic construct
  source 1..39: mol_type = other RNA
gcatttctctttaggttgtggacgaagtgtttatttatc

SEQ ID NO: 1296 - RNA, 32 nt, synthetic construct; identical to SEQ ID NO: 1035
  source 1..32: mol_type = other RNA

SEQ ID NO: 1297 - RNA, 85 nt, synthetic construct; identical to SEQ ID NO: 1279
  source 1..85: mol_type = other RNA

SEQ ID NO: 1298 - RNA, 32 nt, synthetic construct; identical to SEQ ID NO: 1035
  source 1..32: mol_type = other RNA

SEQ ID NO: 1299 - RNA, 88 nt, synthetic construct; identical to SEQ ID NO: 1001
  source 1..88: mol_type = other RNA

SEQ ID NO: 1300 - RNA, 37 nt, synthetic construct; identical to SEQ ID NO: 1265
  source 1..37: mol_type = other RNA

SEQ ID NO: 1301 - RNA, 85 nt, synthetic construct; identical to SEQ ID NO: 1279
  source 1..85: mol_type = other RNA

SEQ ID NO: 1302 - RNA, 63 nt, synthetic construct; identical to SEQ ID NO: 1276
  source 1..63: mol_type = other RNA

SEQ ID NO: 1303 - RNA, 54 nt, synthetic construct; identical to SEQ ID NO: 1277
  source 1..54: mol_type = other RNA

SEQ ID NO: 1304 - RNA, 85 nt, synthetic construct; identical to SEQ ID NO: 1279
  source 1..85: mol_type = other RNA

SEQ ID NO: 1305 - RNA, 32 nt, synthetic construct; identical to SEQ ID NO: 1035
  source 1..32: mol_type = other RNA

SEQ ID NO: 1306 - RNA, 85 nt, synthetic construct; identical to SEQ ID NO: 126
  source 1..85: mol_type = other RNA

SEQ ID NO: 1307 - RNA, 24 nt, synthetic construct
  source 1..24: mol_type = other RNA
ccgcctgtctgcaccggcagcacc

SEQ ID NO: 1308 - RNA, 92 nt, synthetic construct
  source 1..92: mol_type = other RNA
tcgacagtccacaaaggcacagtataaaaacggtgggaatcagagcacttcagctccaattgctctagttttagaattgcctctttttcaag

SEQ ID NO: 1309 - RNA, 50 nt, synthetic construct
  source 1..50: mol_type = other RNA
gtagtttcattacattttcttgtggcgattttcccttatcagaagtagtt

SEQ ID NO: 1310 - RNA, 67 nt, synthetic construct; identical to SEQ ID NO: 156
  source 1..67: mol_type = other RNA

SEQ ID NO: 1311 - RNA, 37 nt, synthetic construct; identical to SEQ ID NO: 1265
  source 1..37: mol_type = other RNA

SEQ ID NO: 1312 - RNA, 55 nt, synthetic construct; identical to SEQ ID NO: 1197
  source 1..55: mol_type = other RNA

SEQ ID NO: 1313 - RNA, 42 nt, synthetic construct
  source 1..42: mol_type = other RNA
ccaccaccagcaaacaaggggcagcgtggaggagtggacaca

SEQ ID NO: 1314 - RNA, 12 nt, synthetic construct
  source 1..12: mol_type = other RNA
accggcagcacc

SEQ ID NO: 1315 - RNA, 37 nt, synthetic construct; identical to SEQ ID NO: 1265
  source 1..37: mol_type = other RNA

SEQ ID NO: 1316 - RNA, 30 nt, synthetic construct; identical to SEQ ID NO: 965
  source 1..30: mol_type = other RNA

SEQ ID NO: 1317 - RNA, 24 nt, synthetic construct
  source 1..24: mol_type = other RNA
ccgcctgtctgcaccggcagcacc

SEQ ID NO: 1318 - RNA, 44 nt, synthetic construct; identical to SEQ ID NO: 769
  source 1..44: mol_type = other RNA

SEQ ID NO: 1319 - RNA, 53 nt, synthetic construct
  source 1..53: mol_type = other RNA
gccttatctgactcttgttttcaacagagtttgtcctctcacttctggagaag

SEQ ID NO: 1320 - RNA, 25 nt, synthetic construct
  source 1..25: mol_type = other RNA
actcagtgtaccttctagtcccgcc

SEQ ID NO: 1321 - RNA, 73 nt, synthetic construct; identical to SEQ ID NO: 1183
  source 1..73: mol_type = other RNA

SEQ ID NO: 1322 - RNA, 64 nt, synthetic construct; identical to SEQ ID NO: 1288
  source 1..64: mol_type = other RNA

SEQ ID NO: 1323 - RNA, 61 nt, synthetic construct; identical to SEQ ID NO: 1116
  source 1..61: mol_type = other RNA

SEQ ID NO: 1324 - RNA, 59 nt, synthetic construct; identical to SEQ ID NO: 1290
  source 1..59: mol_type = other RNA

SEQ ID NO: 1325 - RNA, 67 nt, synthetic construct; identical to SEQ ID NO: 1167
  source 1..67: mol_type = other RNA

SEQ ID NO: 1326 - RNA, 57 nt, synthetic construct
  source 1..57: mol_type = other RNA
agtctccttcaccacccagctgggatctcagggcttccttttctgtcctcctccagg

SEQ ID NO: 1327 - RNA, 48 nt, synthetic construct
  source 1..48: mol_type = other RNA
tcaccaagtcacagacccagaggacccctccatcctgcagttcctgcc

SEQ ID NO: 1328 - RNA, 20 nt, synthetic construct
  source 1..20: mol_type = other RNA
attctttcttcaaagcagcc

SEQ ID NO: 1329 - RNA, 66 nt, synthetic construct
  source 1..66: mol_type = other RNA
ctttcaaagccaatttgaataaacaactcttccctctcaaagtggcaagagtaactcttcagcacc

SEQ ID NO: 1330 - RNA, 73 nt, synthetic construct; identical to SEQ ID NO: 1183
  source 1..73: mol_type = other RNA

SEQ ID NO: 1331 - RNA, 99 nt, synthetic construct; identical to SEQ ID NO: 868
  source 1..99: mol_type = other RNA

SEQ ID NO: 1332 - RNA, 90 nt, synthetic construct; identical to SEQ ID NO: 869
  source 1..90: mol_type = other RNA

SEQ ID NO: 1333 - RNA, 50 nt, synthetic construct
  source 1..50: mol_type = other RNA
ctggagtctccgcgggcagatctcatattttggattctggatatattata

SEQ ID NO: 1334 - RNA, 20 nt, synthetic construct
  source 1..20: mol_type = other RNA
acaccttctgaggcagcggc

SEQ ID NO: 1335 - RNA, 55 nt, synthetic construct
  source 1..55: mol_type = other RNA
acacactcgcttctggaacgtctgaggttatcaataagctcctagtccagacgcc

SEQ ID NO: 1336 - RNA, 53 nt, synthetic construct; identical to SEQ ID NO: 1088
  source 1..53: mol_type = other RNA

SEQ ID NO: 1337 - RNA, 42 nt, synthetic construct; identical to SEQ ID NO: 777
  source 1..42: mol_type = other RNA

SEQ ID NO: 1338 - RNA, 88 nt, synthetic construct; identical to SEQ ID NO: 1001
  source 1..88: mol_type = other RNA

SEQ ID NO: 1339 - RNA, 58 nt, synthetic construct
  source 1..58: mol_type = other RNA
ttcgcagcggaccgaagagaagaaaagtaggccagagccgaactctcttcctgccaag

SEQ ID NO: 1340 - RNA, 27 nt, synthetic construct
  source 1..27: mol_type = other RNA
acacttttgtgtgtgctctcattgcaa

SEQ ID NO: 1341 - RNA, 94 nt, synthetic construct
  source 1..94: mol_type = other RNA
acctcaccatcaagaaaggccctggtccaggagttgcaaggtgtgagtaaatcacagggctggggaaggtcagctcggggtcacaagaagcctg

SEQ ID NO: 1342 - RNA, 42 nt, synthetic construct; identical to SEQ ID NO: 546
  source 1..42: mol_type = other RNA